(19) United States
(12) Patent Application Publication
(10) Pub. No.: US 2026/0366049 A1
(43) Pub. Date: Nov. 19, 2026

(54) SYNTHESIS OF ISOPRENOIDS AND DERIVATIVES

(71) Applicant: William Marsh Rice University, Houston, TX (US)

(72) Inventors: Ramon GONZALEZ, Tampa, FL (US); James M. CLOMBURG, Houston, TX (US); Seokjung CHEONG, Emeryville, CA (US)

(21) Appl. No.: 19/253,274

(22) Filed: Jun. 27, 2025

Related U.S. Application Data
(63) Continuation of application No. 18/330,944, filed on Jun. 7, 2023, now Pat. No. 12,460,234.
Application No. 18/330,944 is a division of application No. 17/234,398, filed on Apr. 19, 2021, now abandoned.
Application No. 17/234,398 is a division of application No. 16/081,756, filed on Mar. 15, 2017, now Pat. No. 11,046,978.
(60) Provisional application No. 62/308,937, filed on Mar. 16, 2016.
(60) Provisional application No. 62/343,598, filed on May 31, 2016.

(51) Int. Cl. C12P 7/42

(52) U.S. Cl. CPC C12P 7/42

(57) ABSTRACT

This disclosure generally relates to the use of enzyme combinations or recombinant microbes comprising same to make isoprenoid precursors, isoprenoids and derivatives thereof including prenylated aromatic compounds. Novel metabolic pathways exploiting Claisen, aldol, and acyloin condensations are used instead of the natural mevalonate (MVA) pathway or 1-deoxy-d-xylulose 5-phosphate (DXP) pathways for generating isoprenoid precursors such as isopentenyl pyrophosphate (IPP), dimethylallyl pyrophosphate (DMAPP), and geranyl pyrophosphate (GPP). These pathways have the potential for better carbon and or energy efficiency than native pathways. Both decarboxylative and non-carboxylative condensations are utilized, enabling product synthesis from a number of different starting compounds. These condensation reactions serve as a platform for the synthesis of isoprenoid precursors when utilized in combination with a variety of metabolic pathways and enzymes for carbon rearrangement and the addition/removal of functional groups. Isoprenoid alcohols are key intermediary products for the production of isoprenoid precursors in these novel synthetic metabolic pathways. These precursors can be modified to various isoprenoid products through prenyl transferase, terpene synthase, or terpene cyclases. The production of prenylated aromatic compounds is achieved through prenyl transfer of the hydrocarbon units of isoprenoid precursors to polyketides.

### PRIOR RELATED APPLICATIONS

This application is a continuation of U.S. Ser. No. 18/330,944, filed Jun. 7, 2023 (now US patent U.S. Ser. No. 12/460,234), which is a divisional application of U.S. Ser. No. 17/234,398, filed Apr. 19, 2021 which is a divisional application of U.S. Ser. No. 16/081,756, filed Aug. 31, 2018 (now US patent U.S. Ser. No. 11/046,978) which is a National Phase filing under 35 U.S.C. § 371 of International Application PCT/US2017/022581, filed Mar. 15, 2017 which claims priority to US Ser. No. 62/308,937, filed Mar. 16, 2016, titled SYNTHESIS OF ISOPRENOIDS AND DERIVATIVES THROUGH THIOLASE-CATALYZED NON-DECARBOXYLATIVE CONDENSATION REACTIONS, and 62/343,598, filed May 31, 2016, titled BIOLOGICAL SYNTHESIS OF ISOPRENOIDS AND PRENYLATED AROMATICS, each of which are incorporated by reference herein in its entirety for all purposes.

### REFERENCE TO ELECTRONIC SEQUENCE LISTING

The application contains Sequence Listing which has been submitted electronically in .XML format and is hereby incorporated by reference in its entirety. Said .XML copy, created on Oct. 6, 2023, is named “RICE2016-060-08USDIV2.xml” and is 189,059 bytes in size. The sequence listing contained in this .XML file is part of the specification and is hereby incorporated by reference herein in its entirety.

### FIELD OF THE DISCLOSURE

This disclosure generally relates to the use of enzyme combinations or recombinant microorganisms comprising same to make various isoprenoid precursors, isoprenoids and derivatives thereof including prenylated aromatic compounds. Novel enzymes and cells for making cannabinoids and olivoteic acid are also provided.

### BACKGROUND OF THE DISCLOSURE

The biosynthesis of fatty acids, polyketides, isoprenoids, and many other molecules with applications ranging from biofuels and green chemicals to therapeutic agents, rely on reactions catalyzing the formation of carbon-carbon bonds. Small precursor metabolites serve as building blocks for these pathways, which are subsequently condensed and modified until the desired chain length and functionality are achieved.

Isoprenoids represent one of the largest and most diverse classes of these natural products, with more than 40,000 different structures found in all kingdoms of life. These natural products have a wide range of ecological, physiological and structural functions and have been utilized for their very different properties in applications such as medicines, flavors, and fragrances. Furthermore, modern industry has harnessed these compounds as pharmaceuticals, components of personal hygiene and cosmetic products, antimicrobial agents, solvents, and commodity materials such natural rubbers and biofuels.

Despite this high diversity and product functionality, all isoprenoids are formed from the 5-carbon (C₅) building blocks isopentenyl pyrophosphate (IPP) and dimethylallyl pyrophosphate (DMAPP). These two building blocks are formed from one of two native pathways: the mevalonate (MVA) pathway (native to most archaea and eukaryotes) or the 2-C-methyl-D-erythritol 4-phosphate/1-deoxy-D-xylulose 5-phosphate (DXP/MEP) pathway (native to most bacteria). The MVA pathway utilizes 3 acetyl-CoA molecules for the formation of the C₅ intermediates, while the 3-carbon intermediates pyruvate and glyceraldehyde-3-phosphate serve as the starting point in the DXP pathway. Following the synthesis of IPP and DMAPP through either pathway, these intermediates are condensed and modified by various combinations of for example geranyl-, farnesyl- or, geranylgeranyl-diphosphate synthases, prenyl transferase, terpene synthases, or terpene cyclases to form thousands of products.

While these native pathways have been exploited for the synthesis of various isoprenoid products, there are limitations in using the native pathways. For example, the synthesis of the required C₅ building blocks from either the MVA or DXP pathway results in the inevitable loss of carbon from the starting intermediates (3 acetyl-CoA molecules or pyruvate and glyceraldehyde-3-phosphate). Furthermore, both the MVA and DXP pathways are energy (ATP) intensive, with the net consumption of 3 ATP equivalents from starting intermediates. Thus, there exists a need for methods to overcome the inherently low carbon and energy efficiency of natural isoprenoid precursor synthesis pathways. Preferably, such pathways would further diversify the range of products, and provide a more carbon and energy efficient route.

### SUMMARY OF THE DISCLOSURE

This disclosure generally relates to the use of either enzyme combinations or recombinant microbes expressing those enzyme combinations to make isoprenoid precursors, isoprenoids and derivatives thereof including prenylated aromatic compounds through novel synthetic metabolic pathways instead of the natural mevalonate (MVA) pathway or 1-deoxy-d-xylulose 5-phosphate (DXP) pathways, that can be exploited to achieve better carbon and or energy efficiency than the natural pathways.

Several approaches are described herein. In one approach, the enzymes are made and combined in one or more in vitro reactions to make the desired products. In another approach, recombinant cells are harvested and used as temporary bioreactors containing the enzymes to do all or part of the reactions for as long as the ensymes remain active. In another approach, the cells are lysed, and the lysate is used to catalyze the needed reactions. In yet another approach, recombinant cells are used in a growing, living system to continually make products. Combinations of the various approaches can also be used.

Further, there are three basic products types made herein, a) isoprenoid precursors, b) isoprenoids and derivatives thereof including prenylated aromatic compounds, and c) polyketides. Prenylated aromatic compounds are made by condensing isoprenoid precursors and aromatic polyketides, which can be made either by the methods of the invention or can be purchased or made by prior art recombinant synthesis methods.

As described herein, the novel pathways for the synthesis of these products exploit enzymes catalyzing Claisen, aldol, or acyloin condensation reactions for the generation of longer chain length intermediates from central carbon metabolites (FIG. 1). Both decarboxylative and non-carboxylative condensations are utilized, enabling product synthesis from a number of different starting compounds. These condensation reactions serve as a platform for the synthesis of isoprenoid precursors, isoprenoids and derivatives thereof, polyketides, and prenylated aromatic compounds when utilized in combination with a variety of metabolic pathways and enzymes for carbon rearrangement and the addition/removal of functional groups (FIG. 1).

One aspect of the invention is a CoA-dependent elongation platform based on the use of Claisen condensations, which accept functionalized acyl-CoAs as primers and extender units in a reverse beta-oxidation like pathway. Products can be pulled out at any point, and further modified if desired. In other aspects of the invention, aldol or acyloin condensations serve as the starting condensation reaction to enable product synthesis from central carbon metabolites such as pyruvate through various enzyme combinations (FIG. 1). Isoprenoid acyl-CoAs, such as 3-methyl-but-2-enoyl-CoA and 3-methyl-but-3-enoyl-CoA, and isoprenoid alcohols, such as prenol and isoprenol, are key pathway intermediates that can be converted to isoprenoid precursors, such as isopentenyl phosphate (IP), dimethylallyl phosphate (DMAP), IPP and DMAPP, through phosphorylation enzymes (FIG. 1). As above, any of the products can be further modified if desired.

In one embodiment, native or engineered thiolases catalyze the condensation between an acyl-CoA primer and another acyl-CoA serving as the extender unit, forming a beta-keto acyl-CoA. FIG. 1 demonstrates the general CoA-dependent elongation platform, which can also utilize decarboxylative Clasien condensation reactions catalyzed by ketoacyl-CoA synthases. Primers and extender units can be omega-functionalized to add required functionalities to the carbon chain. The beta-keto group of the beta-keto acyl-CoA formed via condensation can be reduced and modified step-wise by the beta-reduction reactions catalyzed by dehydrogenase(s), dehydratase(s) and/or reductase(s). Dehydrogenases reduce the beta-keto group of a CoA intermediate synthesized by the condensation(s) to a beta-hydroxy group. Dehydratases catalyze the dehydration of beta-keto group to an alpha, beta double bond. Reductases reduce the alpha, beta double bond to the single bond. Furthermore, various carbon re-arrangement enzymes, such as acyl-CoA mutases, can be employed to modify the carbon structure and branching of the acyl-CoAs. These CoA intermediates can then serve as the primer for the next round of condensation with the extender unit or as direct intermediates to IP, DMAP, IPP, DMAPP, or other isoprenoid precursors. After termination by spontaneous or enzyme-catalyzed CoA removal, reduction, and/or phosphorylation, and subsequent structure re-arrangement, isoprenoids precursors (e.g., IPP, DMAPP, GPP, GGPP, FPP) are produced, and isoprenoids and derivatives thereof can be produced from those. Examples of pathways based on these Clasien condensation reactions are shown in FIGS. 2-6.

In another embodiment, either non-decarboxylative or decarboxylative Claisen condensation is used to form acetoacetyl-CoA as an intermediate. In one such pathway, acetoacetyl-CoA is subsequently converted to 3-hydroxy-3-methylglutaryl-CoA, which is then dehydrated and decarboxylated to form the isoprenoid acyl-CoA 3-methyl-2-butenoyl-CoA (FIG. 7). In another pathway from acetoacetyl-CoA, acetone generated from the decarboxylation of acetoacetic acid is converted to 3-methyl-3-hydroxy-butyryl-CoA through a non-decarboxylative Claisen condensation, which is then dehydrated to form 3-methyl-2-butenoyl-CoA (FIG. 8). 3-methyl-2-butenoyl-CoA can then be converted to prenol through various alcohol forming termination pathways (FIG. 7 and FIG. 8). This 5-carbon isoprenoid alcohol is then converted to DMAPP through a two-step phosphorylation with DMAP as an intermediate, or a one step diphosphorylation catalyzed by an alcohol diphosphokinase. DMAPP can be isomerized into IPP, generating the two required C₅ isoprenoid precursors.

Isoprenoid precursors, such as DMAPP, IPP, and GPP, can be condensed and modified by various combinations of geranyl-, farnesyl- or, geranylgeranyl-diphosphate synthases, prenyl transferase, terpene synthases, or terpene cyclases to form numerous isoprenoid products and derivatives thereof (FIG. 15). Combining this route for isoprenoid precursor formation for example with a route to aromatic polyketides enables the production of prenylated aromatic compounds through prenyl transfer of hydrocarbon units of isoprenoid intermediates to aromatic polyketides.

Examples of routes to polyketides include those based on thiolase-catalyzed condensation reactions or polyketide synthases. The route to polyketides via condensation and beta-reduction reactions involves the use of native or engineered thiolases that catalyze the non-decarboxylative condensation in an iterative manner (i.e. a single or multiple turns of the cycle) between two either unsubstituted or functionalized acyl-CoAs each serving as the primer and the extender unit to generate and elongate polyketide CoA intermediates. Before the next round of thiolase reaction, the beta-keto group of the polyketide chain can be reduced and modified step-wise by dehydrogenase or dehydratase or reductase reactions. Dehydrogenase reaction converts the beta-keto group to beta-hydroxy group. Dehydratase reaction converts the beta-hydroxy group to alpha-beta-double-bond. Reductase reaction converts the alpha-beta-double-bond to a single bond. Spontaneous or enzymatically catalyzed termination reaction(s) terminate the elongation of polyketide chain at any point through CoA removal and spontaneous rearrangement of the structure, generating the final functional polyketide products. This approach is the subject of patent application WO2017020043, BIOSYNTHESIS OF POLYKETIDES, filed Aug. 1, 2016, and 62/198,764, filed Jul. 30, 2015.

Alternatively, polyketide molecules can be formed through polyketide synthases (PKS). This large class of secondary metabolites formed by bacteria, fungi and plant are synthesized through these multi-domain enzymes or enzyme complexes. From a relatively small set of starting and extending molecules, these enzymes are capable of producing a vast array of complex metabolites through combinatorial and iterative carbon-carbon bond formation. Here, PKSs can be exploited for the synthesis of targeted polyketide molecules that can be further combined with isoprenoids and isoprenoid precursors synthesized through various pathways to form different molecules. This includes prenyl transfer of the hydrocarbon moiety of isoprenoid precursors to aromatic polyketides, forming prenylated aromatic compounds.

This disclosure also relates to the use of enzyme combinations or recombinant microbes to make isoprenoid precursors, isoprenoids and derivatives thereof including prenylated aromatic compounds through acyloin condensation reactions (FIG. 1). Certain examples involve using valine biosynthetic enzymes through acetolactate as an intermediate (FIG. 9 and FIG. 10). The pathway begins from a central carbon intermediate, in which two molecules of pyruvate are combined to form acetolactate through decarboxylative acyloin condensation, followed by subsequent isomeroreduction and dehydration to form 3-methyl-2-oxobutanoate. These reactions, catalyzed by acetolactate synthase, acetohydroxyacid isomeroreductase, and dihydroxyacid dehydratase, respectively, are part of the ubiquitous valine biosynthesis pathway.

Following initial use of this amino acid synthesis pathway for the generation of 3-methyl-2-oxobutanoate, several metabolic routes to isoprenoid precursors can be exploited. One such pathway involves a keto-reduction and combinations of dehydration and phosphorylation, either converting the free acid intermediate or its CoA derivative to prenol (FIG. 9). Alternatively, the addition of 2-carbons to 3-methyl-2-oxobutanoate, followed by subsequent isomerization, and decarboxylation results in the generation of isovaleryl-CoA, which can then be converted to prenol through a series of reactions (FIG. 10). For either pathway, prenol is then converted to DMAPP, which can be isomerized into IPP generating the two required C₅ isoprenoid precursors. As with the above pathways, DMAPP and IPP can be condensed and modified by various combinations of geranyl-, farnesyl- or, geranylgeranyl-diphosphate synthases, prenyl transferase, terpene synthases, or terpene cyclases to form numerous isoprenoid products and derivatives thereof, including prenylated aromatic compounds.

In another embodiment, the non-decarboxylative acyloin condensation of isobutanal and formyl-CoA to 3-methyl-2-hydroxybutanoyl-CoA catalyzed by 2-hydroxyacyl-CoA lyase is utilized (FIG. 11). Isobutanal is generated through the use of Claisen condensation and beta-reduction reactions, with carbon rearrangement and an aldehyde forming termination pathway. Formyl-CoA can be generated directly from formate or formaldehyde. Following acyloin condensation, 3-methyl-2-hydroxybutanoyl-CoA is converted to prenol through various pathways (FIG. 11). As with the above pathways, prenol is subsequently converted into DMAPP and IPP, which can be condensed and modified by various combinations of geranyl-, farnesyl- or, geranylgeranyl-diphosphate synthases, prenyl transferase, terpene synthases, or terpene cyclases to form numerous isoprenoid products and derivatives thereof, including prenylated aromatic compounds.

This disclosure also relates to the use of enzyme combinations or recombinant microbes to make isoprenoid precursors, isoprenoids and derivatives thereof including prenylated aromatic compounds through aldol condensation reactions (FIG. 1). Pathways exploiting this reaction utilize an aldolase catalyzing the aldol condensation of a ketone, aldehyde, or carboxylic acid with an aldehyde to produce an aldol product. Depending on the compounds undergoing aldol condensation, a variety of metabolic pathways and enzymes for carbon rearrangement and the addition/removal of functional groups can be utilized for the synthesis of key isoprenoid intermediates including isoprenoid acyl-CoAs, such as 3-methyl-but-2-enoyl-CoA and 3-methyl-but-3-enoyl-CoA, and isoprenoid alcohols, such as prenol and isoprenol (FIG. 1). These intermediates are subsequently converted to isoprenoid precursors.

In one embodiment, an aldolase catalyzes the aldol condensation of pyruvate and acetaldehyde forming 4-hydroxy-2-oxopentanoate (FIG. 12 and FIG. 13). Carbon rearrangement catalyzed by a mutase and reduction through the action of a 2-hydroxyacid dehydrogenase converts 4-hydroxy-2-oxopentanoate to 2,3-dihydroxy-3-methylbutanoate, an intermediate of the aformentoned valine biosythensis pathway. Following dehydration to 3-methyl-2-oxobutanoate, several metabolic routes to isoprenoid precursors can be exploited, including keto-reduction and combinations of dehydration and phosphorylation, either converting the free acid intermediate or its CoA derivative to prenol (FIG. 12). Alternatively, the addition of 2-carbons to 3-methyl-2-oxobutanoate, followed by subsequent isomerization, and decarboxylation results in the generation of isovaleryl-CoA, which can then be converted to prenol through a series of reactions (FIG. 13). For either pathway, prenol is then converted to DMAPP, which can be isomerized into IPP generating the two required C₅ isoprenoid precursors.

In another embodiment, an aldolase catalyzes the aldol condensation of 2-oxobutanoate and acetaldehyde forming 4-hydroxy-2-oxo-3-methylpentanoate (FIG. 14). Conversion of this intermediate to 4-methyl-2-oxopent-4-enoate, through the action of a mutase and a dehydratase, enables the use of a number of pathways to generate isoprenol from 4-methyl-2-oxopent-4-enoate. This 5-carbon isoprenoid alcohol is then converted to IPP through a two-step phosphorylation with IP as an intermediate, or a one step diphosphorylation catalyzed by an alcohol diphosphokinase. IPP can be isomerized into DMAPP generating the two C₅ isoprenoid precursors. As with the above pathways, IPP and DMAPP can be condensed and modified by various combinations of geranyl-, farnesyl- or, geranylgeranyl-diphosphate synthases, prenyl transferase, terpene synthases, or terpene cyclases to form numerous isoprenoid products and derivatives thereof, including prenylated aromatic compounds.

The in vivo process involves for example performing traditional fermentations using industrial organisms (for example bacteria or yeast, such as E. coli, B. subtilus, S. cerevisiae, P. pastoris and the like) that convert different feedstocks into isoprenoid precursors, isoprenoids, and derivatives thereof including prenylated aromatic compounds. These organisms are considered workhorses of modern biotechnology. Media preparation, sterilization, inoculum preparation, fermentation and product recovery are some of the main steps of the process.

As an alternative to the in vivo expression of the pathway(s), a cell free, in vitro, version of the pathway(s) can be constructed. By purifying, or partially purifying, the relevant enzyme for each reaction step, the overall pathway can be assembled by combining the necessary enzymes. Alternatively, crude protein extract of cells expressing the pathway(s) can be utilized. With the addition of the relevant cofactors and substrates, the pathway can be assessed for its performance independently of a host. As yet another alternative, whole wet or dried cells can be used as bioreactors.

As used herein, a “primer” is a starting molecule for a Claisen condensation reaction to add one or multiple carbon extender units to a growing acyl-CoA. The reactions can be performed once or can be repeated in a cycle for increased carbon chain length. The typical “initial” or “initiating” primer is either acetyl-CoA or propionyl-CoA, but as the chain grows by adding extender units in each cycle, the primer will accordingly increase in size. In some cases, recombinant microbes or enzyme systems can also be provided with larger primers, e.g., C₄ primers, etc. added to the media or obtained from other cell pathways. In this invention, non-traditional primers can also be used in which the primer is functionalized, e.g., the terminal omega carbon has been functionalized (i.e., omega-hydroxylated, omega-carboxylated, etc).

It should be noted that there is a second type of primer used herein, which are the short oligonucleotides used in amplification reactions. These should not be confused with the “primer” used in the carbon chain elongation cycles described herein.

As used herein, the “extender unit” is an acyl-CoA that reacts with the primer in one or more condensations to add carbons on the acyl-CoA primer. In biological systems, the extender unit is typically acetyl-CoA. In this invention, traditional extenders or non-traditional extender units can be used, for example, when the terminal omega carbon has been functionalized (e.g., omega-hydroxylated extender unit, omega-carboxylated extender unit, etc).

Thiolases are ubiquitous enzymes that have key roles in many vital biochemical pathways, including the beta-oxidation pathway of fatty acid degradation and various biosynthetic pathways. Members of the thiolase family can be divided into two broad categories: degradative thiolases (EC 2.3.1.16), and biosynthetic thiolases (EC 2.3.1.9). The forward and reverse reactions are shown below:

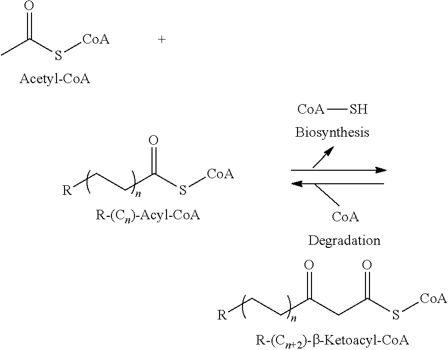

These two different types of thiolases are found both in eukaryotes and prokaryotes: for example acetoacetyl-CoA thiolase (EC:2.3.1.9) and 3-ketoacyl-CoA thiolase (EC:2.3.1.16). 3-ketoacyl-CoA thiolase (also called thiolase I) has a broad chain-length specificity for its substrates and is involved in degradative pathways such as fatty acid beta-oxidation. Acetoacetyl-CoA thiolase (also called thiolase II) is specific for the thiolysis of acetoacetyl-CoA and is involved in biosynthetic pathways such as poly beta-hydroxybutyric acid synthesis.

The degradative thiolases can be made to run in the forward direction by building up the level of left-hand side reactants (primer and extender unit), thus driving the equilibrium in the forward direction and/or by overexpressing same or by expressing a mutant of same.

As used herein, a “thiolase” is an enzyme that catalyzes the condensation of an either unsubstituted or functionalized acyl-CoA as the primer and another either unsubstituted or functionalized acyl-CoA for chain elongation to produce a beta-keto acyl-CoA in a non-decarboxylative condensation reaction:

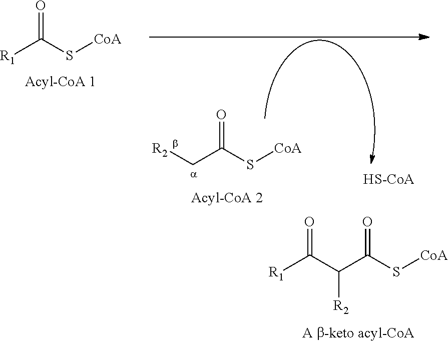

wherein R₁ or R₂ throughout are independantly an hydrogen, alkyl group, hydroxyl group, carboxyl group, aryl group, halogen, amino group, hydroxyacyl group, carboxyacyl group, aminoacyl group, ketoacyl group, halogenated acyl group, or any other functionalized acyl groups.

As used herein, a “ketoacyl-CoA synthase” is an enzyme that catalyzes the condensation of an either unsubstituted or functionalized acyl-CoA as the primer and either unsubstituted or functionalized beta-carboxylic acyl-CoA for chain elongation to produce a beta-keto acyl-CoA in a decarboxylative condensation reaction:

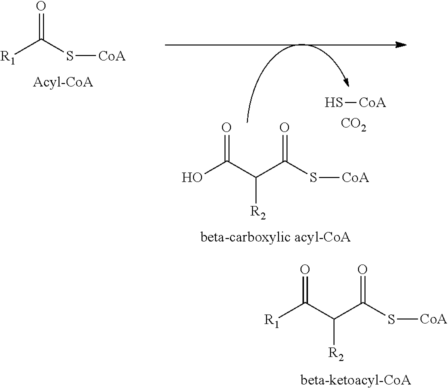

As used herein, a “hydroxyacyl-CoA dehydrogenase (HACD)” is an enzyme that catalyzes the reduction of a beta-keto acyl-CoA to a beta-hydroxy acyl-CoA:

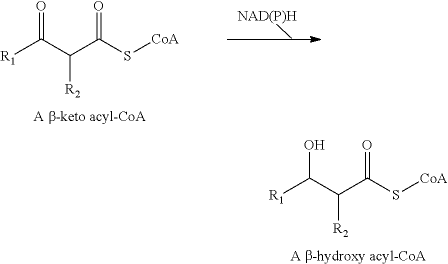

As used herein, “enoyl-CoA hydratase (ECH)” is an enzyme that catalyzes the dehydration of a beta-hydroxy acyl-CoA to an enoyl-CoA:

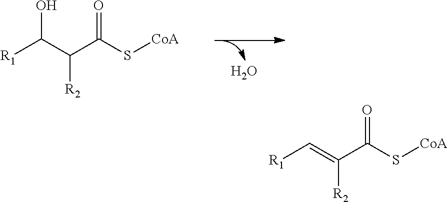

As used herein, an “enoyl-CoA reductase (ECR)” is an enzyme that catalyzes the reduction of an enoyl-CoA to an acyl-CoA:

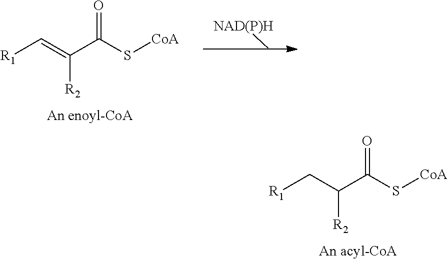

As used herein, the “beta-reduction enzymes” include HACD, ECH and ECR.

As used herein, an “acyloin condensation enzyme” is an enzyme that catalyzes the acyloin condensation of a ketone or aldehyde with either an alpha-ketoacid or an aldehyde to produce an acyloin product:

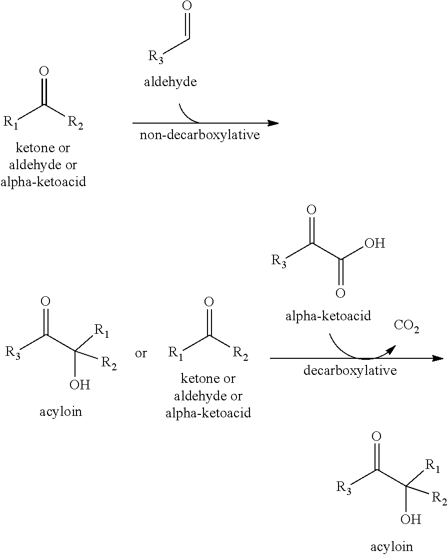

As used herein, “acetalactate synthase” or “ALS” enzyme (also known as acetohydroxy acid synthase, or AHAS) (EC 2.2.1.6) is a protein found in plants and microorganisms. ALS catalyzes the first step in the synthesis of the branched-chain amino acids (valine, leucine, and isoleucine) through a decarboxylative acyloin condensation between two pyruvate molecules. “Acetohydroxyacid isomeroreductase” or “AHAIR” (EC1.1.1.86) (also known as (ketol-acid reductoisomerase or “KARI”) is the second enzyme in the pathway for valine production. “Dihydroxyacid dehydratase” (EC 4.2.1.9) is the third enzyme in the valine pathway. Table E provides a variety of examples of these enzymes.

As used herein, an “aldolase” is an enzyme that catalyzes the aldol condensation of a ketone, aldehyde, or carboxylic acid with an aldehyde to produce an aldol product:

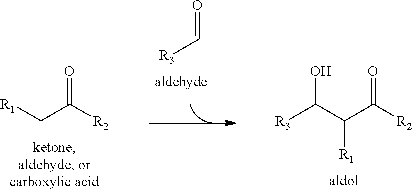

As used herein, a “termination pathway” or “termination enzymes” refers to one or more enzymes (or genes encoding same) that convert a CoA intermediate to a direct product (e.g. acid, alcohol, etc.)

As used herein, an “alcohol forming termination enzyme” refers to one or more enzymes (or genes encoding same) that converts an acyl-CoA to an alcohol, for example:
a) Alcohol forming acyl-CoA reductase;
b) Aldehyde forming acyl-CoA reductase plus alcohol dehydrogenase;
c) The transformation of acyl-CoA to a carboxylic acid (for example through a thioesterase, acyl-CoA transferase or phosphostransacyclase plus carboxylate kinase), a carboxylic acid reductase plus an alcohol dehydrogenase;
d) Aldehyde forming acyl-CoA reductase, an aldehyde decarboxylase, plus an omega-oxidation enzyme.

As used herein, a “phosphorylation enzyme” refers to one or more enzymes (or genes encoding same) that convert an alcohol to a phosphate or diphosphate. For example, an alcohol kinase, an alcohol kinase plus a phosphate kinase, or an alcohol diphosphokinase.

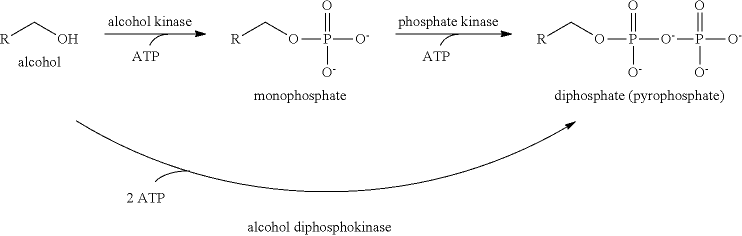

As used herein, “isoprenoid acyl-CoAs” are a class of intermediate products including 3-methyl-but-2-enoyl-CoA (3-methylcrotonyl-CoA), 3-methyl-but-3-enoyl-CoA, and intermediates with one or more prenyl (3-methyl-but-2-en-1-yl) or isoprenyl (3-methyl-but-3-en-1-yl) units attached to 3-methyl-but-2-enoyl-CoA or 3-methyl-but-3-enoyl-CoA:

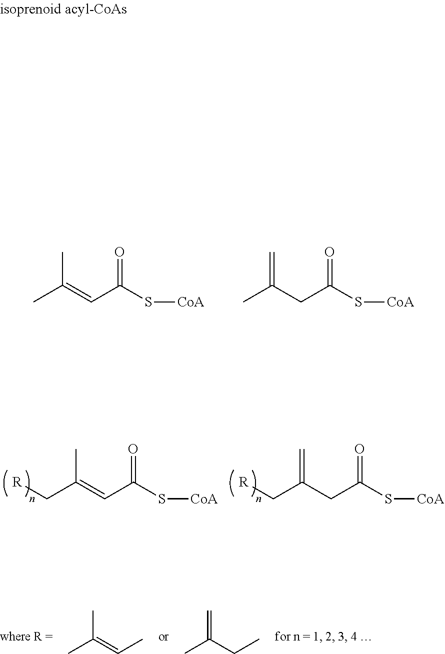

As used herein, “isoprenoid alcohols” are a class of intermediate products including 3-methyl-but-2-en-1-ol (prenol), 3-methyl-but-3-en-1-ol (isoprenol), and products with one or more prenyl (3-methyl-but-2-en-1-yl) or isoprenyl (3-methyl-but-3-en-1-yl) units attached to 3-methyl-but-2-en-1-ol or 3-methyl-but-3-en-1-ol:

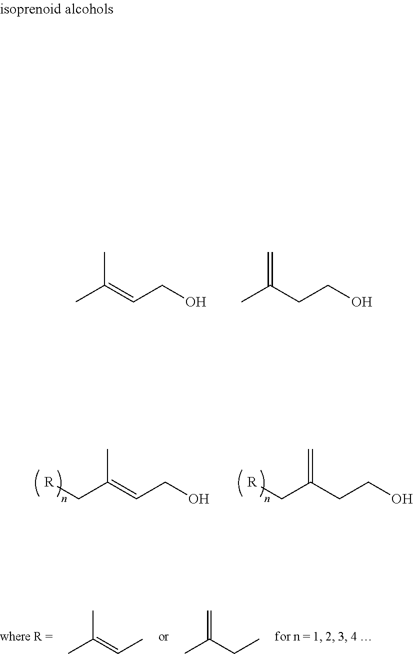

As used herein, “dimethylallyl pyrophosphate” or “DMAPP” is an intermediate product of both mevalonic acid (MVA) pathway and the 2-C-methyl-D-erythritol 4-phosphate/1-deoxy-D-xylulose 5-phosphate pathway (DXP/MEP) pathway. It is an isomer of isopentenyl pyrophosphate (IPP) and exists in virtually all life forms.

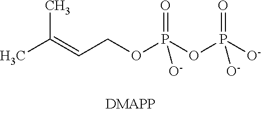

As used herein, “isopentenyl pyrophosphate” or “IPP” is an intermediate product of both mevalonic acid (MVA) pathway and the 2-C-methyl-D-erythritol 4-phosphate/1-deoxy-D-xylulose 5-phosphate pathway (DXP/MEP) pathway.

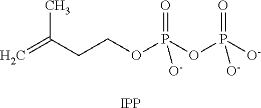

Isopentenyl pyrophosphate isomerase (IPP isomerase) catalyzes the interconversion of the relatively un-reactive IPP and the more-reactive electrophile DMAPP:

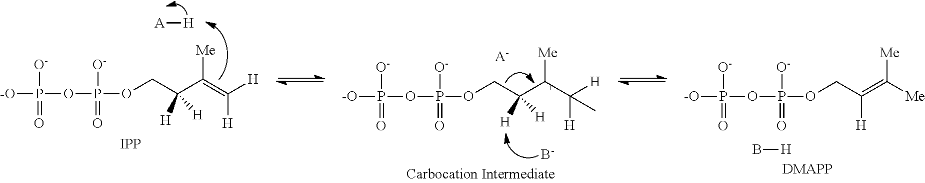

As used herein, “geranyl pyrophosphate” or “GPP”, also known as geranyl diphosphate (GDP), is an intermediate used by organisms in the biosynthesis of farnesyl pyrophosphate, geranylgeranyl pyrophosphate, cholesterol, terpenes, prenylated aromatic compounds, terpenoids and the like:

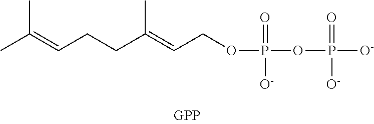 [SMILES: CC(C)=CCC/C(C)=C/COP(=O)([O-])OP(=O)([O-])[O-]]

IPP and DMAPP are condensed to make GPP:

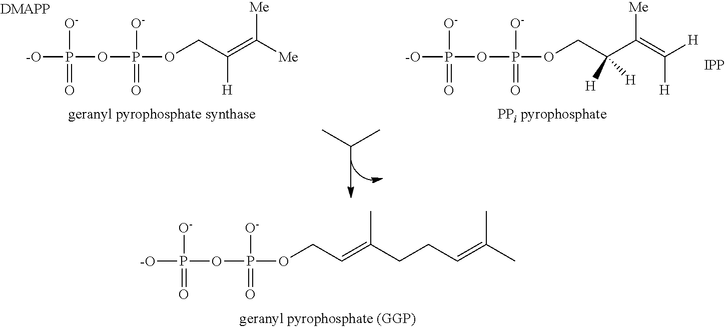

DMAPP and IPP—also known as “isoprenoid precursors” herein—can be further condensed and modified to make a wide range of products, including prenylated aromatic compounds and terpenoids. “Isoprenoid precursors” also includes isoprenoid monophosphates, such as dimethylallyl phosphate (DMAP) and isopentenyl phosphate (IP), as well as longer chain length intermediates with a hydrocarbon chain bound to a mono- or pyrophosphate, such as geranyl pyrophophosphate (GPP), farnesyl pyrophosphate (FPP), and geranylgeranyl pyrophosphate (GGPP) which can be formed through iterative condensation(s) of DMAPP and IPP. The terpenoids—also called “isoprenoids”—are a large and diverse class of naturally occurring organic chemicals derived from five-carbon isoprene units assembled and modified in thousands of ways.

As used herein, a “prenylated aromatic compound” is a derivative of an isoprenoid containing one or more prenyl units (3-methyl-but-2-en-1-yl) attached to a compound containing one or more aromatic group.

As used herein, a “cannabinoid” is a prenylated aromatic compound naturally found in the Cannabis sativa L plant, or a derivative thereof. Over 60 cannabinoids have been identified to date. Many of the more common cannabinoids have either 21 or 22 carbon atoms. Examples of cannabinoids include (CBGA), cannabigerol (CBG), cannabigerovarinic acid (CBGVA), cannabigerovarin (CBGV), tetrahydrocannabinolic acid (THCA), tetrahydrocannabinol (THC), cannabidiolic acid (CBDA), cannabidiol (CBD), cannbichromenic acid (CBCA), cannbichromene (CBC), tetrahydrocannabivarinic acid (THCVA), tetrahydrocannabivarin (THCV), cannabidivarinic acid (CBDVA), cannabidivarin (CBDV), cannabichrovarinic acid (CBCVA), and cannabichrovarin (CBCV).

As used herein, references to cells or bacteria or strains and all such similar designations include progeny thereof. The use of the singular “cell” does not imply that a single cell is to be used in any method, but includes all progeny produced by growing such cell. It is also understood that all progeny may not be precisely identical in DNA content, due to deliberate or inadvertent mutations that have been added to the parent. Mutant progeny that have the same function or biological activity as screened for in the originally transformed cell are included. Where distinct designations are intended, it will be clear from the context.

The terms “operably associated” or “operably linked,” as used herein, refer to functionally coupled nucleic acid sequences.

As used herein “recombinant” or “engineered” is relating to, derived from, or containing genetic material that has been intentionally altered by the action on man.

“Reduced activity” or “inactivation” is defined herein to be at least a 75% reduction in protein activity, as compared with an appropriate control species, usually wild type of that gene. Preferably, at least 80, 85, 90, 95% reduction in activity is attained, and in the most preferred embodiment, the activity is eliminated (100%, aka a “knock-out” or “null” mutants). Proteins can be inactivated with inhibitors, by mutation, or by suppression of expression or translation, and the like. Use of a frame shift mutation, early stop codon, deletions or insertions, gene editing, e.g., with CRISPR/cas9 and the like, or point mutations of critical residues, and the like, can completely inactivate (100%) of a gene product by completely preventing transcription and/or translation of the active protein.

“Overexpression” or “overexpressed” is defined herein to be at least 150% of protein activity as compared with an appropriate control species (e.g., wild type of the gene in question), and preferably 200, 500, 1000% or more. Any expression in a host species that otherwise lacks the gene would be overexpression. Overexpression can be achieved by mutating the protein to produce a more active form or a form that is resistant to inhibition, by removing inhibitors, by gene editing, e.g, with CRISPR/cas9 and the like, or adding activators, and the like. Overexpression can also be achieved by removing repressors, adding multiple copies of the gene to the cell, or upregulating the endogenous gene, and the like.

The term “heterologous” as used herein means containing or derived from a polymer of nucleic acids wherein at least one of the following is true: (a) the sequence of nucleic acids is foreign to (i.e., not naturally found in) a given cell; (b) the sequence may be naturally found in a given cell, but in an unnatural (e.g., greater than expected) amount; or (c) the sequence of nucleic acids comprises two or more subsequences that are not naturally found in the same relationship to each other in a given host. For example, regarding instance (c), a heterologous nucleic acid sequence that is recombinantly produced will have two or more sequences from unrelated genes arranged to make a new functional nucleic acid. The unrelated genes of part (c) may be either foreign to or naturally found in the recombinant microorganism. A heterologous enzyme is one that is produced by the transcription and translation of heterologous DNA. Overexpression and reduced expression is typically achieved through heterologous DNA

The microbes of the invention are generally made by transforming the host cell with an expression vector encoding one or more of the proteins, but the genes can also be added to the chromosome by recombineering, homologous recombination, and similar techniques. Where the needed protein is endogenous, as is the case in some instances, it may suffice as is, but it is usually overexpressed for better functionality and control over the level of active enzyme. The symbol “@” is used to indicate where a gene is inserted into the genome, otherwise it is placed into the native locus.

The term “endogenous” or “native” means that a gene originated from the species in question, without regard to subspecies or strain, although that gene may be naturally or intentionally mutated, or placed under the control of a promoter that results in overexpression or controlled expression of said gene. Thus, genes from Clostridia would not be endogenous to Escherichia, but a plasmid expressing a gene from E. coli would be considered to be endogenous to any genus of Escherichia, even though it may now be overexpressed. By contrast, a “heterogenous” gene would come from a different species.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, background mutations that do not effect the invention, and the like.

As used herein, reference to the accession number of an enzyme or its gene is intended to include the sequence data incorporated therein, as well as all known homologs linked thereto. Furthermore, reference to any protein by accession number includes all those homologs that catalyze the same reaction, although Km and Kcat can vary. Bacterial homologs preferably have >50% amino acid identity, but mammalian homologs are typically >80%.

In calculating “% identity,” the unaligned terminal portions of the query sequence are not included in the calculation. The identity is calculated over the entire length of the reference sequence, thus short local alignments with a query sequence are not relevant (e.g., % identity=number of aligned residues in the query sequence/length of reference sequence). Alignments are performed using BLAST homology alignment as described by Tatusova TA & Madden TL (1999) FEMS Microbiol. Lett. 174:247-250. The default parameters were used, except the filters were turned OFF. As of Jan. 1, 2001 the default parameters were as follows: BLASTN or BLASTP as appropriate; Matrix=none for BLASTN, BLOSUM62 for BLASTP; G Cost to open gap default=5 for nucleotides, 1 1 for proteins; E Cost to extend gap [Integer] default=2 for nucleotides, 1 for proteins; q Penalty for nucleotide mismatch [Integer] default=−3; r reward for nucleotide match [Integer] default=1; e expect value [Real] default=10; W word size [Integer] default=1 1 for nucleotides, 3 for proteins; y Dropoff (X) for blast extensions in bits (default if zero) default=20 for blastn, 7 for other programs; X dropoff value for gapped alignment (in bits) 30 for blastn, 15 for other programs; Z final X dropoff value for gapped alignment (in bits) 50 for blastn, 25 for other programs. This program is available online at NCBI™ (ncbi.nlm.nih.gov/BLAST/).

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

FIG. 1A-B: Synthesis of isoprenoid precursors, isoprenoids and derivatives thereof, and prenylated aromatic compounds using Claisen, aldol, or acyloin condensation reactions. Thiolases catalyze the non-decarboxylative condensation between an acyl-CoA, serving as the primer, and another acyl-CoA, serving as the extender unit, forming beta-keto acyl-CoA. Ketoacyl-CoA synthases catalyze the decarboxylative condensation between acyl-CoA and a beta-carboxylic acyl-CoA to form a beta-ketoacyl-CoA forming a beta-keto acyl-CoA. Aldolases or 2-hydroxyacyl-CoA lyases catalyze the aldol condensation of an aldehyde and a ketone, or an aldehyde and a second aldehyde, or an aldehyde and a carboxylic acid to form an aldol. Acyloin synthases or acetolactate synthase catalyze the non-decarboxylative acyloin condensation of a ketone and an aldehyde, or an aldehyde and a second aldehyde, or the decarboxylative acyloin condensation of a ketone and an alpha-keto acid, an aldehyde and an alpha keto acid, or an alpha-keto acid and a second alpha-keto acid to form an acyloin. Following condensation of starting compounds to initiate a given pathway, a variety of metabolic pathways and enzymes (dotted lines or multiple arrows) for carbon rearrangement and the addition/removal of functional groups can be utilized for the synthesis of key isoprenoid intermediates including isoprenoid acyl-CoAs, such as 3-methyl-but-2-enoyl-CoA and 3-methyl-but-3-enoyl-CoA, and isoprenoid alcohols, such as prenol and isoprenol. Isoprenoid alcohols are then converted to isoprenoid precursors such as DMAPP, IPP, and GPP. Prenylated aromatic compounds are formed from the prenyl transfer of the hydrocarbon units of isoprenoid precursors to aromatic polyketides. Isoprenoids and derivatives thereof can be formed from the isoprenoid precursors via prenyl transferase, terpene synthase, or terpene cyclases.

FIG. 2A-B: Generation of isoprenoid precursor GPP through non-decarboxylative condensations, beta-reductions, acyl-CoA mutases, and termination pathways starting with acetyl-CoA as the primer and propionyl-CoA as the extender unit.

FIG. 3A-B: Generation of isoprenoid precursors IPP, DMAPP, and GPP through non-decarboxylative condensation, beta-reductions, and termination pathways starting with glycolyl-CoA as the primer and propionyl-CoA as the extender unit.

FIG. 4A-B: Generation of isoprenoid precursors IPP, DMAPP and GPP through non-decarboxylative condensation, beta-reductions, acyl-CoA mutase, and termination pathways starting with propionyl-CoA as the primer and glycolyl-CoA as the extender unit.

FIG. 5A-B: Generation of isoprenoid precursors IPP, DMAPP and GPP through non-decarboxylative condensations, beta-reductions, acyl-CoA mutase, and termination pathways starting with propionyl-CoA as the primer and acetyl-CoA as the extender unit.

FIG. 6A-B: Generation of isoprenoid precursors IPP, DMAPP and GPP through non-decarboxylative condensation, beta-reductions, acyl-CoA mutase, and termination pathways starting with acetyl-CoA as the primer and propionyl-CoA as the extender unit.

FIG. 7A-B: Pathways for the synthesis of isoprenoid precursors IPP, DMAPP and GPP from the central carbon intermediate acetyl-CoA through decarboxylative or non-decarboxylative Claisen condensation. Conversion of 2 acetyl-CoA or an acetyl-CoA and a malonyl-CoA to acetoacetyl-CoA initiates the pathway, which then procceds through 3-hydroxy-3-methylglutaryl-CoA as an intermediate. Exemplary enzymes for each step shown in Table C.

FIG. 8A-B: Pathways for the synthesis of isoprenoid precursors IPP, DMAPP and GPP from the central carbon intermediate acetyl-CoA CoA through decarboxylative or non-decarboxylative Claisen condensation. Conversion of 2 acetyl-CoA or an acetyl-CoA and a malonyl-CoA to acetoacetyl-CoA initiates the pathway, which proceeds through 3-hydroxy-3-methylbutyryl-CoA as an intermediate. Exemplary enzymes for each step shown in Table D.

FIG. 9A-B: Pathways for the synthesis of isoprenoid precursors IPP, DMAPP and GPP from the acyloin condensation of the central carbon intermediate pyruvate. Conversion of 2 pyruvate to acetolactate initiates the pathway, which proceeds through 2-hydroxyisovalerate as an intermediate. Exemplary enzymes for each step shown in Table E.

FIG. 10A-B: Pathways for the synthesis of isoprenoid precursors IPP, DMAPP and GPP from the acyloin condensation of the central carbon intermediate pyruvate. Conversion of 2 pyruvate to acetolactate initiates the pathway, which proceeds through 2-isopropylmalate as an intermediate. Exemplary enzymes for each step shown in Table F.

FIG. 11A-C: Pathways for the synthesis of isoprenoid precursors IPP, DMAPP and GPP from the non-decarboxylative acyloin condensation of isobutanol and formyl-CoA. Exemplary enzymes for each step shown in Table G.

FIG. 12A-B: Pathways for the synthesis of isoprenoid precursors IPP, DMAPP and GPP from the aldol condensation of acetaldehyde and pyruvate. Condensation to 4-hydroxy-2-oxopentanote initiates the pathway, which proceeds through 2-hydroxyisovalerate as an intermediate. Exemplary enzymes for each step shown in Table H.

FIG. 13A-B: Pathways for the synthesis of isoprenoid precursors IPP, DMAPP and GPP from the aldol condensation of acetaldehyde and pyruvate. Condensation to 4-hydroxy-2-oxopentanote initiates the pathway, which proceeds through 2-isopropylmalate as an intermediate. Exemplary enzymes for each step shown in Table I.

FIG. 14A-B: Pathways for the synthesis of isoprenoid precursors IPP, DMAPP and GPP from the aldol condensation of acetaldehyde and 2-oxobutanoate. Examplary enzymes for each step shown in Table J.

FIG. 15: Pathways for the synthesis of isoprenoids from isoprenoid precursors such as DMAPP, IPP, GPP, and FPP. Generation of isoprenoid precursors through described routes can be combined with various isoprenoid forming enzymes such as prenyl transferases, terpene synthases, or terpene cyclases to synthesize isoprenoids and derivatives thereof. Examplary enzymes for each step shown in Table K.

FIG. 16A-B: Pathways for the synthesis of polyketides, olivetolic acid and olivetol, through thiolases-catalyzed non-decarboxylative condensations, beta-reductions, and termination pathways.

FIG. 17: Synthesis of prenylated aromatic compound cannabigerolic acid through olivetolic acid prenylation with the hydrocarbon unit of geranyl pyrophosphate. Geranyl pyrophosphate generated through various example routes as shown in FIG. 2-14, or through native pathways such as MVA or DXP pathway or commercial sources. Olivetolic acid generated through thiolases-catalyzed non-decarboxylative condensations, beta-reductions, and termination pathways, with examples shown in FIG. 16 or through alternative pathways or from commercial sources. Exemplary enzymes prenyl transfer step shown in Table L.

FIG. 18: Titers of tiglic acid of JST06(DE3) strain overexpressing thiolase FadAx, hydroxyacyl-CoA dehydrogenase FadB2x and enoyl-CoA hydratase FadB1x along with acyl-CoA transferase Pct with or without thioesterase YdiI in shake flasks or bioreactor.

FIG. 19: Total ion GC-MS chromatogram showing peak of synthesized 2,3-dihydroxybutyric acid synthesized by MG1655(DE3) ΔglcD pET-P1-bktB-phaB1-P2-phaJ pCDF-P1-pct-P2-tdter.

FIG. 20: Results of in vitro enzymatic assays of acyl-CoA transferases Pct and Pct540 on different substrates.

FIG. 21: 2-hydroxyisovaleric acid titer of JST06(DE3) expressing alsS, ilvC, ilvD and panE when grown on various carbon sources.

FIG. 22: Absorbance at 340 nm of in vitro assay samples and controls on dehydration of ethylene glycol to acetaldehyde by PddABC, coupled with actaldehyde oxidization to acetyl-CoA by Lmol179. Red: control without B12 coenzyme; Blue: control without cell lysates; Green: reaction sample with lysate and B12 coenzyme.

FIG. 23: Butyric acid production of JC01 strain overexpressing AtoB, FadB and EgTer in combination with overexpression of different thioesterase through pZS vector.

FIG. 24A-D: in vitro characterization of HACL1. FIG. 24A, the result of assay on degradation of 2-hydroxyhexadecaonyl-CoA to formyl-CoA and pentadecanal; FIG. 24B: the result of assay on acyloin condensation of pentadecanal and formyl-CoA to 2-hydroxyhexadecanoyl-CoA, which is hydrolyzed to 2-hydroxyhexadecanoic acid; FIG. 24C: the result of assay on acyloin condensation between formyl-CoA and acetaldehyde to lactyl-CoA, which is hydrolyzed to lactate; FIG. 24D: the result of assay on acyloin condensation between formaldehyde and formyl-CoA to glycolyl-CoA, which is hydrolyzed to glycolate.

FIG. 25: NADH oxidization of samples and controls of in vitro formate activation assay by E. coli acyl-CoA synthase ACS (EcAcs) coupled by Listeria monocytogenes acyl-CoA reductase Lmo1179 (LmACR).

FIG. 26: Prenol production in E. coli through the pathway via HMG-CoA with usage of different acyl-CoA reductases and alcohol dehydrogenase and different number of vectors.

FIG. 27: Geraniol production of E. coli strains harboring novel GPP synthesis pathway via HMG-CoA and prenol with usage of acyl-CoA reductases AdhE2 or CbjALD and alcohol dehydrogenase YahK.

FIG. 28: GC-MS spectra of olivetolic produced in vivo in comparison to olivetolic acid standard.

FIG. 29: GC-MS spectra of cannabigerolic acid (CBGA) produced in vivo in comparison to CBGA standard.

FIGS. 30A-1 to 30A-3, 30B-1 to 30B-11, 30C-1 to 30C-26: Embodiments of the invention.

### DETAILED DESCRIPTION

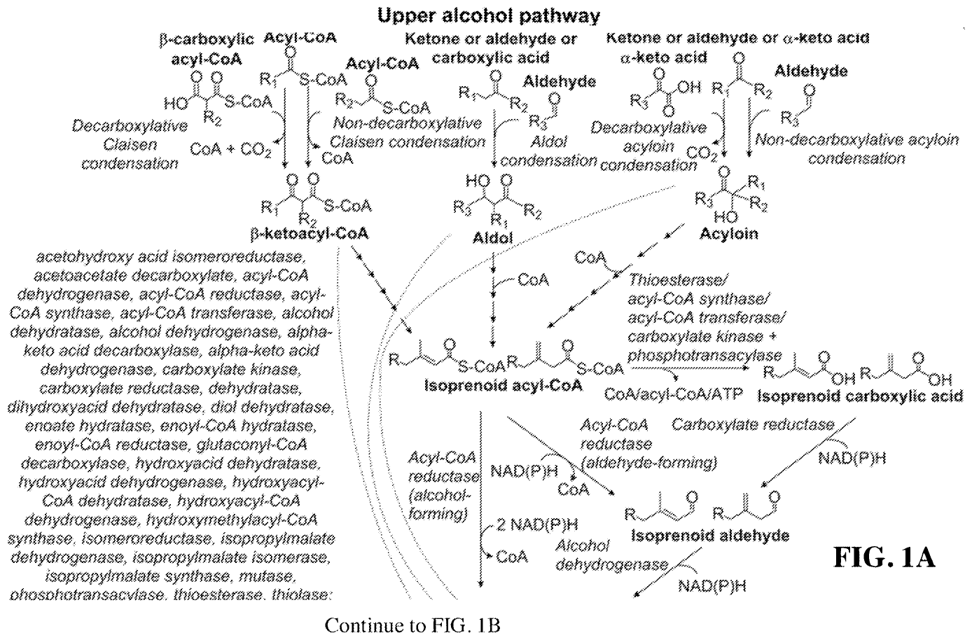

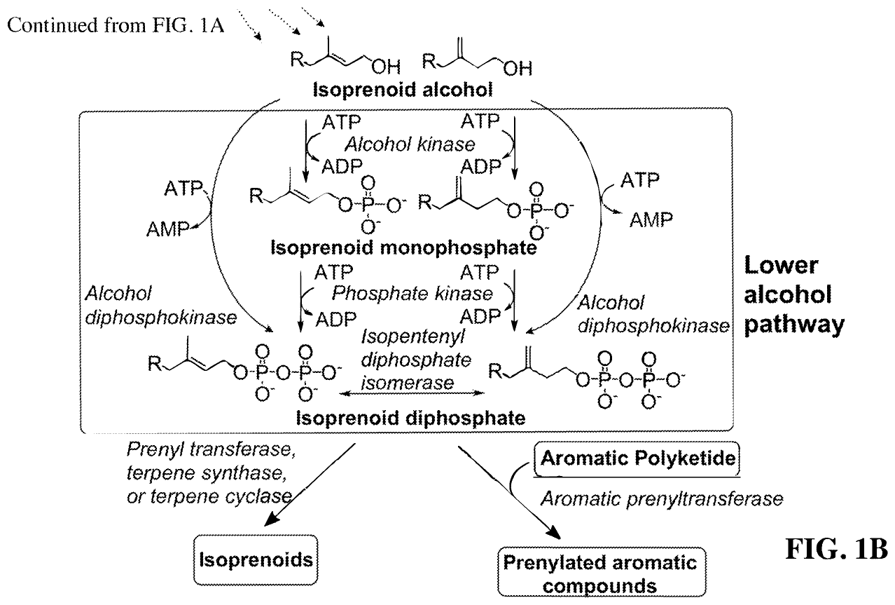

This disclosure generally relates to the use of enzyme combinations or recombinant microbes comprising the same to make isoprenoid precursors, isoprenoids and derivatives thereof including prenylated aromatic compounds through novel synthetic metabolic pathways. As described herein, the novel pathways for the synthesis of these products exploit enzymes catalyzing Claisen, aldol, or acyloin condensation reactions for the generation of longer chain length intermediates from central carbon metabolites (FIG. 1). Both decarboxylative and non-carboxylative condensations are utilized, enabling product synthesis from a number of different starting compounds. These condensation reactions serve as a platform for the synthesis of isoprenoid precursors, isoprenoids and derivatives thereof, polyketides, and prenylated aromatic compounds when utilized in combination with a variety of metabolic pathways and enzymes for carbon rearrangement and the addition/removal of functional groups (FIG. 1). Isoprenoid alcohols are key intermediary products for the production of isoprenoid precursors in these novel synthetic metabolic pathways.

One such pathway employs native or engineered thiolases that catalyze the condensation between an acyl-CoA, serving as the primer, and another acyl-CoA, serving as the extender unit, enabling the formation of beta-keto acyl-CoA intermediate (FIG. 1). Primers and extender units can be omega-functionalized to add required functionalities to the carbon chain, which can be further modified to form isoprenoid intermediates. The beta-keto group of the beta-keto acyl-CoA formed via condensation can be reduced and modified step-wise by one or more of the beta-reduction enzymes-dehydrogenase, dehydratase, and/or reductase reactions. Furthermore, various carbon re-arrangement enzymes, such as acyl-CoA mutases, can be employed to modify the carbon structure and branching of the acyl-CoAs. These CoA intermedites can then serve as the primer for the next round of condensation with the extender unit or as direct precursors to IPP, DMAPP, or other isoprenoid intermediates. After the termination by spontaneous or enzyme-catalyzed CoA removal, reduction, and/or phosphorylation, and subsequent structure re-arrangement, isoprenoids precursors (e.g. IPP and DMAPP), isoprenoids and derivatives thereof are produced. Many examples of thiolase enzymes which can potentially catalyze the condensation of an acyl-CoA primer and acyl-CoA extender unit are provided herein and the following Table A provides several additional examples which can also serve as templates for engineered variants. In another embodiment, ketoacyl-CoA syntheases can be employed in place of thiolases, catalyzing decarboxylative Claisen condensations.

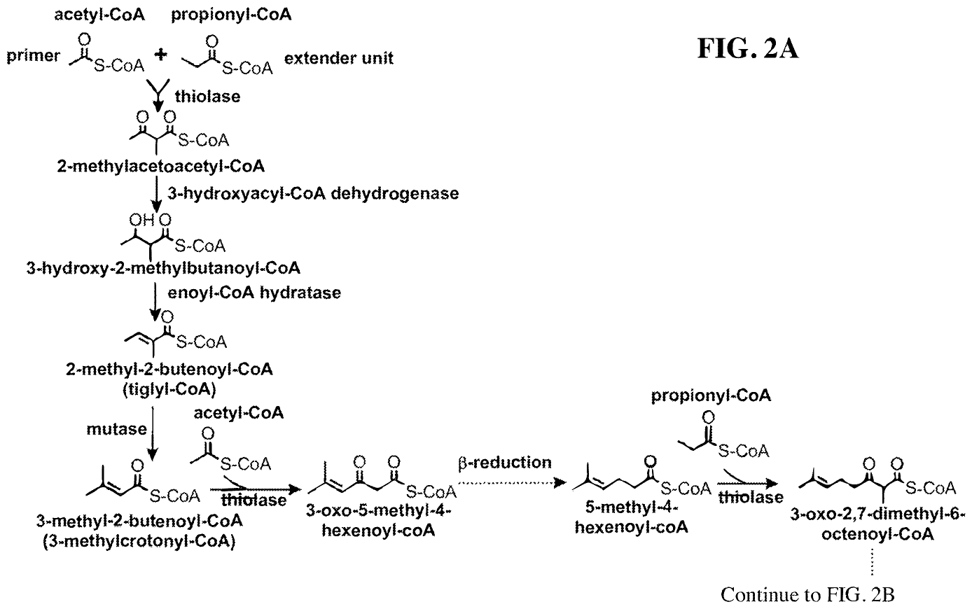

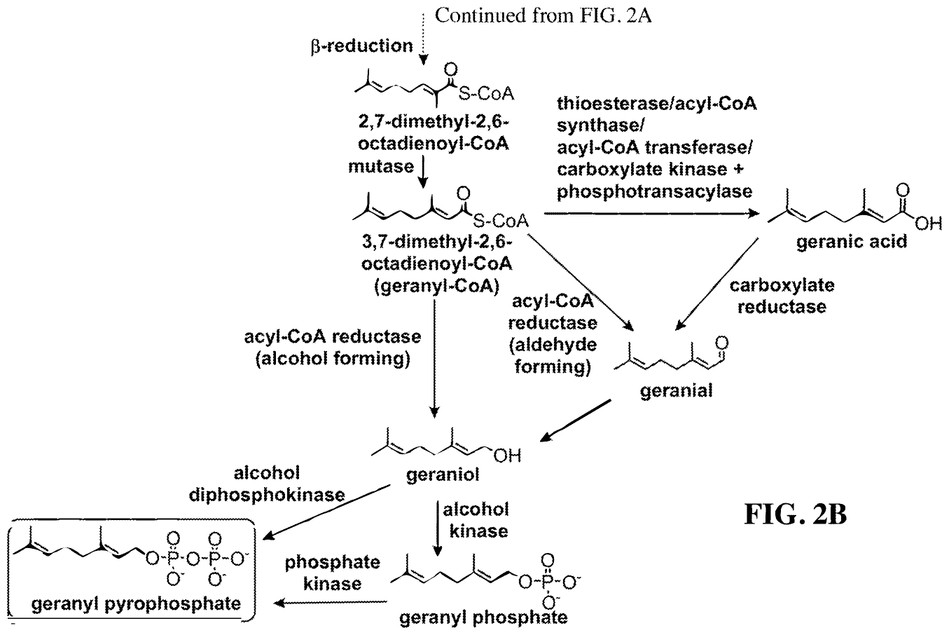

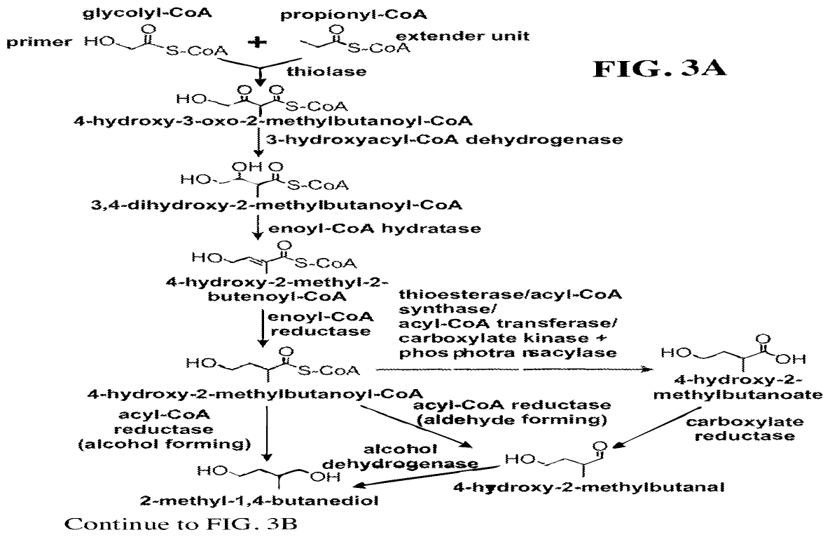

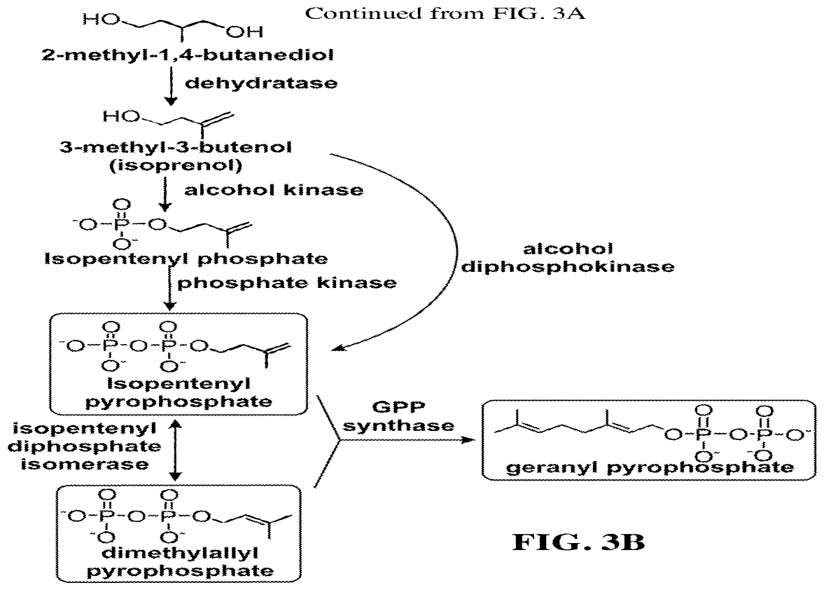

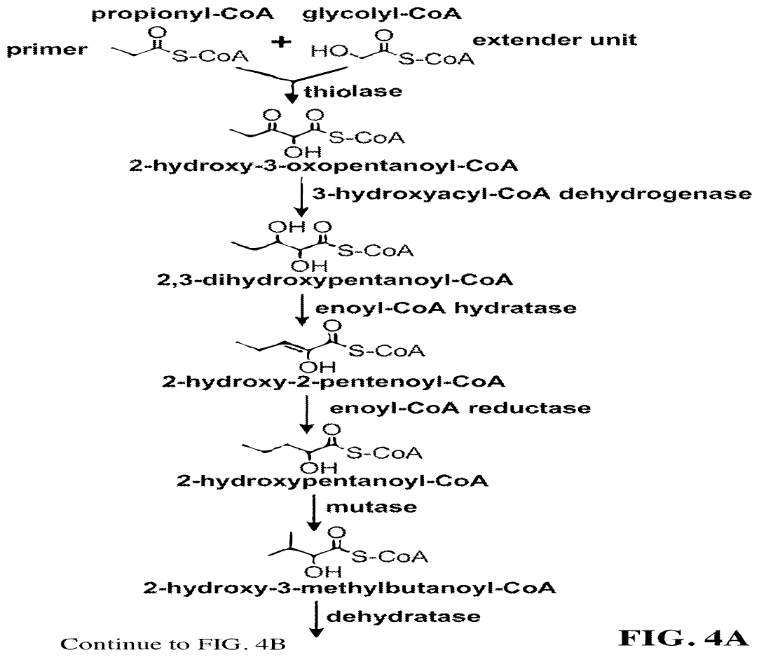

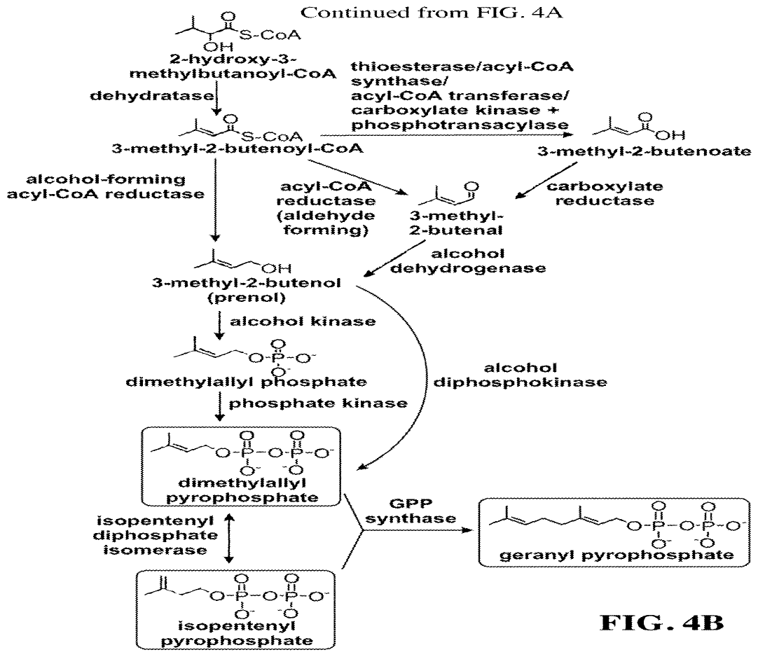

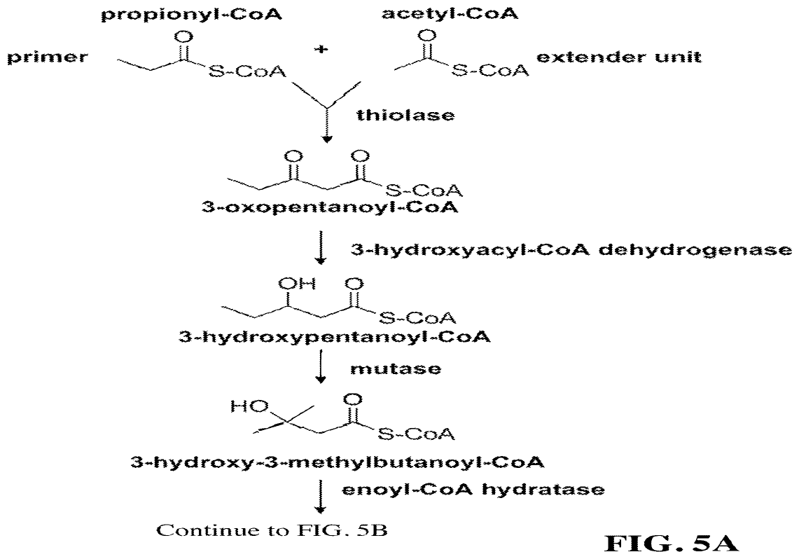

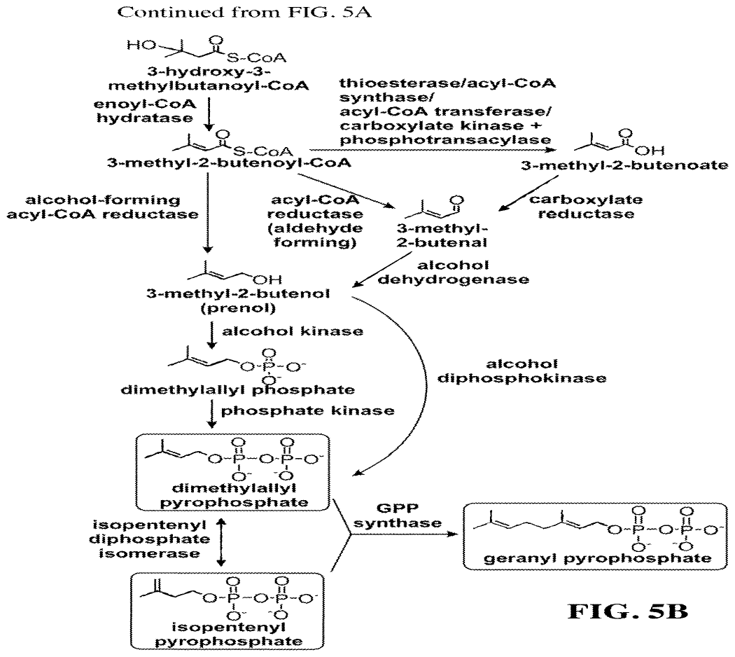

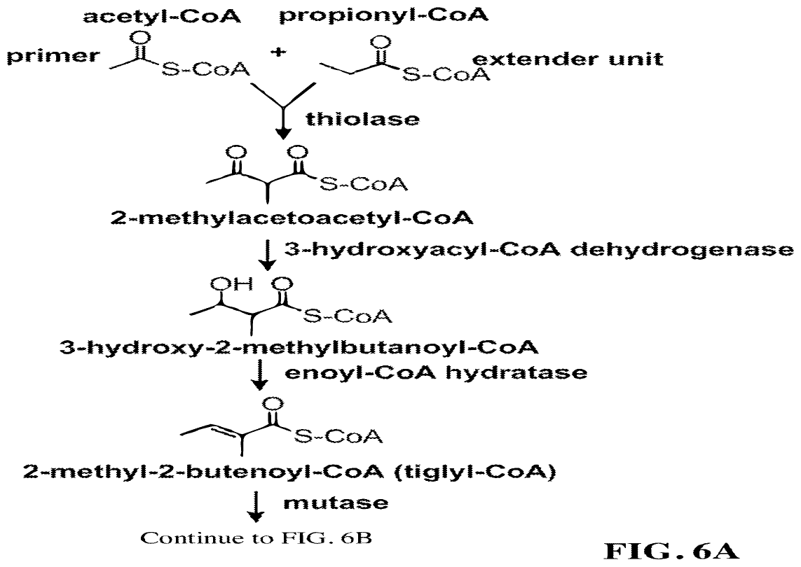

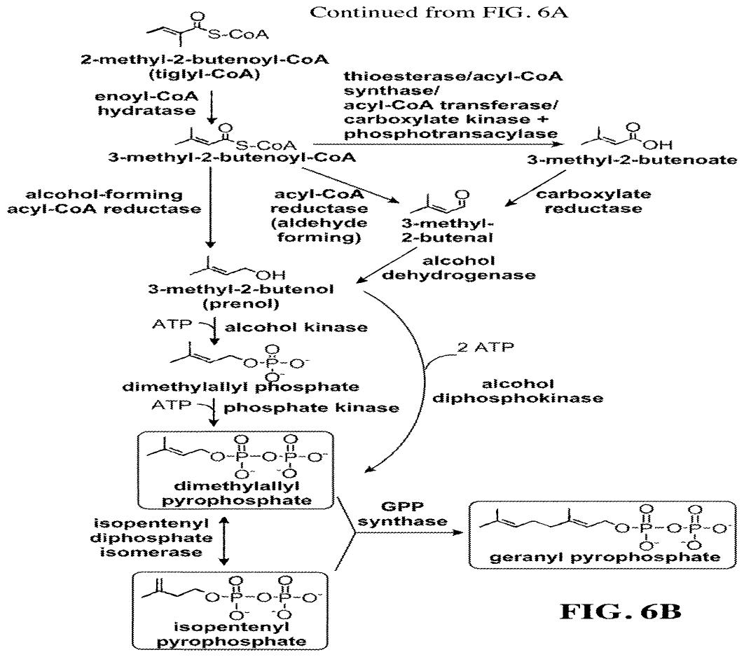

By employing these thiolase- or ketoacyl-CoA synthase catalyzed condensations with unsubstituted or functionalized acyl-CoAs serving as the primer and the extender unit, various beta-keto acyl-CoAs can be generated that through additional beta-reduction and carbon rearrangement modifications serve as direct precursors to the C₅ isoprenoid intermediates IPP or DMAPP. For example, FIGS. 2-6 depict various primer/extender unit combinations that through condensation and beta-reduction/carbon rearrangement reaction form CoAs that can be converted to IPP and DMAPP through various termination pathways. These building blocks can then be converted to longer chain length isoprenoid intermediates and products through, for example, known geranyl-, farnesyl- or, geranylgeranyl-diphosphate synthases, such as the formation of the C₁₀ intermediate geranyl pyrophosphate (GPP) from IPP and DMAPP by GPP synthase.

In addition to serving as precursors to IPP and DMAPP, the above described acyl-CoA intermediates can also serve as a primer for the next round of condensation with an extender unit enabling the synthesis of longer chain beta-keto acyl-CoAs. Additional rounds of elongation/beta-reduction/carbon rearrangement result in CoA intermediates that can be converted to longer chain length (e.g. C₁₀, C₁₅, etc.) isoprenoid intermediates. For example, FIG. 2 depicts the direct synthesis of GPP through condensation and beta-reduction/carbon rearrangement formation of an isoprenoid acyl-CoA that can be converted to GPP. This type of strategy can be utilized to target not only C₁₀ isoprenoid intermediates, but also longer chain length compounds as well. Following either route to isoprenoid precursors various prenyl transferases, terpene synthases, or terpene cyclases can be used to convert the isoprenoid precursors into desired isoprenoid products and derivatives thereof. Exemplary materials that can be used with the invention include those in Tables A and B.

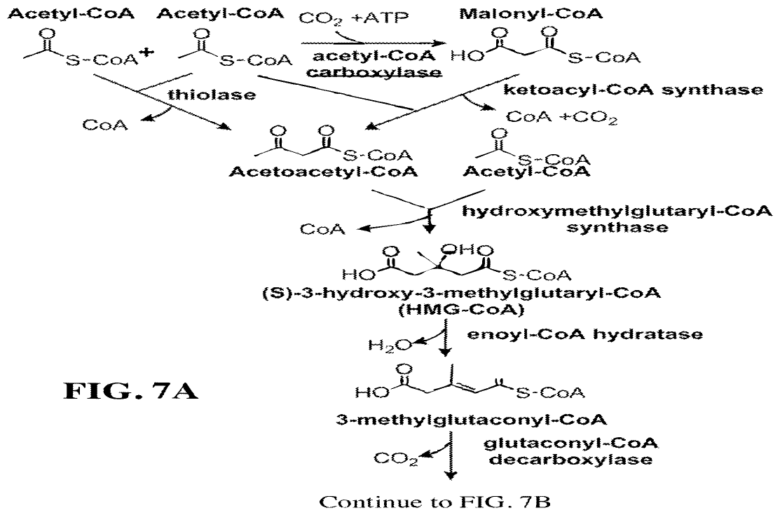

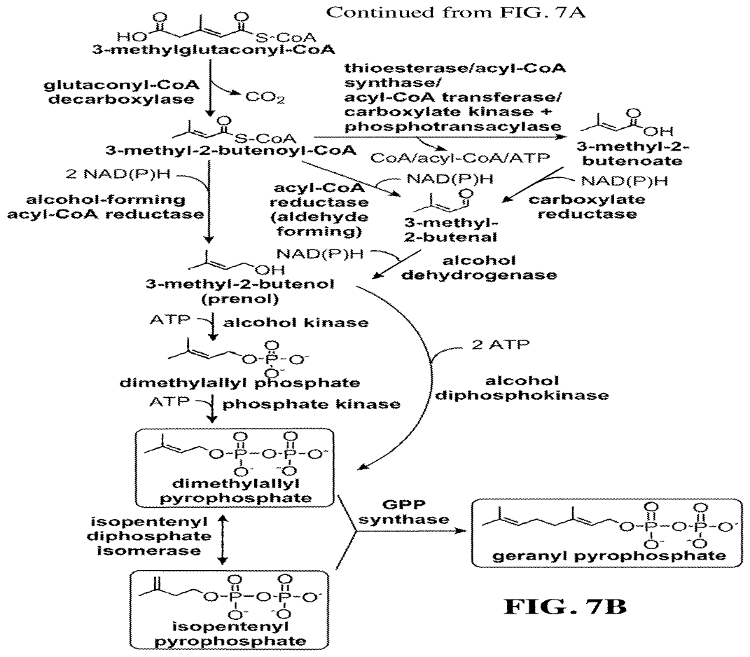

In another embodiment, the formation of isoprenoid precursors, isoprenoids and derivatives thereof including prenylated aromatic compounds proceeds from acetoacetyl-CoA formed as an intermediate through the non-decarboxylative condensation of 2 acetyl-CoA molecules catalyzed by thiolase(s) or decarboxylative condensation of acetyl-CoA and malonyl-CoA catalyzed by keto-acyl-CoA synthase(s). In one such pathway, acetoacetyl-CoA is first converted to 3-hydroxy-3-methylglutaryl-CoA by hydroxymethylglutaryl-CoA synthase (FIG. 7). 3-hydroxy-3-methylglutaryl-CoA is then dehydrated and decarboxylated through the action of an enoyl-CoA hydratase and glutaconyl-CoA decarboxylase, respectively, to form 3-methyl-2-butenoyl-CoA (FIG. 7). From 3-methyl-2-butenoyl-CoA, a number of routes are available leading to the formation of dimethylallyl phosphate. The formation of the isoprenoid precursors IPP and DMAPP then proceeds as described. These pathways are depicted in FIG. 7 and Table C provides examples of enzymes that can be used.

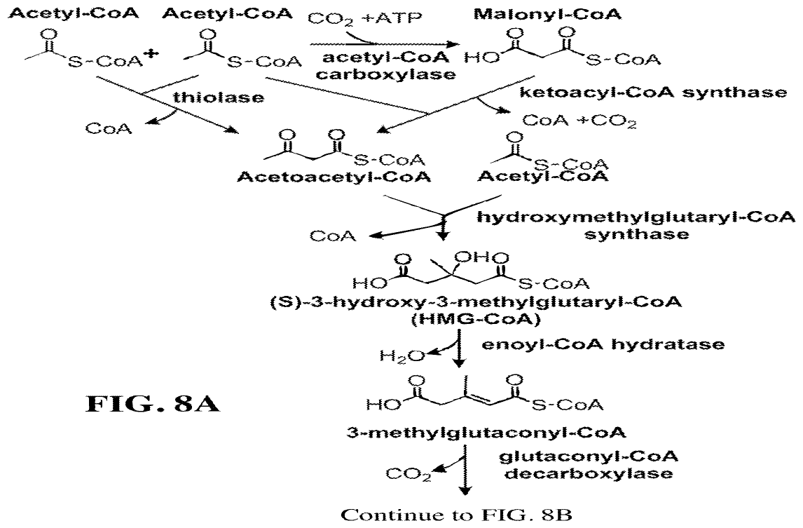

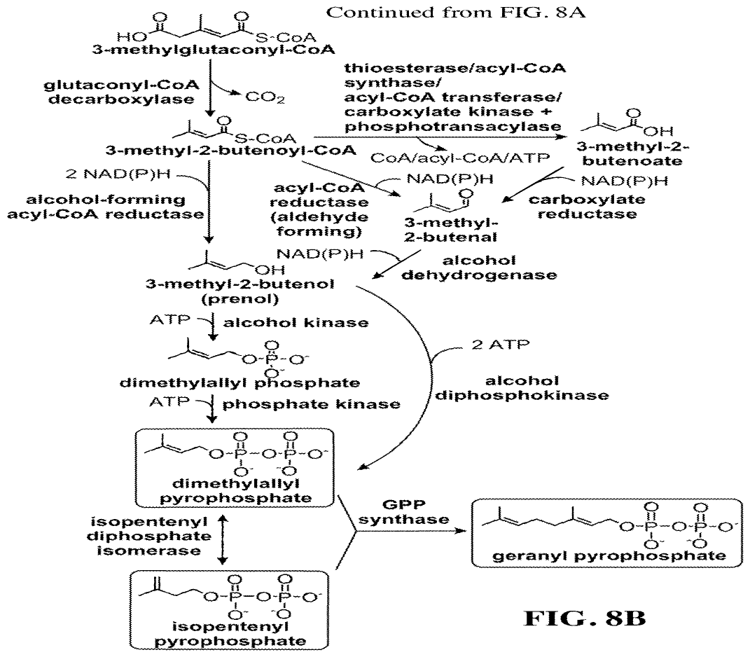

In another pathway from acetoacetyl-CoA, acetone generated from the decarboxylation of acetoacetic acid is converted to 3-methyl-3-hydroxy-butyryl-CoA through a condensation (FIG. 8). Dehydration of 3-methyl-3-hydroxy-butyryl-CoA through the action of an enoyl-CoA hydratase then forms 3-methyl-2-butenoyl-CoA. From 3-methyl-2-butenoyl-CoA, a number of routes are available leading to the formation of dimethylallyl phosphate, and then to IPP and DMAPP as described. These pathways are depicted in FIG. 8 and Table D provides examples of enzymes that can be used.

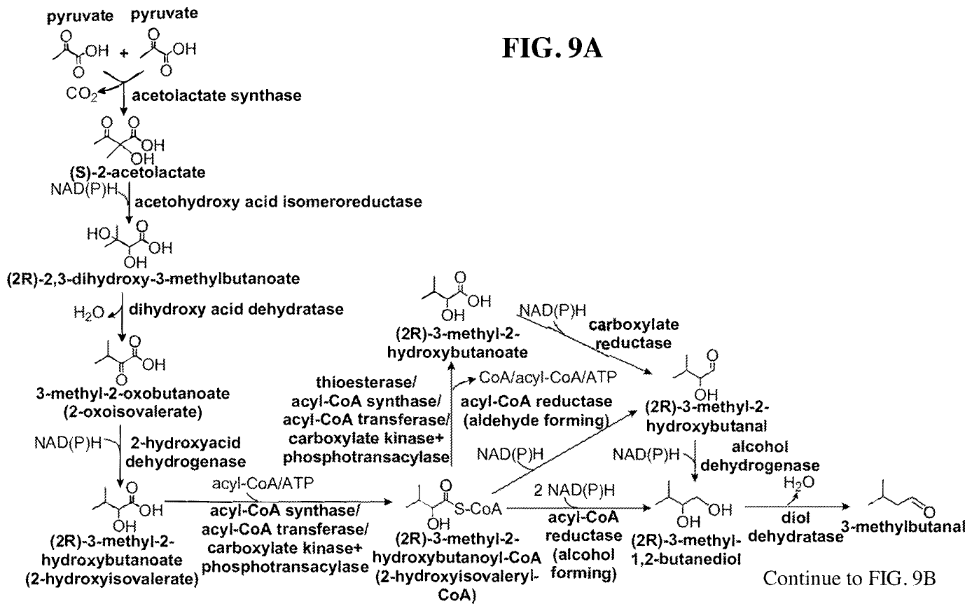

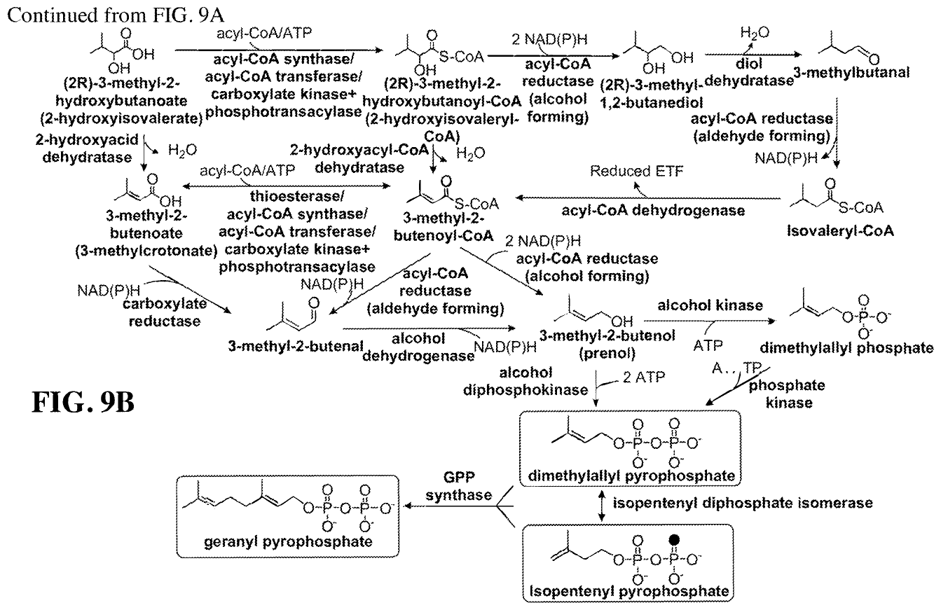

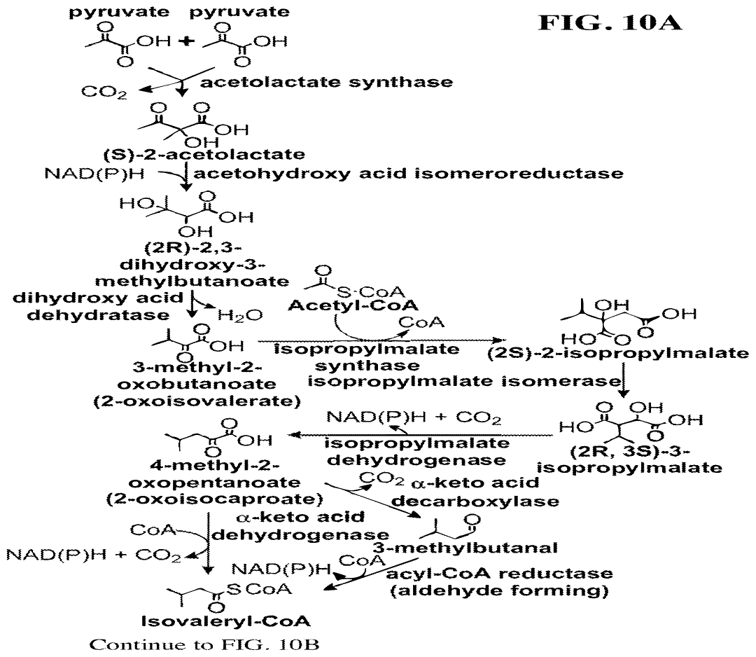

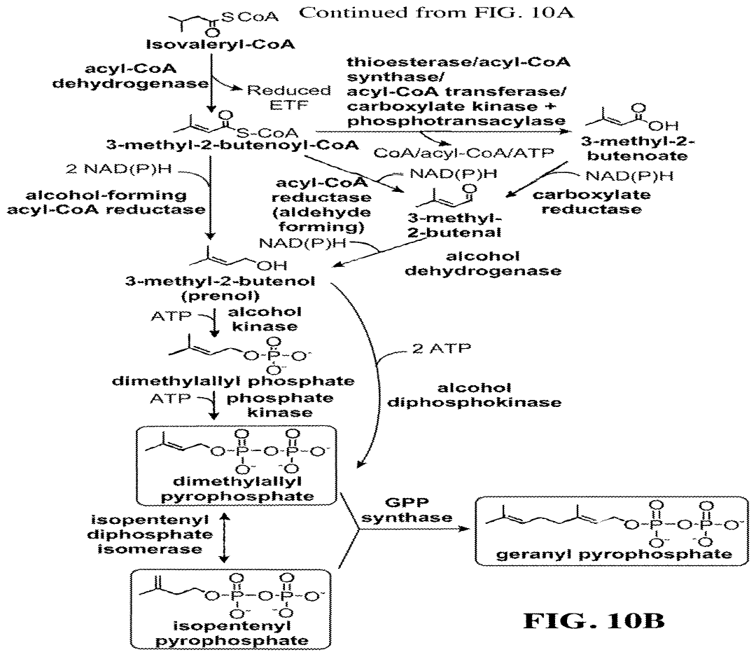

This disclosure also relates to the use of enzyme combinations or recombinant microbes to make isoprenoid precursors, isoprenoids and derivatives thereof including prenylated aromatic compounds through acyloin condensation reactions (FIG. 1). In one embodiment, the pathway begins from the central carbon intermediate pyruvate, with a decarboxylative acyloin condensation of 2 molecules of pyruvate forming acetolactate. Subsequent isomeroreduction and dehydration convert acetolactate to 3-methyl-2-oxobutanoate (FIG. 9 and FIG. 10). These first 3 reactions are catalyzed by acetalactate synthase, acetohydroxyacid isomeroreductase, and dihydroxyacid dehydratase, respectively.

Following the formation of 3-methyl-2-oxobutanoate, several potential pathways can be exploited for the conversion of 3-methyl-2-oxobutanoate to isoprenoid precursors. One such pathway to isoprenoid precursors involves a keto-reduction to 3-methyl-2-hydroxybutanoate, catalyzed by 2-hydroxyacid dehydrogenase. A series of different reactions can then be employed to convert 3-methyl-2-hydroxybutanoate into prenol (FIG. 9). In general, these steps involve the dehydration and phosphorylation of either the acid intermediate (3-methyl-2-hydroxybutanoate) or its CoA derivative to into prenol. Conversion of the acid intermediate requires a 2-hydroxyacid dehydratase for the formation of an alpha-beta-double bond, and the subsequent conversion to 3-methyl-2-butenoyl-CoA through the action of any of an acyl-CoA synthetase, an acyl-CoA transferase, or the combination of a carboxylate kinase and phosphotransacylase (FIG. 9). From 3-methyl-2-butenoyl-CoA, a number of routes are available leading to the formation of prenol. The formation of the isoprenoid precursors IPP and DMAPP then proceeds from prenol through an alcohol kinase and phosphate kinase or an alcohol diphosphokinase to form DMAPP, with isopentenyl diphosphate isomerase able to interconvert DMAPP and IPP. These pathways are also depicted in FIG. 9 and Table E.

Alternatively, 3-methyl-2-hydroxybutanoate can be converted into its CoA derivative (3-methyl-2-hydroxybutanoyl-CoA) before the dehydration reaction. This can be accomplished through any of an acyl-CoA synthetase, an acyl-CoA transferase, or the combination of a carboxylate kinase and phosphotransacylase. Following activation to 3-methyl-2-hydroxybutanoyl-CoA, the dehydration reaction forms 3-methyl-2-butenoyl-CoA, which is catalyzed by a 2-hydroxyacyl-CoA dehydratase, for which a number of candidate enzymes are available (Table E). From 3-methyl-2-butenoyl-CoA, a number of routes are available leading to the formation of prenol. The formation of the isoprenoid precursors IPP and DMAPP proceeds as described. These pathways are also depicted in FIG. 9 and Table E.

An alternative route from 3-methyl-2-oxobutanoate involves the addition of 2 carbons (with acetyl-CoA as the donor) through the action of isopropylmalate synthases to form (2S)-isopropylmalate (FIG. 10). Isopropylmalate isomerase and isopropylmalate dehydrogenase then convert (2S)-isopropylmalate to 4-methyl-2-oxopentanoate, which is subsequently converted to 3-methyl-2-butenoyl-CoA through a branched chain alpha-keto acid dehydrogenase and an acyl-CoA dehydrogenase (FIG. 10). From 3-methyl-2-butenoyl-CoA, a number of routes are available leading to the formation of prenol. The formation of the isoprenoid precursors IPP and DMAPP is as described above. These pathways are depicted in FIG. 10 and Table F provides examples of enzymes that can be used.

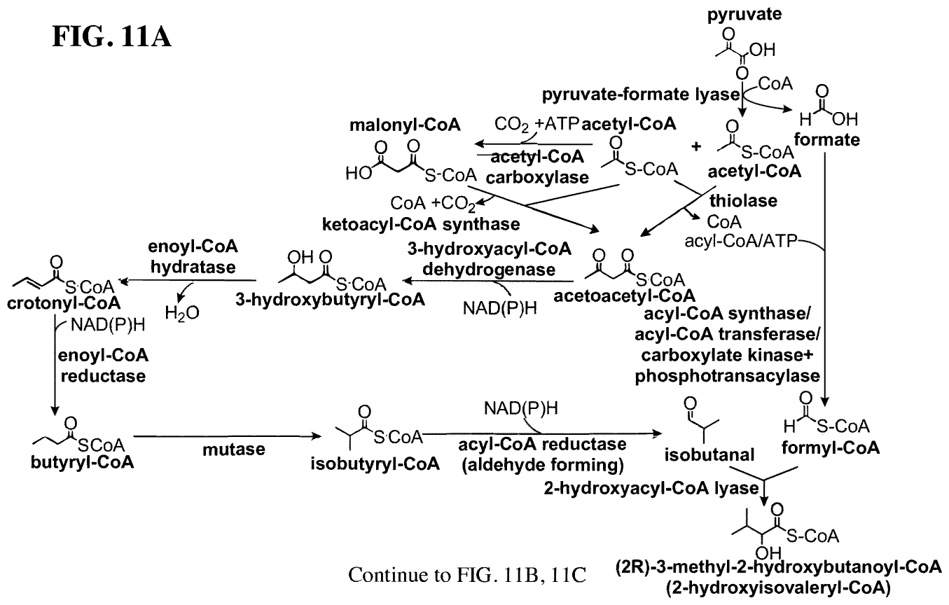

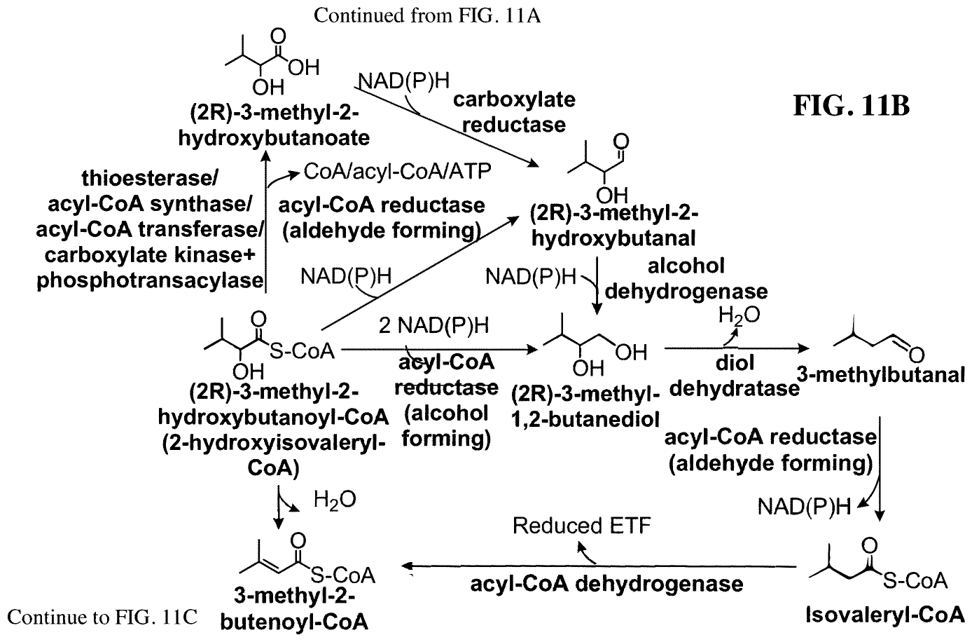

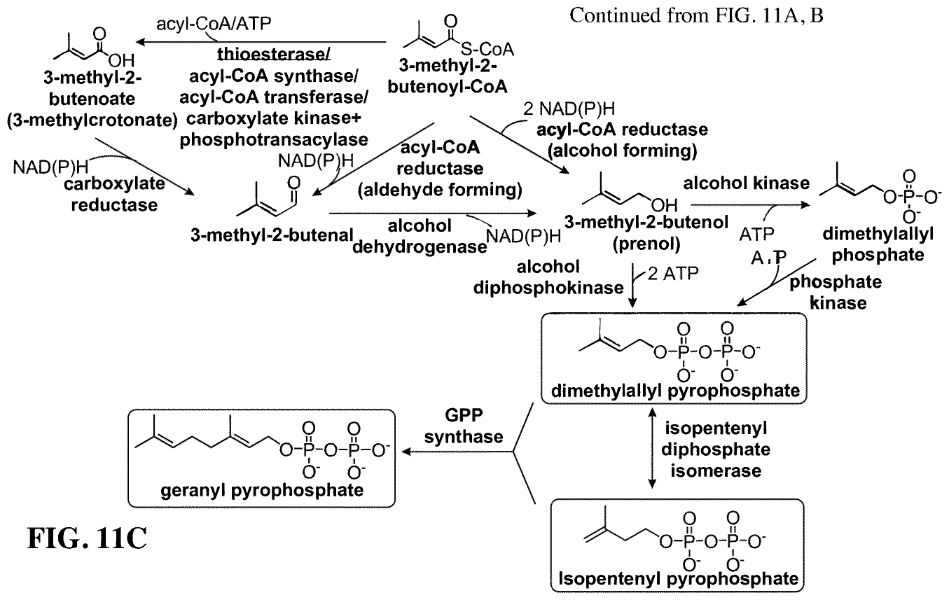

In another embodiment, the non-decarboxylative acyloin condensation of isobutanal and formyl-CoA to 3-methyl-2-hydroxybutanoyl-CoA catalyzed by 2-hydroxyacyl-CoA lyase is utilized (FIG. 11). Isobutanal is generated through the use of Claisen condensation and beta-reduction reactions, with carbon rearrangement and an aldehyde forming termination pathway. Formyl-CoA can be generated directly from formate or formaldehyde. Following acyloin condensation, 3-methyl-2-hydroxybutanoyl-CoA is converted to prenol through various pathways (FIG. 11). Prenol is subsequently converted into DMAPP and IPP. These pathways are depicted in FIG. 11 and Table G provides examples of enzymes that can be used.

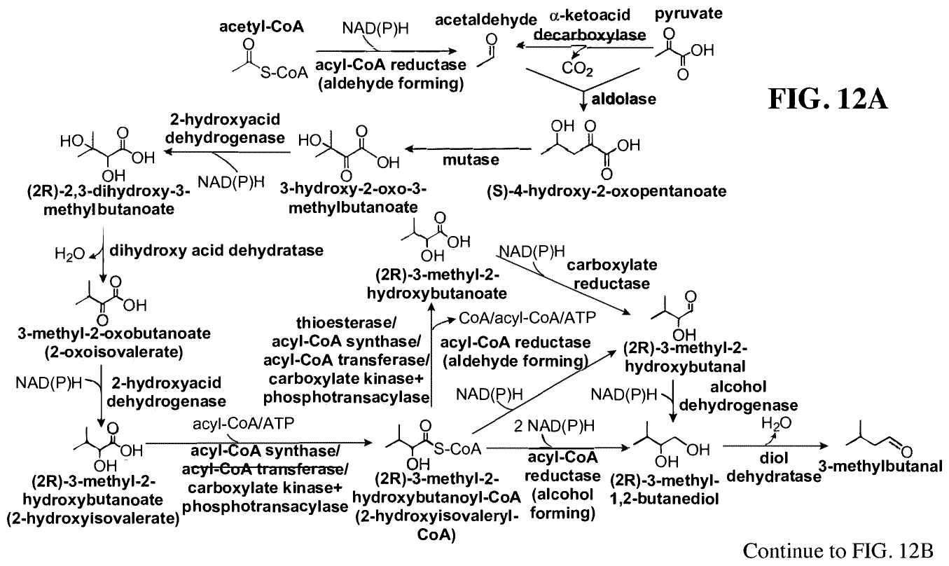

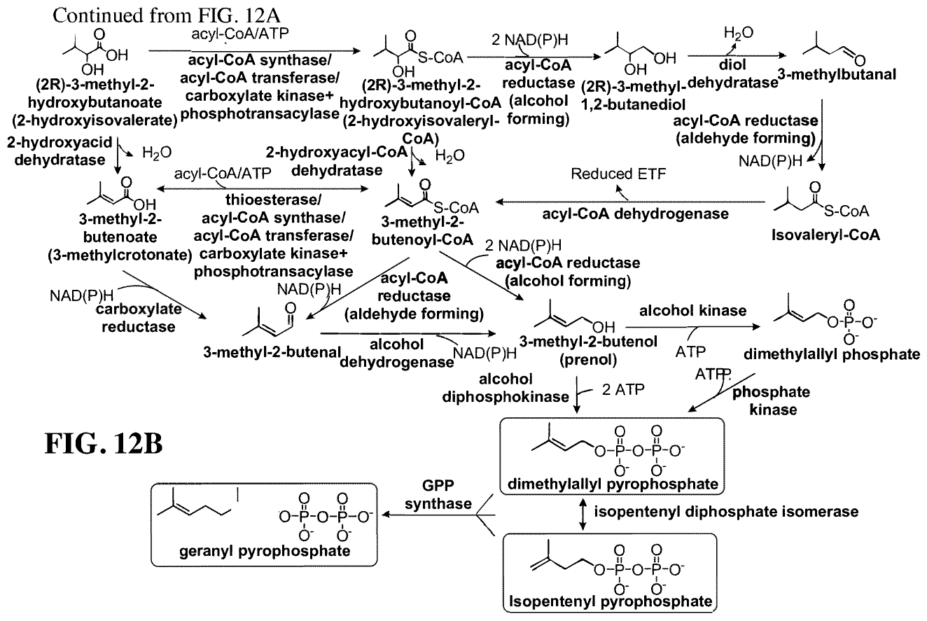

This disclosure also relates to the use of enzyme combinations or recombinant microbes to make isoprenoid precursors, isoprenoids and derivatives thereof including prenylated aromatic compounds through acyloin condensation reactions (FIG. 1). In one embodiment, the pathway begins from the central carbon intermediate pyruvate, which is condensed with acetealdeyhde in an aldol condensation to form 4-hydroxy-2-oxopentanoate (FIG. 12). Carbon rearrangement catalyzed by a mutase and reduction through the action of a 2-hydroxyacid dehydrogenase converts 4-hydroxy-2-oxopentanoate to 2,3-dihydroxy-3-methylbutanoate, an intermediate of the aformentoned valine biosythensis pathway. Following dehydration to 3-methyl-2-oxobutanoate, several metabolic routes to isoprenoid precursors can be exploited, including keto-reduction and combinations of dehydration and phosphorylation, either converting the free acid intermediate or its CoA derivative to prenol (FIG. 12). Prenol is subsequently converted into DMAPP and IPP. These pathways are depicted in FIG. 12 and Table H below provides examples of enzymes that can be used.

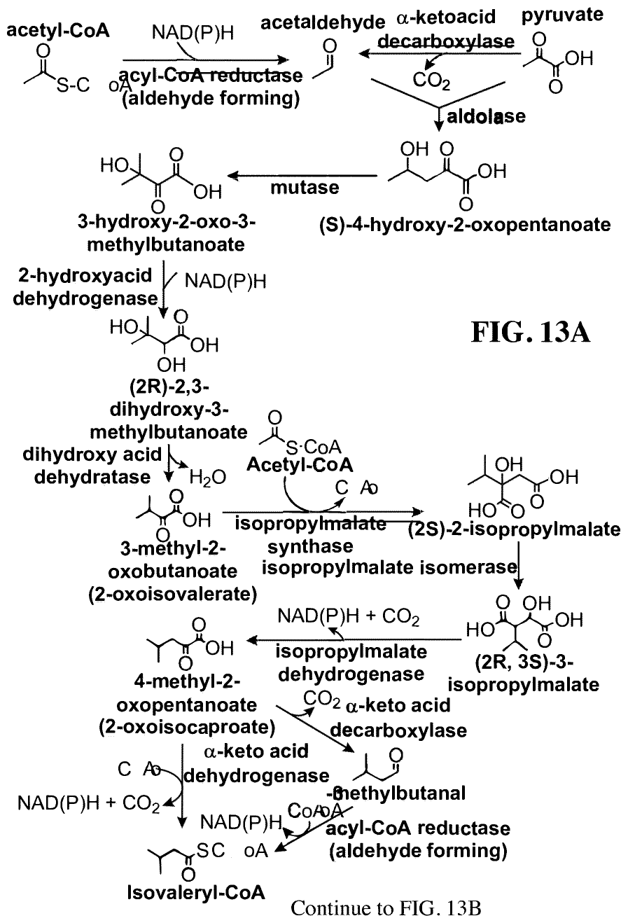

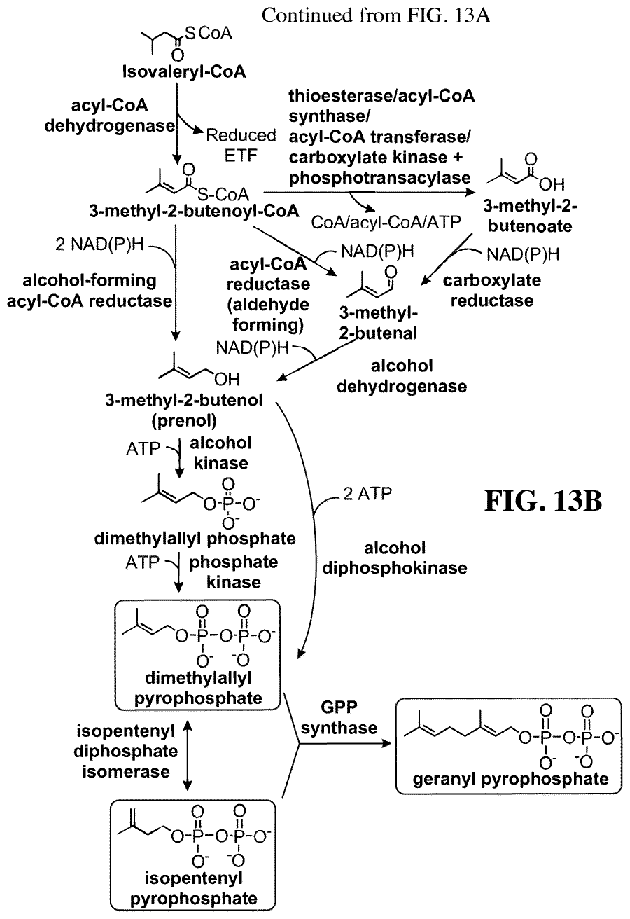

Alternatively, the addition of 2-carbons to 3-methyl-2-oxobutanoate, followed by subsequent isomerization, and decarboxylation results in the generation of isovaleryl-CoA, which can then be converted to prenol through a series of reactions (FIG. 13). Prenol is then converted to DMAPP, which can be isomerized into IPP generating the two C₅ isoprenoid precursors. These pathways are depicted in FIG. 13 and Table I below provides examples of enzymes that can be used.

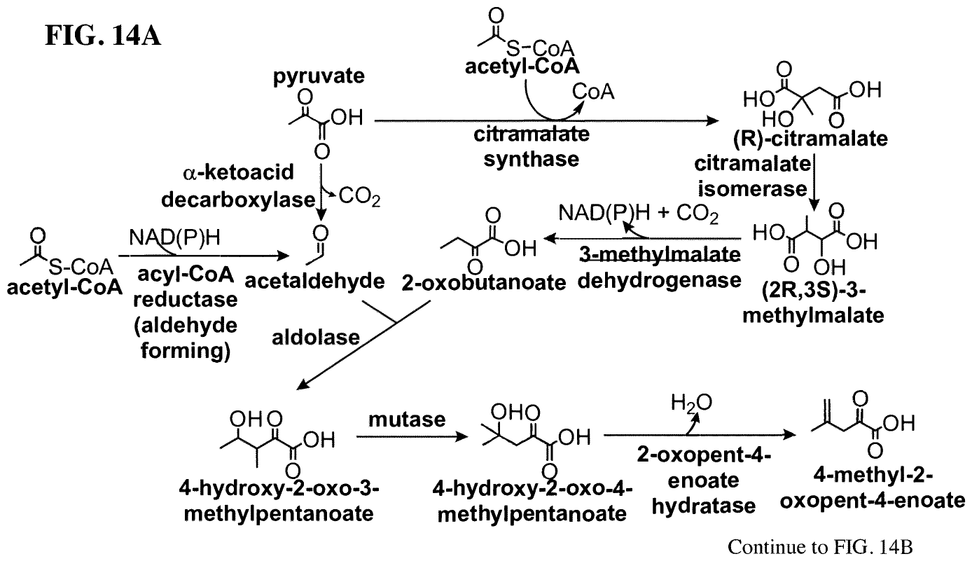

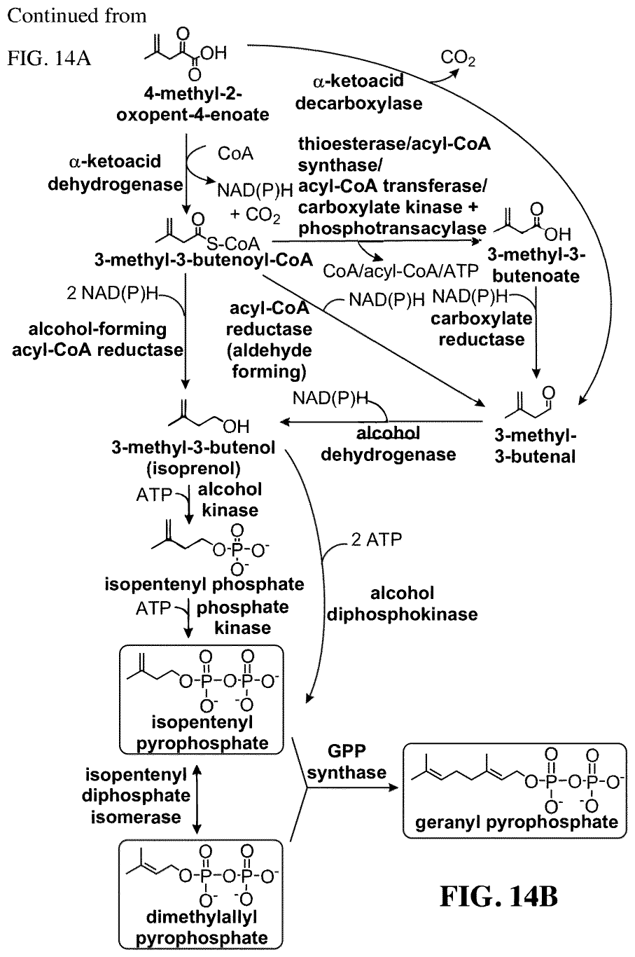

In another embodiment, an aldolase catalyzes the aldol condensation of 2-oxobutanoate and acetaldehyde forming 4-hydroxy-2-oxo-3-methylpentanoate (FIG. 14). Conversion of this intermediate to 4-methyl-2-oxopent-4-enoate, through the action of a mutase and a dehydratase, enables the use of a number of pathways to generate isoprenol from 4-methyl-2-oxopent-4-enoate. This 5-carbon isoprenoid alcohol is then converted to IPP through a two-step phosphorylation with IP as an intermediate, or a one step diphosphorylation catalyzed by an alcohol diphosphokinase. IPP can be isomerized into DMAPP generating the two C₅ isoprenoid precursors. These pathways are depicted in FIG. 14 and Table J below provides examples of enzymes that can be used.

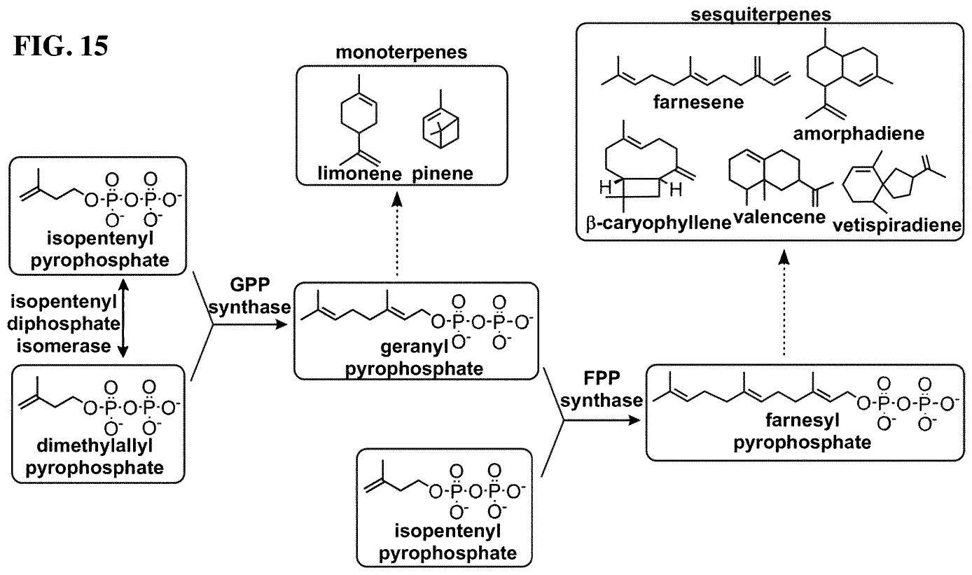

The synthesis of IPP, DMAPP, GPP, FPP or other isoprenoid precursors can then be combined with the rearrangement of these intermediates into the desired isoprenoid product. The 5-carbon isomers IPP and DMA-PP are the fundamental building blocks of isoprenoid products. From these C₅ units, an immense number of products can be synthesized through the action of for example prenyl transferases, terpene synthases, or terpene cyclases, which involves the prenyl transfer, head-to-tail condensation, head-to-head condensation, tail-to-tail condensation, or cyclization, among other biochemical reactions, of IPP, DMAPP, and other longer chain isoprenoid precursors synthesized from the C₅ building blocks. As such, the generation of these intermediates can enable the synthesis of for example a variety of monoterpenes (C₁₀), sesquiterpenes (C₁₅), diterpenes (C₂₀), sesterterpenes (C₂₅), triterpenes (C₃₀), sesquarterpenes (C₃₅), and tetraterpenes (C₄₀), among other isoprenoid compounds and derivatives thereof (FIG. 15). Table K below provides examples of enzymes that can be used.

The isoprenoid precursors synthesized through these routes can also be exploited for the synthesis of hybrid products, which contain as an example, the C₅ (dimethylallyl), C₁₀ (geranyl), or C₁₅ (farnesyl) isoprenoid attached to an aromatic core structure. The prenylation of these aromatic compounds with the isoprenoid units offers another route to diverse products. One route to polyketides involves native or engineered thiolases catalyzing the condensation in an iterative manner (i.e. one or multiple rounds) between two either unsubstituted or functionalized acyl-CoAs each serving as the primer and the extender unit to generate and elongate polyketide CoAs. Before an optional next round of thiolase reaction, the beta-keto group of the polyketide chain can be reduced and modified step-wise by the beta-reduction reactions. Spontaneous or enzymatically catalyzed termination reaction terminates the elongation of the polyketide chain at any point through CoA removal and reactions rearranging the structure, generating the final functional polyketide products. Examples of enzymes that can be used for these key reactions are shown in Tables A and B. This approach is the subject of patent application WO2017020043, BIOSYNTHESIS OF POLYKETIDES, filed Aug. 1, 2016, and 62/198,764, filed Jul. 30, 2015.

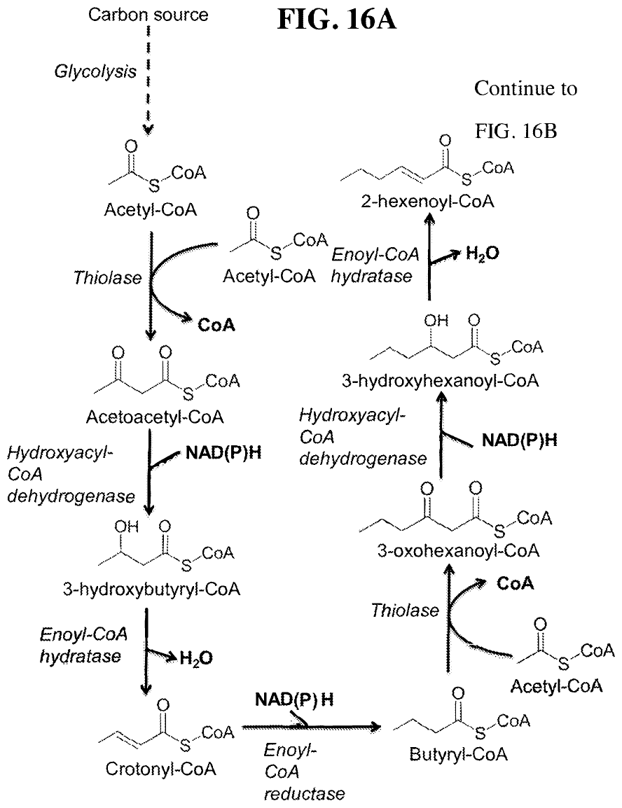

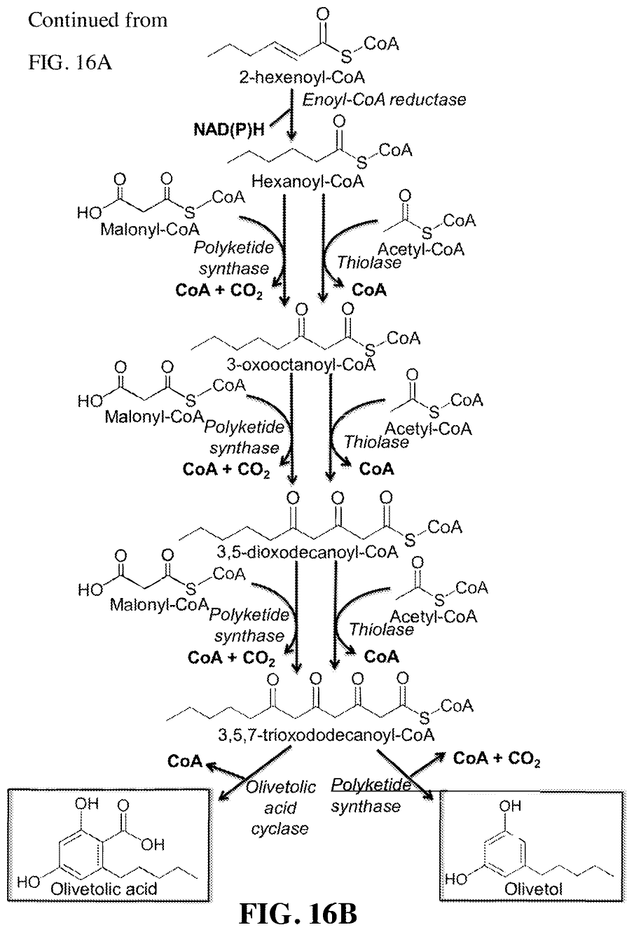

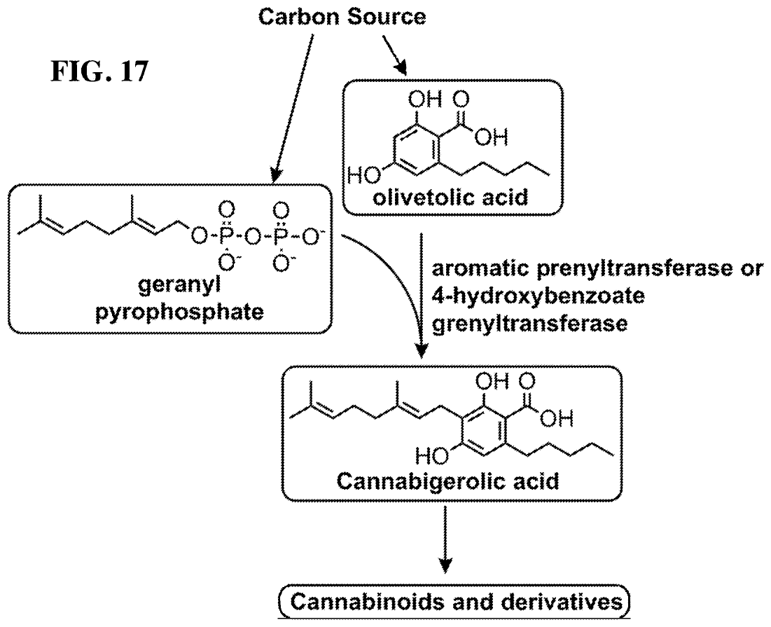

The polyketides synthesized through this route or other routes such as polyketide synthases can be combined with isoprenoid precursors for the formation of prenylated aromatic compounds. For example, FIG. 16 demonstrates olivetolic acid generation through condensation and beta-reduction reactions and generation of isoprenoid precursor geranyl pyrophosphate, which when combined through the action of an aromatic prenyltransferase or 4-hydroxybenzoate grenyltransferase, enables the synthesis of the cannabinoid cannabigerolic acid (FIG. 17). Cannabigerolic acid can then be converted into a number of other cannabinoids, including Δ⁹-tetrahydrocannabinolic acid, cannabidiolic acid, and cannabichromenic acid. Examples of enzymes that can be used for these key reactions are shown in Table L.

As such, through the use of these novel pathways based on Claisen, aldol, or acyloin condensation, this platform can be exploited to make not only isoprenoids precursors, isoprenoids and derivatives thereof, but also diverse hybrid products with wide ranging applications.

### (Prophetic) GPP Biosynthesis Through Utilization of Beta-Oxidation Reversal and Methyl Group Transferring Mutase

The purpose of this example is to demonstrate the biosynthesis of GPP through a novel pathway that recruits condensation and beta-reduction reactions as well as a mutase that moves the methyl group by one carbon. E. coli serves as the host organism. This pathway starts from non-decarboxylative Claisen condensation between acetyl-CoA, which serves as the primer, and propionyl-CoA, which serves as the extender unit, by thiolase FadAx (AAK18171.1) from P. putida. In the pathway, propionyl-CoA is activated from propionic acid, which is either supplemented or synthesized through overexpressed native pathway of conversion of succinate to propionic acid, catalyzed by M. elsdenii acyl-CoA transferase Pct (BAU59368.1). After two beta-reduction steps catalyzed by hydroxyacyl-CoA dehydrogenase FadB2x (AAK18170.1) and enoyl-CoA hydratase FadB1x (AAK18173.1), both from P. putida, 2-methylcrotonyl-CoA (tiglyl-CoA) is generated. Then, mutase moves the methyl group from alpha-site to beta-site on tiglyl-CoA, generating 3-methyl-2-butenoyl-CoA (3-methylcrotonyl-CoA). 3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. SE19 ChnD (BAC80217.1).

Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP_010900530.1, V73I, Y141V and K204G mutations to increase specificity on prenol. Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. Then, DMAPP and IPP are condensed to GPP catalyzed by E. coli GPP synthase IspA (NP_414955.1, with S80F mutation to make the enzyme exclusive active on GPP synthesis, Reiling et al. 2004) or Abies grandis GPP synthase GPPS2 (AAN01134.1, N-terminal 84 aa truncation to improve the activity). 3-methylcrotonyl-CoA can also serve as the primer for the next iteration composed of reactions by thiolase, hydroxyacyl-CoA dehydrogenase, enoyl-CoA hydratase and enoyl-CoA reductase, with acetyl-CoA as the extender unit, generating 5-methyl-4-hexenoyl-CoA. 5-methyl-4-hexenoyl-CoA, serving as the primer, is condensed with extender unit propionyl-CoA through condensation by P. putida thiolase FadAx. After two beta-reduction steps catalyzed by P. putida hydroxyacyl-CoA dehydrogenase FadB2x and enoyl-CoA hydratase FadB1x, 2,7-dimethyl-2,6-octadienoyl-CoA is formed. Then, mutase moves the methyl group from alpha-site to beta-site, converting 2,7-dimethyl-2,6-octadienoyl-CoA to 3,7-dimethyl-2,6-octadienoyl-CoA, namely geranyl-CoA. Geranyl-CoA is converted to geraniol by alcohol-forming acyl-CoA reductase or aldehyde forming acyl-CoA reductase and alcohol dehydrogenase. Geraniol is then converted to GPP by one or two steps of phosphorylation. If phosphorylated through two steps, the first step is catalyzed by Arabidopsis thaliana alcohol kinase AT5G58560 (NP_200664.1) and the second step is catalyzed by Thermoplasma acidophilum phosphate kinase ThaIPK (WP_010900530.1, Y70A, V130A and I130A mutations to increase specificity on geranyl phosphate over isopentenyl phosphate, Mabanglo et al. 2012). The one-step phosphorylation is catalyzed by alcohol diphosphokinase. Ocimum basilicum geraniol synthase GES (AR11765.1, with N-terminal 65 aa truncated to improve the activity, Iijima et al. 2004) converts GPP to geraniol, which serves as the proxy product of GPP to demonstrate the synthesis pathway.

JST06(DE3) serves as the E. coli host strain for demonstration of this novel pathway. JST06(DE3) (MG1655(DE3) ΔIdhA ΔpoxB Δpta ΔadhE ΔfrdA ΔyciA ΔybgC ΔydiI ΔtesA ΔfadM ΔtesB) (Cheong et al. 2016) is an E. coli strain deficient in mixed-acid fermentation pathways due to deletions of genes ldhA, poxB, pta, adhE and frdA, which maximize the supply of acetyl-CoA, and deletions of genes encoding major thioesterases (yciA, ybgC, ydiI, tesA, fadM and tesB), which minimize the hydrolysis of CoA intermediates.

The genes for overexpression are either cloned into appropriate vectors or inserted into chromosome with strong synthetic constitutive promoter, such as M1-93. When cloned into vectors, these genes are amplified through PCR using appropriate primers to append homology on each end for recombination into the vector backbone with e.g., Phusion polymerase (Thermo Scientific, Waltham, MA) to serve as the gene insert. Plasmids are linearized by the appropriate restriction enzymes (New England Biolabs, Ipswich, MA, USA) and recombined with the gene inserts using the In-Fusion HD Eco-Dry Cloning system. The mixture is subsequently transformed into Stellar competent cells. Transformants that grow on solid media (LB+Agar) supplemented with the appropriate antibiotic are isolated and screened for the gene insert by PCR. Plasmids from verified transformants are isolated and the sequence of the gene insert is further confirmed by DNA sequencing. The sequence confirmed plasmids are then introduced to host strain through electroporation.

When inserted into chromosome, CRISPR is used and genetic sites of tesB, adhE and ldhA are suitable loci, although others could be used. CRISPR method is based on the method developed by Jiang et al. (Jiang et al. 2015). First, the host strain is transformed with plasmid pCas, the vector for expression of Cas9 and k-red recombinase. The resulting strain is grown under 30 °C with L-arabinose for induction of X-red recombinase expression, and when OD reaches ~0.6, competent cells are prepared and transformed with pTargetF (AddGene 62226) expressing sgRNA and N20 spacer targeting the locus and template of insertion of target gene. The template is the inserted gene plus M1-93 promoter with ~500 bp sequences homologous with upstream and downstream of the insertion locus, constructed through overlap PCR with usage of Phusion polymerase or synthesized by GenScript (Piscataway, NJ) or GeneArt® (Life Technologies, Carlsbad, CA). The way to switch N20 spacer of pTargetF plasmid is inverse PCR with the modified N20 sequence hanging at the 5′ end of primers with usage of Phusion polymerase and followed by self-ligation with usage of T4 DNA ligase and T4 polynucleotide kinase (New England Biolabs, Ipswich, MA, USA). Transformants that grow under 30 °C on solid media (LB+Agar) supplemented with spectinomycin and kanamycin (or other suitable antibiotic) are isolated and screened for the chromosomal gene insert by PCR. The sequence of the gene insert, which is amplified from genomic DNA through PCR using Phusion polymerase, is further confirmed by DNA sequencing. The pTargetF can then be cured through IPTG induction, and pCas can be cured through growth under higher temperature like 37-42 °C.

All molecular biology techniques are performed with standard methods (Miller, 1972; Sambrook et al., 2001) or by manufacturer protocol. Strains are stored in glycerol stocks at −80 °C. Plates are prepared using LB medium containing 1.5% agar, and appropriate antibiotics are included at the following concentrations: ampicillin (100 μg/mL), kanamycin (50 μg/mL), spectinomycin (50 μg/mL) and chloramphenicol (12.5 μg/mL).

MOPS minimal medium (Neidhardt et al., 1974) with 125 mM MOPS and Na₂HPO₄ in place of K₂HPO₄ (2.8 mM), supplemented with 20 g/L glycerol or 40 g/L glucose, 10 g/L tryptone, 5 g/L yeast extract, 100 μM FeSO₄, 5 mM calcium pantothenate, 5 mM (NH₄)₂SO₄, and 30 mM NH₄Cl is used for fermentations. If required, 55 g/L of CaCO₃ is also supplemented as pH buffer. 20 mM propionic acid is supplemented, if it is not synthesized intracellularly and needed for the experiment. Antibiotics (50 μg/mL carbenicillin, 50 μg/mL spectinomycin and 50 μg/mL kanamycin) are included when appropriate. All chemicals are obtained from Fisher Scientific Co. (Pittsburgh, PA) and Sigma-Aldrich Co. (St. Louis, MO).

Fermentations are performed in 25 mL Pyrex Erlenmeyer flasks (narrow mouth/heavy duty rim, Corning Inc., Corning, NY) filled with appropriate volume of fermentation medium and sealed with foam plugs filling the necks. A single colony of the desired strain is cultivated overnight (14-16 hrs) in LB medium with appropriate antibiotics and used as the inoculum with initial OD₅₅₀ as ~0.05. After inoculation, flasks are incubated in a NBS 124 Benchtop Incubator Shaker (New Brunswick Scientific Co., Inc., Edison, NJ) at 200 rpm and 37 °C or 30 °C. When optical density (550 nm, OD₅₅₀) reached ~0.3-0.5, appropriate concentration of isopropyl beta-D-1-thiogalactopyranoside (IPTG) (or other suitable inducer) is added for plasmid gene induction. Additional fermentations are conducted in a SixFors multi-fermentation system (Infors HT, Bottmingen, Switzerland) with an air flowrate of 2 N L/hr, independent control of temperature (37 °C), pH (controlled at 7.0 with NaOH and H₂SO₄), and appropriate stirrer speed. Pre-cultures are grown in 25 mL Pyrex Erlenmeyer flasks as described above and incubated for 4 hours post-induction. An appropriate amount of this pre-culture is centrifuged, washed twice with fresh media, and used for inoculation (400 mL initial volume). The fermentations in bioreactor use described fermentation media with 30 g/L glycerol or 40 g/L glucose, with the optional inclusion of 5 μM sodium selenite to promote FHL activity, and appropriate IPTG and antibiotics. If required, propionic acid (20 mM) is added at 0, 24, and 48 hours.

After the fermentation, the supernatant obtained through 5000 g, 5 min centrifuge in an Optima L-80XP Ultracentrifuge (Beckman-Coulter, Schaumburg, IL) of 2 mL culture is prepared for GC-FID/GC-MS analysis of geraniol. The supernatant aliquots of 2 mL are transferred to 5 mL glass vials (Fisher Scientific Co., Pittsburgh, PA). Then, organic solvent (typically hexane) is added at a 1:1 ratio to a fermentation broth sample (e.g. 2 mL for a 2 mL aqueous solution) for extraction. Following an appropriate extraction (vortex samples for 15 seconds, spin on a rotator at 60 rpm for 2 hours, and vortex again for 15 seconds), 1 mL of the organic phase is removed. 50 μL pyridine and 50 uL BSTFA are then added to the 1 mL organic phase for derivatization, with the reaction allowed to proceed at 70 °C for 30 minutes. After cooling to room temperature, this mixture is used for GC analysis.

GC analysis is conducted on an Agilent 7890B Series Custom Gas Chromatography system equipped with a 5977B Inert Plus Mass Selective Detector Turbo EL Bundle (for identification) or a Flame Ionization Detector (for quantification) and an Agilent HP-5 capillary column (0.25 mm internal diameter, 0.25 μm film thickness, 30 in length). The following temperature profile is used with helium as the carrier gas at a flowrate of 1.5 mL/min: Initial 50 °C (hold 3 min); ramp at 20 °C/min to 270 °C (hold 6 min). The injector and detector temperature are 250 °C and 350 °C, respectively. 1 uL of sample is injected with a 4:1 split ratio.

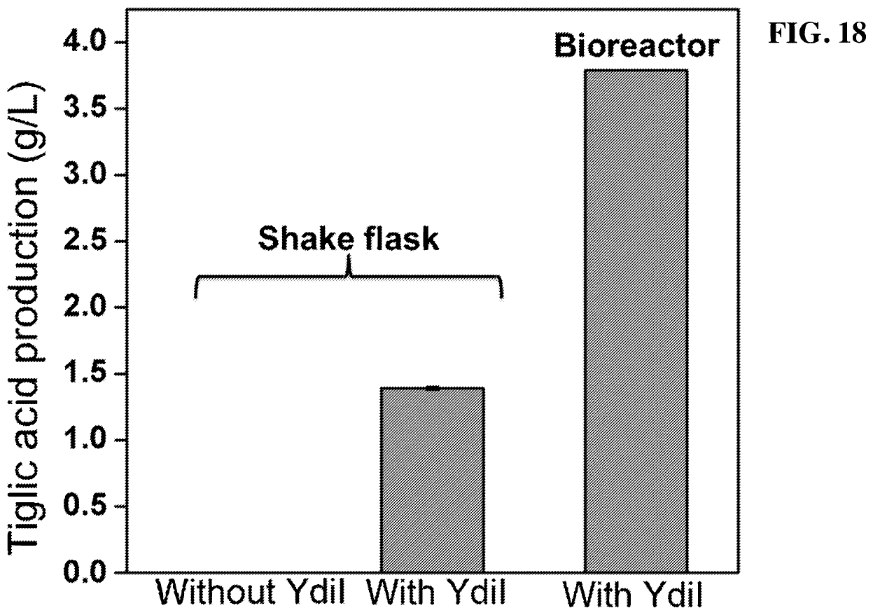

Among above enzymes, the activities of thiolase FadAx, hydroxyacyl-CoA dehydrogenase FadB2x and enoyl-CoA hydratase FadB1x, required for the above described GPP synthesizing reverse beta-oxidation pathways, have already been demonstrated in vivo. JST06(DE3) overexpressing these enzymes along with E. coli thioesterase YdiI (NP_416201.1) and Pct have been grown in shake flasks with 20 g/L glycerol and 20 mM propionic acid for 48 hours at 20 mL volume and in a controlled bioreactor for 72 hours with 30 g/L glycerol and supplementation of 20 mM propionic acid every 24 hours, both induced by induced by 5 μM IPTG at 37 °C, leading to production of 1.39 g/L of 2-methyl-2-butenoic acid or tiglic acid in shake flasks, and 3.79 g/L of tiglic acid in bioreactors (FIG. 18). If YdiI is not overexpressed, no tiglic acid production was detected, indicating that YdiI is able to hydrolyze 2-methyl-2-butenoyl-CoA (tiglyl-CoA), generated through FadAx condensation between primer acetyl-CoA and extender unit propionyl-CoA and subsequent beta-reduction steps by FadB2x and FadB1x, to tiglic acid.

In the above demonstration, the genes encoding FadAx and Pct were expressed from pCDF-P1-pct-fadAx and the genes encoding FadB1x, FadB2x and YdiI were expressed from pET-P1-fadB2x-fadB1x-P2-ydiL The primers used in constructions of these plasmids are listed in Table M. For the construction of pCDF-P1-pct-fadAx, the pct gene insert was first PCR amplified with pct-f1/pct-r1 primers and inserted into vector pCDFDuet-1 (Novagen, Darmstadt, Germany) cleaved by NcoI and EcoRI through In-Fusion HD Eco-Dry Cloning system (Clontech Lab., CA) to construct pCDF-P1-pct. Then, the fadAx gene insert was PCR amplified with fadAx-f1/fadAx-r1 and inserted into vector pCDF-P1-pct cleaved by EcoRI through In-Fusion cloning, generating pCDF-P1-pct-fadAx. For the construction of pET-Pt-fadB2x-fadB1x-P2-ydiI, the fadB2x gene insert was first PCR amplified with fadB2x-f1/fadB2x-r1 primers and inserted into vector pETDuet-1 (Novagen, Darmstadt, Germany) cleaved by NcoI and EcoRI through In-Fusion cloning, generating pET-P1-fadB2x. Then, the fadB1x gene insert was PCR amplified with fadB1x-f1/fadB1x-r1 primers and inserted into pET-P1-fadB2x cleaved by EcoRI through In-Fusion cloning, generating pET-P1-fadB2x-fadB1x. Finally, the ydiI gene insert was PCR amplified with ydiI-f1/ydiI-r1 primers and inserted into pET-P1-fadB2x-fadB1x cleaved by NdeI (New England Biolabs, Ipswich, MA, USA) through In-Fusion cloning, generating pET-P2-fadB2x-fadB1x-P2-ydiI. Before the introduction to host strain, the sequences of constructed plasmids were confirmed by DNA sequencing.

Two plasmids for expressing the pathway that converts prenol to GPP and geraniol (or “Lower alcohol pathway” as shown in FIG. 1) and can be used in above pathway have been constructed and are listed in Table N. To construct pET-P1-idi-trGPPS2-P2-ges-ychB-mtipk and pET-P1-idi-trGPPS2-P2-ges-thaipk-mtipk, the gene inserts encoding Idi and trGPPS2 (“tr” means “truncated” as first 84 aa of GPPS2 was truncated to improve the activity) were PCR amplified with idi-f1/idi-r1 and trgpps2-f1/trgpps2-r1 respectively and inserted together into pETDuet-1 cleaved by NcoI through In-Fusion HD Eco-Dry Cloning system to generate pET-P1-idi-trGPPS2. Then, the gene insert encoding GES was PCR amplified with ges-f1/ges-r1 primers and inserted into vector pET-P1-idi-trGPPS2 cleaved by NdeI and KpnI through In-Fusion HD Eco-Dry Cloning system to generate pET-P1-idi-trGPPS2-P2-ges. When constructing pET-P1-idi-trGPPS2-P2-ges-ychB-mtipk, the gene inserts encoding YchB and MtIPK were PCR amplified with ychB-f1/ychB-r1 and mtipk-f1/mtipk-r1 respectively and inserted together into pET-P1-idi-trGPPS2-P2-ges cleaved by XhoI through In-Fusion HD Eco-Dry Cloning system to generate pET-P1-idi-trGPPS2-P2-ges-ychB-mtipk. When constructing pET-P1-idi-trGPPS2-P2-ges-thaipk-mtipk, the gene insert encoding ThaIPK (with V73I, Y141V and K204G mutations) was PCR amplified with thaipk-f1/thaipk-r1 and inserted into pET-P1-idi-trGPPS2-P2-ges cleaved by XhoI through In-Fusion HD Eco-Dry Cloning system to generate pET-P1-idi-trGPPS2-P2-ges-thaipk, and then the gene encoding MtIPK was PCR amplified with mtipk-f2/mtipk-r1 and inserted into pET-P1-idi-trGPPS2-P2-ges-thaipk cleaved by XhoI through In-Fusion HD Eco-Dry Cloning system to generate pET-P1-idi-trGPPS2-P2-ges-thaipk-mtipk. The sequences of required primers can be seen in Table N. The sequences of constructed plasmids were further confirmed by DNA sequencing. Then, the sequence confirmed plasmids were introduced to competent cells of the host strain.

Among above enzymes, in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned below, they did not show the activity on prenol oxidization.

For the tested enzymes, E. coli enzymes were expressed in pCA24N-gene (-gfp) plasmids from the ASKA collection (Kitagawa et al., 2005). Gene encoding Maqu_2507 and ChnD were codon optimized and synthesized by either GeneArt or GenScript. The gene encoding CbjALD was amplified from the genomic DNA of C. beijerinckii. The primers required for cloning of these genes are listed in Table O. The cbjALD gene insert was PCR amplified from the genomic DNA of C. beijerinckii with cbjALD-f1 and cbjALD-r1 primers and inserted into vector pCDFDuet-1 cleaved by EcoRI through In-Fusion HD Eco-Dry Cloning system to construct pCDF-ntH6-cbjALD. The sequence of the cbjALD gene insert was further confirmed by DNA sequencing. The protein was expressed with an n-terminal 6 His-tag.

The codon-optimized maqu_2507 gene insert was PCR amplified with maqu_2507-f1 and maqu_2507-r1 primers and inserted into vector pCDFDuet-1 (Novagen, Darmstadt, Germany) cleaved by EcoRI through In-Fusion HD Eco-Dry Cloning system to construct pCDF-ntH6-maqu_2507. The sequence of the maqu_2507 gene insert was further confirmed by DNA sequencing. The protein was expressed with an n-terminal 6 His-tag.

The codon-optimized chnD gene insert was PCR amplified with chnD-f1 and chnD-r1 primers and inserted into vector pCDFDuet-1 (Novagen, Darmstadt, Germany) cleaved by EcoRI through In-Fusion HD Eco-Dry Cloning system to construct pCDF-ntH6-chnD. The sequence of the chnD gene insert was further confirmed by DNA sequencing. The protein was expressed with an n-terminal 6 His-tag.

For expression of enzymes, cultures were grown in 25 mL of LB media in 125 mL flasks (Wheaton Industries, Inc., Millville, NJ) at 37 °C. A single colony of the desired strain was cultivated overnight (14-16 hrs) in 10 mL of LB medium in baffled flasks (Wheaton Industries, Inc., Millville, NJ) with appropriate antibiotics and used as the inoculum (3%). The cells were induced with 0.1 mM IPTG at an OD550~0.6.

After post-induction growth for 4 h for ASKA strains, or 16 for other strains, the cells were collected and washed twice by 9 g/L sodium chloride solution. Cells were then re-suspended in lysis buffer (50 mM NaH₂PO₄, 300 mM NaCl, 10 mM imidazole, pH 8.0) to an OD ~40. After re-suspension, the cells were disrupted using glass beads and then centrifuged at 4 °C, 13000 g, 10 min in an Optima L-80XP Ultracentrifuge (Beckman-Coulter, Schaumburg, IL). The resultant supernatant is the crude enzyme extract. The His-tagged enzymes were then purified from crude extract by using Ni-NTA spin kit (Qiagen, Valencia, CA). The crude extracts are centrifuged (270 g, 5 min) in spin columns that were equilibrated with lysis buffer and then washed twice by wash buffer (50 mM NaH₂PO₄, 300 mM NaCl, 20 mM imidazole, pH 8.0). After washing, the enzyme was eluted twice in elution buffer (50 mM NaH₂PO₄, 300 mM NaCl, 500 mM imidazole, pH 8.0). Both washing and elution steps are centrifuged at 890 g for 2 min. The purified enzyme extracts were then further concentrated and dialyzed through Amicon® Ultra 10K Device (Millipore, Billerica, MA). The enzymes were first filtered by centrifugation at 4 °C, 14000 g, 10 min, and then washed with 100 mM potassium phosphate, pH 7 buffer under the same centrifugation conditions. Finally, the concentrated and dialyzed enzymes were recovered through 4 °C, 1000 g, 2 min centrifugation. The protein concentration was established using the Bradford Reagent (Thermo Scientific, Waltham, MA) using BSA as the protein standard. SDS-PAGE monitor of purified proteins was performed through XCell SureLock™ Mini-cell system (Invitrogen, Carlsbad, CA) with gels (12% acrylamide resolving gel and 4% acrylamide stacking gel) prepared through SureLock™ Mini-cell system (Invitrogen, Carlsbad, CA). The composition of the running buffer for SDS-PAGE was 3 g/L tris base, 14.4 g/L glycine and 1 g/L SDS in water.

Enzymatic reactions were monitored on either a Synergy HT plate reader (BioTek Instruments, Inc., Winooski, VT) or a Biomate 5 Spectrophotometer (Thermo Scientific, Waltham, MA) according to established protocols. Measurement of 3-methylcrotonyl-CoA reduction by acyl-CoA reductases was measured by following the decrease (oxidation of NAD(P)H) in absorbance at 340 nm from a reaction mixture containing 100 mM Tris-HCl (pH 7.5). 5 mM DTT, 0.3 mM NAD(P)H, and 1 or 5 mM 3-methylcrotonyl-CoA. Measurement of alcohol dehydrogenase activity on prenol was measured by following the increase (reduction of NAD(P)+) in absorbance at 340 nm from a reaction mixture containing 100 mM Tris-HCl (pH 8.0), 1 mM NAD(P) %, and 1 mM prenol.

For assays of acyl-CoA reductases, the crude extract of CbjALD did not show the detectable reduction activity on 1 mM 3-methylcrotonyl-CoA, but the activity was detected (0.008 μmol/mg/min) when the enzyme was purified and the concentration of 3-methylcrotonyl-CoA was 5 mM. The crude extract of Maqu 2507 showed 0.08±0.01 μmol/mg/min towards 1 mM 3-methylcrotonyl-CoA. These results indicate that CbjALD and Maqu_2507 are suitable for reduction of 3-methylcrotonyl-CoA to prenol. CbjALD uses NADH as cofactor, while Maqu_2507 uses NADPH as cofactor.

Among the assayed alcohol dehydrogenases, YahK, YjgB and ChnD showed the activity on oxidization of prenol to 3-methyl-1-butenal. They should be suitable for catalyzing the required reverse reduction reaction of 3-methyl-1-butenal, which is converted from 3-methylcrotonyl-CoA by CbjALD, to prenol. The results are shown in Table P.

3-methylcrotonyl-CoA, which is then converted to GPP via prenol through the pathway described above, can also be supplied through two different versions of reverse beta-oxidation pathways incorporated with methyl-group transferring mutase. The first pathway starts from non-decarboxylative Claisen condensation between propionyl-CoA, which serves as the primer, and glycolyl-CoA, which serves as the extender unit, catalyzd by thiolase. In this pathway, propionyl-CoA is activated from propionic acid, which is either supplemented or synthesized through overexpressed native pathway of conversion of succinate to propionic acid, while glycolyl-CoA is activated from glycolic acid, which is either supplemented or synthesized through overexpressed native pathway of conversion of glyoxylate, the intermediate of glyoxylate shunt, to glycolic acid. The activations of both propionic acid and glycolic acid are catalyzed by Pct. After three beta-reduction steps catalyzed by hydroxyacyl-CoA dehydrogenase, enoyl-CoA hydratase and enoyl-CoA reductases, 2-hydroxypentanoyl-CoA is generated. Then, mutase moves the methyl group from γ-site to beta-site on 2-hydroxypentanoyl-CoA, generating 2-hydroxy-3-methylbutanoyl-CoA, and 2-hydroxyacyl-CoA dehydratase converts 2-hydroxy-3-methylbutanoyl-CoA to 3-methylcrotonyl-CoA. The second pathway starts from non-decarboxylative Claisen condensation between propionyl-CoA, which serves as the primer, and acetyl-CoA, which serves as the extender unit, catalyzd by thiolase. In the pathway, propionyl-CoA is activated from propionic acid, which is either supplemented or synthesized through overexpressed native pathway of conversion of succinate to propionic acid, catalyzed by Pct. After two beta-reduction steps catalyzed by hydroxyacyl-CoA dehydrogenase and enoyl-CoA hydratase, 3-hydroxypentanoyl-CoA is generated. Then, mutase moves the methyl group from -site to beta-site on 3-hydroxypentanoyl-CoA, generating 3-hydroxy-3-methylbutanoyl-CoA, and enoyl-CoA hydratase converts 3-hydroxy-3-methylbutanoyl-CoA to 3-methylcrotonyl-CoA.

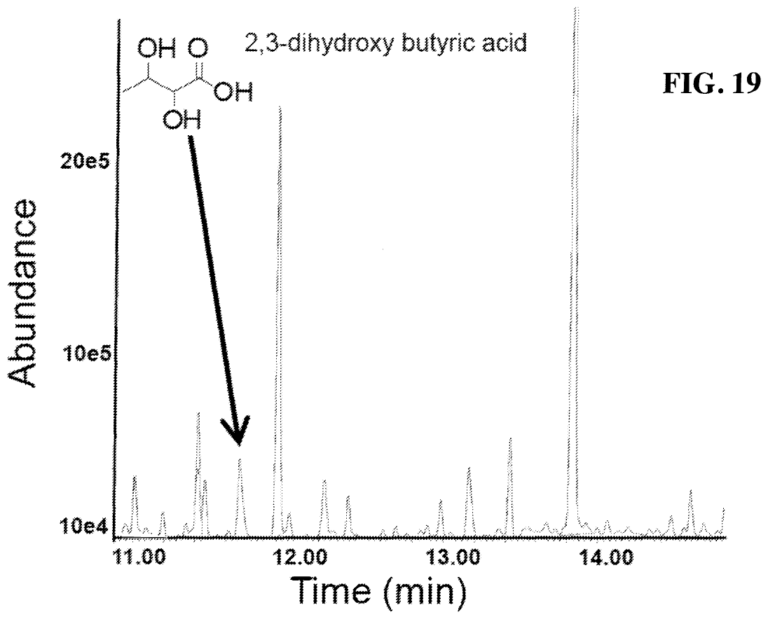

The non-decarboxylative Claisen condensation between primer acetyl-CoA, similar to propionyl-CoA required in the above described pathway, and extender unit glycolyl-CoA, and the subsequent beta-reduction by hydroxyacyl-CoA dehydrogenase have been in vivo demonstrated in E. coli. MG1655(DE3) ΔglcD (glcD gene encoding a subunit of glycolate oxidase was deleted to block degradation of glycolic acid) strain overexpressing thiolase BktB (AAC38322.1) from Ralstonia eutropha, hydroxyacyl-CoA dehydrogenase PhaB1 (P14697.1) from R. eutropha, enoyl-CoA hydratase PhaJ (032472.1) from Aeromonas caviae and enoyl-CoA reductase TdTer (4GGO_A) from Treponema denticola along with activation enzyme Pct, which was supposed to produce 4-hydroxybutyric acid through reverse beta-oxidation pathway starting from non-decarboxylative Claisen condensation between primer glycolyl-CoA and extender unit acetyl-CoA, was also found to produce 2,3-dihydroxybutyric acid detected by GC-MS, after 96 h growth under 30 °C in LB supplemented with glucose and glycolic acid. The GC-MS chromatogram showing the peak of 2,3-dihydroxybutyric acid is shown in FIG. 19. This result indicates that thiolase BktB can accept glycolyl-CoA as extender unit and acetyl-CoA as primer in the condensation, generating 2-hydroxy-3-oxobutanoyl-CoA, and PhaB1 can reduce 2-hydroxy-3-oxobutanoyl-CoA to 2,3-dihydroxybutanoyl-CoA, which is hydrolyzed to 2,3-dihydroxybutyric acid by native E. coli enzymes.

In the strain producing 2,3-dihydroxybutyric acid, genes encoding BktB, PhaB1 and PhaJ were overexpressed from pET-P1-bktB-phaB1-P2-phaJ and genes encoding Pct and TdTer wre overexpressed from pCDF-P1-pct-P2-tdter, as shown in Table Q, along with primer sequences required for construction of these plasmids. The genes used for 2,3-dihydroxybutyric acid production were codon optimized and synthesized by either GeneArt or GenScript, except bktB and phaB1, which were amplified from the genomic DNA of R. eutropha, and pct, which was amplified from the genomic DNA of M. elsdenii. To construct pET-P1-bktB-phaB1-P2-phaJ, the gene insert encoding phaJ was amplified with phaJ-f1/phaJ-r1 and inserted into pETDuet-1 cleaved by NdeI through In-Fusion HD Eco-Dry Cloning system to generate pET-P2-phaJ. Then, the gene insert encoding BktB was PCR amplified with bktB-f1/bktB-r1 and inserted into pET-P2-phaJ cleaved by NcoI and EcoRI through In-Fusion HD Eco-Dry Cloning system to generate pET-P1-bktB-P2-phaJ. Then, the gene insert encoding PhaB1 was PCR amplified with phaB1-f1/phaB1-r1 primers and inserted into vector pET-P1-bktB-P2-phaJ cleaved by EcoRI through In-Fusion HD Eco-Dry Cloning system to generate pET-P1-bktB-phaB1-P2-phaJ. To construct pCDF-P1-pct-P2-tdter, the gene encoding TdTer was was amplified with tdter-f1/tdter-r1 and inserted into pCDFDuet-1 cleaved by NdeI through In-Fusion HD Eco-Dry Cloning system to generate pCDF-P2-tdter. Then, the gene insert encoding pct was PCR amplified with pct-f1/pct-r1 primers and inserted into vector pCDF-P2-tdter cleaved by NcoI and EcoRI through In-Fusion HD Eco-Dry Cloning system to generate pCDF-P1-pct-P2-tdter. The sequences of required primers can be seen in Table Q. The sequences of constructed plasmids were further confirmed by DNA sequencing. Then, the sequence confirmed plasmids were introduced to competent cells of the host strain.

Fermentations for 2,3-dihydroxybutric acid production were conducted in 250 mL Erlenmeyer Flasks filled with 50 mL LB media supplemented with 10 g/L glucose and appropriate antibiotics. A single colony of the desired strain was cultivated overnight (14-16 h) in LB medium with appropriate antibiotics and used as the inoculum (2%). After inoculation, cells were cultivated at 30 °C and 250 rpm in a NBS 124 Benchtop Incubator Shaker (New Brunswick Scientific Co., Inc., Edison, NJ) until an optical density of ~0.8 was reached, at which point IPTG (0.1 mM) and neutralized glycolic acid (40 mM) were added. Flasks were then incubated under the same conditions for 96 hours.

Besides above pathways, there is also a novel pathway of GPP synthesis employing beta-oxidation reversal without usage of methyl-group transferring muase and via 3-methyl-3-butenol (isoprenol) instead of prenol. This pathway starts from non-decarboxylative Claisen condensation between glycolyl-CoA, which serves as the primer, and propionyl-CoA, which serves as the extender unit, catalyzd by thiolase. In this pathway, propionyl-CoA is activated from propionic acid, which is either supplemented or synthesized through overexpressed native pathway of conversion of succinate to propionic acid, while glycolyl-CoA is activated from glycolic acid, which is either supplemented or synthesized through overexpressed native pathway of conversion of glyoxylate, the intermediate of glyoxylate shunt, to glycolic acid. The activations of both propionic acid and glycolic acid are catalyzed by Pct. After three beta-reduction steps catalyzed by hydroxyacyl-CoA dehydrogenase, enoyl-CoA hydratase and enoyl-CoA reductases, 4-hydroxy-2-methylbutanoyl-CoA is generated. 4-hydroxy-2-methylbutanoyl-CoA is converted to 2-methyl-1,4-butanediol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Then, an alcohol dehydratase converts 2-methyl-1,4-butanediol to 3-methyl-3-butenol (isoprenol). Isorenol is then converted to IPP by one or two steps of phosphorylation. If phosphorylated by two steps, the first step is catalyzed by E. coli alcohol kinase YchB (NP_415726.1) and the second step is catalyzed by M. thermautotrophicus phosphate kinase MtIPK (AAB84554.1) or Thermoplasma acidophilum phosphate kinase ThaIPK (WP_010900530.1) or Methanocaldococcus jannaschii phosphate kinase MjIPK (3K4Y_A). The one step phosphorylation is catalyzed by alcohol diphosphokinase. E. coli isopentenyl pyrophosphate isomerase Idi (NP_417365.1) converts DMAPP to IPP. DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimuin basilicuni geraniol stynthase GES (AR11765.1, N-terminal 65 aa truncation) converts GPP to geraniol, the proxy product for the synthesis pathway. For this pathway, JST06(DE3) serves as the E. coli host strain for demonstration. Vector creation, strain creation, growth and analysis of supernatant are conducted as described above.

(Prophetic) GPP Biosynthesis Via 2-Hydroxyisovaleric Acid and Prenol Starting from Decarboxylative Acyloin Condensation Between Two Pyruvates

The purpose of this example is to demonstrate the biosynthesis of GPP through a novel pathway that starts from decarboxylative acyloin condensation between two pyruvates 2-hydroxyisovaleric acid and prenol, using E. coli as the host organism. This pathway starts from decarboxylative acyloin condensation of two pyruvates to (S)-2-acetolactone by B. subtilis acetolactate synthase AlsS (NP_391482.2). E. coli acetohydroxy acid isomeroreductase IlvC (NP_418222.1) converts (S)-2-acetolactone to (2R)-2,3-dihydroxy-3-methylbutyric acid. E. coli dihydroxy acid dehydratase IlvD (YP_026248.1) dehydrates (2R)-2,3-dihydroxy-3-methylbutanoate to 3-methyl-2-oxobutyric acid (2-oxoisovaleric acid). Then, L. lactis 2-hydroxyacid dehydrogenase PanE (AIS03659.1) reduces 2-oxoisovaleric acid to (2R)-3-methyl-2-hydroxybutyric acid (2-hydroxyisovaeleric acid). 2-hydroxyisovaleric acid is then activated to (2R)-3-methyl-2-hydroxybutanoyl-CoA (2-hydroxyisovaleryl-CoA) by acyl-CoA transferase selected from the group consisting M. elsdenii Pet (BAU59368.1) and C. propionicum Pct540 (CAB77207.1, with V193A mutation to enhance the expression in E. coli, Choi et al. 2016). 2-hydroxyisovaleryl-CoA can be directly dehydrated to 3-methyl-2-butenoyl-CoA (3-methylcrotonyl-CoA) by C. difficile 2-hydroxyacyl-CoA dehydratase HadBCI (AJP10092.1, AJP10093.1, AJP10091.1 or C. propionicum 2-hydroxyacyl-CoA dehydratase LcdABC (G3KIM4.1, G3KIM3.1, G3KIM5.1. HadBCI is originally a 2-hydroxyisocaproyl-CoA dehydratase (Kim et al. 2005). LcdABC is originally a lactonyl-CoA dehydratase. (Hofmeister et al. 1992).

2-hydroxyisovaleryl-CoA can also be converted to 3-methylcrotonyl-CoA by a multi-step pathway. In that pathway, 2-hydroxyisovaleryl-CoA is first reduced to (2R)-3-methyl-1,2-butanediol catalyzed by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. (2R)-3-methyl-1,2-butanediol is dehydrated to 3-methylbutanal by diol dehydratase which is then converted to isovaleryl-CoA by aldehyde-forming acyl-CoA reductase. Isovaleryl-CoA is converted to 3-methylcrotonyl-CoA by P. aeruginosa acyl-CoA dehydrogenase acyl-CoA dehydrogenase LiuA (APJ52511.1).

3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. ChnD (BAC80217.1).

In another route, 2-hydroxyisovaleric acid is dehydrated to 3-methylcrotonic acid by 2-hydroxyacid dehydratase. 3-methylcrotonic acid is either activated to 3-methylcrotonyl-CoA, which is then converted to prenol by alcohol-forming acyl-CoA reductase or aldehyde forming acyl-CoA reductase and alcohol dehydrogenase, or directly converted to prenol by two step reductions by carboxylate reductase and alcohol dehydrogenase. Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP_010900530.1, V73I, Y141V and K204G mutations to increase specificity on prenol (Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimum basilicum geraniol stynthase GES (AR11765.1, N-terminal 65 aa truncation) converts GPP to geraniol, the proxy product for the synthesis pathway. Because 2-hydroxyacyl-CoA dehydratase is oxygen-sensitive, the strain harboring this pathway is grown under microaerobic or anoxic or anaerobic conditions.

As above, JST06(DE3) serves as the E. coli host strain for demonstration of novel pathway. The genes for overexpression are either cloned into appropriate vectors or inserted into chromosome with strong synthetic constitutive promoter M1-93, as described in the previous example. Transformed cells are grown, and supernatant analyzed, also as described in the previous example.

Among above enzymes, as mentioned in the previous example, the in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned above, they did not show the activity on prenol oxidization. The results of assays on alcohol dehydrogenases can be seen in Table P, and the results of assays on acyl-CoA reductases and relevant enzyme preparation and assay methods are described in the previous example.

The in vitro activities of acyl-CoA transferases Pct and Pct540 on activation of 2-hydroxyisovaleric acid to 2-hydroxyisovaleryl-CoA have also been proven through enzymatic spectrophotometric assay.

Genes encoding Pct540 was codon optimized and synthesized by GeneArt. The gene encoding Pct was PCR amplified from the genomic DNA of M. elsdenii. The primers required for cloning of these genes are listed in Table R. The pct gene insert was PCR amplified from the genomic DNA of Megasphaera elsdenii with pct-f2 and pct-r2 primers and inserted into vector pUCBB-ctH6-eGFP (Vick et al. 2011) cleaved by BglII and XhoI through In-Fusion HD Eco-Dry Cloning system to construct pUCBB-ctH6-pct. The sequence of the pct gene insert was further confirmed by DNA sequencing. The protein was expressed with a c-terminal 6 His-tag.

The codon-optimized pct540 gene insert was PCR amplified with pct540-f1 and pct540-r1 primers and inserted into vector pTrcHis2A (Invitrogen, Carlsbad, CA) cleaved by NcoI and SalI through In-Fusion HD Eco-Dry Cloning system to construct pTH-ctH6-pct540. The sequence of the pct540 gene insert was further confirmed by DNA sequencing. The protein was expressed with a c-terminal 6 His-tag. The sequence-confirmed plasmids were introduced into BL21(DE3) (Studier et al. 1986).

For expression of enzymes, cultures were grown in 25 mL of LB media in 125 mL flasks (Wheaton Industries, Inc., Millville, NJ) at 37 °C. A single colony of the desired strain was cultivated overnight (14-16 hrs) in 10 mL of LB medium in baffled flasks (Wheaton Industries, Inc., Millville, NJ) with appropriate antibiotics and used as the inoculum (3%). Except for the expression of pct, the cells were induced with 0.1 mM IPTG at an OD550~0.6, while pct was expressed constitutively.

After post-induction growth for 16 hours, the cells were collected and washed twice by 9 g/L sodium chloride solution. Cells were then re-suspended in lysis buffer (50 mM NaH₂PO₄, 300 mM NaCl, 10 mM imidazole, pH 8.0) to an OD ~40. After re-suspension, the cells were disrupted using glass beads and then centrifuged at 4 °C, 13000 g, 10 min in an Optima L-80XP Ultracentrifuge (Beckman-Coulter, Schaumburg, IL). The resultant supernatant is the crude enzyme extract. The His-tagged enzymes were then purified from crude extract by using Ni-NTA spin kit (Qiagen, Valencia, CA). The crude extracts are centrifuged (270 g, 5 min) in spin columns that were equilibrated with lysis buffer and then washed twice by wash buffer (50 mM NaH₂PO₄, 300 mM NaCl, 20 mM imidazole, pH 8.0). After washing, the enzyme was eluted twice in elution buffer (50 mM NaH₂PO₄, 300 mM NaCl, 500 mM imidazole, pH 8.0). Both washing and elution steps are centrifuged at 890 g for 2 min. The purified enzyme extracts were then further concentrated and dialyzed through Amicon® Ultra 10K Device (Millipore, Billerica, MA). The enzymes were first filtered by centrifugation at 4 °C, 14000 g, 10 min, and then washed with 100 mM potassium phosphate, pH 7 buffer under the same centrifugation conditions. Finally, the concentrated and dialyzed enzymes were recovered through 4 °C, 1000 g, 2 min centrifugation. The protein concentration was established using the Bradford Reagent (Thermo Scientific, Waltham, MA) using BSA as the protein standard. SDS-PAGE monitor of purified proteins was performed through XCell SureLock™ Mini-cell system (Invitrogen, Carlsbad, CA) with gels (12% acrylamide resolving gel and 4% acrylamide stacking gel) prepared through SureLock™ Mini-cell system (Invitrogen, Carlsbad, CA). The composition of the running buffer for SDS-PAGE was 3 g/L tris base, 14.4 g/L glycine and 1 g/L SDS in water.

Enzymatic reactions were monitored on either a Synergy HT plate reader (BioTek Instruments, Inc., Winooski, VT) or a Biomate 5 Spectrophotometer (Thermo Scientific, Waltham, MA) according to established protocols.

Measurement of acyl-CoA transferase activity was conducted in a two-step reaction in which the residual amount of acetyl-CoA after incubation of the enzyme with the substrate of interest was measured. Each assay was carried out in 100 mM Tris-HCL (pH 7.4). First, 0.1 mM acetyl-CoA and 1 or 10 mM of the substrate was incubated with purified enzyme for 15 min at 30 °C. After denaturation of the enzyme (90 s at 95 °C), 0.1 mM oxaloacetate, 5 μg citrate synthase and 0.5 mM DTNB were added, and the reaction was further incubated for 15 min at 30 °C. The amount of generated CoASH was determined by measuring the absorbance at 412 nm.

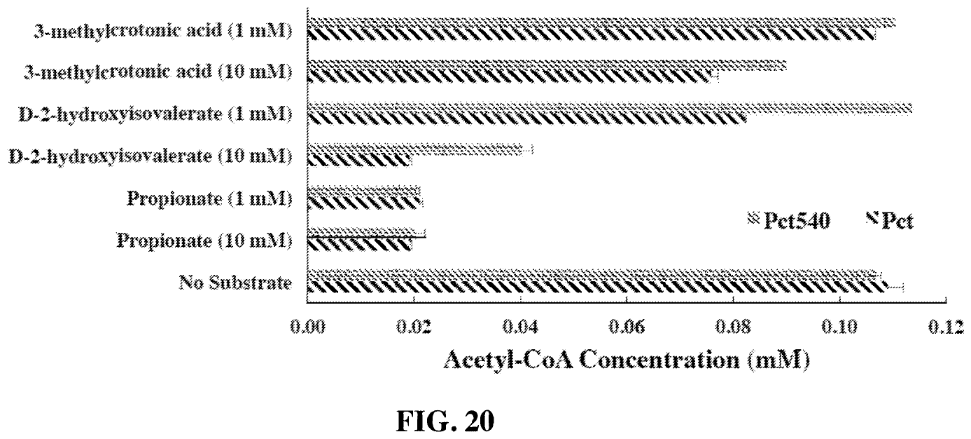

Pet and Pct540 were assayed on CoA transfer from acetyl-CoA to three different substrates: original substrate propionic acid, 2-hydroxyisovaleric acid and 3-methylcrotonic acid, which are required for this novel GPP synthesis pathway. The results of activation of different substrates by Pct and Pct540 are shown in FIG. 20. Pct and Pct540 were shown to have slight activity towards 3-methylcrotonic acid. These enzymes have higher activity towards 2-hydroxyisovaleric acid, and the activity of Pct is higher than that of Pct540, though their activities on 2-hydroxyisovaleric acid are lower than those on original substrate propionic acid. Thus, Pet and Pct540 are suitable acyl-CoA transferases for activation of 2-hydroxyisovaleric acid.

Also, JST06(DE3) strain overexpressing B. subtilis acetolactate synthase AlsS, E. coli acetohydroxy acid isomeroreductase IlvC, E. coli dihydroxy acid dehydratase IlvD and Lactococcus lactis 2-hydroxyacid dehydrogenase PanE—the enzymes of first four steps of the pathway—have been grown in shake flasks with 20 mL LB-like MOPS supplemented with 20 g/L glycerol or 32 g/L glucose (55 g/L CaCO₃ was also added when glucose was used) for 48 hours under 37 °C with 5 μM IPTG induction. The genes encoding AlsS, IlvC, IlvD and PanE were expressed from the plasmid pET-P1-ilvC-ilvD-P2-alsS-panE. The genes encoding AlsS and PanE were codon optimized and synthesized by either GeneArt or GenScript, while the genes encoding IlvC and IlvD were amplified from genomic DNA of wild type E. coli MG1655 strain. The plasmids used for the construction of plasmid are listed in Table R. The codon-optimized alsS and panE gene inserts were PCR amplified with alsS-f1/alsS-r1 and panE-f1/panE-r1 primers respectively and inserted together into vector pETDuet-1 cleaved by NdeI through In-Fusion HD Eco-Dry Cloning system, resulting in pET-P2-alsS-panE plasmid. The ilvC and ilvD gene inserts were PCR amplified from the genomic DNA of E. coli with ilvC-f1/ilvC-r1 and ilvD-f1/ilvD-r1 primers respectively, and inserted together into vector pET-P2-alsS-panE cleaved by NeoI and EcoRI through In-Fusion HD Eco-Dry Cloning system, generating pET-P1-ilvC-ilvD-P2-alsS-panE. The sequences of constructed plasmids are further confirmed by DNA sequencing. The quantification of 2-hydroxyisovaleric acid was performed via ion-exclusion HPLC using a Shimadzu Prominence SIL 20 system (Shimadzu Scientific Instruments, Inc., Columbia, MD) equipped with an HPX-87H organic acid column (Bio-Rad, Hercules, CA) with operating conditions to optimize peak separation (0.3 mL/min flow rate, 30 mM H₂SO₄ mobile phase, column temperature 42 °C). Concentration of 2-hydroxyisovaleric acid in fermentation samples was determined through calibration to known 2-hydroxyisovaleric acid standards (5, 1, 0.5, 0.2 and 0.1 g/L).

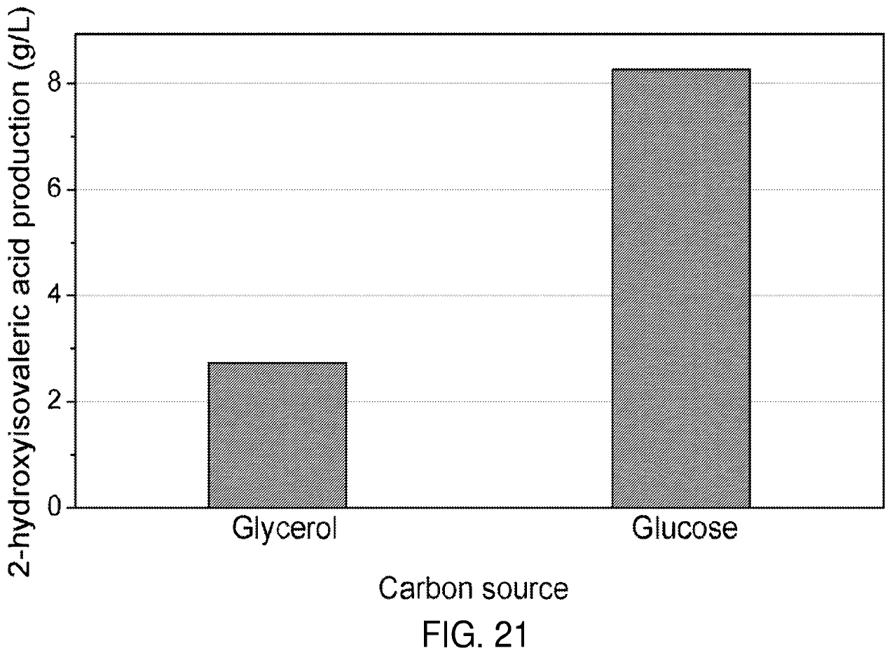

As shown in FIG. 21, this strain shows high production of 2-hydroxyisovaleric acid, especially when glucose was used as carbon source, in which the titer was 8.27 g/L. This indicates that AlsS, IlvC, IlvD and PanE can supply 2-hydroxyisovaleric acid with high flux, providing sufficient intermediates supply for the subsequent conversion into prenol and GPP.

Plasmids containing the codon optimized gene encoding 6×HIS-tagged Lmo1179 and PddABC were constructed. The resulting construct was transformed into E. coli BL21(DE3) for expression. The resulting strain was cultured in 50 mL of TB media containing appropriate antibiotics in a 250 mL flask. When the culture reached an OD550 of approximately 0.6, expression was induced by the addition of 0.1 mM IPTG, and the cells were harvested by centrifugation after overnight incubation at room temperature.

The HIS-tagged Lmo1179 protein was purified from the cell extract using Talon Metal Affinity Resin (Clontech lab., CA). In short, a 250 μL resin bed was equilibrated twice using 2.5 mL of a buffer containing 50 mM sodium phosphate, 300 mM NaCl, and 10 mM imidazole at pH 7.5 (NPI-10). The cell extract was added to the resin and the mixture shaken gently for 20 minutes on ice. The resin was then washed twice with 2.5 mL buffer NPI-20 (same as NPI-10 but with 20 mM imidazole), shaking gently on ice for 15 minutes each wash. The resin was then transferred to a gravity column and washed once with 1.25 mL NPI-20. Finally, the desired protein was eluted using 1.25 mL of buffer NPI-250 (same as buffer NPI-10 but with 250 mM imidazole), and the eluate collected in 500 μL fractions.

Clarified cell lysates of BL21(DE3) strain overexpressing His-tagged PddABC was prepared by resuspending a saved pellet in 50 mM potassium phosphate buffer pH 7.5 containing 0.2 M ethylene glycol. The resuspened cells were broken by glass beads and supernatant was reserved after centrifugation. Assays were performed by coupling the dehydration of ethylene glycol to acetaldehyde to acyl-CoA reductase Lmo1179 to give acetyl-CoA with the reduction of NAD to NADH, which was monitored at 340 nm. The final assay mixture was 250 μL and contained 50 mM potassium phosphate buffer pH 7.5, 5 mM CoASH, 0.5 mM NAD⁺, 0.2 M ethylene glycol, 7 μL purified Lmol179, 50 μL cell lysate, and 15 μM coenzyme B12 (the cofactor of PddABC). The relevant controls included were no cell lysates (replaced with 50 μL of buffer) and no coenzyme B12.

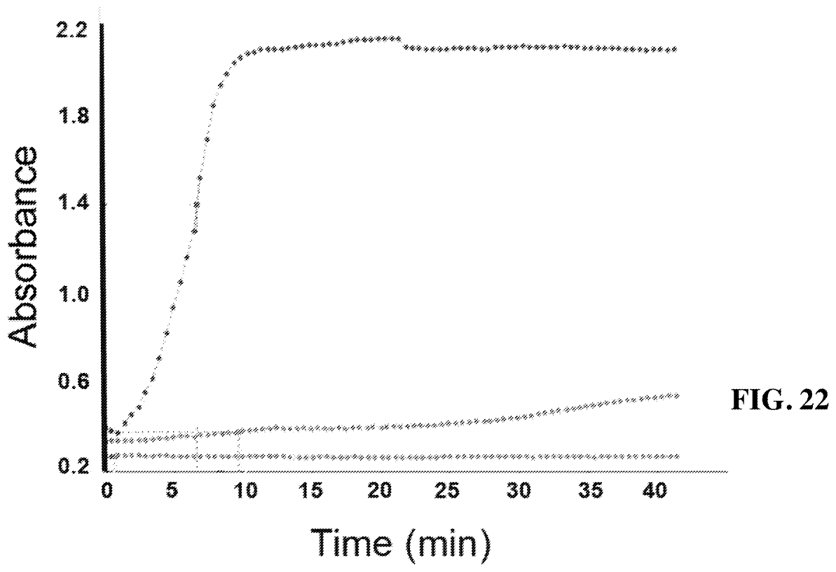

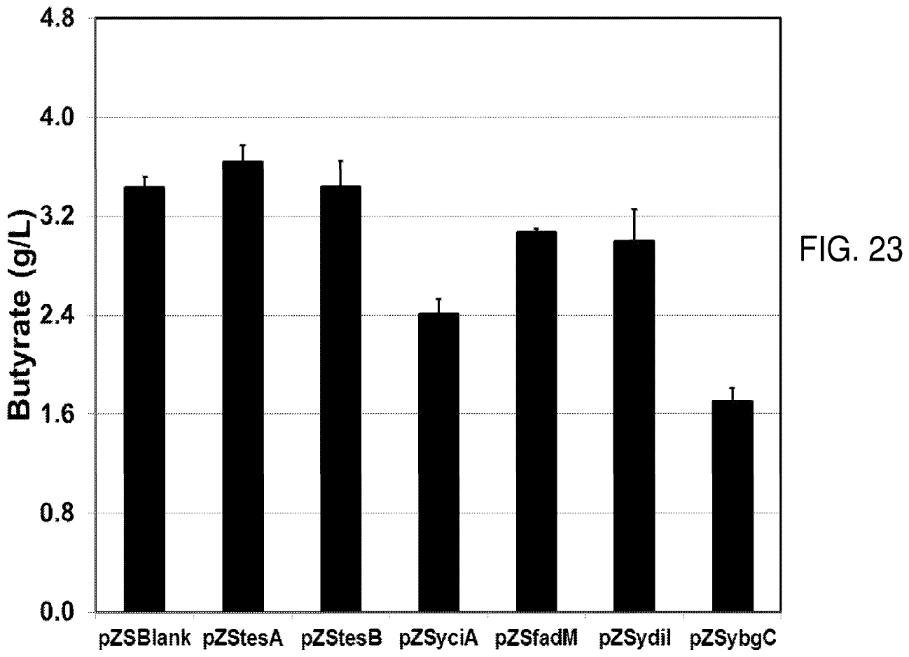

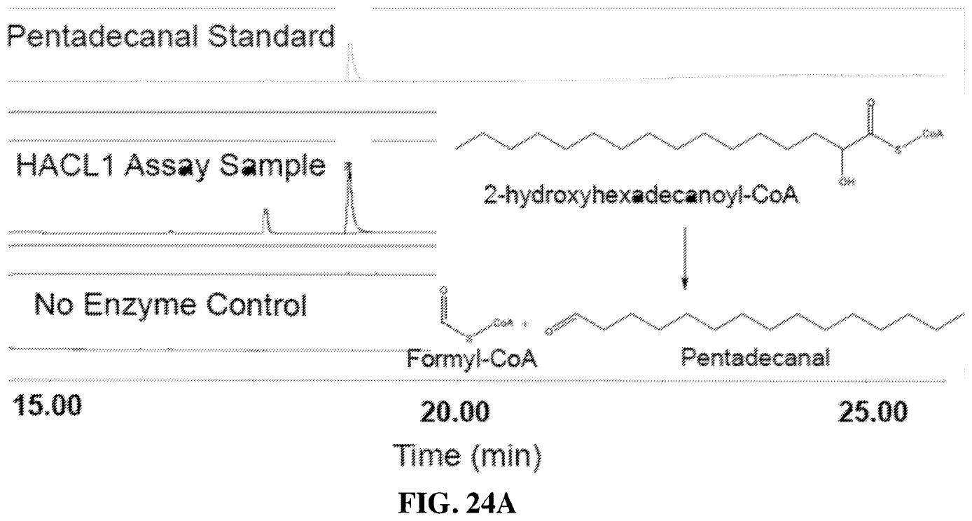

The in vitro activity of dehydration of ethylene glycol to acetaldehyde by diol dehydratae PddABC (AFJ04717.1, AFJ04718.1, AFJ04719.1) from Klebsiella oxytoca has been proven, as shown in FIG. 22. This assay was coupled with oxidization of resultant acetaldehyde to acetyl-CoA by Listeria monocytogenes acyl-CoA reductase (ACR) Lmo1179 (CAC99257.1) and the activity was measured through observation of increased NADH absorbance. Based on these results, PddABC should be a good candidate of diol dehydratase for dehydration of (2R)-3-methyl-1,2-butanediol required for GPP synthesis pathway.

(Prophetic) GPP Biosynthesis Via 2-Hydroxyisovaleric Acid and Prenol Starting from Aldol Condensation Between Acetaldehyde and Pyruvate

The purpose of this experiment is to demonstrate the biosynthesis of GPP through a novel pathway that starts from aldol condensation between pyruvate and acetaldehyde via 2-hydroxyisovaleric acid and prenol, using E. coli as the host organism. This pathway starts from aldol condensation between pyruvate and acetaldehyde to (S)-4-hydroxy-2-oxopentaonoic acid by E. coli aldolase MhpE (NP_414886.1). Acetaldehyde is supplied either through decarboxylation of pyruvate by Saccharomyces cerevisiae alpha-keto acid decarboxylase PDC1 (CAA97573.1) or through reduction of acetyl-CoA by E. coli aldehyde forming acyl-CoA reductase MhpF (NP_414885.1). Then, a mutase moves the —(C═O)COOH group of (S)-4-hydroxy-2-oxopentaonic acid from C-3 site to C-4 site, forming 3-hydroxy-2-oxo-3-methylbutyric acid. 2-hydroxyacid dehydrogenase converts 3-hydroxy-2-oxo-3-methylbutyric acid to (2R)-2,3-dihydroxy-3-methylbutyric acid. E. coli dihydroxy acid dehydratase IlvD (YP_026248.1) dehydrates (2R)-2,3-dihydroxy-3-methylbutanoate to 3-methyl-2-oxobutyric acid (2-oxoisovaleric acid). Then, L. lactis 2-hydroxyacid dehydrogenase PanE (AIS03659.1) reduces 2-oxoisovaleric acid to (2R)-3-methyl-2-hydroxybutyric acid (2-hydroxyisovaeleric acid). 2-hydroxyisovaleric acid is then activated to (2R)-3-methyl-2-hydroxybutanoyl-CoA (2-hydroxyisovaleryl-CoA) by acyl-CoA transferase selected from the group consisting M. elsdenii Pct (BAU59368.1) and C. propionicum Pct540 (CAB77207.1, with V193A mutation to enhance the expression in E. coli, Choi et al. 2016). 2-hydroxyisovaleryl-CoA can be directly dehydrated to 3-methyl-2-butenoyl-CoA (3-methylcrotonyl-CoA) by C. difficile 2-hydroxyacyl-CoA dehydratase HadBCI (AJP10092.1, AJP10093.1, AJP10091.1. HadBCI is originally a 2-hydroxyisocaproyl-CoA dehydratase, Kim et al. 2005) or C. propionicum 2-hydroxyacyl-CoA dehydratase LcdABC (G3KIM4.1, G3KIM3.1, G3KIM5.1. LcdABC is originally a lactonyl-CoA dehydratase, Hofmeister et al. 1992). 2-hydroxyisovaleryl-CoA can also be converted to 3-methylcrotonyl-CoA by a multi-step pathway.

In that pathway, 2-hydroxyisovaleryl-CoA is first reduced to (2R)-3-methyl-1,2-butanediol catalyzed by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. (2R)-3-methyl-1,2-butanediol is dehydrated to 3-methylbutanal by diol dehydratase which is then converted to isovaleryl-CoA by aldehyde-forming acyl-CoA reductase. Isovaleryl-CoA is converted to 3-methylcrotonyl-CoA by P. aeruginosa acyl-CoA dehydrogenase acyl-CoA dehydrogenase LiuA (APJ52511.1). 3-methylcrotonyl-CoA is converted to prenol by an alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. ChnD (BAC80217.1).

In another route, 2-hydroxyisovaleric acid is dehydrated to 3-methylcrotonic acid by 2-hydroxyacid dehydratase. 3-methylcrotonic acid is either activated to 3-methylcrotonyl-CoA, which is then converted to prenol by alcohol-forming acyl-CoA reductase or aldehyde forming acyl-CoA reductase and alcohol dehydrogenase, or directly converted to prenol by two step reductions by carboxylate reductase and alcohol dehydrogenase. Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP 010900530.1, V73I, Y141V and K204G mutations to increase specificity on prenol. Liu et al. 2016) and the second is by M7 thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimum basilicum geraniol stynthase GES (AR11765.1, N-terminal 65 aa truncation) converts GPP to geraniol, the proxy product for the synthesis pathway. Because 2-hydroxyacyl-CoA dehydratase is oxygen-sensitive, the strain harboring this pathway is grown under microaerobic or anoxic or anaerobic conditions.

JST06(DE3) serves as the E. coli host strain for demonstration of this novel pathway. Vector creation, strain creation, growth and analysis of supernatant are as described above in previous examples.

Among above enzymes, as mentioned in the previous example, The in vitro activities of acyl-CoA transferases Pct and Pct540 on activation of 2-hydroxyisovaleric acid to 2-hydroxyisovaleryl-CoA, the in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned above, they did not show the activity on prenol oxidization. The results of assays on alcohol dehydrogenases and acyl-CoA transferases can be seen in Table R and FIG. 20 respectively, and the results of assays on acyl-CoA reductases and relevant enzyme preparation and assay methods are described in the previous example.

The in vitro activity of dehydration of ethylene glycol to acetaldehyde by diol dehydratae PddABC (AFJ04717.1, AFJ04718.1, AFJ04719.1) from Klebsiella oxytoca has been proven, as shown in FIG. 22. The assay method is described in the previous examples.

(Prophetic) GPP Biosynthesis Via 2-Hydroxyisovaleryl-COA and Prenol Starting from Non-Decarboxylative Acyloin Condensation Between Isobutanal and Formyl-COA

The purpose of this experiment is to demonstrate the biosynthesis of GPP through a novel pathway that starts from non-decarboxylative acyloin condensation between formyl-CoA and isobutanal via 2-hydroxyisovaleryl-CoA and prenol, using E. coli as the host organism. This pathway starts from non-decarboxylative acyloin condensation between isobutanal and formyl-CoA to (2R)-3-methyl-2-hydroxybutanoyl-CoA (2-hydroxyisovaleryl-CoA) by Homo sapiens 2-hydroxyacyl-CoA lyase HACL1 (NP_036392.2). Formyl-CoA is activated from formate, which is a byproduct of conversion of pyruvate to acetyl-CoA by E. coli pyruvate-formate lyase PflB (NP_415423.1), catalyzed by activation enzymes selected from the group consisting acyl-CoA synthase, acyl-CoA transferase, carboxylate kinase plus phosphotransacylase. Isobutanal is reduced from isobutyryl-CoA by aldehyde forming acyl-CoA reductase. Isobutyryl-CoA is converted from butyryl-CoA by mutase. Butyryl-CoA can be supplied from butyric acid, either supplemented or intracellularly synthesized through beta-oxidation reversal starting from two acetyl-CoAs composed of ketoacyl-CoA thiolase BktB (AAC38322.1) from R. eutropha or thiolase AtoB (NP_416728.1) from E. coli, hydroxyacyl-CoA dehydrogenase and enoyl-CoA hydratase multifunctional enzyme FadB from E. coli (NP_418288.1) and enoyl-CoA reductase EgTer from E. gracilis (Q5EU90.1) or fatty acid biosynthesis pathway starting from acetyl-CoA and malonyl-CoA composed of beta-ketoacyl-ACP synthase FabH (NP_415609.1), beta-ketoacyl-ACP reductase FabG (NP_415611.1), 3-hydroxyacyl-ACP dehydratase FabZ (NP_414722.1) and enoyl-ACP reductase FabI (NP_415804.1), all from E. coli, with termination by E. coli thioesterase TesA (NP_415027.1, with truncation of 26 aa leader sequence) and activation by E. coli acyl-CoA synthetase FadD (NP_416319.1), or directly synthesized through overexpressed beta-oxidation reversal pathway without termination. If malonyl-CoA is used to enhance its supply, E. coli acetyl-CoA carboxylase AccABCD (NP_414727.1, NP_417721.1, NP_417722.1, NP_416819.1) is overexpressed.

2-hydroxyisovaleryl-CoA can be directly dehydrated to 3-methyl-2-butenoyl-CoA (3-methylcrotonyl-CoA) by C. difficile 2-hydroxyacyl-CoA dehydratase HadBCI (AJP10092.1, AJP10093.1, AJP10091.1. HadBCI is originally a 2-hydroxyisocaproyl-CoA dehydratase. Kim et al. 2005) or C. propionicum 2-hydroxyacyl-CoA dehydratase LcdABC (G3KIM4.1, G3KIM3.1, G3KIM5.1. LcdABC is originally a lactonyl-CoA dehydratase. Hofmeister et al. 1992). 2-hydroxyisovaleryl-CoA can also be converted to 3-methylcrotonyl-CoA by a multi-step pathway. In that pathway, 2-hydroxyisovaleryl-CoA is first reduced to (2R)-3-methyl-1,2-butanediol catalyzed by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. (2R)-3-methyl-1,2-butanediol is dehydrated to 3-methylbutanal by diol dehydratase which is then converted to isovaleryl-CoA by aldehyde-forming acyl-CoA reductase. Isovaleryl-CoA is converted to 3-methylcrotonyl-CoA by P. aeruginosa acyl-CoA dehydrogenase acyl-CoA dehydrogenase LiuA (APJ52511.1). 3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. ChnD (BAC80217.1).

Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP_010900530.1, V73I, Y141V and K204G mutations to increase specificity on prenol (Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimum basilicum geraniol stynthase GES (AR11765.1, N-terminal 65 aa truncation) converts GPP to geraniol, the proxy product for the synthesis pathway. Because diol dehydratase is oxygen-sensitive, the strain harboring this pathway is grown under microaerobic or anoxic or anaerobic conditions.

JST06(DE3) serves as the E. coli host strain for demonstration of this novel pathway. Vector creation, strain creation, growth and analysis of supernatant are as described above in previous examples.

The in vivo butyryl-CoA and butyric acid synthesis through beta-oxidation reversal composed of AtoB, FadB and EgTer has been demonstrated in E. coli. JC01 (MG1655 ΔldhA ΔpoxB Δpta ΔadhE ΔfrdA, an E. coli strain with removal of mixed-acid fermentation for improved supply of acetyl-CoA), overexpressing AtoB, FadB and EgTer produced 3.3 g/L of butyric acid when grown in LB-like MOPS media with glycerol as carbon source for 48 hours, indicating that beta-oxidation reversal composed of AtoB, FadB and EgTer is functional of supplying butyric acid with acetyl-CoA as primer and extender unit, and native endogenous thioesterases are able to hydrolyze butyryl-CoA to butyric acid. Overexpression of different E. coli thoesterases FadM (NP_414977.1), TesA (NP_415027.1), TesB (NP_414986.1), YciA (NP_415769.1), YdiI (NP_416201.1) and YbgC (NP_415264.1) was added, but as seen in FIG. 23, it did not greatly improve butyric acid production. The detailed methods of fermentation conditions and HPLC analysis for butyric acid are described in previous examples.

The vectors and primers used in overexpression of AtoB, FadB, EgTer and thioesterases are listed in Table S. The E. coli genes were PCR amplified from genomic DNA of wild type E. coli strain, while the gene encoding EgTer was codon-optimized and synthesized by GenScript. For the construction of pTH-atoB-fadB-egter, the atoB gene insert was first PCR amplified with atoB-f1/atoB-r1 primers and inserted into vector pTrcHis2A (Invitrogen, Carlsbad, CA) cleaved by NcoI and EcoRI through In-Fusion HD Eco-Dry Cloning system to construct pTH-atoB. Then, the fadB gene insert was PCR amplified with fadB-f1/fadB-r1 primers and inserted into vector pTH-atoB cleaved by HindIII through In-Fusion HD Eco-Dry Cloning system to generate pTH-atoB-fadB. Finally, the egter gene insert was PCR amplified with egter-f1/egter-r1 primers and inserted into vector pTH-atoB-fadB cleaved by HindIII through In-Fusion HD Eco-Dry Cloning system to generate pTH-atoB-fadB-egter. The thioesterases were overexpressed from pZS vector backbone (Invitrogen, Carlsbad, CA). The genes encoding thioesterases were PCR amplified with relevant primers (fadM-f1/fadM-r1, tesA-f1/tesB-r1, tesA-f1/tesA-r1, ydiI-f1/ydiI-r1, ybgC-f1/ybgC-r1, yciA-f1/yciA-r1) and inserted into pZS cleaved by KpnI and MluI through In-Fusion HD Eco-Dry Cloning system.

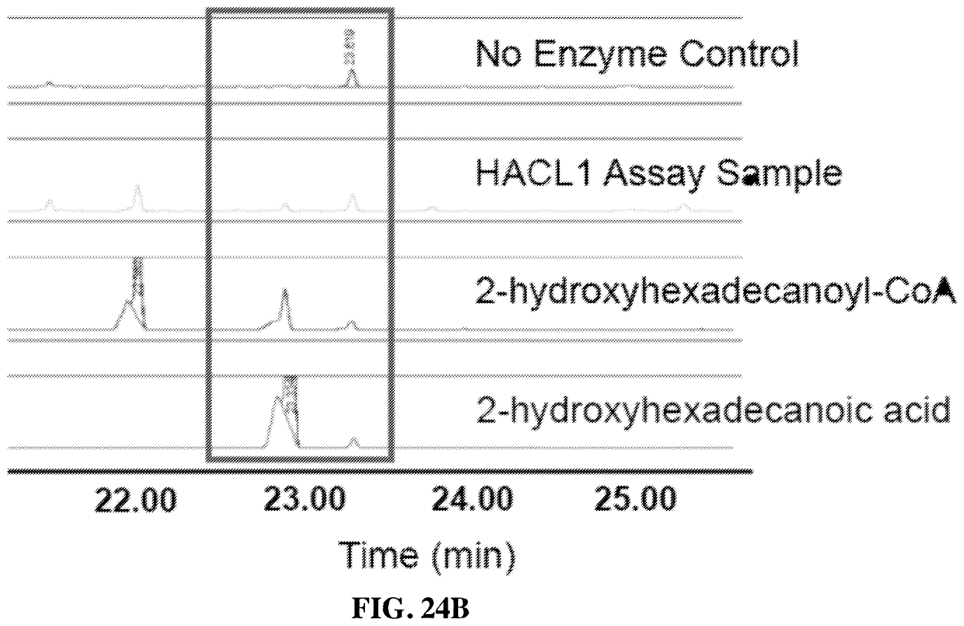

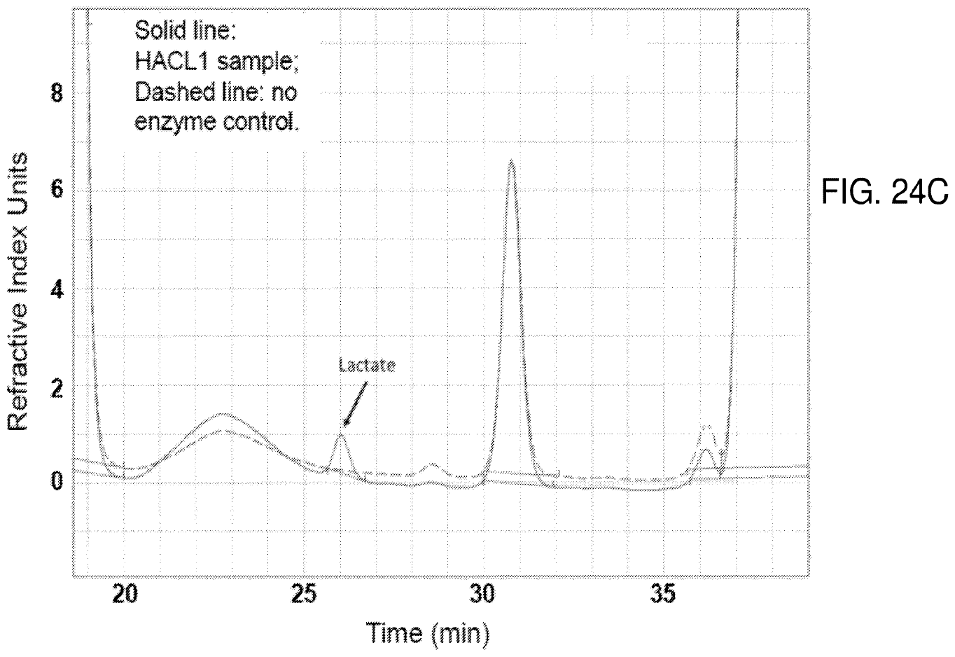

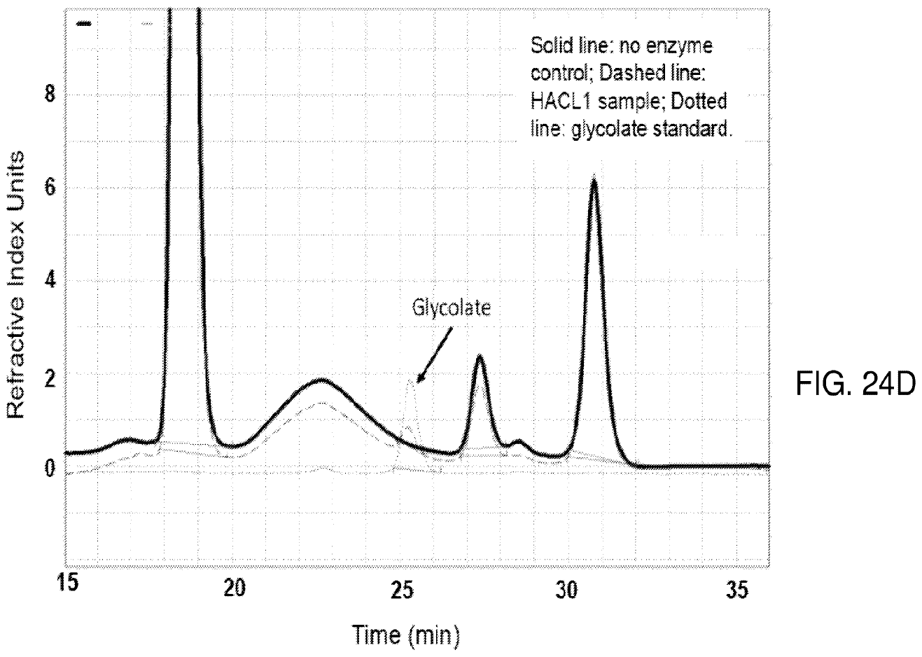

The condensations between formyl-CoA and different kinds of aldehyde (pentadecanal, acetaldehyde, formaldehyde) by 2-hydroxyacyl-CoA lyase HACL1 have been proven in vitro, as shown in FIG. 24A-D. HACL1 is a good candidate to also accept required isobutanal as the substrate for acyloin condensation.

2-hydroxyhexadecanoyl-CoA was prepared by the n-hydroxysuccinimide method (Blecher, 1981). In summary, the n-hydroxysuccinimide ester of 2-hydroxyhexadecanoic acid is prepared by reacting n-hydroxysuccinimide with the acid in the presence of dicyclohexylcarbodiimide. The product is filtered and purified by recrystallization from methanol to give pure n-hydroxysuccinimide ester of 2-hydroxyhexadecanoic acid. The ester is reacted with CoA-SH in presence of thioglycolic acid to give 2-hydroxyhexadecanoyl-CoA. The 2-hydroxyhexadecanoyl-CoA is purified precipitation using perchloric acid, filtration, and washing the filtrate with perchloric acid, diethyl ether, and acetone.

Formyl-CoA was prepared by first forming formic ethylcarbonic anhydride as previously described (Parasaran & Tarbell, 1964). Briefly, formic acid (0.4 mmol) and ethyl chloroformate (0.4 mmol) were combined in 4 mL anhydrous diethyl ether and cooled to −20 °C. 0.4 mmol triethylamine was added to the mixture and the reaction was allowed to proceed at −20 °C for 30 minutes. The reaction mixture was filtered over glass wool to give a solution containing formic ethylcarbonic anhydride in diethyl ether. To obtain formyl-CoA, 7 μmol CoASH was dissolved in 5 mL 3:2 water:tetrahydrofuran, to which 10 mg of sodium bicarbonate were added. The solution of formic ethylcarbonic anhydride was added dropwise to the CoASH solution with vigorous agitation, after which the organic phase was evaporated under a stream of nitrogen. The mixture was kept at 4 °C for two hours, after which any remaining diethyl ether was evaporated under nitrogen. Solid phase extraction using a Cis column was used to purify formyl-CoA from the reaction mixture. Formyl-CoA was eluted from the C₁₈ column in methanol and stored in 2:1 methanol:ammonium acetate pH 5.5.

The resulting cell pellet was resuspended in Bacterial Protein Extraction Reagent (B-PER) (THERMO SCIE., MA) to an OD550 of approximately 40, to which approximately 5000 U of lysozyme and approximately 250 U of Benzonase nuclease (Sigma-Aldrich CO., MO) were added. The cell mixture was left at room temperature until completely clarified to give the cell extract. 1 M stock solution of imidazole was added to provide a final concentration of 10 mM imidazole in the cell extract.

A plasmid containing the codon optimized gene encoding human HIS-tagged HACL1 was constructed as described. The resulting construct, was transformed into S. cerevisiae InvSC1 (Life Technologies, Carlsbad, CA). The resulting strain was cultured in 50 mL of SC-URA media containing 2% glucose at 30 °C for 24 hours. The cells were pelleted and the required amount of cells were used to inoculate a 250 mL culture volume of SC-URA media containing 0.2% galactose, 1 mM MgCl₂, and 0.1 mM thiamine to 0.4 OD600. After 20 hours of incubation with shaking at 30 °C, the cells were pelleted and saved.

When needed, the cell pellets were resuspended to an OD600 of approximately 100 in a buffer containing 50 mM potassium phosphate pH 7.4, 0.1 mM thiamine pyrophosphate, 1 mM MgCl₂, 0.5 mM AEBSF, 10 mM imidazole, and 250 units of Benzonase nuclease. To the cell suspension, approximately equal volumes of 425-600 μm glass beads were added. Cells were broken in four cycles of 30 seconds of vortexing at 3000 rpm followed by 30 seconds on ice. The glass beads and cell debris were pelleted by centrifugation and supernatant containing the cell extract was collected. The HIS-tagged HACL1 was purified from the cell extract using Talon Metal Affinity Resin as described above, with the only modification being the resin bed volume and all subsequent washes were halved. The eluate was collected in two 500 μL fractions.

Human HACL1 was cloned, expressed, and purified in S. cerevisiae as described above. Purified HACL1 was tested for its native catabolic activity by assessing its ability to cleave 2-hydroxyhexadecanoyl-CoA to pentadecanal and formyl-CoA. Enzyme assays were performed in 50 mM tris-HCl pH 7.5, 0.8 mM MgCl₂, 0.02 mM TPP, 6.6 μM BSA, and 0.3 mM 2-hydroxyhexadecanoyl-CoA. The assay mixtures were incubated for one hour at 37 °C, after which the presence of pentadecanal was assessed by extraction with hexane and analysis by GC-FID. As shown in FIG. 24A-D, pentadecanal was produced in the sample containing HACL1, but not in the control sample, which did not contain HACL1, indicating that the protein was expressed and purified in an active form.

The ability of purified HACL1 to run in the anabolic direction (reverse from the physiological direction) was also determined. An aldehyde and formyl-CoA were tested for ligation in a buffer comprised of 60 mM potassium phosphate pH 5.4, 2.5 mM MgCl₂, 0.1 mM TPP, 6.6 μM BSA, 5 mM aldehyde, 20% DMSO, approximately 1 mM freshly prepared formyl-CoA, and approximately 0.5 mg/mL purified HACL1. The reaction was allowed to take place at room temperature for 16 hours, after which acyl-CoAs were hydrolyzed to their corresponding acids by adjusting to pH >12.0. For situations in which a short carbon chain product was expected, for example lactate production from acetaldehyde, samples were analyzed by HPLC. In the case of longer products, for example the production of 2-hydroxyhexadecanoic acid from pentadecanal, samples were acidified with HCl and extracted with diethyl ether. The extracted diethyl ether was evaporated to dryness under a stream of nitrogen and derivatized by the addition of 1:1 BSTFA: pyridine. After incubation at 70 °C for 30 min, these samples were analyzed by GC-FID.

When the purified enzyme was supplied with pentadecanal and formyl-CoA, as in FIG. 24A-D, HACL1 was shown to catalyze the ligation of these molecules to 2-hydroxyhexadecanoyl-CoA as hypothesized. After hydrolysis of acyl-CoAs, the chromatogram of the sample containing enzyme shows similar peaks to the 2-hydroxyhexadecanoyl-CoA spiked standard, which are absent from the sample containing no enzyme.

The purified HACL1 was further tested for activity on shorter aldehydes, such as the ligation of acetaldehyde or formaldehyde with formyl-CoA to produce lactoyl-CoA or glycolyl-CoA, respectively. After hydrolysis of acyl-CoAs to their acid forms, these samples were analyzed by HPLC. The presence of lactate from elongation of acetaldehyde and formyl-CoA was identified in the sample containing HACL1, but not in the no enzyme control as shown in FIG. 24A-D. Similar results were observed for glycolate from formaldehyde and formyl-CoA as shown in FIG. 24A-D. The presence of lactate in the relevant samples was confirmed by NMR. This demonstrates that HACL1 is capable of catalyzing the ligation of aldehydes with chain lengths ranging at least from C1-C15 with formyl-CoA, making it suitable for acyloin condensation between C5 aldehyde isobutanal with formyl-CoA, required for the GPP synthesis pathway.

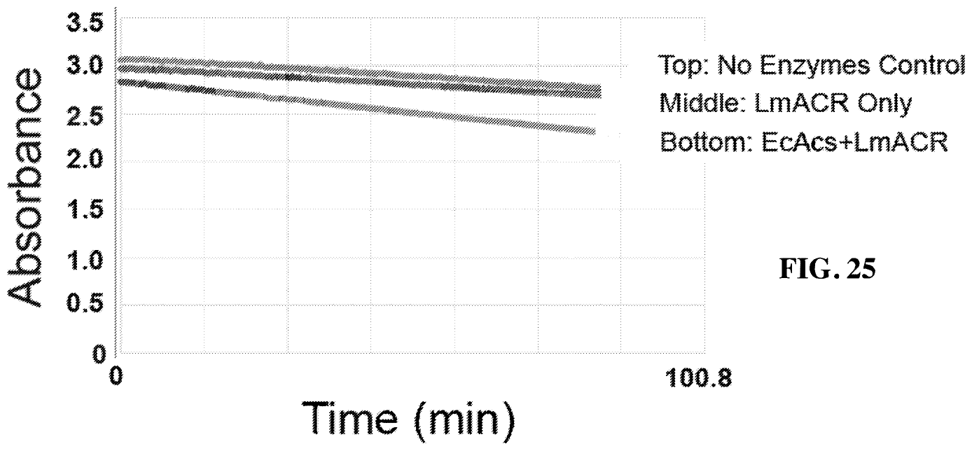

Also, the required activity of activation of formate to formyl-CoA by E. coli acyl-CoA synthase ACS (NP_418493.1) was also proven in vitro as shown in FIG. 25. This assay was coupled with reduction of resultant formyl-CoA to formaldehyde by Listeria monocytogenes acyl-CoA reductase (ACR) Lmol179 (CAC99257.1) and the activity was measured through observation of NADH oxidization.

A plasmid containing the codon optimized gene encoding 6×HIS-tagged Lmo1179 from Lysteria monocytogenes was constructed. The resulting construct was transformed into E. coli BL21(DE3) for expression. The resulting strain was cultured in 50 mL of TB media containing appropriate antibiotics in a 250 mL flask. When the culture reached an OD550 of approximately 0.6, expression was induced by the addition of 0.1 mM IPTG, and the cells were harvested by centrifugation after overnight incubation at room temperature.

The HIS-tagged Lmo1179 protein was purified from the cell extract using Talon Metal Affinity Resin (Clontech lab., CA). In short, a 250 μL resin bed was equilibrated twice using 2.5 mL of a buffer containing 50 mM sodium phosphate, 300 mM NaCl, and 10 mM imidazole at pH 7.5 (NPI-10). The cell extract was added to the resin and the mixture shaken gently for 20 minutes on ice. The resin was then washed twice with 2.5 mL buffer NPI-20 (same as NPI-10 but with 20 mM imidazole), shaking gently on ice for 15 minutes each wash. The resin was then transferred to a gravity column and washed once with 1.25 mL NPI-20. Finally, the desired protein was eluted using 1.25 mL of buffer NPI-250 (same as buffer NPI-10 but with 250 mM imidazole), and the eluate collected in 500 μL fractions.

E. coli ACS was cloned, expressed, and purified in E. coli as described above. The purified enzyme was evaluated for its ability to convert formate into the extender unit formate. Enzyme assays were performed in 23 mM potassium phosphate buffer pH 7.0, 1 mM CoASH, 0.5 mM NADH, 5 mM ATP, 2.5 mM MgCl₂, 50 mM formate. E. coli ACS was added along with Lysteria monocytogenes Lmo1179, and the reduction of resulting formyl-CoA was monitored by measuring absorbance of NADH at 340 nm. The sample containing ACS resulted in an increased rate of NADH oxidation, indicating that formyl-CoA was produced by ACS.

Among above enzymes, as mentioned in the previous example, the in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned above, they did not show the activity on prenol oxidization. The results of assays on alcohol dehydrogenases can be seen in Table P, and the results of assays on acyl-CoA reductases and relevant enzyme preparation and assay methods are described in the previous example.

The in vitro activity of dehydration of ethylene glycol to acetaldehyde by diol dehydratae PddABC (AFJ04717.1, AFJ04718.1, AFJ04719.1) from Klebsiella oxytoca has been proven, as shown in FIG. 22. The assay method is described in the previous examples.

### (Prophetic) GPP Biosynthesis Via 4-Methyl-2-Oxo-4-Pentenoic Acid and Isoprenol Starting Aldol Condensation Between Acetaldehyde and 2-Oxobutyric Acid

The purpose of this experiment is to demonstrate the biosynthesis of GPP through a novel pathway that starts from aldol condensation between 2-oxobutyric acid and acetaldehyde via 4-methyl-2-oxo-4-pentenoic acid and isoprenol, using E. coli as the host organism. This pathway starts from aldol condensation between pyruvate and acetaldehyde to 4-hydroxy-2-oxo-3-methylpentaonoic acid by E. coli aldolase MhpE (NP_414886.1). Acetaldehyde is supplied either through decarboxylation of pyruvate by Saccharomyces cerevisiae alpha-keto acid decarboxylase PDC1(CAA97573.1) or through reduction of acetyl-CoA by E. coli aldehyde forming acyl-CoA reductase MhpF (NP_414885.1). 2-oxobutyric acid is elongated from pyruvate through alpha-keto acid pathway composed of: citramalate synthase CimA from Methanocaldococcus jannaschii (WP_010870909.1) or Leptospira interrogans serovar Lai str. 56601 (NP_712531.1); citramalate isomerase LeuCD from E. coli (NP_414614.1, NP_414613.1) or Methanocaldococcus jannaschii (AAB98487.1, AAB99283.1) or Leptospira interrogans serovar Lai str. 56601 (NP_712276.1, NP_712277.1); Methanocaldococcus jannaschii 3-methylmalate dehydrogenase MJ0720 (WP_010870225.1) or Leptospira interrogans serovar Lai str. 56601 3-methylmalate dehydrogenase LeuB (NP_712333.1).

After the aldol condensation, a mutase transfers the methyl group of 4-hydroxy-2-oxo-3-methylpentaonoic acid from C-3 to C-4 site, generating 4-hydroxy-2-oxo-4-methylpentaonoic acid. Then, E. coli 2-oxopent-4-enoate dehydratase MhpD (NP_414884.2) dehydrates 4-hydroxy-2-oxo-4-methylpentaonoic acid into 4-methyl-2-oxo-4-pentenoic acid. 4-methyl-2-oxo-4-pentenoic acid can be converted to 3-methyl-3-butenoyl-CoA by alpha-keto acid dehydrogenase and 3-methyl-3-butenoyl-CoA is converted to isoprenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. 4-methyl-2-oxo-4-pentenoic acid can also be converted to isoprenol by two steps of reactions catalyzed by alpha-keto acid decarboxylase and alcohol dehydrogenase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methyl-3-butenoyl-CoA to isoprenol. Alcohol dehydrogenase is selected from the group consisting E. coli YahK (NP_414859.1), E. coli YjgB (NP_418690.4) and Acinetobacter sp. SE19 ChnD (BAC80217.1).

Isorenol is then converted to IPP by one or two steps of phosphorylation. If phosphorylated by two steps, the first step is catalyzed by E. coli alcohol kinase YchB (NP_415726.1) and the second is catalyzed by M. thermautotrophicus phosphate kinase MtIPK (AAB84554.1) or Thermoplasma acidophilum phosphate kinase ThaIPK (WP_010900530.1) or Methanocaldococcus jannaschii phosphate kinase MjIPK (3K4Y_A). The one step phosphorylation is catalyzed by alcohol diphosphokinase. E. coli isopentenyl pyrophosphate isomerase Idi (NP_417365.1) converts DMAPP to IPP. DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimum basilicum geraniol stynthase GES (AR11765.1, N-terminal 65 aa truncation) converts GPP to geraniol, the proxy product for the synthesis pathway.

JST06(DE3) serves as the E. coli host strain for demonstration of this novel pathway. Vector creation, strain creation, growth and analysis of supernatant are as described above in previous examples.

### (Prophetic) GPP Biosynthesis Via 2-Oxoisovaleric Acid, 2-Oxoisocaproic Acid and Prenol Starting from Decarboxylative Acyloin Condensation Between Two Pyruvates

The purpose of this example is to demonstrate the biosynthesis of GPP through a novel pathway via 2-oxoisovaleric acid, 2-oxoisocaproic acid and prenol. E. coli serves as the host organism. This pathway starts from decarboxylative acyloin condensation of two pyruvates to (S)-2-acetolactone by B. subtilis acetolactate synthase AlsS (NP_391482.2). E. coli acetohydroxy acid isomeroreductase IlvC (NP_418222.1) converts (S)-2-acetolactone to (2R)-2,3-dihydroxy-3-methylbutyric acid. E. coli dihydroxy acid dehydratase IlvD (YP_026248.1) dehydrates (2R)-2,3-dihydroxy-3-methylbutanoate to 3-methyl-2-oxobutyric acid (2-oxoisovaleric acid). Then, 2-oxoisovaleric acid is elongated into 2-oxoisocaproic acid through alpha-keto acid pathway composed of: E. coli isopropylmalate synthase LeuA (NP_414616.1, with a G462D mutation to maximize 2-oxoisocaprate production and minimize 2-oxoisovalerate, Connor et al. 2008) which condenses 2-oxoisovaleric acid and acetyl-CoA to (2S)-2-isopropylmalate; E. coli isopropyl isomerase LeuCD (NP_414614.1, NP_414613.1) which converts (2S)-2-isopropylmalate to (2R, 3S)-3-isopropylmalate; E. coli isopropylmalate dehydrogenase LeuB (NP_414615.4) which oxidizes and decarboxylates (2R, 3S)-3-isopropylmalate, generating 4-methyl-2-oxopentanoic acid (2-oxoisocaproic acid). Then, S. avermitilis alpha-keto acid dehydrogenase complex BkdFGH-LpdA1 (BAC72088.1, BAC72089.1, BAC72090.1, KUN54417.1) converts 2-oxoisocaproic acid into isovaleryl-CoA. Overexpression of heterologous branched alpha-keto acid dehydrogenase complex requires improved lipoylation, which can be realized though supplementation of lipoic acid accompanied with overexpression of E. coli lipoate-protein ligase LplA (NP_418803.1), or overexpression of E. coli endogenous lipoylation pathway consisting lipolate synthase LipA (NP_415161.1) and lipoyl(octanoyl) transferase LipB (NP_415163.2). P. aeruginosa acyl-CoA dehydrogenase LiuA (APJ52511.1) converts isovaleryl-CoA to 3-methylcrotonyl-CoA. 3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting of E. coli YahK (NP_414859.1), E. coli YjgB (NP_418690.4) and Acinetobacter sp. SE19 ChnD (BAC80217.1). Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP_010900530.1, V73I, Y141V and K204G mutations to increase specificity on prenol. Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. Then, DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimum basilicum geraniol stynthase GES (AR11765.1, with N-terminal 65 aa truncation) converts GPP to geraniol, which serves as a proxy to demonstrate a functioning pathway.

JST06(DE3) serves as the E. coli host strain for demonstration of this novel pathway. Vector creation, strain creation, growth and analysis of supernatant is conducted as described in previous examples.

The required plasmids and primers for this example are listed in Table T. The genes encoding E. coli enzymes are PCR amplified from the genomic DNA of wild type strain, while genes encoding other enzymes are codon optimized and synthesized by either GeneArt or GenScript. For construction of pET-P1-ilvC-ilvD-P2-alsS-liuA, the codon-optimized alsS and liuA gene inserts were first PCR amplified with alsS-f1/alsS-r2 and liuA-f1/liuA-r1 primers respectively, and inserted together into vector pETDuet-1 cleaved by NdeI and KpnI through In-Fusion HD Eco-Dry Cloning system, resulting in pET-P2-alsS-panE plasmid. The ilvC and ilvD gene inserts were then PCR amplified from the genomic DNA of E. coli with ilvC-f1/ilvC-r1 and ilvD-f1/ilvD-r1 primers respectively, and inserted together into vector pET-P2-alsS-liuA cleaved by NcoI and EcoRI through In-Fusion HD Eco-Dry Cloning system, generating pET-P1-ilvC-ilvD-P2-alsS-liuA. For construction of pCDF-P1-bkdF-bkdG-bkdH-P2-lplA-lpdA1, the lplA and lpdA1 gene inserts were first PCR amplified with lplA-f1/lplA-r1 and lpdA1-f1/lpdA1-r1 primers respectively, and inserted together into vector pCDFDuet-1 cleaved by NdeI and KpnI through In-Fusion HD Eco-Dry Cloning system, resulting in pCDF-P2-lplA-lpdA1. The codon-optimized bkdF, bkdG and bkdH gene inserts were then PCR amplified with bkdF-f1/bkdF-r1, bkdG-f1/bkdG-r1, bkdH-f1/bkdH-r1 respectively and inserted together into pCDF-P2-lplA-lpdA1 cleaved by NcoI and EcoRI through In-Fusion HD Eco-Dry Cloning system, generating pCDF-P1-bkdF-bkdG-bkdH-P2-lplA-lpdA1. For construction of pRSF-P1-leuA(G462D)-leuB-P2-leuC-leuD, the leuA and leuB genes were PCR amplified together into two pieces from the genomic DNA of E. coli with leuA(G462D)B-f11/leuA(G462D)B-r11 and leuA(G462D)B-f12/leuA(G462D)B-r12 respectively, and attached together through overlap PCR with leuA(G462D)B-f2/leuA(G462D)B-r2 to generate G462D mutation. The overlap PCR product was inserted into pRSFDuet-1 cleaved by NcoI and EcoRI through In-Fusion HD Eco-Dry Cloning system, generating pRSF-P1-leuA(G462D)-leuB. Then leuC and leuD genes were amplified together from from the genomic DNA of E. coli with leuCD-f1/leuCD-r1 and the resulting gene insert was inserted into pRSF-P1-leuA(G462D)-leuB cleaved by KpnI, generating pRSF-P1-leuA(G462D)-leuB-P2-leuC-leuD. Before the introduction to host strain, the sequences of constructed plasmids were confirmed by DNA sequencing.

Among above enzymes, as mentioned in the previous example, the in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned above, they did not show the activity on prenol oxidization. The results of assays on alcohol dehydrogenases can be seen in Table P, and the results of assays on acyl-CoA reductases and relevant enzyme preparation and assay methods are described in the previous example.

(Prophetic) GPP Biosynthesis Via 2-Oxoisovaleric Acid, 2-Oxoisocaproic Acid and Prenol Starting from Aldol Condensation Between Pyruvate and Acetaldehyde

The purpose of this experiment is to demonstrate the biosynthesis of GPP through a novel pathway that starts from aldol condensation between pyruvate and acetaldehyde via 2-oxoisovaleric acid, 2-oxoisocaproic acid and prenol, using E. coli as the host organism. This pathway starts from aldol condensation between pyruvate and acetaldehyde to (S)-4-hydroxy-2-oxopentaonoic acid by E. coli aldolase MhpE (NP_414886.1). Acetaldehyde is supplied either through decarboxylation of pyruvate by Saccharomyces cerevisiae alpha-keto acid decarboxylase PDC1(CAA97573.1) or through reduction of acetyl-CoA by E. coli aldehyde forming acyl-CoA reductase MhpF (NP_414885.1). Then, a mutase moves the —(C═O)COOH group of (S)-4-hydroxy-2-oxopentaonic acid from C-3 site to C-4 site, forming 3-hydroxy-2-oxo-3-methylbutyric acid. 2-hydroxyacid dehydrogenase converts 3-hydroxy-2-oxo-3-methylbutyric acid to (2R)-2,3-dihydroxy-3-methylbutyric acid. E. coli dihydroxy acid dehydratase IlvD (YP_026248.1) dehydrates (2R)-2,3-dihydroxy-3-methylbutanoate to 3-methyl-2-oxobutyric acid (2-oxoisovaleric acid). Then, 2-oxoisovaleric acid is elongated into 2-oxoisocaproic acid through alpha-keto acid pathway composed of: E. coli isopropylmalate synthase LeuA (NP_414616.1, with a G462D mutation to maximize 2-oxoisocaprate production and minimize 2-oxoisovalerate, Connor et al. 2008) which condenses 2-oxoisovaleric acid and acetyl-CoA to (2S)-2-isopropylmalate; E. coli isopropyl isomerase LeuCD (NP_414614.1, NP_414613.1) which converts (2S)-2-isopropylmalate to (2R, 3S)-3-isopropylmalate; E. coli isopropylmalate dehydrogenase LeuB (NP_414615.4) which oxidizes and decarboxylates (2R, 3S)-3-isopropylmalate, generating 4-methyl-2-oxopentanoic acid (2-oxoisocaproic acid). Then, S. avermitilis alpha-keto acid dehydrogenase complex BkdFGH-LpdA1 (BAC72088.1, BAC72089.1, BAC72090.1, KUN54417.1) converts 2-oxoisocaproic acid into isovaleryl-CoA.

Overexpression of heterologous branched alpha-keto acid dehydrogenase complex requires improved lipoylation, which can be realized though supplementation of lipoic acid accompanied with overexpression of E. coli lipoate-protein ligase LplA (NP_418803.1), or overexpression of E. coli endogenous lipoylation pathway consisting lipolate synthase LipA (NP_415161.1) and lipoyl(octanoyl) transferase LipB (NP_415163.2). P. aeruginosa acyl-CoA dehydrogenase LiuA (APJ52511.1) converts isovaleryl-CoA to 3-methylcrotonyl-CoA. 3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. SE19 ChnD (BAC80217.1). Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP 010900530.1, V73I, Y141V and K204G mutations to increase specificity on prenol. Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. Then, DMAPP and IPP are condensed to GPP catalyzed by E. coli GPP synthase IspA (NP_414955.1, S80F) or A. grandis GPP synthase GPPS2 (AAN01134.1, N-terminal 84 aa truncation). Ocimum basilicum geraniol stynthase GES (AR11765.1, with N-terminal 65 aa truncation) converts GPP to geraniol, which serves as a proxy to demonstrate a functioning pathway.

JST06(DE3) serves as the E. coli host strain for demonstration of this novel pathway. Vector creation, strain creation, growth and analysis of supernatant are largely as described in previous examples.

The plasmids listed in Table T can be used for required gene expression. The primers required for construction of these plasmids are also listed in Table T and their construction process is described in previous examples.

Among above enzymes, as mentioned in the previous example, the in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned above, they did not show the activity on prenol oxidization. The results of assays on alcohol dehydrogenases can be seen in Table P, and the results of assays on acyl-CoA reductases and relevant enzyme preparation and assay methods are described in the previous example.

GPP Biosynthesis Via 3-Hydroxy-3-Methylglutaryl-COA (HMG-COA) and Prenol Starting from Non-Decarboxylative Claisen Condensation Between Two Acetyl-COAS or Decarboxylative Claisen Condensation Between Acetyl-COA and Malonyl-COA

The purpose of this example is to demonstrate the biosynthesis of GPP through a novel pathway via HMG-CoA and prenol. E. coli serves as the host organism. This pathway starts from non-decarboxylative Claisen condensation between two acetyl-CoAs to acetoacetyl-CoA catalyzed by E. coli thiolase AtoB (NP_416728.1) or decarboxylative Claisen condensation between acetyl-CoA and malonyl-CoA by ketoacyl-CoA synthase. Malonyl-CoA is supplied from acetyl-CoA by E. coli acetyl-CoA carboxylase AccABCD (NP_414727.1, NP_417721.1, NP_417722.1, NP_416819.1). Then, S. aureus 3-hydroxy-3-methylglutaryl-CoA synthase HMGS (BAU36102.1) condenses acetoacetyl-CoA with another acetyl-CoA to generate 3-hydroxy-3-methylglutaryl-CoA (HMG-CoA). HMG-CoA is dehydrated to 3-methylglutaconyl-CoA by M. xanthus enoyl-CoA hydratase LiuC (WP_011553770.1). M. xanthus glutaconyl-CoA decarboxylase AibAB (WP_011554267.1, WP_011554268.1) decarboxylates 3-methylglutaconyl-CoA to 3-methylcrotonyl-CoA. 3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. SE19 ChnD (BAC80217.1). Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP_010900530.1, V73I, Y141V and K204G mutations to increase specificity on prenol. Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP. Then, DMAPP and IPP are condensed to GPP catalyzed by E. coli GPP synthase IspA (NP_414955.1, S80F) or A. grandis GPP synthase GPPS2 (AAN01134.1, N-terminal 84 aa is truncated). Ocimum basilicum geraniol stynthase GES (AR11765.1, N-terminal 65 aa truncation) converts GPP to geraniol, which serves as the proxy for pathway function.

JST06(DE3) atoB^{CT5} ΔfadB serves as the E. coli host strain for demonstration of novel pathway. The genotype atoB^{CT5} refers to chromosomal atoB gene, encoding the thiolase that condenses acetyl-CoA to acetoacetyl-CoA, under the p^{CT5} promoter for controlled induction by cumate. To enable the cumate-inducible chromosomal expression of atoB gene in JST06(DE3), E. coli atoB gene was first PCR amplified from genomic DNA extracted through Genomic DNA Purification kit (Promega, Fitchburg, WI, USA), digested with BglII and NotI, and ligated by T4 ligase (Invitrogen, Carlsbad, CA) into pUCBB-ntH6-eGFP (Vick et al. 2011) that was previously digested with BglII and NotI to produce pUCBB-P^{CT5}-atoB. The resulting ligation products were used to transform E. coli DH5alpha (Invitrogen, Carlsbad, CA), and positive clones identified by PCR were confirmed by DNA sequencing. To integrate the cumate-controlled atoB construct into the chromosome of JST06(DE3), first the cumate repressor (cymR), promoter/operator regions (P^{CT5}), and respective ORFs were PCR amplified, as was the kanamycin drug construct via pKD4 (Datsenko and Wanner, 2000). These respective products were linked together via overlap extension PCR to create a final chromosomal targeting construct. Integration of the cumate-controlled constructs was achieved via standard recombineering protocols by using strain HME45 and selection on LB drug plates (Thomason et al. 2001). The primers used in the construction of JST06(DE3) atoB^{CT5} are listed in Table U.

The gene fadB, encoding hydroxyacyl-CoA dehydrogenase and enoyl-CoA hydratase is deleted to minimize the flux of acetoacetyl-CoA entering the competing beta-oxidation reversal pathway. The gene deletion is performed using P1 phage transduction (Yazdani et al. 2008) with single gene knockout mutants from the National BioResource Project (NIG, Japan, Baba et al. 2006) as the specific deletion donor.

The other genes for overexpression are made, put into cells and tested as described above. The quantification of intermediate prenol is also performed via ion-exclusion HPLC using a Shimadzu Prominence SIL 20 system equipped with an HPX-87H organic acid column (Bio-Rad, Hercules, CA) with operating conditions to optimize peak separation (0.3 mL/min flow rate, 30 mM H₂SO₄ mobile phase, column temperature 42 °C). Concentration of 2-oxoisovaleric acid in fermentation samples is determined through calibration to known prenol standards (5, 1, 0.5, 0.2 and 0.1 g/L).

The first part of pathway to prenol has demonstrated been in vivo. The plasmids used for demonstration of in vivo prenol production are listed in Table V and the primers required for constructions of these plasmids are listed in Table W. First, the pathway to prenol was expressed in two vectors: the genes encoding acyl-CoA reductases were inserted into pETDuet-1 vector, while other genes were expressed from plasmid pCDF-P1-HMGS-aibA-aibB-P2-liuC. When using CbjALD, endogenous alcohol dehyrogenases without overexpression was used. Except for genes encoding E. coli enzymes YjgB and YahK, which were PCR amplified from the genomic DNA of wild type E. coli MG1655 strain, and the gene encoding CbjALD, which was PCR amplified from the genomic DNA of C. beijerinckii, the genes were codon optimized and synthesized by either GeneArt or GenScript. The adhE2, cbjALD and maqu_2507 gene inserts were PCR amplified with adhE2-f1/adhE2-r1, cbjALD-f2/cbjALD-r2 and maqu_2507-f2/maqu_2507-r2 primers respectively and inserted into vector pETDuet-1 cleaved by NdeI through In-Fusion HD Eco-Dry Cloning system to construct pET-P2-adhE2, pET-P2-cbjALD, pET-P2-maqu_2507 respectively. For construction of pCDF-P1-HMGS-aibA-aibB-P2-liuC, the codon-optimized liuC gene insert was first PCR amplified with liuC-f1/liuC-r1 primers and inserted into vector pCDFDuet-1, cleaved by NdeI through In-Fusion HD Eco-Dry Cloning system to generate pCDF-P2-liuC. Then, the codon-optimized hmgs gene insert was PCR amplified with hmgs-f1/hmgs-r1 primers and inserted into vector pCDF-P2-liuC cleaved with NcoI and EcoRI through In-Fusion HD Eco-Dry Cloning system to generate pCDF-P1-HMGS-P2-liuC. Finally, the codon optimized aibA and aibB gene inserts were PCR amplified with aibA-f1/aibA-r1 and aibB-f1/aibB-r1 primers respectively and inserted into vector pCDF-P2-liuC cleaved with EcoRI and SalI through In-Fusion HD Eco-Dry Cloning system to generate pCDF-P1-HMGS-aibAB-P2-liuC. The sequences of constructed plasmids were further confirmed by DNA sequencing. Then, the sequence confirmed plasmids were introduced to competent cells of host strain JST06(DE3) atoB^{CT5} ΔfadB.

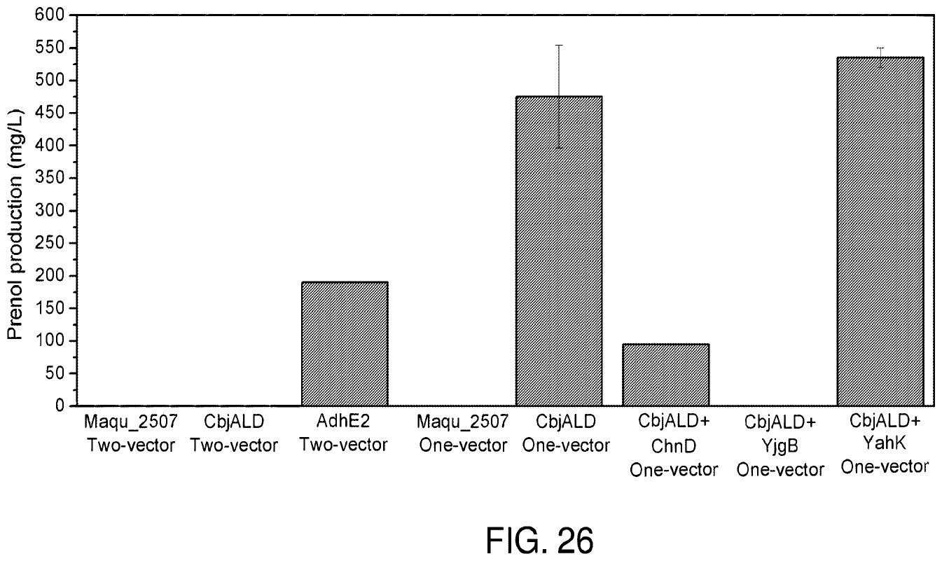

As shown in FIG. 26, under the two-vector system, the strain expressing AdhE2 showed 190 mg/L of prenol production when grown under 37 °C for 48 hours in shake flasks with 20 mL LB-like MOPS media supplemented with 20 g/L glycerol, induced under 10 μM IPTG and 100 μM cumate, while prenol production was not detected when expressing CbjALD and Maqu 2507. To test whether the burden caused by multiple vector system led to undetected prenol production when using CbjALD and Maqu_2507, the cbjALD and maqu_2507 gene inserts were PCR amplified with cbjALD-f2/cbjALD-r3 and maqu_2507-f2/maqu_2507-r3 primers respectively and inserted into vector pCDF-P2-HMGS-aibAB-P2-liuC cleaved by NdeI through In-Fusion HD Eco-Dry Cloning system to generate pCDF-P2-HMGS-aibAB-P2-cbjALD-liuC and pCDF-P2-HMGS-aibAB-P2-maqu_2507-liuC respectively, so that whole prenol supplying pathway is expressed through single vector. As a result, while the strain JST06(DE3) atoB^{CT5} ΔfadB pCDF-P2-HMGS-aibAB-P2-maqu_2507-liuC still did not produce detectable prenol, JST06(DE3) atoB^{CT5} ΔfadB pCDF-P2-HMGS-aibAB-P2-cbjALD-liuC produced 475 mg/L of prenol production, higher than the strain with two-vector system using AdhE2, when grown under same conditions as above, possibly due to the added metabolic burden of maintaining two plasmids in the cell.

To test whether co-expression of alcohol dehydrogenases YahK, YjgB and ChnD, which had been proven to be active on oxidizing prenol to 3-methyl-1-butenal through in vitro assay according to the second experiment, can improve prenol production with usage of CbjALD, the chnD, yjgB, yahK gene inserts were PCR amplified with chnD-f2/chnD-r2, yjgB-f1/yjgB-r1 and yahK-f1/yahK-r1 primers respectively and inserted into vector pCDF-P2-HMGS-aibAB-P2-cbjALD-liuC cleaved by BglII and XhoI through In-Fusion HD Eco-Dry Cloning system to generate pCDF-P2-HMGS-aibAB-P2-cbjALD-liuC-chnD, pCDF-P2-HMGS-aibAB-P2-cbjALD-liuC-yjgB and pCDF-P2-HMGS-aibAB-P2-cbjALD-liuC-yahK respectively, and the resultant plasmids were introduced into JST06(DE3) atoB^{CT5} ΔfadB. As a result, the strain overexpressing ChnD and YjgB did not show the detectable prenol production, while the strain overexpressing YahK produced 535 mg/L of prenol, higher than that of JST06(DE3) atoB^{CT5} ΔfadB pCDF-P2-HMGS-aibAB-P2-cbjALD-liuC, which uses endogenous alcohol dehydrogenase without overexpression, when grown under same conditions as above. To summarize, the pathway to prenol is effective in vivo when using acyl-CoA reductases CbjALD and AdhE2, and co-expression of YahK can further improve prenol production when using CbjALD.

After demonstrating the in vivo prenol production, the rest of the pathway, which converts prenol to geraniol, was added. A three-vector system was first used as shown in Table V. The pathway to 3-methylcrotonyl-CoA was expressed through pCDF-P1-HMGS-aibA-aibB-P2-liuC; acyl-CoA reductases AdhE2 or CbjALD were expressed through pRSF-P2-adhE2 or pRSF-P2-cbjALD; the rest of the pathway converting prenol to geraniol was expressed through pET-P1-idi-trGPPS2-P2-ges-ychB-mtipk. For construction of other plasmids, the E. coli genes encoding Idi and YchB were PCR amplified from the genomic DNA of wild type E. coli MG1655 strain, while the other genes were codon optimized and synthesized by either GeneArt or GenScript. The adhE2 and cbjALD gene inserts were PCR amplified with were PCR amplified with adhE2-f1/adhE2-r1 and cbjALD-f2/cbjALD-r2 primers respectively and inserted into vector pRSFDuet-1 cleaved by NdeI through In-Fusion HD Eco-Dry Cloning system to construct pRSF-P2-adhE2 and pRSF-P2-cbjALD respectively. To construct pET-P1-idi-trGPPS2-P2-ges-ychB-mtipk, the gene inserts encoding Idi and trGPPS2 (“tr” means “truncated” as first 84 aa of GPPS2 was truncated to improve the activity) were PCR amplified with idi-f1/idi-r1 and trgpps2-f1/trgpps2-r1 respectively and inserted together into pETDuet-1 cleaved by NcoI through In-Fusion HD Eco-Dry Cloning system to generate pET-P1-idi-trGPPS2. Then, the gene insert encoding GES was PCR amplified with ges-f1/ges-r1 primers and inserted into vector pET-P1-idi-trGPPS2 cleaved by NdeI and KpnI through In-Fusion HD Eco-Dry Cloning system to generate pET-P1-idi-trGPPS2-P2-ges. Finally, the gene inserts encoding YchB and MtIPK were PCR amplified with ychB-f1/ychB-r1 and mtipk-f1/mtipk-r1 respectively and inserted together into pET-P1-idi-trGPPS2-P2-ges cleaved by XhoI through In-Fusion HD Eco-Dry Cloning system to generate pET-P1-idi-trGPPS2-P2-ges-ychB-mtipk. The sequences of required primers can be seen in Table W. The sequences of constructed plasmids were further confirmed by DNA sequencing. Then, the sequence confirmed plasmids were introduced to competent cells of host strain JST06(DE3) atoB^{CT5} ΔfadB.

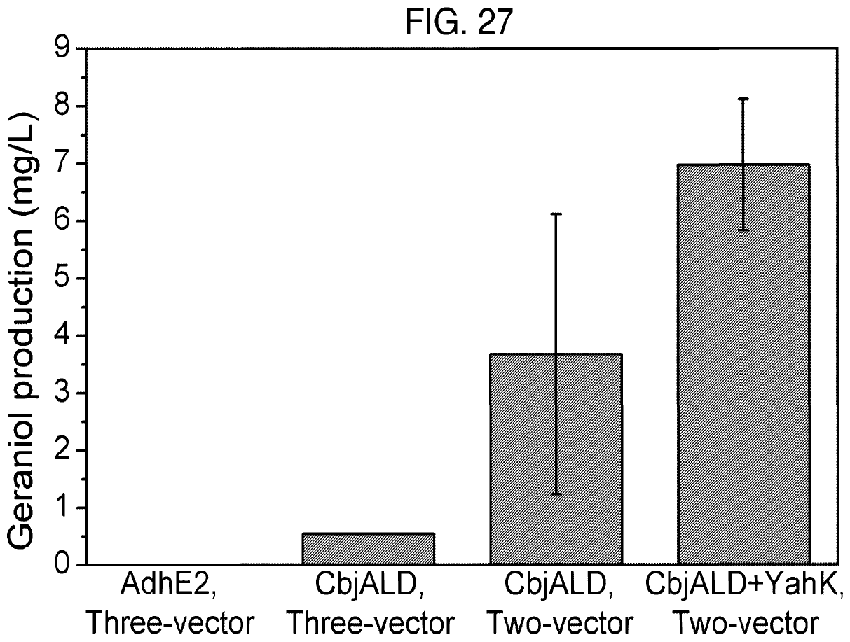

As shown in FIG. 27, the resultant strain using AdhE2 did not show detectable geraniol production, while the strain using CbjALD and endogenous alcohol dehydrogenases without overexpression had 0.54 mg/L of geraniol production when grown under 30 °C for 48 hours in shake flasks with 20 mL LB-like MOPS media supplemented with 20 g/L glycerol, induced under 10 μM IPTG and 100 μM cumate. Though the titer was small and further improvement measures, like decreasing the vector number and optimizing fermentation conditions, were required, this result indicates that the claimed novel GPP synthesis pathway via HMG-CoA and prenol is effective in vivo when using acyl-CoA reductase CbjALD.

A two-vector system was also tested for geraniol production with usage of acyl-CoA reductase CbjALD with or without alcohol dehydrogenase YahK. One plasmid was pCDF-P2-HMGS-aibAB-P2-cbjALD-liuC or pCDF-P2-HMGS-aibAB-P2-cbjALD-liuC-yahK that expresses the pathway from acetoacetyl-CoA to prenol (or most of “upper alcohol pathway”, as shown in FIG. 1), and the other plasmid was pET-P1-idi-trGPPS2-P2-ges-ychB-mtipk that expresses the pathway converting from prenol to geraniol (or “lower alcohol pathway”, as shown in FIG. 1). As shown in FIG. 27, the resultant strain using two-vector system and CbjALD and endogenous alcohol dehydrogenases without overexpression had 3.7 mg/L of geraniol production when grown under 30 °C for 48 hours in shake flasks with 20 mL LB-like MOPS media supplemented with 20 g/L glycerol, induced under 50 μM IPTG and 100 μM cumate, indicating that reduction of expression vector can improve the geraniol production. The addition of YahK overexpression further improved the titer to 7.0 mg/L. When YahK was overexpressed, the strain was grown under 30 °C for 48 hours in shake flasks with 15 mL LB-like MOPS media supplemented with 20 g/L glycerol, induced under 10 μM IPTG and 100 μM cumate.

(Prophetic) GPP Biosynthesis Via 3-Methyl-3-Hydroxybutyryl-COA and Prenol Starting from Non-Decarboxylative Claisen Condensation Between Two Acetyl-COAS or Decarboxylative Claisen Condensation Between Acetyl-COA and Malonyl-COA

The purpose of this example is to demonstrate the biosynthesis of GPP through a novel pathway via 3-methyl-3-hydroxybutyryl-CoA and prenol. E. coli serves as the host organism. This pathway starts from non-decarboxylative Claisen condensation between two acetyl-CoAs to acetoacetyl-CoA catalyzed by E. coli thiolase AtoB (NP_416728.1) or decarboxylative Claisen condensation between acetyl-CoA and malonyl-CoA by ketoacyl-CoA synthase. Malonyl-CoA is supplied from acetyl-CoA by E. coli acetyl-CoA carboxylase AccABCD (NP_414727.1, NP_417721.1, NP_417722.1, NP_416819.1). Then, acetoacetyl-CoA is hydrolyzed to acetoacetic acid by enzymes selected from the group consisting thioesterase, acyl-CoA transferase and phosphotransacylase plus carboxylate kinase. Acetoacetate decarboxylase removes the carboxyl group of acetoacetic acid, generating acetone. 3-hydroxy-3-methylglutaryl-CoA (HMG-CoA) synthase then performs a condensation between acetone and another acetyl-CoA, generating 3-methyl-3-hydroxybutyryl-CoA. Enoyl-CoA hydratase dehydrates 3-methyl-3-hydroxybutyryl-CoA to 3-methylcrotonyl-CoA. 3-methylcrotonyl-CoA is converted to prenol by alcohol-forming acyl-CoA reductase or aldehyde forming acyl-CoA reductase and alcohol dehydrogenase or carboxylate reductase and the hydrolysis enzyme selected from the group consisting thioesterase, acyl-CoA synthase, acyl-CoA transferase and carboxylate kinase plus phosphotransacylase. Alcohol-forming acyl-CoA reductase is selected from the group consisting C. acetobutylicum AdhE2 (YP_009076789.1) and M. aquaeolei VT8 Maqu_2507 (YP_959769.1). CbjALD from C. beijerinckii aldehyde forming acyl-CoA reductase (AAT66436.1) is selected for conversion of 3-methylcrotonyl-CoA to prenol. Alcohol dehydrogenase is selected from the group consisting E. coli YahK (NP_414859.1), E. coli YjgB (NP_418690.4) and Acinetobacter sp. SE19 ChnD (BAC80217.1).

Prenol is then converted to DMAPP by one or two steps of phosphorylation. If phosphorylated by two steps, the first step is catalyzed by E. coli alcohol kinase YchB (NP_415726.1) or Thermoplasma acidophilum phosphate kinase ThaIPK (WP_010900530.1, V73I, Y141V and K204G mutations to increase specificity on prenol, Liu et al. 2016) and the second is by M. thermautotrophicus phosphate kinase MtIPK (AAB84554.1). The one step phosphorylation is catalyzed by alcohol diphosphokinase. E. coli isopentenyl pyrophosphate isomerase Idi (NP_417365.1) converts DMAPP to IPP, which is condensed with DMAPP to form GPP catalyzed by E. coli GPP synthase IspA (NP_414955.1, S80F) or A. grandis GPP synthase GPPS2 (AAN01134.1, N-terminal 84 aa truncation). Ocimum asilicum geraniol stynthase GES (AR11765.1, N-terminal 65 aa truncation) converts GPP to geraniol, which serves as the proxy product.

JST06(DE3) atoB^{CT5} ΔfadB serves as the E. coli host strain for demonstration of novel pathway. The genes for overexpression are made and put into cells, which are gown and the supernatants analyzed as described above.

The quantifications of intermediates prenol and acetone are also performed via ion-exclusion HPLC using a Shimadzu Prominence SIL 20 system equipped with an HPX-87H organic acid column with operating conditions to optimize peak separation (0.3 mL/min flow rate, 30 mM H₂SO₄ mobile phase, column temperature 42 °C). Concentration of 2-oxoisovaleric acid in fermentation samples is determined through calibration to known acetone and prenol standards (5, 1, 0.5, 0.2 and 0.1 g/L).

The in vivo production of acetone has been demonstrated in E. coli. The JC01 (MG1655 ΔldhA ΔpoxB Δpta ΔadhE ΔfrdA, an E. coli strain with removal of mixed-acid fermentation for improved supply of acetyl-CoA) strain overexpressing thiolase AtoB and thioesterase YbgC showed 53 mg/L of acetone production when grown in LB-like MOPS media with glycerol under 37 °C for 48 hours. This result indicates that YbgC can hydrolyze acetoaceetyl-CoA, the product of non-decarboxylative Claisen condensation between two acetyl-CoAs by AtoB, to acetoacetic acid, and aetoacetic acid can be decarboxylated to acetone spontaneously or by endogenous E. coli enzymes. These enzymes can be used in this GPP synthesis pathway. The media, fermentation conditions and method of HPLC analysis on acetone are described in previous example. In this fermentation, the atoB gene was expressed from pTH-atoB, while the ybgC gene was expressed from pZS-ybgC. The primers required for construction of these plasmids can be seen in Table S, and the process of construction of these plasmids is described in the previous example.

Among above enzymes, as mentioned in the previous example, the in vitro activities of acyl-CoA reductases CbjALD and Maqu_2507 on reduction of 3-methylcrotonyl-CoA and the in vitro activities of alcohol dehydrogenases ChnD, YjgB and YahK on oxidization of prenol have been proven through enzymatic spectrophotometric assay. E. coli alcohol dehydrogenases FucO (NP_417279.2), YqhD (NP_417484.1), YiaY (YP_026233.1) were also assayed on prenol, but as mentioned above, they did not show the activity on prenol oxidization. The results of assays on alcohol dehydrogenases can be seen in Table P, and the results of assays on acyl-CoA reductases and relevant enzyme preparation and assay methods are described in the previous example.

### (Prophetic) Synthesis of Isoprenoids

The purpose of this example is to demonstrate the biosynthesis of isoprenoids other than geraniol from isoprenoid precursor GPP or others, which are supplied from claimed novel pathways. E. coli serves as the host strain. The possible isoprenoid products are monoterpenes like limonene and pinene, which are derived from GPP, and sesquiterpenes like beta-caryophyllene, valencene, vetispiradiene, amorphadiene and farnesene, which are derived from farnesyl diphosphate (FPP), as shown in FIG. 15. FPP is an isoprenoid precursor with five more carbons than GPP and supplied through condensation between GPP and IPP, which are supplied from above novel claimed pathways, by E. coli FPP synthase IspA (NP_414955.1). These mentioned isoprenoids are with great industrial importance and can be used as biofuels and solvents and be used in the fields of cosmetics, pharmaceutics, and perfumery. The conversion of GPP to limonene is caalzyed by Mentha spicata limonene synthase LS (AGN90914.1). The conversion of GPP to pinene is catalyzed by Pinus taeda pinene synthase Pt30 (AAO61228.1). The conversion of FPP to beta-caryophyllene is catalyzed by Artemisia annua beta-caryophyllene synthase QHS1 (AAL79181.1). The conversion of FPP to valencene is catalyzed by Callitropsis nootkatensis valenecene synthase VALC (AFN21429.1). The conversion of FPP to vetispiradiene is catalyzed by Hyoscyamus muticus vetispiradiene synthase VS1 (Q39978.2). The conversion of FPP to amorphadiene is catalyzed by Artemisia annua amorphadiene synthase ADS (AAF61439.1). The conversion of FPP to farnesene is catalyzed by Malus domestica farnesene synthase FS (NP_001280822.1). The genes encoding enzymes for productions of above isoprenoids are separately cloned into pACYCDuet-1 vector (Novagen, Darmstadt, Germany), and the resultant plasmids can be directly used and introduced to GPP-synthsizing strains as described in previous examples to realized productions of isoprenoids. The resultant vectors are listed in Table X. Except ispA, which is PCR amplified from the genomic DNA of wild type E. coli, the genes encoding synthases of isoprenoids are codon-optimized and synthesized by GenScript (Piscataway, NJ) or GeneArt® (Life Technologies, Carlsbad, CA).

In Vivo Synthesis of Olivetolic Acid in E. coli

The purpose of this example is to demonstrate in vivo synthesis of olivetolic acid with E. coli as host organism. Olivetolic acid is a suitable aromatic acceptor of geranyl group donated from GPP, which is synthesized by claimed novel pathways, MVA, MEP/DXP, or other pathways, the prenylation reaction generating the valuable cannabinoid, cannabigerolic acid (CBGA). Olivetolic acid is synthesized through multiple possible pathways. The first pathway starts from three series of decarboxylative Claisen condensation with hexanoyl-CoA as the initial primer and malonyl-CoA as the extender unit by e.g., C. sativa olivetol synthase OLS (BAG14339.1), generating 3,5,7-trioxododecanoyl-CoA. Then, C. sativa olivetolic acid cyclase OAC (AFN42527.1, several non-conservative substitutions of residues are performed to improve the activity) cyclizes 3,5,7-trioxododecanoyl-CoA to olivetolic acid.

The second pathway also starts from three series of decarboxylative Claisen condensation with hexanoyl-CoA as the initial primer and malonyl-CoA as the extender unit, but catalyzed by other polyketide synthases selected from e.g., H. macrophylla stilbenecarboxylate synthase STCS (AAN76183.1, with a subset of mutations of T135S, T198M and I200C to accept hexanoyl-CoA as the active substrate), a type III polyketide synthase, and type I polyketide synthases AviM from Streptomyces viridochromogenes Tue57 (AAK83194.1), ArmB from Armillaria mellea (AFL91703.1) and CalO5 from Micromonospora echinospora ssp. Calichensis (AAM70355.1). These polyketide synthases directly perform the cyclization of 3,5,7-trioxododecanoyl-CoA to olivetolic acid.

The third pathway starts from three series of condensations with hexanoyl-CoA as the initial primer and acetyl-CoA as the extender unit by polyketoacyl-CoA thiolase selected e.g., from the group consisting FadAx (AAK18171.1) and PcaF (AAA85138.1) from P. putida, DcaF (CAG68532.1) from Acinetobacter sp. ADP1, and ScFadA (AAL10298.1) from S. collinus, generating 3,5,7-trioxododecanoyl-CoA, which is then cyclized to olivetolic acid by OAC.

Hexanoyl-CoA can be supplied from hexanoic acid, either supplemented or intracellularly synthesized through beta-oxidation reversal composed of e.g., ketoacyl-CoA thiolase BktB (AAC38322.1) from R. eutropha, 3-hydroxyacyl-CoA dehydrogenase and enoyl-CoA hydratase multifunctional enzyme FadB from E. coli (NP_418288.1) and enoyl-CoA reductase EgTer from E. gracilis (Q5EU90.1) or fatty acid biosynthesis pathway composed of beta-ketoacyl-ACP synthases FabH (NP_415609.1) and FabB (NP_416826.1), acetoacetyl-ACP reductase FabG (NP_415611.1), 3-hydroxyacyl-ACP dehydratase FabZ (NP_414722.1) and enoyl-ACP reductase FabI (NP_415804.1), all from E. coli, with termination by E. coli thioesterase TesA (NP_415027.1, with truncation of 26 aa leader sequence) and activation by E. coli acyl-CoA synthetase FadD (NP_416319.1), or directly synthesized through overexpressed beta-oxidation reversal pathway without termination.

If malonyl-CoA is used as the extender unit, to enhance its supply, e.g., E. coli acetyl-CoA carboxylase AccABCD (NP_414727.1, NP_417721.1, NP_417722.1, NP_416819.1) is overexpressed. Also, to improve acetyl-CoA supply, e.g., E. coli pyruvate dehydrogenase complex AceEF-Lpd is overexpressed (e.g., NP_414658.1, NP_414656.1, NP_414657.1, A358V mutation in Lpd subunit to increase the activity of pyruvate dehydrogenase by reducing inhibition by NADH, Chen et al. 2014).

JST06(DE3) ΔfadE bktB^{CT5} ΔatoB fadB^{CT5} ΔfadA egter^{CT5}, which is able to intracellularly supply hexanoyl-CoA and hexanoic acid through beta-oxidation reversal, can serve as the host strain for the in vivo production of olivetolic acid. JST06(DE3) is described in previous examples and is selected to maximize the flux of beta-oxidation reversal for hexanoyl-CoA supply required for the synthesis of olivetolic acid via polyketoacyl-CoA thiolases. ΔatoB fadB^{CT5} are as described above. BktB, FadB and EgTer are chromosomally expressed under p^{CT5} promoter with control by cumate. To integrate the cumate-controlled bktB construct into the chromosome of the target strain, first the cumate repressor (cymR), promoter/operator regions (P^{CT5}) and respective ORFs are PCR amplified using appropriate primers, as is chloramphenicol drug construct via pKD4 (Datsenko and Wanner, 2000). These respective products are linked together via overlap extension PCR to create a final chromosomal targeting construct. The fadA gene was separately deleted via recombineering in the HME45 derivative harboring the cumate-controlled fadBA construct by replacement of the fadA ORF with a zeocin resistance marker amplified from pKDzeo (Magner et al. 2007).

For the creation of the cumate-controlled egTER, the cat gene, cymR repressor gene, hybrid cumate-controlled phage T5 promoter, and egTER gene are PCR amplified from genomic DNA of a strain with egTER seamlessly replacing fadBA at the cumate controlled fadBA locus (See below for details). This product is recombineered into strain HME45 at the end of the fabI locus, selecting on chloramphenicol (12.5 μg/ml) LB plates. Integration is done in a manner to duplicate the last 22 bp of fabI (including stop codon) so as retain an overlapping promoter for the next native downstream gene.

Construction of the strain serving as the PCR template for egTER described above was accomplished by first creating a kan-sacB fusion cassette via overlap extension PCR using pKD4 and genomic DNA, respectively. This kan-sacB cassette was integrated between fadB and fadA of the fadBA^{CT5} strain formerly constructed (Vick et al., 2015) through subsequent recombineering. Seamless replacement of the kan-sacB cassette to create the cat-cymR-P^{CT5}-egTER at the fadBA locus was done via recombineering and subsequent sucrose selection with codon optimized egter (Genscript) PCR product. The primers for construction of this strain are listed in Table Y.

The gene fadE, encoding acyl-CoA dehydrogenase is deleted to block the degradation of hexanoyl-CoA through beta-oxidation. The gene deletion is performed using P1 phage transduction (Yazdani et al. 2008) with single gene knockout mutants from the National BioResource Project (NIG, Japan, Baba et al. 2006) as the specific deletion donor.

The constructed vectors for expression of different routes of olivetolic acid synthesis pathways are listed in Table Z. To construct pET-P1-OLS-P2-OAC, the OLS gene insert was first PCR amplified with OLS-BamHI-F/OLS-EcoRI-R primers and inserted into vector pETDuet-1 cleaved by BamHI and EcoRI through Gibson Assembly cloning system, generating pET-P1-OLS. Then, the OAC gene insert was PCR amplified with OAC-NdeI-Up/OAC-XhoI-Dn primers and inserted into pET-P1-OLS cleaved by NdeI and XhoI through Gibson Assembly cloning system, generating pET-P1-OLS-P2-OAC.

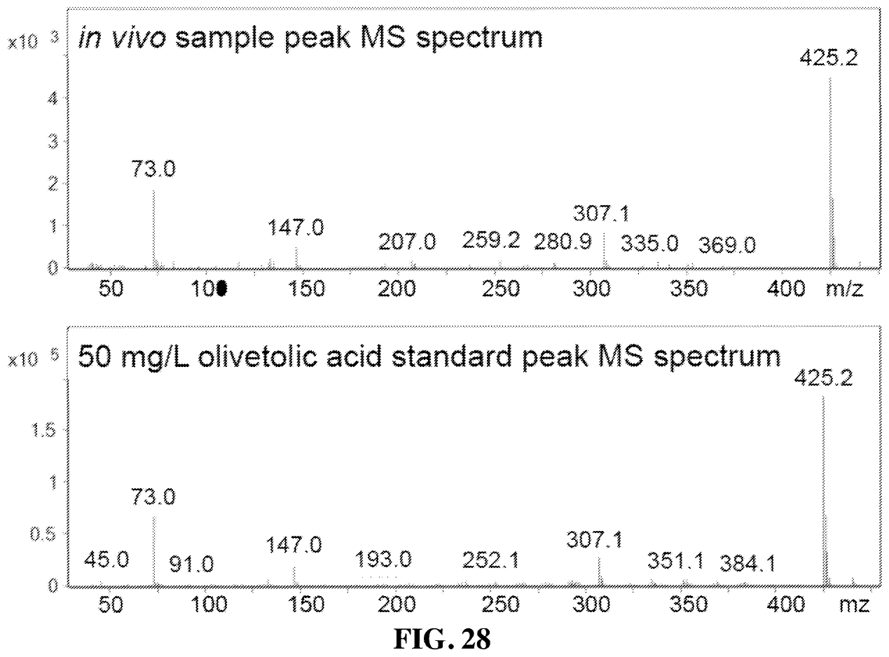

The in vivo synthesis of olivetolic acid in E. coli has been demonstrated by using C. sativa olivetol synthase OLS and olivetolic acid cyclase OAC. JST06(DE3) ΔfadE bktB^{CT5} ΔatoB fadB^{CT5} ΔfadA egter^{CT5} @fabI served as the host strain containing plasmid pET-P1-OLS-P2-OAC. The genes encoding OLS and OAC were codon optimized and synthesized by either GeneArt or GenScript. The resultant strain for olivetolic acid production was grown in shake flasks with 15 mL LB-like MOPS media supplemented with 20 g/L glycerol and 55 g/L CaCO₃ at 30 °C for 48 hours. Extracellular olivetolic acid was extracted and derivatized following the protocols described in previous examples and the resulting sample analyzed via GC-MS. FIG. 28 shows GC-MS identification of in vivo olivetolic acid synthesis through comparsion with an olivetolic acid standard. This result demonstrates that OLS and OAC are effective for in vivo biosynthesis of olivetolic acid which can be used as the acceptor group donated from GPP synthesized through claimed pathways for production of valued compound cannabigerolic acid (CBGA).

(Prophetic) In Vivo Synthesis of Divarinolic Acid in E. coli

The purpose of this example is to demonstrate in vivo synthesis of divarinolic acid with E. coli as host organism. Divarinolic acid is a suitable aromatic acceptor of geranyl group donated from GPP, which is synthesized by claimed novel pathways or the known MVA, MEP/DXP pathways, or otherwise, in the prenylation reaction generating cannabinoid cannabigerovarinic acid (CBGVA). Divarinolic acid is synthesized through multiple possible pathways. The first pathway starts from three series of condensation with butyryl-CoA as the initial primer and malonyl-CoA as the extender unit by e.g., C. sativa olivetol synthase OLS (BAG14339.1), generating 3,5,7-trioxodecanoyl-CoA. Then, C. saliva olivetolic acid cyclase OAC (e.g., AFN42527.1, several non-conservative substitutions of residues are performed to improve the activity) cyclizes 3,5,7-trioxodecanoyl-CoA to divarinolic acid.

The second pathway also starts from three series of condensations with butyryl-CoA as the initial primer and malonyl-CoA as the extender unit, but catalyzed by catalyzed by other polyketide synthases selected from e.g., H. macrophylla stilbenecarboxylate synthase STCS (AAN76183.1, with a subset of mutations of T135S, T198M and I200C), a type III polyketide synthase, and type I polyketide synthases AviM from Streptomyces viridochromogenes Tue57 (AAK83194.1), ArmB from Armillaria mellea (AFL91703.1) and CalO5 from Micromonospora echinospora ssp. Calichensis (AAM70355.1). These polyketide synthases then directly perform the cyclization of 3,5,7-trioxodecanoyl-CoA to divarinolic acid.

The third pathway starts from three series of condensations with butyryl-CoA as the initial primer and acetyl-CoA as the extender unit by polyketoacyl-CoA thiolase from e.g., FadAx (AAK18171.1) and PcaF (AAA85138.1) from P. putida, DcaF (CAG68532.1) from Acinetobacter sp. ADP1, and ScFadA (AAL10298.1) from S. collinus, generating 3,5,7-trioxodecanoyl-CoA, which is then cyclized to divarinolic acid by OAC.

Butyryl-CoA can be supplied from butyric acid, either supplemented or intracellularly synthesized through beta-oxidation reversal composed of e.g., ketoacyl-CoA thiolase BktB (AAC38322.1) from R. eutropha or thiolase AtoB (NP_416728.1) from E. coli, 3-hydroxyacyl-CoA dehydrogenase and enoyl-CoA hydratase multifunctional enzyme FadB from E. coli (NP_418288.1) and enoyl-CoA reductase EgTer from E. gracilis (Q5EU90.1) or fatty acid biosynthesis pathway composed of beta-ketoacyl-ACP synthase FabH (NP_415609.1), beta-ketoacyl-ACP reductase FabG (NP_415611.1), 3-hydroxyacyl-ACP dehydratase FabZ (NP_414722.1) and enoyl-ACP reductase FabI (NP_415804.1), all from E. coli, with termination by e.g., E. coli thioesterase TesA (NP_415027.1, with truncation of 26 aa leader sequence) and activation by E. coli acyl-CoA synthetase FadD (NP_416319.1), or directly synthesized through overexpressed beta-oxidation reversal pathway without termination. If malonyl-CoA is used as the extender unit, to enhance its supply, e.g., E. coli acetyl-CoA carboxylase AccABCD is overexpressed. Also, to improve acetyl-CoA supply, e.g., E. coli pyruvate dehydrogenase complex AceEF-Lpd is overexpressed.

JST06(DE3) ΔfadE bktB^{CT5} ΔatoB fadB^{CT5} ΔfadA egter^{CT5} @fabI, which is able to intracellularly supply butyryl-CoA through beta-oxidation reversal, can serve as the host strain for the in vivo production of olivetolic acid. Its construction, growth and analysis of products are as described above in previous examples.

The in vivo butyryl-CoA and butyric acid synthesis through beta-oxidation reversal composed of AtoB, FadB and EgTer has been demonstrated in E. coli. The results are shown in FIG. 23. Strain and vector constructions, fermentation conditions and analysis method are as decribed above in previous examples.

(Prophetic) In Vivo Synthesis of Orsellinic Acid in E. coli

The purpose of this example is to demonstrate in vivo synthesis of orsellinic acid with E. coli as host organism. Orsellinic acid is a suitable aromatic acceptor of geranyl group donated from GPP, which is synthesized by claimed novel pathways or other pathways, in the prenylation reaction. Orsellinic acid is synthesized through multiple possible pathways. The first pathway starts from three series of decarboxylative Claisen condensations with acetyl-CoA as the initial primer and malonyl-CoA as the extender unit by e.g., C. sativa olivetol synthase OLS (BAG14339.1), generating 3,5,7-trioxooctanoyl-CoA. Then, C. sativa olivetolic acid cyclase OAC (AFN42527.1) cyclizes 3,5,7-trioxooctanoyl-CoA to orsellinic acid.

The second pathway also starts from three series of decarboxylative Claisen condensations with acetyl-CoA as the initial primer and malonyl-CoA as the extender unit, but catalyzed by other polyketide synthases selected from e.g., H. macrophylla stilbenecarboxylate synthase STCS (AAN76183.1, with a subset of mutations of T135S, T198M and I200C), a type III polyketide synthase, and type I polyketide synthases AviM from Streptomyces viridochromogenes Tue57 (AAK83194.1), ArmB from Armillaria mellea (AFL91703.1) and CalO5 from Micromonospora echinospora ssp. Calichensis (AAM70355.1). These polyketide synthases then directly perform the cyclization of 3,5,7-trioxooctanoyl-CoA to orsellinic acid.

The third pathway starts from condensation between two acetyl-CoAs to acetoacetyl-CoA catalyzed by E. coli thiolase AtoB (NP_416728.1). Then, two series of condensation reactions with acetoacetyl-CoA as the primer and acetyl-CoA as the extender unit by polyketoacyl-CoA thiolase selected from e.g., FadAx (AAK18171.1) and PcaF (AAA85138.1) from P. putida, DcaF (CAG68532.1) from Acinetobacter sp. ADP1, and ScFadA (AAL10298.1) from S. collinus, generates 3,5,7-trioxooctanoyl-CoA, which is then cyclized to orsellinic acid by OAC. If malonyl-CoA is used as the extender unit E. coli acetyl-CoA carboxylase AccABCD is preferably overexpressed. Also, to improve acetyl-CoA supply, E. coli pyruvate dehydrogenase complex AceEF-Lpd is overexpressed.

JST06(DE3) atoB^{CT5} ΔfadB serves as the E. coli host strain for demonstration of the novel pathway. Vector and strain creation, growth and analysis are as described in previous examples.

In Vivo Synthesis of CBGA in E. coli

The purpose of this example is to demonstrate in vivo synthesis of cannabigerolic acid (CBGA) with E. coli as host organism. In this example, Streptomyces sp. strain CL190 prenyltransferase NphB (BAE00106.1), which is soluble and desirable for functional expression and operation in E. coli, was used to convert GPP, which was synthesized through mevalonate pathway and GPP synthase in this example, and extracellularly supplemented olivetolic acid, into CBGA. Besides NphB, Lithospermum erythrorhizon PGT-1(Q8W405), Lithospermum erythrorhizon PGT-2 (Q8W404), E. coli UbiA (P0AGK1), Arabidopsis thaliana PPT1 (Q93YP7), Schizosaccharomyces pombe Coq2 (Q10252), Cannabis sativa CsPT1, Streptomyces coelicolor SC07190 (BAE00107.1), Streptomyces sp. CNQ-509 CnqP3 (AKH84817.1) and Phleum pretense Phlp4 (ABB78007.1) can be another options of prenyltransferases for transfer of geranyl group from GPP to olivetolic acid forming CBGA.

The mevalonate pathway used herein is composed of 3-hydroxy-3-methylglutaryl-CoA synthase HMGS (BAU36102.1) and 3-hydroxy-3-methylglutaryl-CoA reductase HMGR (OLN67110.1) from S. aureus, mevalonate kinase MK (NP_013935.1), phosphomevalonate kinase PMK (NP_013947.1) and phosphomevalonate decarboxylase PMD (NP_014441.1) from S. cerevisiae and E. coli isopentenyl pyrophosphate isomerase Idi (NP_417365.1). A. grandis GPP synthase TrGPPS2 (AAN01134.1, N-terminal 84 aa truncation) was selected for condensation of IPP and DMAPP to GPP.

Except for the gene encoding Idi, which was amplified from the genomic DNA of E. coli wild type MG1655 strain, the required genes were codon optimized and synthesized by either GeneArt or GenScript. The genes encoding HMGS, HMGR, MK, PMK and PMD were expressed through pCDF-P1-MK-PMK-PMD-P2-HMGS-HMGR, while the genes encoding Idi, TrGPPS2 and NphB were expressed through pET-P1-idi-trGPPS2-CymR-CT5-NphB. The primers used for constructions of these plasmids are listed in Table AA.

Primers NphB-IF-fwd and NphB-IF-rev were used to PCR amplify NphB gene from the synthesized DNA fragment with usage of Phusion polymerase, and the amplified DNA fragment was assembled with NdeI/KpnI digested pETDuet-1 vector by In-Fusion HD Eco-Dry Cloning system, resulting in plasmid pET-P2-NphB. Primers idi-GB-fwd, idi-GB-rev, trGPPS2-IF-fwd, and GPPS2-GB-rev were utilized to PCR amplify DNA fragments containing idi and trGPPS2 with usage of Phusion polymerase, respectively. These two amplified DNA fragments were assembled with NcoI digested pET-P2-NphB by Gibson assembly cloning system, resulting in plasmid pET-P1-idi-trGPPS2-P2-NphB.

Later, primers CymR-GB-fwd and CymR-GB-rev were used to amplify CymR with CT5 promoters, and NphB-cumate-GB-fwd and NphB-cumate-GB-rev were used to PCR amplify NphB fragment with usage of Phusion polymerase. Two amplified DNA fragments were assembled with NotI/XhoI digested pET-P1-idi-trGPPS2-P2-NphB by Gibson assembly, providing plasmid pET-P1-idi-trGPPS2-CymR-CT5-NphB.

For cloning plasmid pCDF-P1-MK-PMK-PMD-P1-HMGS-HMGR, the synthesized DNA fragments containing HMGS genes and HMGR genes were assembled with NdeI digested pCDFDuet-1 vector by In-Fusion HD Eco-Dry Cloning system, resulting plasmid pCDF-P2-HMGS-HMGR. Primers MK-IF-fwd and MK-IF-rev were used to PCR amplify DNA containing MK gene with usage of Phusion polymerase, and the amplified DNA fragment was assembled with NcoI/EcoRI digested pCDF-P2-HMGS-HMGR by in-fusion cloning, producing plasmid pCDF-P1-MK-P2-HMGS-HMGR.

Similarly, primer PMK-IF-fwd and PMK-IF-rev were used to PCR amplify PMK with usage of Phusion polymerase and the DNA fragment was assembled with EcoRI digested pCDF-P1-MK-P2-HMGS-HMGR by In-fusion cloning, resulting in plasmid pCDF-P1-MK-PMK-P2-HMGS-HMGR.

Finally, primers PMD-IF-fwd and PMD-IF-rev were utilized to PCR amplify PMD gene with usage of Phusion polymerase, and the amplified DNA fragments were assembled with EcorI digested pCDF-P1-MK-PMK-P2-HMGS-HMGR by In-fusion cloning, resulting in the plasmid pCDF-P1-MK-PMK-PMD-P2-HMGS-HMGR.

Host strain JST06(DE3) atoB^{CT5} containing plasmid pCDF-P1-MK-PMK-PMD-P2-HMGS-HMGR and pET-P1-idi-trGPPS2-CymR-CT5-NphB was inoculated into 5 ml LB medium in 25 ml flask with antibiotic and shaking under 37 °C with 200 rpm in NBS 124 Benchtop Incubator Shaker for overnight. The overnight culture was used as the seed culture to start the subculture with appropriate volume of LB-like MOPS medium as described above supplied with 20 g/L glucose in 25 ml flask. After 3 hours shaking under 37 °C at 200 rpm, the culture OD550 reached about 0.5. 20 μM IPTG and 100 μM cumate, and 500 mg/L olivetolic acid were added into the culture to induce enzyme expression and supply the substrate. Then, the flasks were transferred into another same type of shaker to grow under 30 °C. After growing for 48 hours, 2 mL of fermentation broths with or without cells were collected for GC-MS identification and GC-FID quantification of CBGA. If without cell, the 5000 g, 5 min centrifuge in an Optima L-80XP Ultracentrifuge (Beckman-Coulter, Schaumburg, IL) was performed to remove the cells.

The fermentation broths of 2 mL were transferred to 5 mL glass vials (Fisher Scientific Co., Pittsburgh, PA). Then, organic solvent (typically hexane) was added at a 1:1 ratio to a fermentation broth sample (e.g. 2 mL for a 2 mL aqueous solution) for extraction. Before extraction, the samples were acidified with sulfuric acid (80 uL per 2 mL sample) and 30% (w/v) NaCl was added (340 uL per 2 mL). Following an appropriate extraction (vortex samples for 15 seconds, spin on a rotator at 60 rpm for 2 hours, and vortex again for 15 seconds), 1 mL of the organic phase was removed and evaporated to dryness under a gentle N2 stream. 100 μL pyridine and 100 μL BSTFA were then added for derivatization, with the reaction allowed to proceed at 70 °C for 60 minutes. After cooling to room temperature, this mixture was used for GC analysis.

GC analysis was conducted on an Agilent 7890B Series Custom Gas Chromatography system equipped with a 5977B Inert Plus Mass Selective Detector Turbo EL Bundle (for identification) or a Flame Ionization Detector (for quantification) and an Agilent HP-5 capillary column (0.25 mm internal diameter, 0.25 μm film thickness, 30 in length). The following temperature profile was used with helium as the carrier gas at a flowrate of 1.2 mL/min: Initial 200 °C (hold 1 min); ramp at 30 °C/min to 300 °C (hold 5 min). The injector and detector temperature were 290 °C and 350 °C, respectively. 1 μL of sample was injected with a 4:1 split ratio.

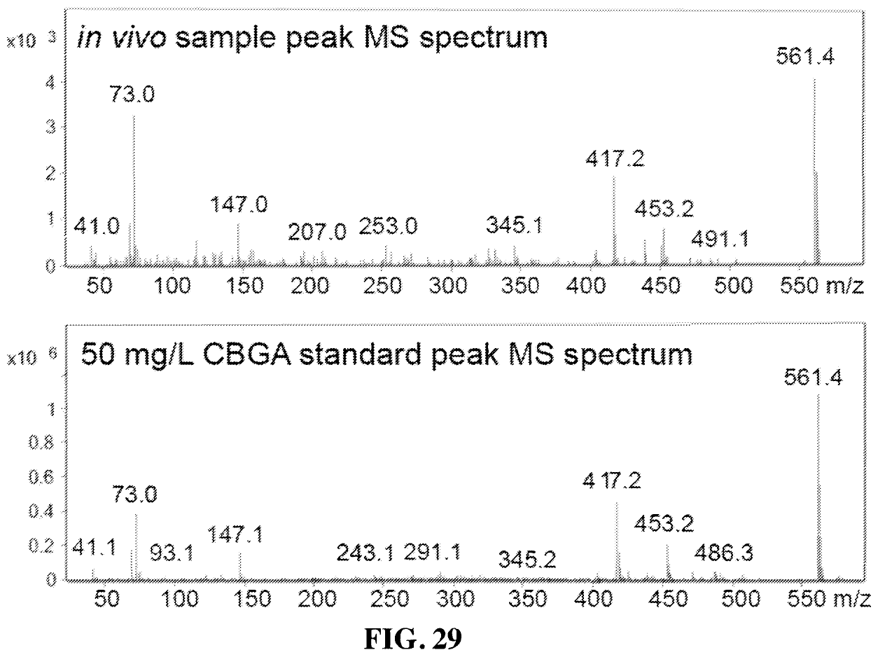

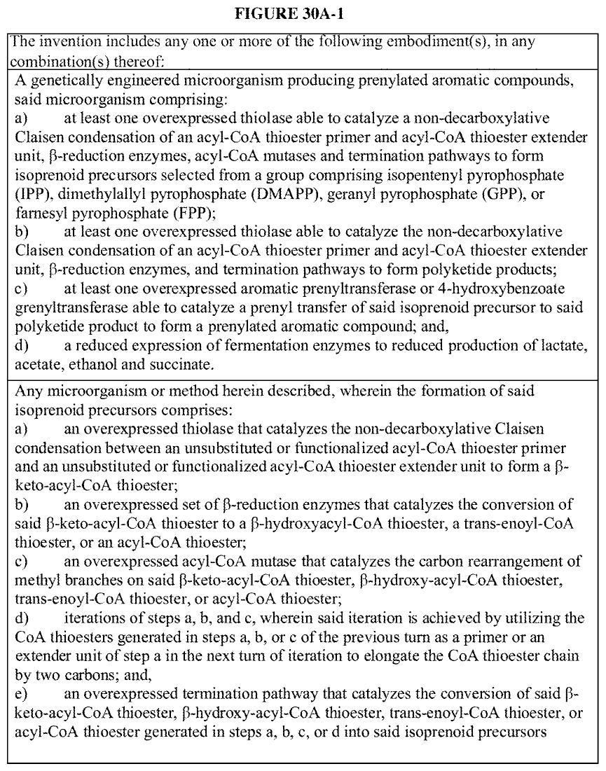

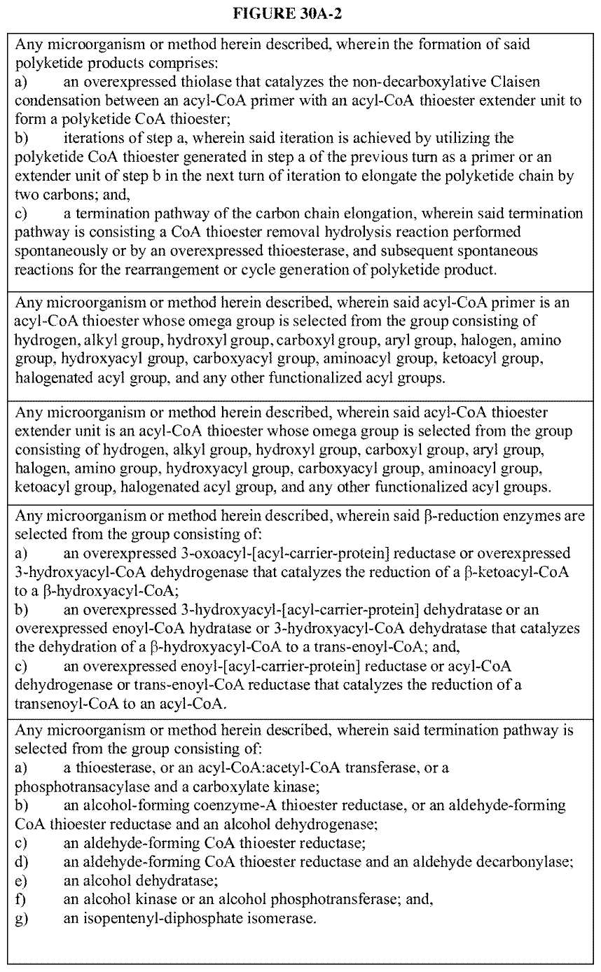

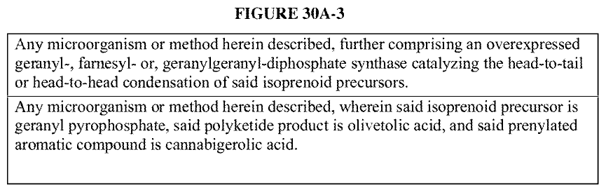

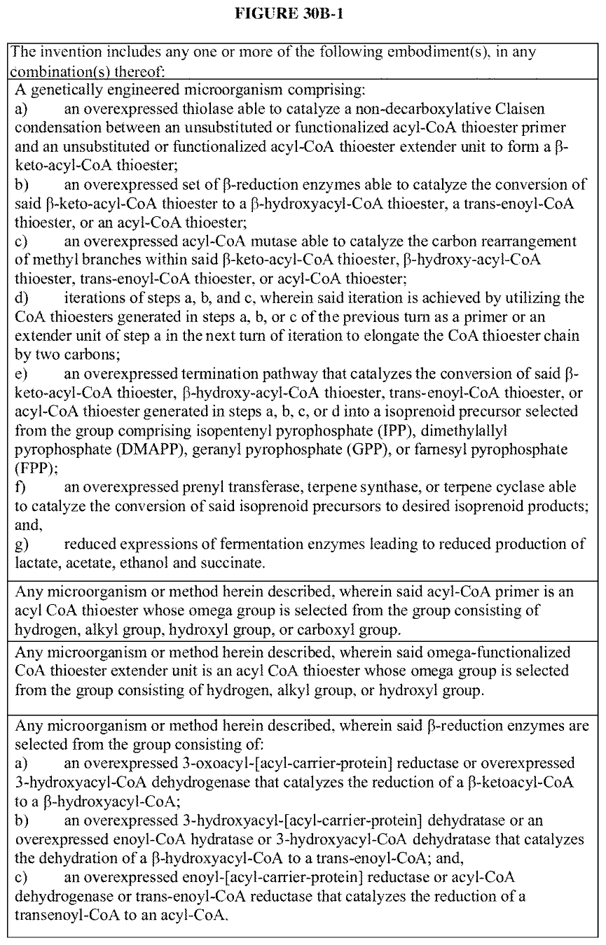

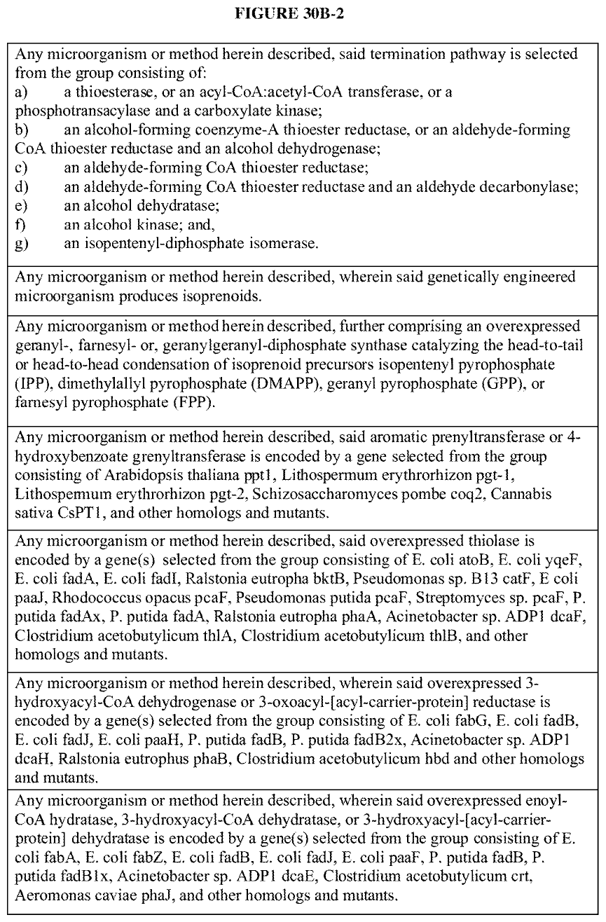

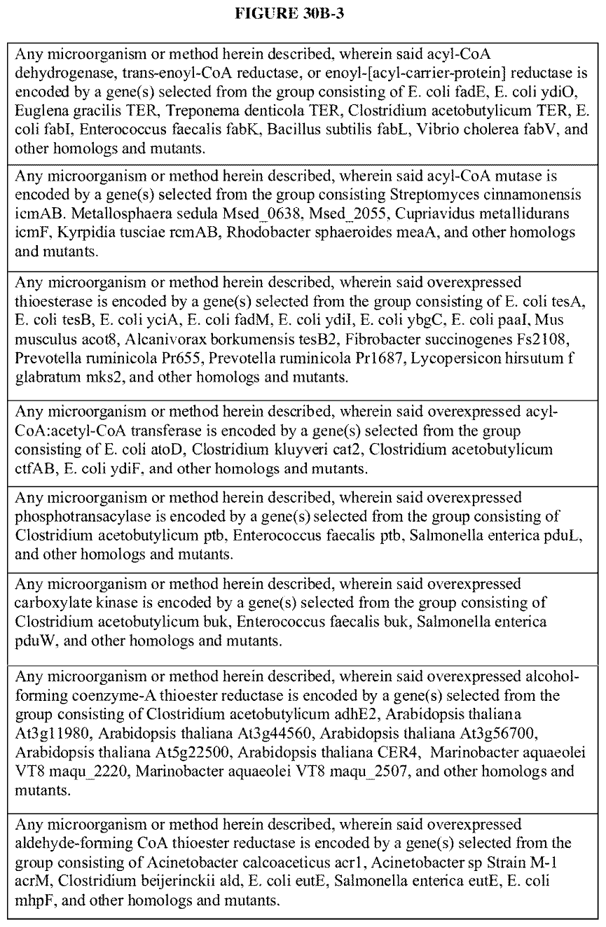

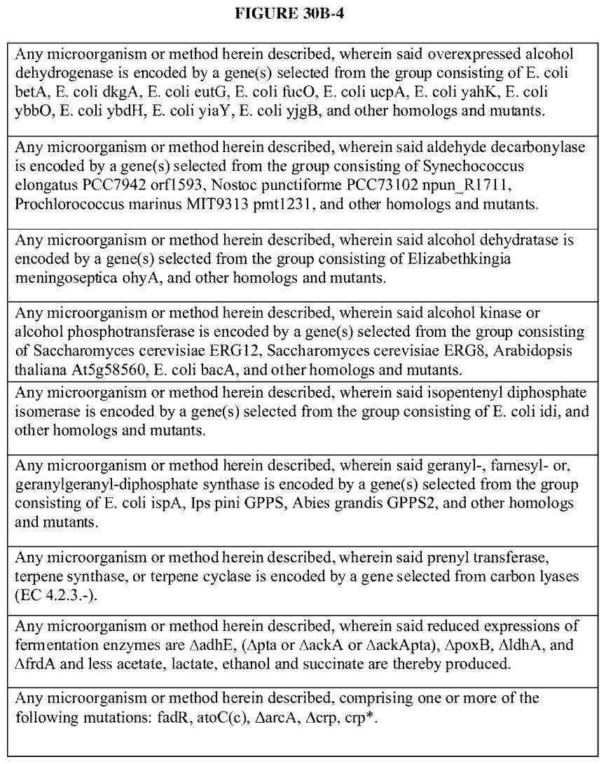

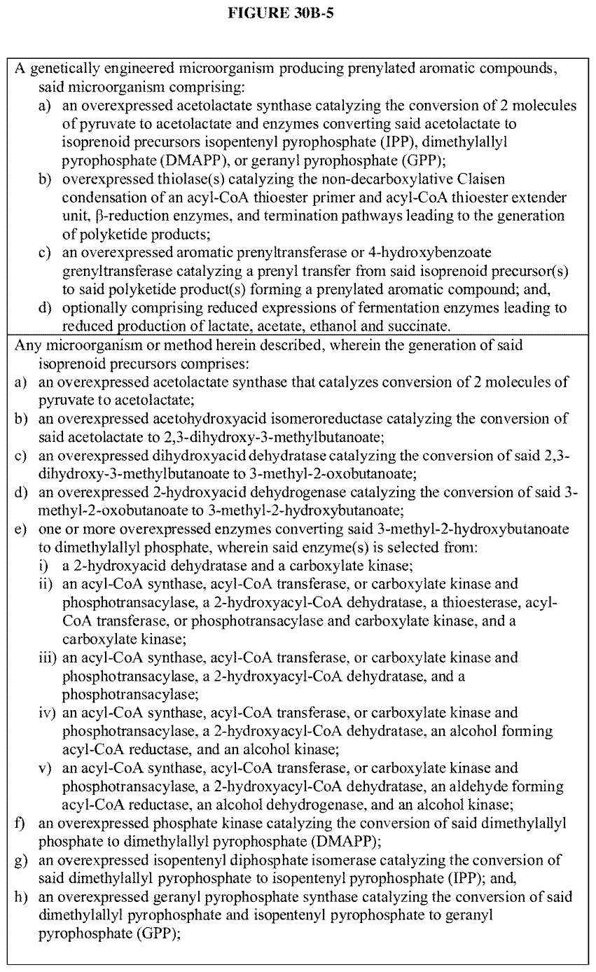

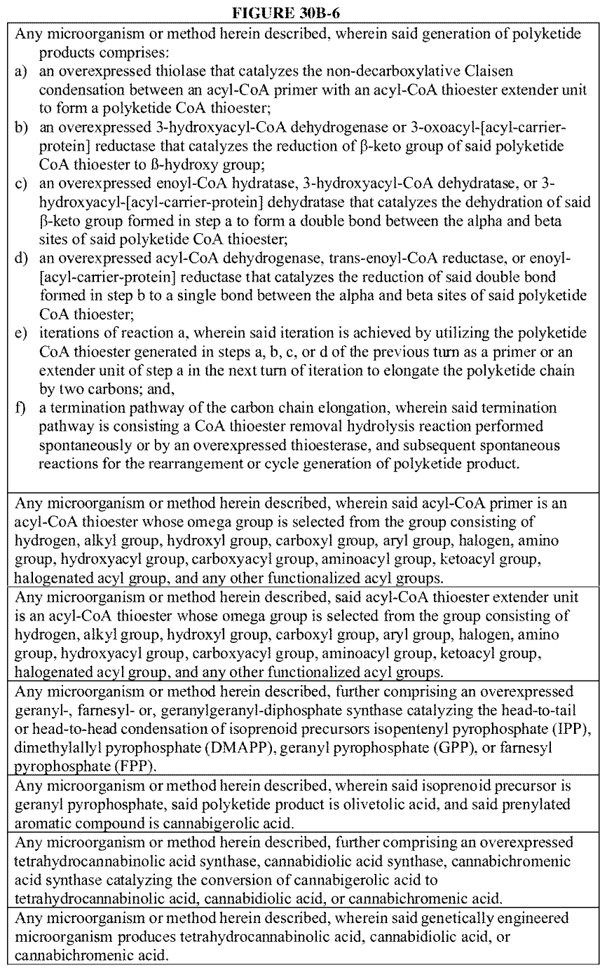

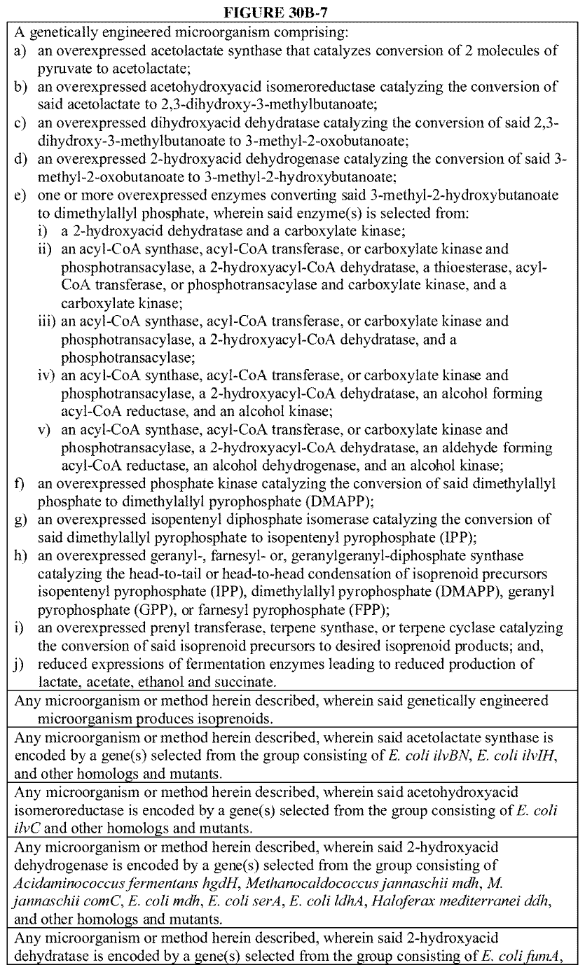

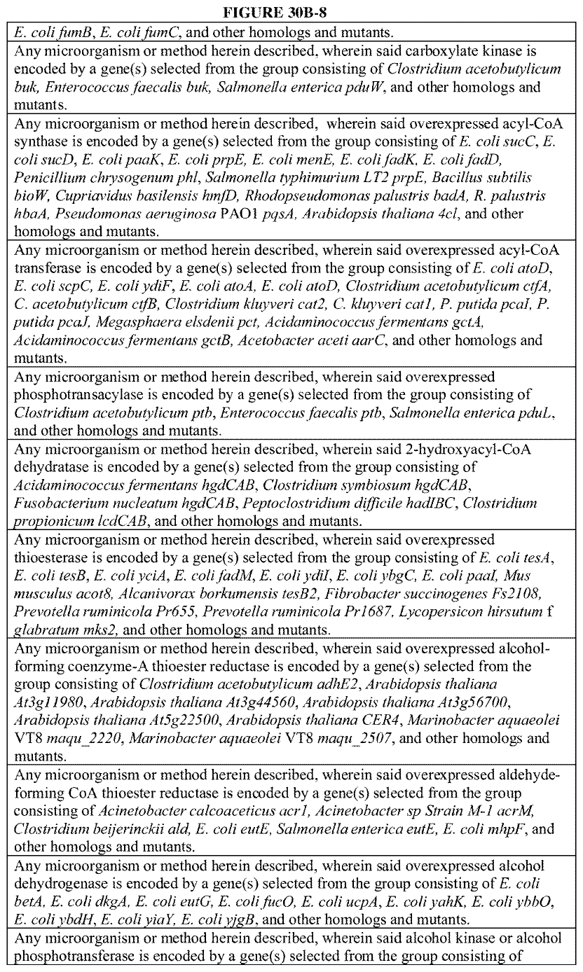

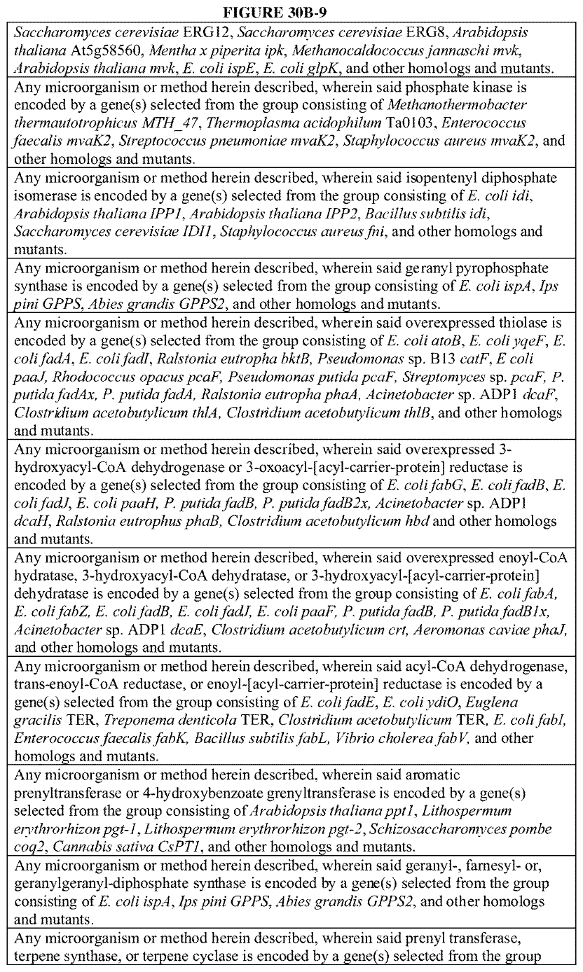

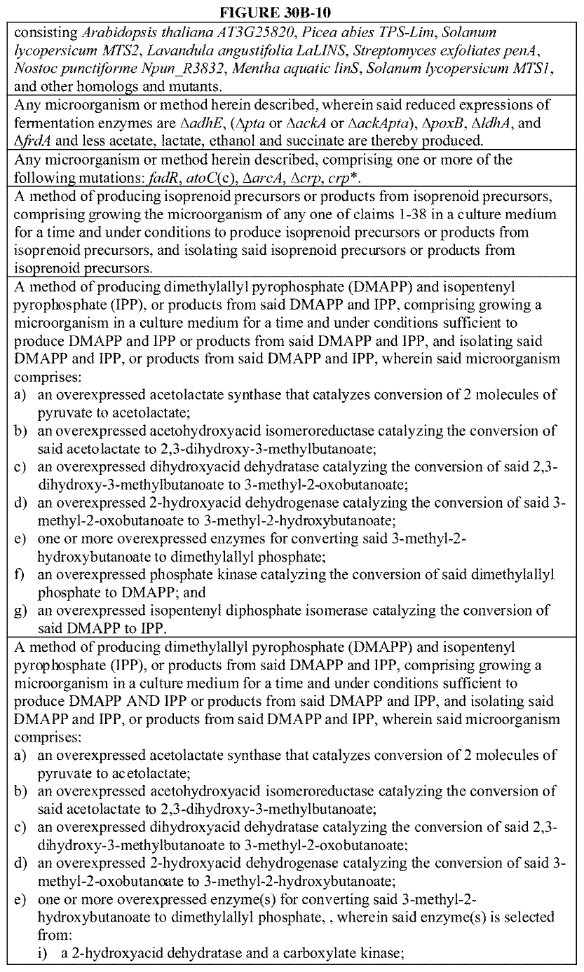

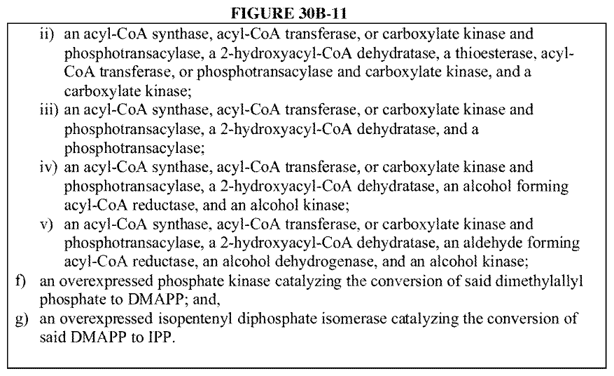

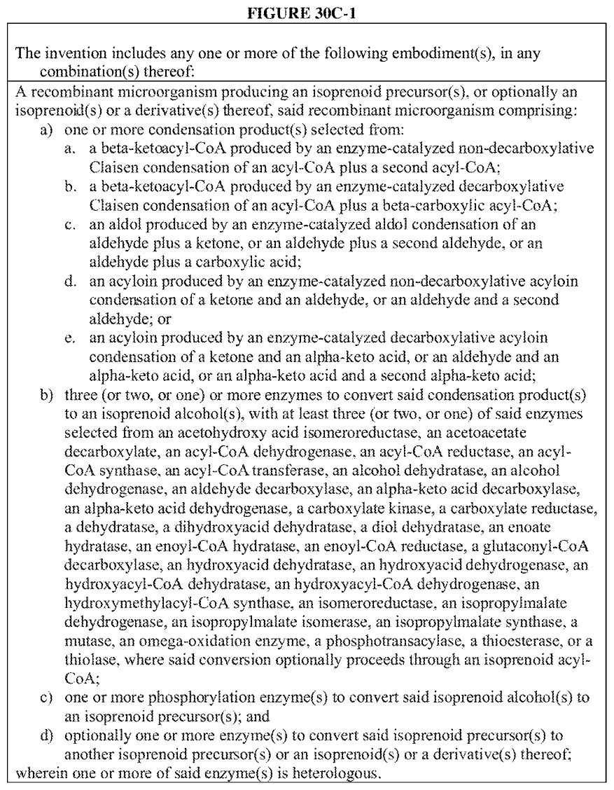

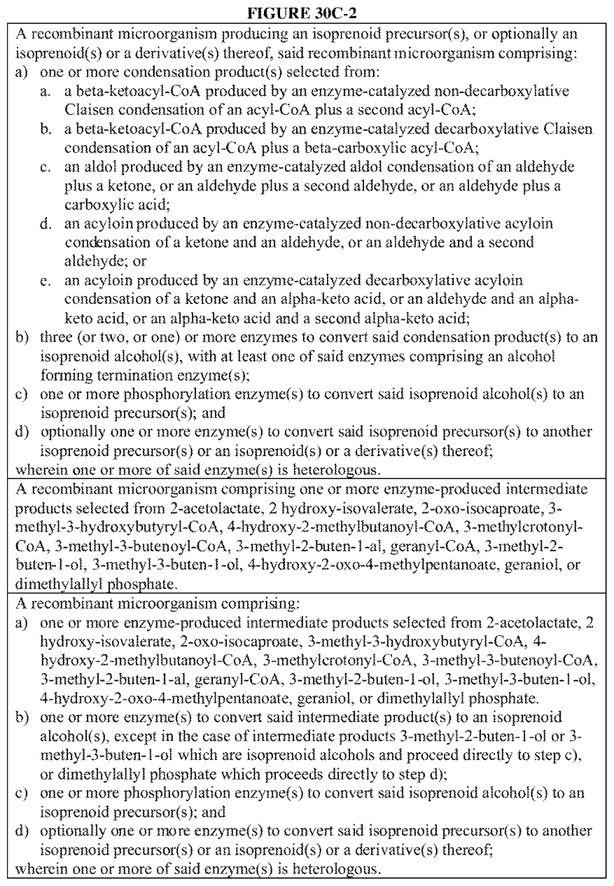

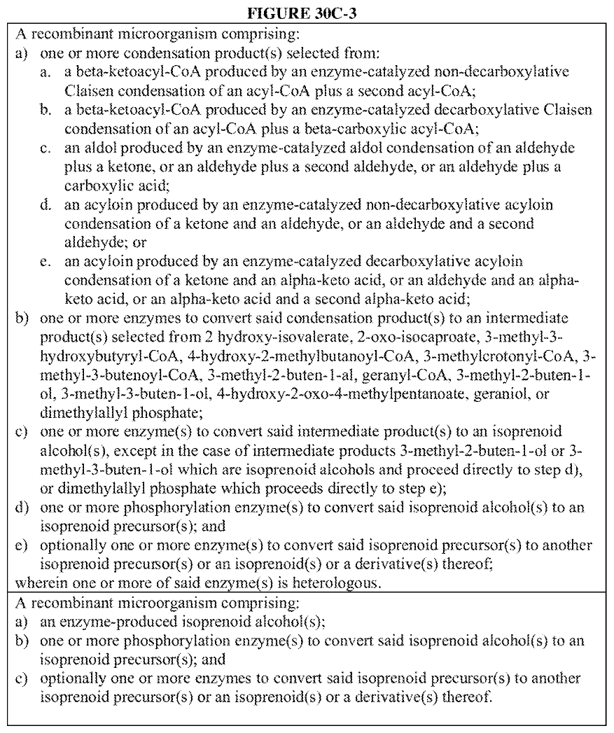

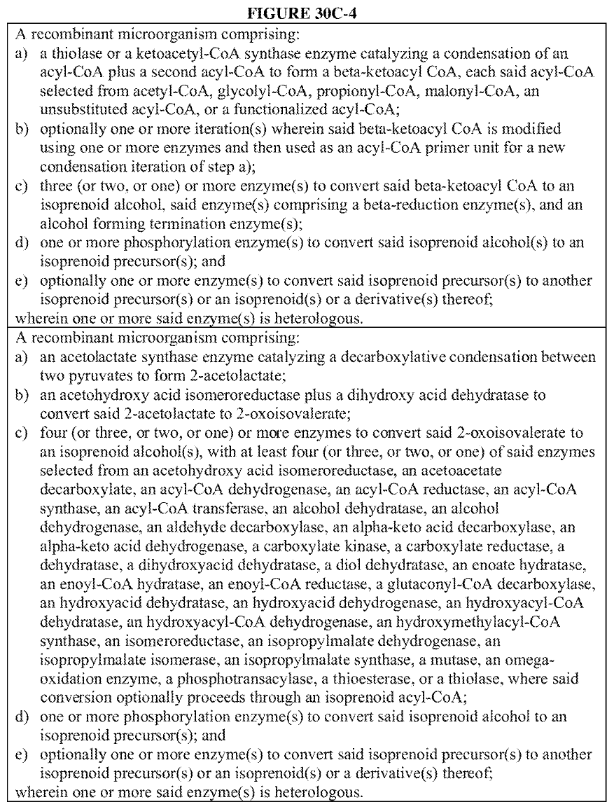

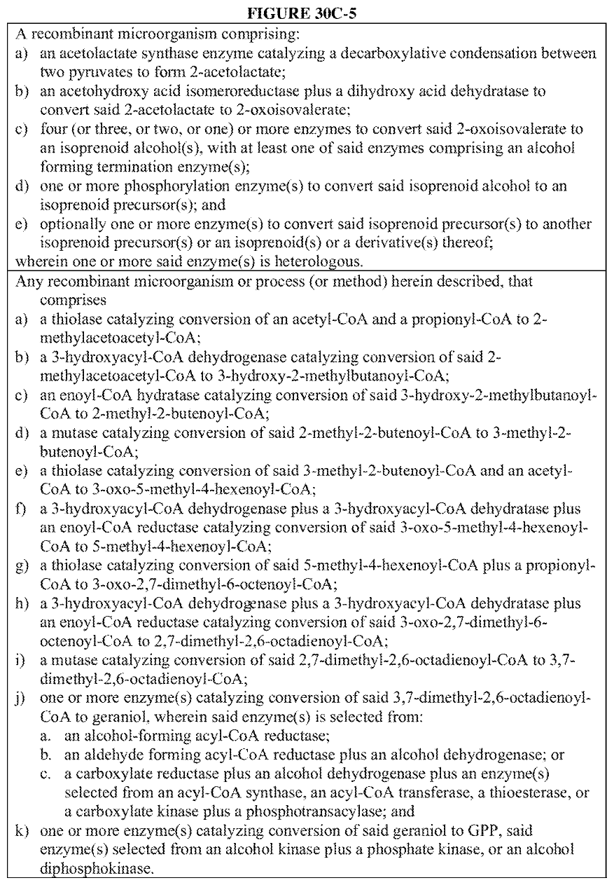

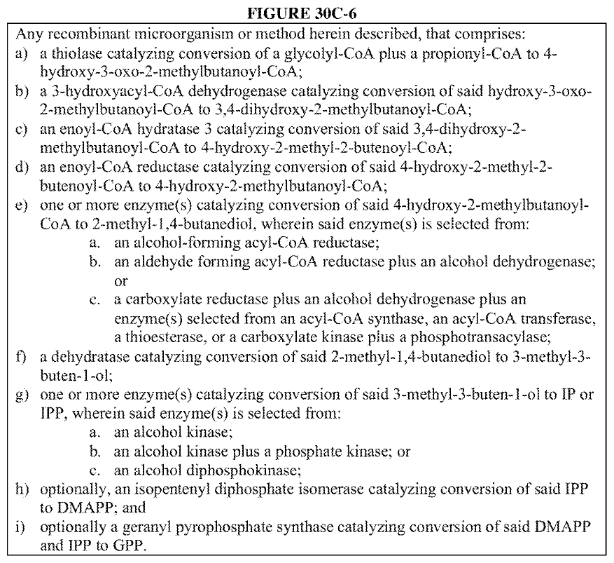

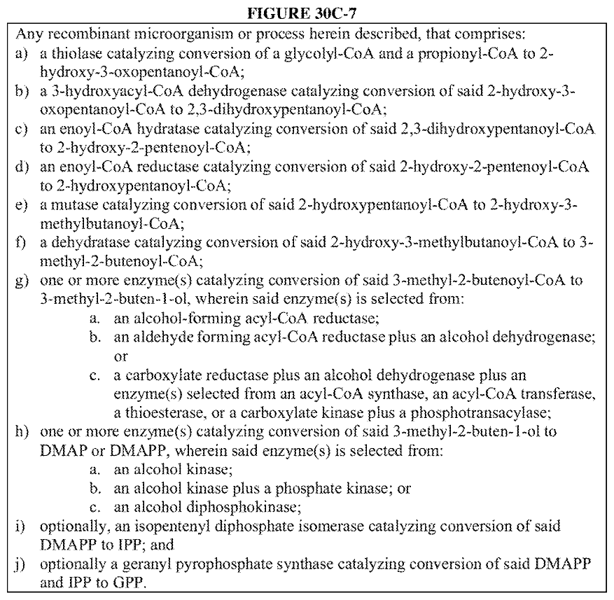

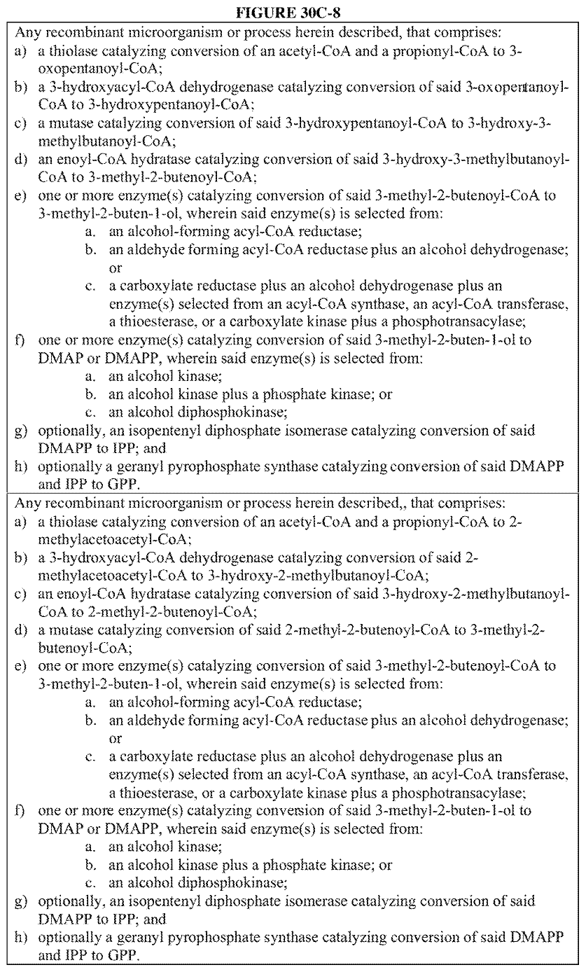

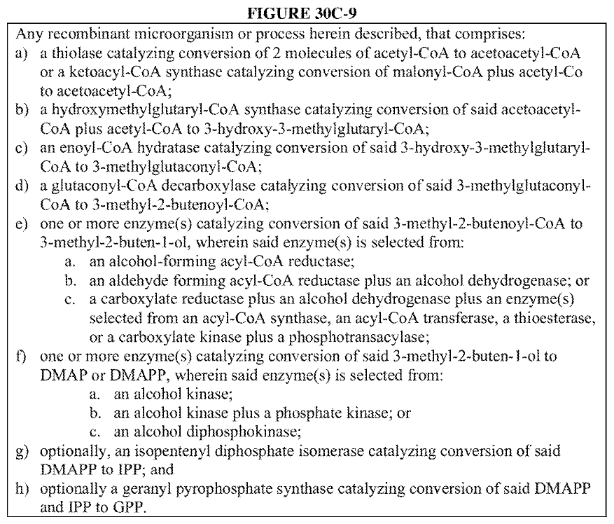

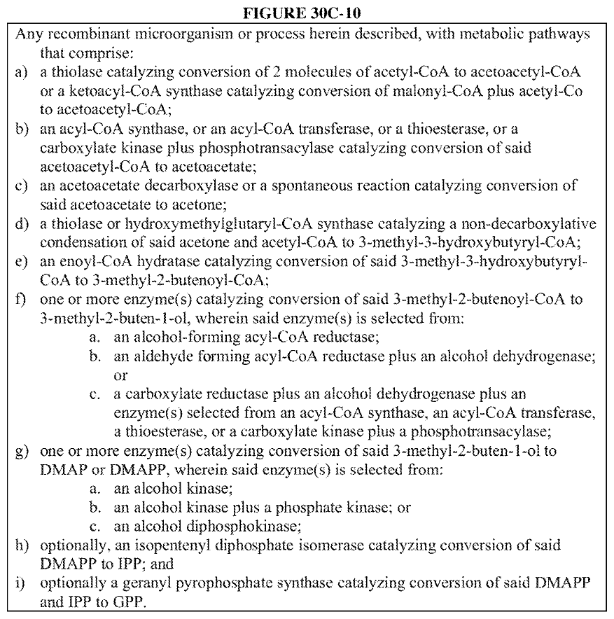

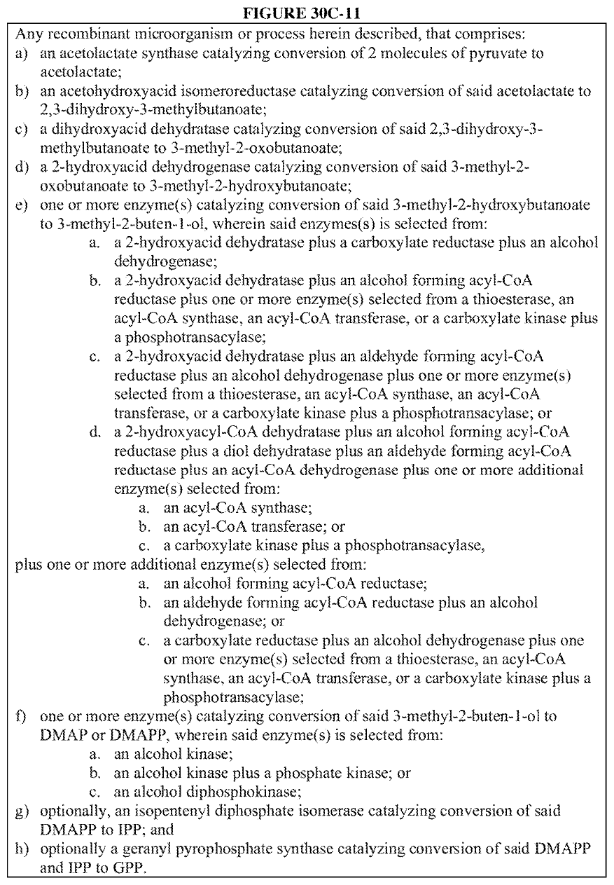

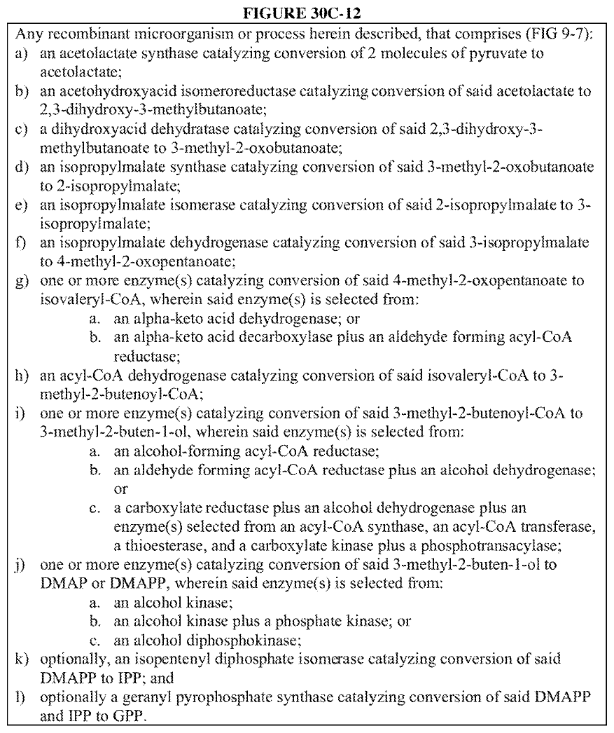

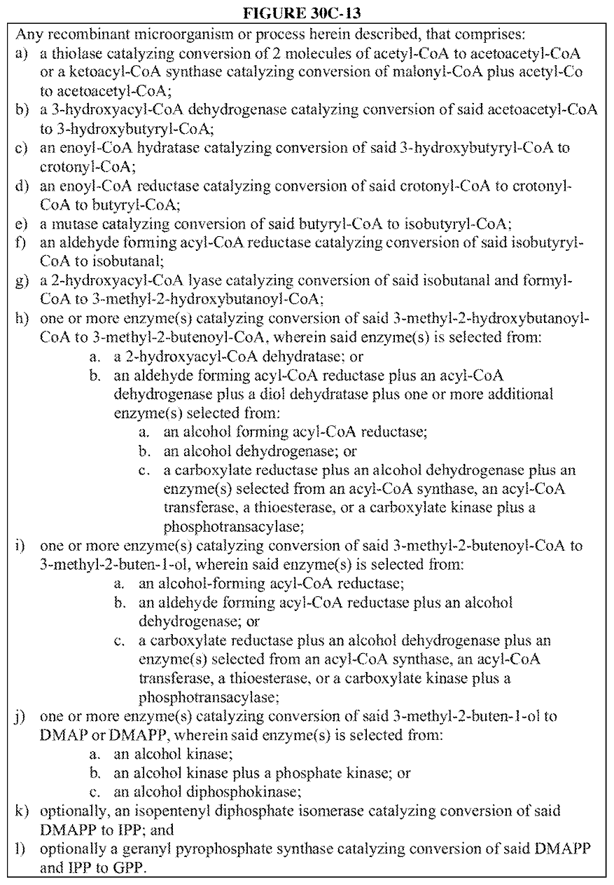

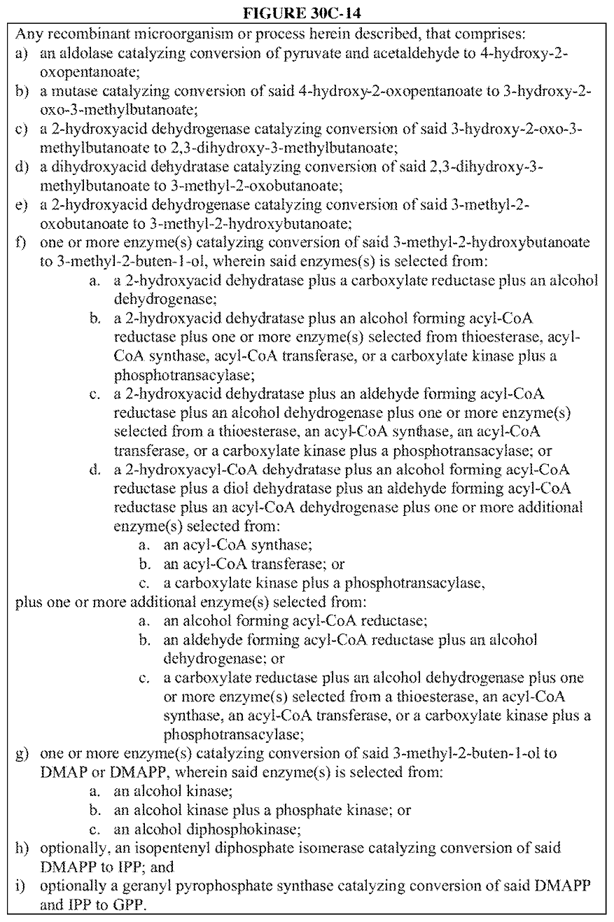

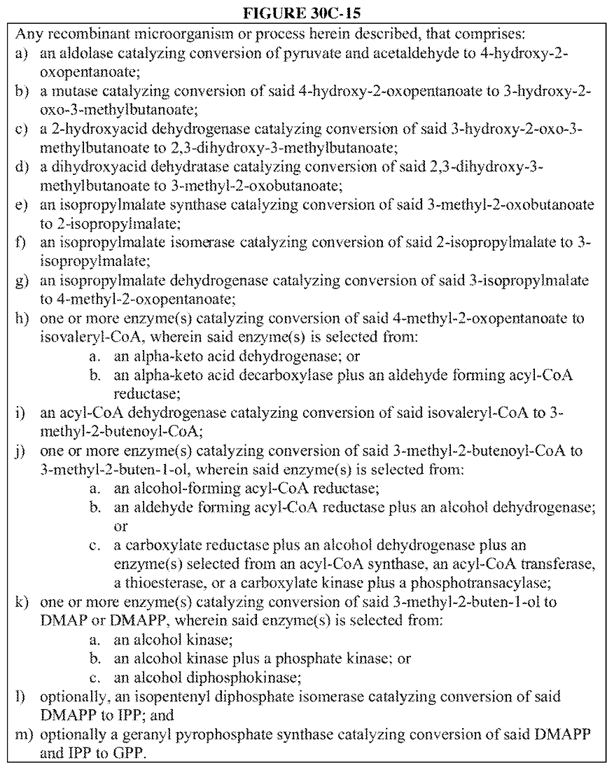

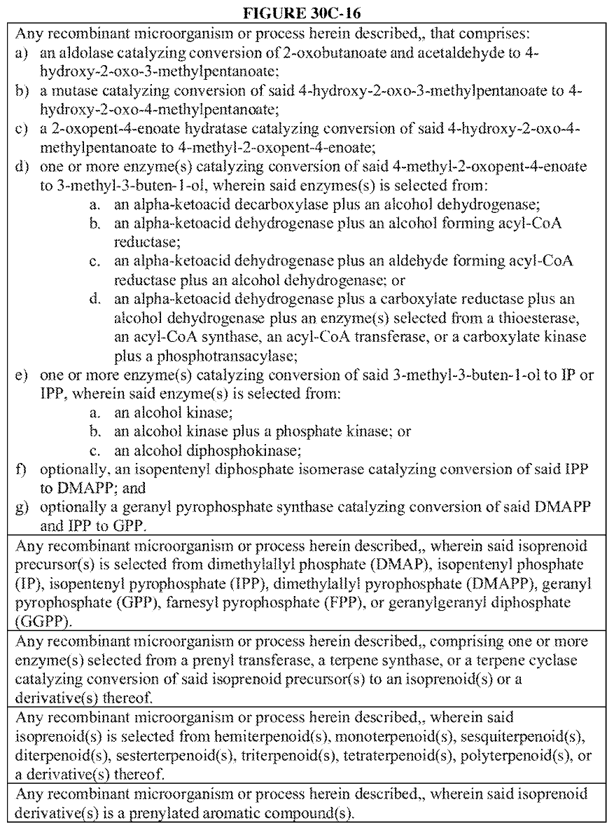

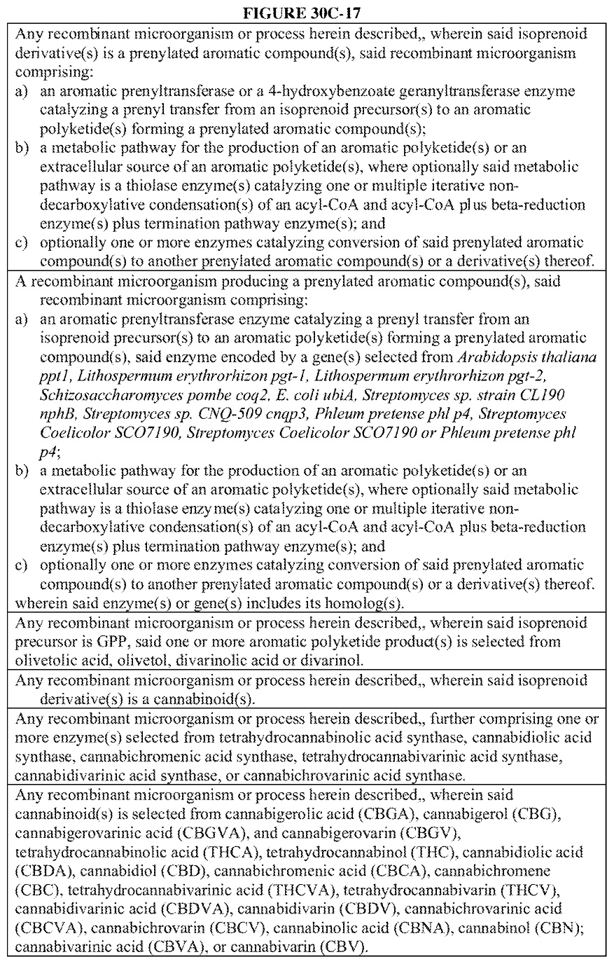

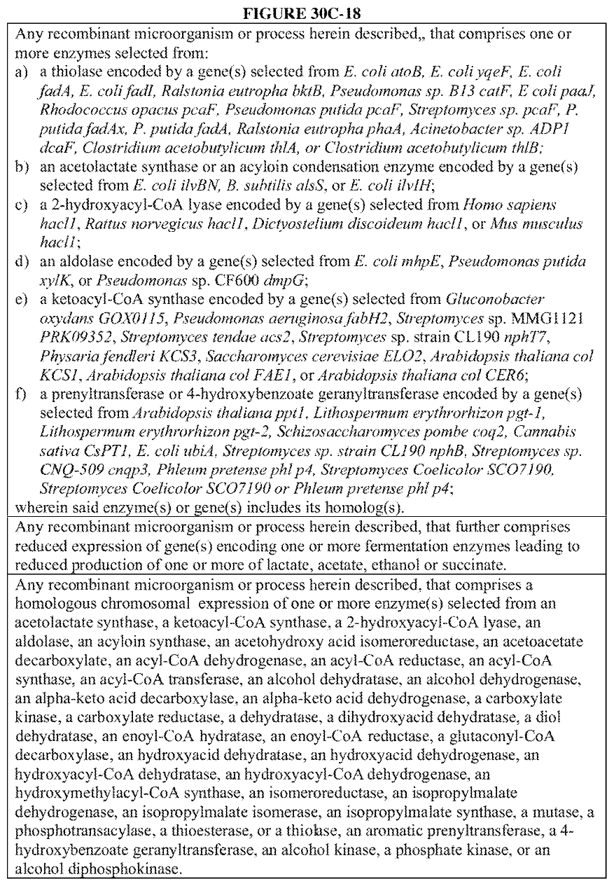

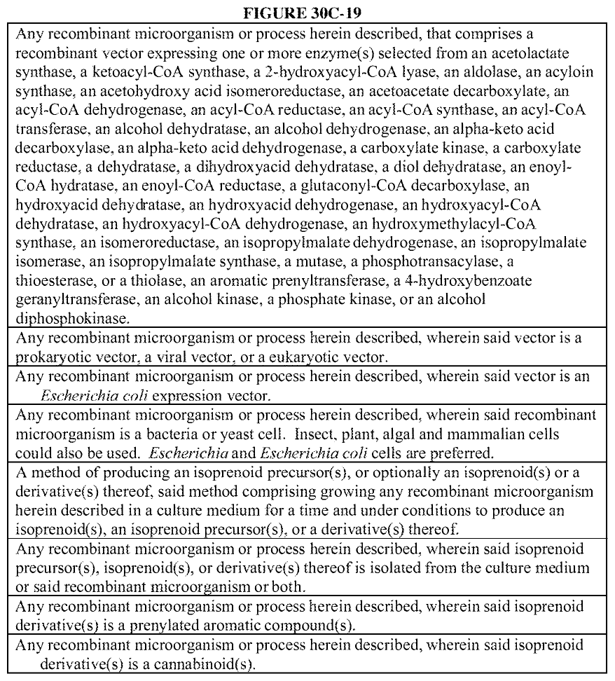

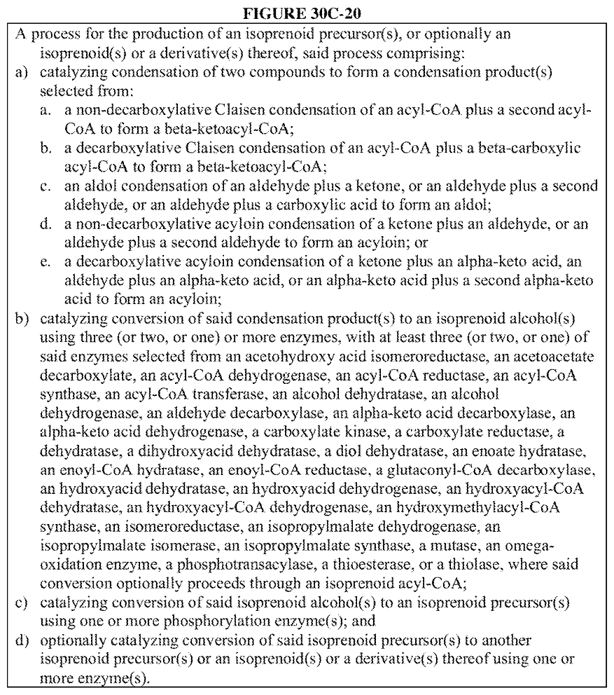

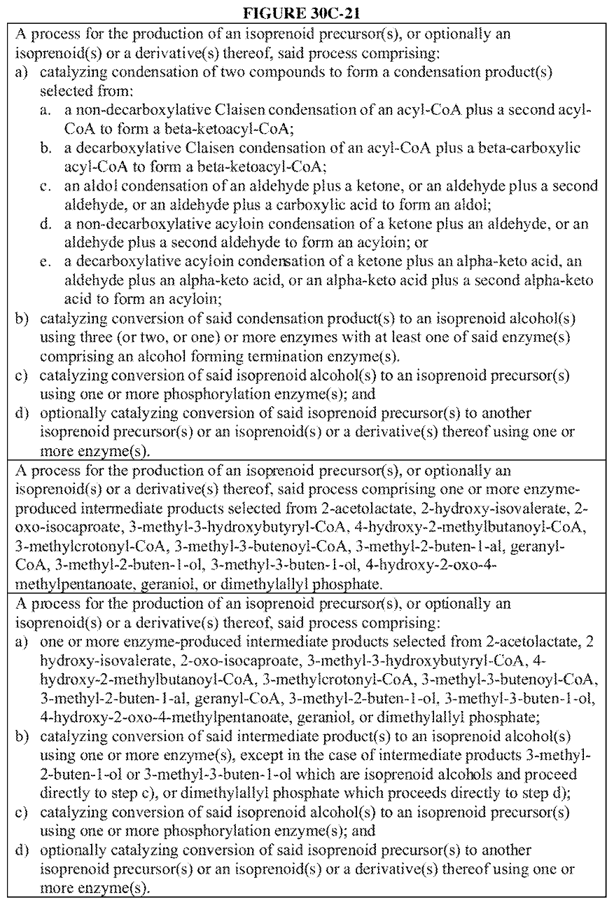

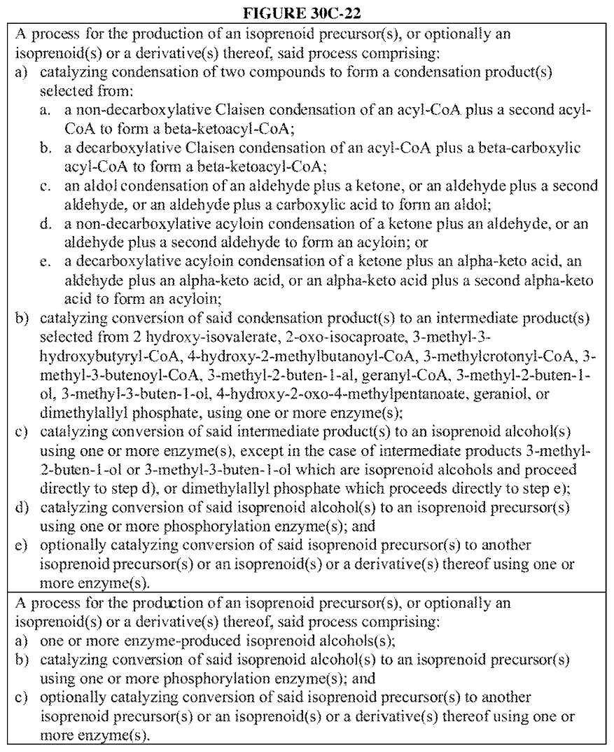

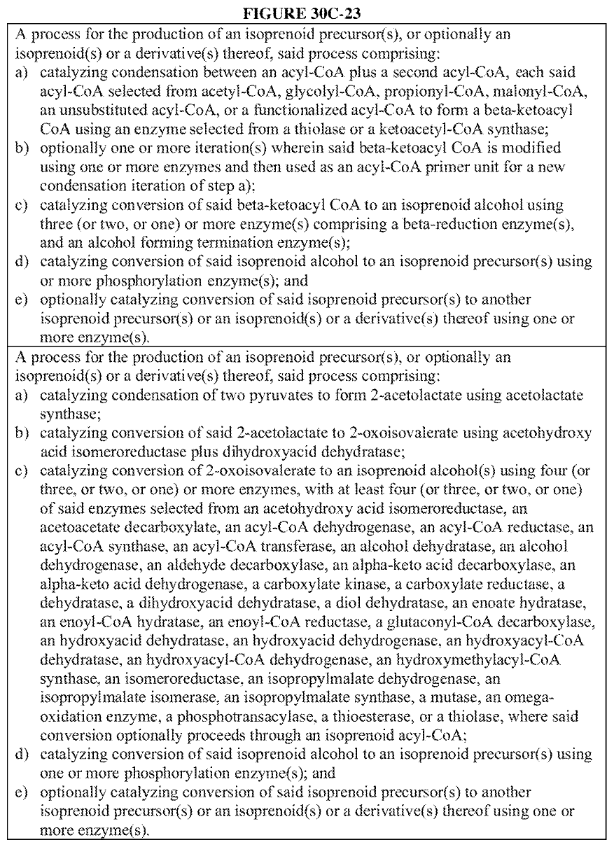

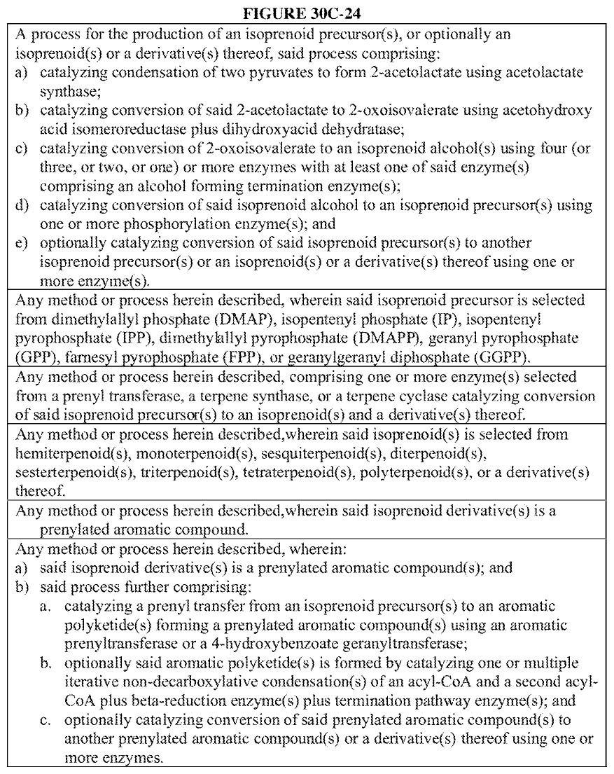

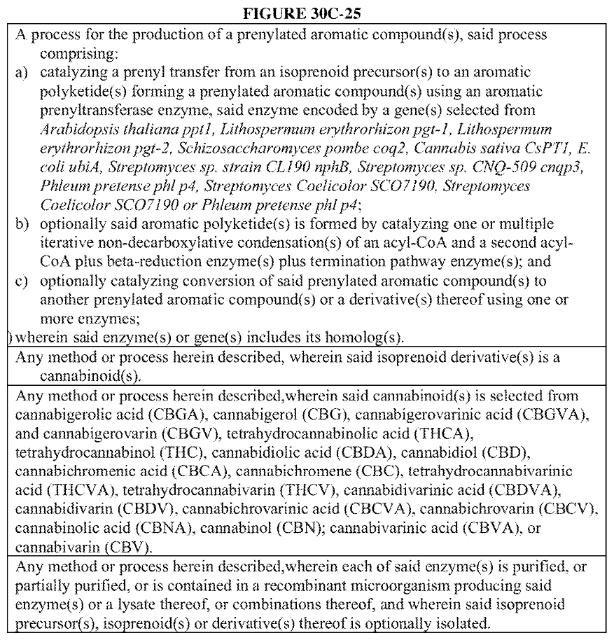

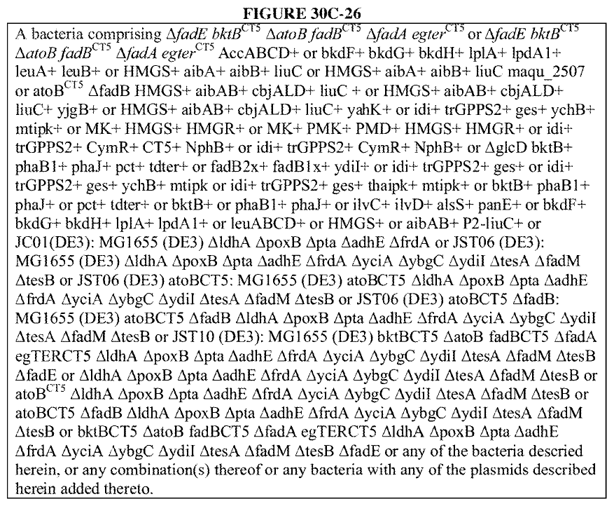

Cells grown with 10 mL medium in 25 ml flasks produced 0.2 mg/L CBGA after 48 hours of fermentation, and cells grown with 5 ml medium in 25 ml flasks produced 0.38 mg/L CBGA. FIG. 29 shows GC-MS identification of in vivo CBGA synthesis. This result indicates that prenyltransferase NphB is well expressed and functional on transferring geranyl group from GPP to olivetolic acid to synthesize CBGA in E. coli.

Although GPP in this example was supplied through traditional mevalonate pathway, the GPP could also be generated through claimed novel pathways or MEP/DXP pathway or commercially supplied. Alternative to the extracellular supplementation in this example, olivetolic acid can also be intracellularly synthesized through the series of condensations priming from hexanoyl-CoA as described in a previous example. Alternative to NphB used in this example, prenyl transfer can be catalyzed by other suitable enzymes such as those examples listed in Table L. The vectors for expression of some of prenyltransferases have been constructed, which are shown in Table AB.

The following are incorporated by reference herein in its entirety for all purposes:
Atsumi, S. et al. Metabolic engineering of Escherichia coli for 1-butanol production. Metab. Eng. 10, 305-311 (2008).
Baba, T. et al. Construction of Escherichia coli K-12 in-frame, single-gene knockout mutants: the Keio collection. Mol Syst Biol 2 (2006).
Blecher, M. (1981). Synthesis of long-chain fatty acyl-CoA thioesters using N-hydroxysuccinimide esters. Methods in Enzymology, 72: 404-408.
Chen, J. et al. Activating C₄-dicarboxylate transporters DcuB and DcuC for improving succinate production. Appl. Microbiol. Biotechnol. 98, 2197-2205 (2014).
Cheong, S., et al. Energy- and carbon-efficient synthesis of functionalized small molecules in bacteria using non-decarboxylative Claisen condensation reactions. Nat Biotech 34, 556-561 (2016).
Choi, S. Y. et al. One-step fermentative production of poly(lactate-co-glycolate) from carbohydrates in Escherichia coli. Nat Biotech 34, 435-440 (2016).
Clomburg, J. M., et al. A Synthetic Biology Approach to Engineer a Functional Reversal of the beta-Oxidation Cycle. ACS Synthetic Biology 1, 541-554 (2012).
Cracan, V. R., et al. Novel B12-dependent acyl-CoA mutases and their biotechnological potential. Biochemistry 51(31), 6039-6046 (2012).
Dellomonaco, C., et al. Engineered reversal of the beta-oxidation cycle for the synthesis of fuels and chemicals. Nature 476, 355-359 (2011).
Gao, Y., et al. Terpenoid synthase structures: a so far incomplete view of complex catalysis. Natural product reports 29, 1153-1175 (2012).
Haapalainen, A. M., et al. The thiolase superfamily: condensing enzymes with diverse reaction specificities. Trends in Biochemical Sciences 31, 64-71 (2006).
Heath, R. J., et al. The Claisen condensation in biology. Nat. Prod. Rep. 19, 581-596 (2002).
Iijima, Y., et al. Characterization of Geraniol Synthase from the Peltate Glands of Sweet Basil. Plant Physiol. 134, 370-379 (2004).
Jiang, C., et al. Divergent evolution of the thiolase superfamily and chalcone synthase family. Molecular Phylogenetics and Evolution 49, 691-701 (2008).
Jiang, Y. et al. Multigene Editing in the Escherichia coli Genome via the CRISPR-Cas9 System. Appl. Environ. Microbiol. 81, 2506-2514 (2015).
Kim, J., et al. W. 2-Hydroxyisocaproyl-CoA dehydratase and its activator from Clostridium difficile. Febs J. 272, 550-561 (2005).
Kitagawa, M. et al. Complete set of ORF clones of Escherichia coli ASKA library (A complete Set of E. coli K-12 ORF archive): Unique resources for biological research. DNA Res. 12, 291-299 (2005).
Kuzuyama, T., et al. Structural basis for the promiscuous biosynthetic prenylation of aromatic natural products. Nature 435, 983-987 (2005).
Liu, Y, et al. Improving the catalytic activity of isopentenyl phosphate kinase through protein coevolution analysis. Scientific Reports 6, 24117 (2016).
Mabanglo, M. F., et al. Mutagenesis of isopentenyl phosphate kinase to enhance geranyl phosphate kinase activity. ACS Chem. Biol. 7, 1241-1246 (2012).
Magner, D. B. et al. RecQ promotes toxic recombination in cells lacking recombination intermediate-removal proteins. Mol. Cell 26, 273-286 (2007).
Miller, J. H. Experiments in molecular genetics. (Cold Spring Harbor Laboratory, Cold Spring Harbor, N.Y.; 1972).
Neidhardt, F. C. et al. Culture medium for enterobacteria. J. Bacteriol. 119, 736-747 (1974).
Parasaran, T, et al. Formic Ethylcarbonic Anhydride. The Journal of Organic Chemistry, 29(11): 3422-3423 (1964).
Reiling, K. K. et al. Mono and diterpene production in Escherichia coli. Biotechnol Bioeng 87 (2004).
Sambrook, J. et al. Molecular cloning: a laboratory manual Edn. 3rd. (Cold Spring Harbor Laboratory Press., Cold Spring Harbor, N.Y.; 2001).
Schrader, J. et al. Biotechnology of Isoprenoids. Vol. 149. Springer, (2015).
Studier, F. W., et al. Use of bacteriophage T7 RNA polymerase to direct selective high-level expression of cloned genes. J. Mol. Biol. 189, 113-130 (1986).
Thomason, L. C., et al. Current Protocols in Molecular Biology (John Wiley & Sons, Inc., 2001).
Vick, J. E. et al. Escherichia coli enoyl-acyl carrier protein reductase (FabI) supports efficient operation of a functional reversal of the beta-oxidation cycle. Appl. Environ. Microbiol. 81, 1406-1416 (2015).
Vick, J. E. et al. Optimized compatible set of BioBrick™ vectors for metabolic pathway engineering. Appl. Microbiol. Biotechnol. 92, 1275-1286 (2011).
Yazdani, S. S. et al. Engineering Escherichia coli for the efficient conversion of glycerol to ethanol and co-products. Metab. Eng. 10, 340-351 (2008).
US20130316413 WO2012109176 61/440,192, Reverse beta oxidation pathway.
US20140273110 WO2013036812 61/531,911 61/440,192, Functionalized carboxylic acids and alcohols by reverse fatty acid oxidation.
WO2015191972 62/011,474, 62/012,113, 62/011,465, 61/531,911, WO2013036812, US20140273110, Omega-carboxylated carboxylic acids and derivitives.
WO2015191422 62/011,465, 62/012,113, 62/011,474, 61/531,911, WO2013036812, 14/199,528, Omega-hydroxylated carboxylic acids.
WO2016007258 62/011,474, 62/012,113, 62/011,465, 61/531,911, WO2013036812, US20140273110, Omega-aminated carboxylic acids.
WO2017020043, BIOSYNTHESIS OF POLYKETIDES, filed Aug. 1, 2016, and 62/198,764, filed Jul. 30, 2015.
U.S. Ser. No. 62/308,937, filed Mar. 16, 2016.
U.S. Ser. No. 62/198,764, filed Jul. 30, 2015.

TABLE A
Example reactions and enzymes of the Claisen condensation platform for the synthesis of isoprenoid precursors

| | | | | Source organism | Protein |
|---|---|---|---|---|---|
| | | EC | Enzyme | and gene/enzyme | Accession |
| Reaction | Illustration | Numbers | names | name | Numbers |
| | | | | Clostridium | AAA95971.1 |
| | | | | acetobutylicum hbd | |
| | | | 3-oxoacyl- | E. coli fabG | NP_415611.1 |
| | | | [acyl-carrier- | | |
| | | | protein] | | |
| | | | reductase | | |
| beta- hydroxyacyl- CoA → enoyl- CoA | 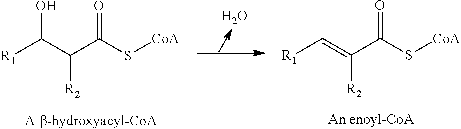 | 4.2.1.17; 4.2.1.119 | enoyl-CoA hydratase | E. coli fadB E. coli fadJ E. coli paaF P. putida fadB P. putida fadB1x Acinetobacter sp. ADP1 dcaE | NP_418288.1 NP_416843.1 NP_415911.1 AAK18167.2 AAK18173.1 CAG68535.1 |
| | | | | Clostridium | AAA95967.1 |
| | | | | acetobutylicum crt | |
| | | | | Aeromonas caviae | O32472.1 |
| | | | | phaJ | |
| | | | 3- | E. coli fabA | NP_415474.1 |
| | | | hydroxyacyl- | E. coli fabZ | NP_414722.1 |
| | | | [acyl-carrier- | | |
| | | | protein] | | |
| | | | dehydratase | | |
| Enoyl-CoA → Acyl-CoA | 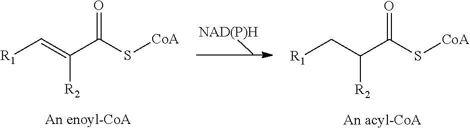 | 1.3.1.44 | enoyl-CoA reductase | Euglena gracilis TER Treponema denticola TER Clostridium acetobutylicum TER | Q5EU90.1 4GGO_A 4EUH_A |
| | | | enoyl-[acyl- | E. coli fabI | NP_415804.1 |
| | | | carrier- | Enterococcus | NP_816503.1 |
| | | | protein] | faecalis fabK | |
| | | | reductase | Bacillus subtilis | KFK80655.1 |
| | | | | fabL | |
| | | | | Vibrio cholerae | ABX38717.1 |
| | | | | fabV | |
| | | | acyl-CoA | E. coli fadE | NP_414756.2 |
| | | | dehydrogenase | E. coli ydiO | NP_416210.4 |
| Carbon rearrangement (select examples) | 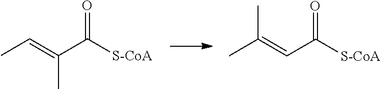 | 5.4.99.- | acyl-CoA mutase | Streptomyces cinnamonensis icmAB | AAC08713.1. CAB59633.1 |
| | 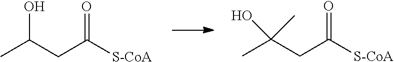 | | | Metallosphaera sedula Msed_0638, Msed_2055 | A4YEG1, A4YIE3 |
| | 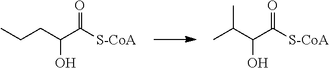 | | | Cupriavidus metallidurans icmF | Q1LRY0 |
| | | | | Kyrpidia tusciae rcmAB | D5WTR7, D5WTR8 |
| | | | | Rhodobacter sphaeroides meaA | ABA80144.1 |

TABLE B
Example termination pathways and enzymes

| | | | | Source organism | Protein |
|---|---|---|---|---|---|
| | | EC | Enzyme | and gene/enzyme | Accession |
| Reaction | Illustration | Numbers | names | name | Numbers |
| | | 3.1.2.- | Thioesterase | E. coli tesA | NP_415027.1 |
| Acyl-CoA → Carboxylic acid | 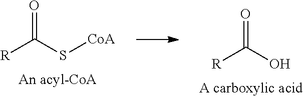 | | | E. coli tesB E. coli yciA E. coli fadM E. coli ydiI E. coli ybgC | NP_414986.1 NP_415769.1 NP_414977.1 NP_416201.1 NP_415264.1 |
| | | | | E. coli paaI | NP_415914.1 |
| | | | | Mus musculus | P58137.1 |
| | | | | acot8 | |
| | | | | Lycopersicon | ADK38536.1 |
| | | | | hirsutum f | |
| | | | | glabratum mks2 | |
| | | | | Alcanivorax | YP_692749.1 |
| | | | | borkumensis tesB2 | |
| | | | | Fibrobacter | YP_005822012.1 |
| | | | | succinogenes | |
| | | | | Fs2108 | |
| | | | | Prevotella | YP_003574018.1 |
| | | | | ruminicola Pr655 | |
| | | | | Prevotella | YP_003574982.1 |
| | | | | ruminicola Pr1687 | |
| | | 2.8.3.8 | Acyl- | E. coli atoD | NP_416725.1 |
| | | | CoA:acetyl- | Clostridium kluyveri | AAA92344.1 |
| | | | CoA | cat2 | |
| | | | transferase | Clostridium | NP_149326.1, |
| | | | | acetobutylicum | NP_149327.1 |
| | | | | ctfAB | |
| | | | | E. coli ydiF | NP_416209.1 |
| | | 2.3.1.-; | Phosphotrans- | Clostridium | NP_349676.1 |
| | | 2.7.2.1; | acylase + | acetobutylicum ptb | |
| | | 2.7.2.15 | Carboxylate | Enterococcus | AAD55374.1 |
| | | | kinase | faecalis ptb | |
| | | | | Salmonella enterica | AAD39011.1 |
| | | | | pduL | |
| | | | | Clostridium | AAK81015.1 |
| | | | | acetobutylicum buk | |
| | | | | Enterococcus | AAD55375.1 |
| | | | | faecalis buk | |
| | | | | Salmonella enterica | AAD39021.1 |
| | | | | pduW | |
| Acyl-CoA → Alcohol | 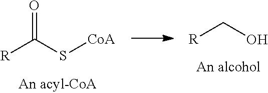 | 1.2.1.84 | Alcohol- forming CoA reductase | Clostridium acetobutylicum adhE2 Arabidopsis thaliana At3g11980 Arabidopsis | YP_009076789.1 AEE75132.1 AEE77915.1 |
| | | | | thaliana At3g44560 | |
| | | | | Arabidopsis | AEE79553.1 |
| | | | | thaliana At3g56700 | |
| | | | | Arabidopsis | AED93034.1 |
| | | | | thaliana At5g22500 | |
| | | | | Arabidopsis | AEE86278.1 |
| | | | | thaliana CER4 | |
| | | | | Marinobacter | YP_959486.1 |
| | | | | aquaeolei VT8 | |
| | | | | maqu_2220 | |
| | | | | Marinobacter | YP_959769.1 |
| | | | | aquaeolei VT8 | |
| | | | | maqu_2507 | |
| Acyl-CoA → Aldehyde | 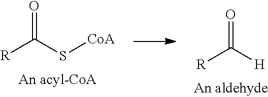 | 1.2.1.10 | Aldehyde forming CoA reductase | Acinetobacter calcoaceticus acr1 Acinetobacter sp Strain M-1 acrM Clostridium beijerinckii ald | AAC45217.1 BAB85476.1 AAT66436.1 |
| | | | | E. coli eutE | NP_416950.1 |
| | | | | Salmonella enterica | AAA80209.1 |
| | | | | eutE | |
| | | | | E. coli mhpF | NP_414885.1 |
| Carboxylic Acid → Aldehyde | 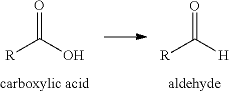 | 1.2.1.- | Carboxylic Acid (Carboxylate) reductase | E. coli PaoABC Mycobacterium marinum Car Nocardia iowensis | NP_414820.1, NP_414819.1, NP_414818.1 WP_012393886.1 AAR91681.1 |
| | | | | Car | |
| | | | | Segniliparus | WP_013138593.1 |
| | | | | rotundus Car | |
| Aldehyde → Alcohol | 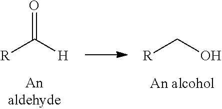 | 1.1.1.- | Alcohol dehydrogen- ase | E. coli betA E. coli dkgA E. coli eutG E. coli fucO E. coli ucpA E. coli yahK | NP_414845.1 NP_417485.4 NP_416948.4 NP_417279.2 NP_416921.4 NP_414859.1 |
| | | | | E. coli ybbO | NP_415026.1 |
| | | | | E. coli ybdH | NP_415132.1 |
| | | | | E. coli yiaY | YP_026233.1 |
| | | | | E. coli yjgB | NP_418690.4 |
| | | | | Acinetobacter sp. | AAG10028.1 |
| | | | | SE19 ChnD | |
| | | | | Marinobacter | YP_959769.1 |
| | | | | aquaeolei VT8 | |
| | | | | maqu_2507 | |
| | | | | Saccharomyces | Q04894.1 |
| | | | | cerevisiae ADH6 | |
| | | | | Clostridium kluyveri | EDK35022.1 |
| | | | | 4hbD | |
| Aldehyde → Alkane | 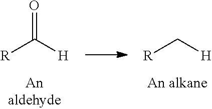 | 4.1.99.5 | Aldehyde decarbonylase | Synechococcus elongatus PCC7942 orf1593 Nostoc punctiforme PCC73102 npun_R1711 | Q54764.1 B2J1M1.1 |
| | | | | Prochlorococcus | Q7V6D4.1 |
| | | | | marinus MIT9313 | |
| | | | | pmt1231 | |
| Alcohol → Alkenyl | 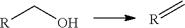 | 4.2.1.- | Dehydratase | Elizabethkingia meningoseptica | GQ144652.1 |
| | | | | ohyA | |
| Alcohol → Phospho | 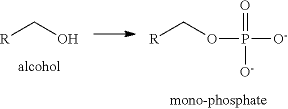 | 2.7.1.- | Alcohol Kinase/ Phosphotrans- ferase | Saccharomyces cerevisiae ERG12 Saccharomyces cerevisiae ERG8 Arabidopsis thaliana At5g58560 Mentha x piperita | P07277 P24521 Q67ZM7 P56848 |
| | | | | ipk | |
| | | | | Methanocaldococcus | Q58487 |
| | | | | jannaschii mvk | |
| | | | | Arabidopsis | AT5G27450.1 |
| | | | | thaliana mvk | |
| | | | | E. coli ychB | NP_415726.1 |
| | | | | E. coli glpK | P0A6F3 |
| | | | | Methanothermobacter | AAB84554.1 |
| | | | | thermautotrophicus | |
| | | | | ipk | |
| | | | | Thermoplasma | WP_010900530.1 |
| | | | | acidophilum ipk | |
| | | | | Methanocaldococcus | 3K4Y_A |
| | | | | jannaschii ipk | |
| Phospho → Diphospho | 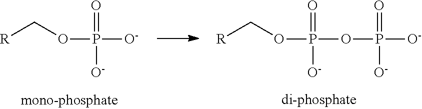 | 2.7.4- | Phosphate Kinase/ Phosphotrans- ferase | Methanothermobacter thermautotrophicus ipk Thermoplasma acidophilum ipk Enterococcus | AAB84554.1 WP_010900530.1 Q9FD67 |
| | | | | faecalis mvaK2 | |
| | | | | Streptococcus | A0A0I7UH23 |
| | | | | pneumoniae | |
| | | | | mvaK2 | |
| | | | | Staphylococcus | A0A0E8GDF5 |
| | | | | aureus mvaK2 | |
| | | | | Methanocaldococcus | 3K4Y_A |
| | | | | jannaschii ipk | |
| Alcohol → Diphospho | 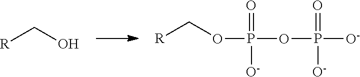 | 2.7.6- | Alcohol diphosphoki- nase | Escherichia coli Prs Mycoplasma pneumoniae M129 PrsA | NP_415725.1 NP_109761.1 |
| | | | | Arabidopsis | BAH19964.1 |
| | | | | thaliana col TPK1 | |
| | | | | Arabidopsis | BAH57065.1 |
| | | | | thaliana col TPK2 | |
| IPP ↔ DMAPP | 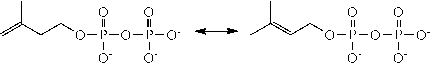 | 5.3.3.2 | isopentenyl diphosphate isomerase | E. coli idi | Q46822 |
| 3,5,7- trioxododecanoyl- CoA → olivetolate | 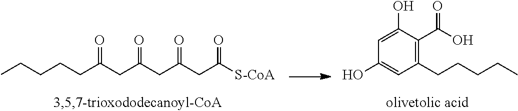 | 4.4.1.26 | olivetolic acid cyclase | Cannabis sativa OAC | I6WU39 |

TABLE C
Example reactions and enzymes of the novel isoprenoid precursor synthesis pathway via 3-hydroxy-3-
methylglutaryl-CoA (HMG-CoA) and prenol based on Claisen condensation reactions

| | | | | Source organism | Protein |
|---|---|---|---|---|---|
| | | EC | Enzyme | and gene/enzyme | Accession |
| Reaction | Illustration | Numbers | names | name | Numbers |
| 2 acetyl- CoA → acetoacetyl- CoA | 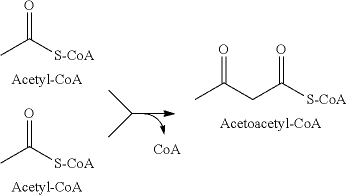 | 2.3.1.- | Thiolase | E. coli atoB E. coli yqeF E. coli fadA E. coli fadI Ralstonia eutropha bktB Pseudomonas sp. Strain B13 catF E coli paaJ Pseudomonas putida pcaF Rhodococcus opacus pcaF | NP_416728.1 NP_417321.2 YP_026272.1 NP_416844.1 AAC38322.1 AAL02407.1 NP_415915.1 AAA85138.1 YP_002778248.1 |
| | | | | Streptomyces sp. | AAD22035.1 |
| | | | | pcaF | |
| | | | | Ralstonia | AEI80291.1 |
| | | | | eutropha phaA | |
| | | | | Clostridium | AAC26023.1 |
| | | | | acetobutylicum | |
| | | | | thIA | |
| | | | | Clostridium | AAC26026.1 |
| | | | | acetobutylicum | |
| | | | | thIB | |
| | | | | Pseudomonas | AAK18168.1 |
| | | | | putida fadA | |
| | | | | P. putida fadAx | AAK18171.1 |
| | | | | Acinetobacter sp. | CAG68532.1 |
| | | | | ADP1 dcaF | |
| | | | | E. coli paaJ | NP_415915.1 |
| Acetyl- CoA + malonyl- CoA → acetoacetyl- CoA + CO₂ | 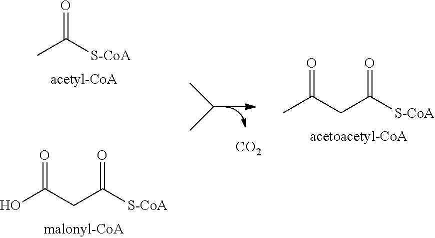 | 2.3.1.- | Ketoacyl- CoA synthase | Gluconobacter oxydans GOX0115 Pseudomonas aeruginosa FabH2 Streptomyces sp. MMG1121 PRK09352 Streptomyces tendae Acs2 Streptomyces sp. strain CL190 NphT7 Physaria fendleri KCS3 | AAW59909.1 NP_252023.1 WP_053666104.1 AFS18568.1 BAJ10048.1 AAK62348.1 |
| | | | | Saccharomyces | NP_009963.1 |
| | | | | cerevisiae ELO2 | |
| | | | | Arabidopsis | NP_171620.2 |
| | | | | thaliana col KCS1 | |
| | | | | Arabidopsis | NP_195178.1 |
| | | | | thaliana col FAE1 | |
| | | | | Arabidopsis | NP_177020.1 |
| | | | | thaliana col CER6 | |
| Acetoacetyl- CoA + acetyl- CoA → 3- hydroxy-3- meth- ylglutaryl- CoA | 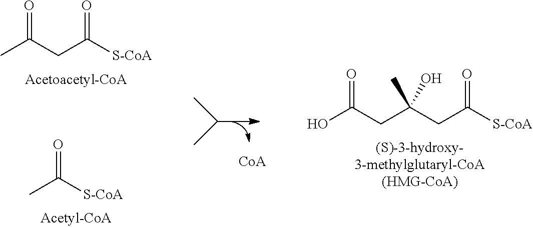 | 2.3.3.10 | Hydroxy methyl- glutaryl- CoA synthase | Staphylococcus aureus HMGS Saccharomyces cerevisiae HMGS Enterococcus faecalis mvaS Ustilago maydis hcs1 Arabidopsis thaliana BAP1 Homo sapiens HMGCS1 Homo sapiens HMGCS2 | BAU36102.1 NP_013580.1 AAG02438.1 KIS66367.1 AAD00297.1 NP_001317592.1 NP_001159579. |
| 3-hydroxy-3- meth- ylglutaryl- CoA → 3- meth- ylglutaconyl- CoA | 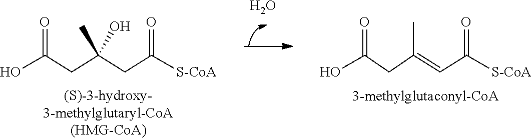 | 4.2.1.17; 4.2.1.119 | Enoyl- CoA hydratase | Myxococcus xanthus liuC E. coli fadB Aeromonas punctata phaJ E. coli fadJ E. coli paaF P. putida fadB P. putida fadB1x | WP_011553770.1 NP_418288.1 O32472.1 NP_416843.1 NP_415911.1 AAK18167.2 AAK18173.1 |
| | | | | Acinetobacter sp. | CAG68535.1 |
| | | | | ADP1 dcaE | |
| | | | | Clostridium | AAA95967.1 |
| | | | | acetobutylicum | |
| | | | | crt | |
| | | | | Aeromonas | O32472.1 |
| | | | | caviae phaJ | |
| | | | | E. coli fabA | NP_415474.1 |
| | | | | E. coli fabZ | NP_414722.1 |
| 3- methyl- glutaconyl- CoA → 3-methyl-2- butenoyl- CoA + CO₂ | 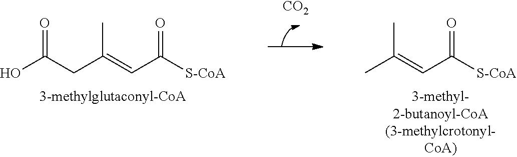 | 4.1.1.70 | Gluta- conyl- CoA decarboxy- lase | Myxococcus xanthus aibAB | WP_011554267.1 WP_011554268.1 |
| 3-methyl-2- butenoyl- CoA → 3- methyl-2- butenoate | 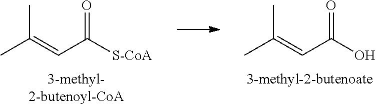 | 3.1.2.- | Thio- esterase | E. coli tesA E. coli tesB E. coli yciA E. coli fadM E. coli ydiI E. coli ybgC E. coli paaI | NP_415027.1 NP_414986.1 NP_415769.1 NP_414977.1 NP_416201.1 NP_415264.1 NP_415914.1 |
| | | | | Mus musculus | P58137.1 |
| | | | | acot8 | |
| | | | | Lycopersicon | ADK38536.1 |
| | | | | hirsutum f | |
| | | | | glabratum mks2 | |
| | | | | Alcanivorax | YP_692749.1 |
| | | | | borkumensis | |
| | | | | tesB2 | |
| | | | | Fibrobacter | YP_005822012.1 |
| | | | | succinogenes | |
| | | | | Fs2108 | |
| | | | | Prevotella | YP_003574018.1 |
| | | | | ruminicola Pr655 | |
| | | | | Prevotella | YP_003574982.1 |
| | | | | ruminicola | |
| | | | | Pr1687 | |
| | | 2.8.3.8 | Acyl- | E. coli atoD | NP_416725.1 |
| | | | CoA: | Clostridium | AAA92344.1 |
| | | | acetyl- | kluyveri cat2 | |
| | | | CoA | Clostridium | NP_149326.1, |
| | | | trans- | acetobutylicum | NP_149327.1 |
| | | | ferase | ctfAB | |
| | | | | E. coli ydiF | NP_416209.1 |
| | | 2.3.1.-; | Phos- | Clostridium | NP_349676.1 |
| | | 2.7.2.1; | photrans- | acetobutylicum | |
| | | 2.7.2.15 | acylase + | ptb | |
| | | | Car- | Enterococcus | AAD55374.1 |
| | | | boxylate | faecalis ptb | |
| | | | kinase | Salmonella | AAD39011.1 |
| | | | | enterica pduL | |
| | | | | Clostridium | AAK81015.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica pduW | |
| 3-methyl-2- butenoate → 3-methyl-2- butenal | 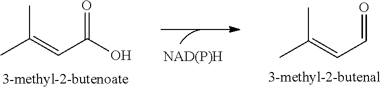 | 1.2.1.- | Car- boxylic Acid (Carboxy- late) reductase | E. coli PaoABC Mycobacterium marinum Car Nocardia | NP_414820.1, NP_414819.1, NP_414818.1 WP_012393886.1 AAR91681.1 |
| | | | | iowensis Car | |
| | | | | Segniliparus | WP_013138593.1 |
| | | | | rotundus Car | |
| 3-methyl-2- butenoyl- CoA → 3-methyl- 2-butenol | 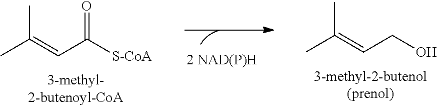 | 1.2.1.- | Alcohol- forming Acyl- CoA reductase | Clostridium acetobutylicum adhE2 Arabidopsis thaliana At3g11980 Arabidopsis | YP_009076789.1 AEE75132.1 AEE77915.1 |
| | | | | thaliana | |
| | | | | At3g44560 | |
| | | | | Arabidopsis | AEE79553.1 |
| | | | | thaliana | |
| | | | | At3g56700 | |
| | | | | Arabidopsis | AED93034.1 |
| | | | | thaliana | |
| | | | | At5g22500 | |
| | | | | Arabidopsis | AEE86278.1 |
| | | | | thaliana CER4 | |
| | | | | Marinobacter | YP_959486.1 |
| | | | | aquaeolei VT8 | |
| | | | | maqu_2220 | |
| | | | | Marinobacter | YP_959769.1 |
| | | | | aquaeolei VT8 | |
| | | | | maqu_2507 | |
| 3-methyl-2- butenoyl- CoA → 3-methyl- 2-butenal | 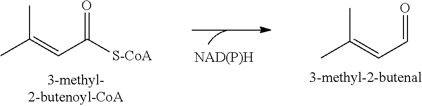 | 1.2.1.- | Aldehyde forming CoA reductase | Acinetobacter calcoaceticus acr1 Acinetobacter sp Strain M-1 acrM Clostridium beijerinckii ald | AAC45217.1 BAB85476.1 AAT66436.1 |
| | | | | E. coli eutE | NP_416950.1 |
| | | | | Salmonella | AAA80209.1 |
| | | | | enterica eutE | |
| | | | | E. coli mhpF | NP_414885.1 |
| 3-methyl-2- butenal → 3-methyl-2- butenol | 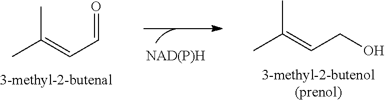 | 1.1.1.- | Alcohol de- hydrogen- ase | E. coli betA E. coli dkgA E. coli eutG E. coli fucO E. coli ucpA E. coli yahK E. coli ybbO | NP_414845.1 NP_417485.4 NP_416948.4 NP_417279.2 NP_416921.4 NP_414859.1 NP_415026.1 |
| | | | | E. coli ybdH | NP_415132.1 |
| | | | | E. coli yiaY | YP_026233.1 |
| | | | | E. coli yjgB | NP_418690.4 |
| | | | | Acinetobacter sp. | AAG10028.1 |
| | | | | SE19 ChnD | |
| | | | | Marinobacter | YP_959769.1 |
| | | | | aquaeolei VT8 | |
| | | | | maqu_2507 | |
| | | | | Saccharomyces | Q04894.1 |
| | | | | cerevisiae ADH6 | |
| | | | | Clostridium | EDK35022.1 |
| | | | | kluyveri 4hbD | |
| 3-methyl-2- butenol → dimethylallyl phosphate | 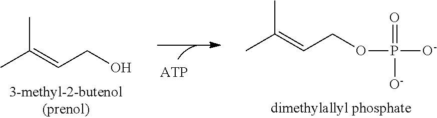 | 2.7.1.- | Alcohol Kinase/ Phos- photrans- ferase | Saccharomyces cerevisiae ERG12 Saccharomyces cerevisiae ERG8 Arabidopsis thaliana | P07277 P24521 Q67ZM7 |
| | | | | At5g58560 | |
| | | | | Mentha x piperita | P56848 |
| | | | | ipk | |
| | | | | Methanocaldo- | Q58487 |
| | | | | coccus | |
| | | | | jannaschii mvk | |
| | | | | Arabidopsis | AT5G27450.1 |
| | | | | thaliana mvk | |
| | | | | E. coli ychB | NP_415726.1 |
| | | | | E. coli glpK | P0A6F3 |
| | | | | Methanothermo- | AAB84554.1 |
| | | | | bacter | |
| | | | | thermautotro- | |
| | | | | phicus ipk | |
| | | | | Thermoplasma | WP_010900530.1 |
| | | | | acidophilum ipk | |
| | | | | Methanocaldo- | 3K4Y_A |
| | | | | coccus | |
| | | | | jannaschii ipk | |
| dimethylallyl phosphate → dimethylallyl pyrophos- phate | 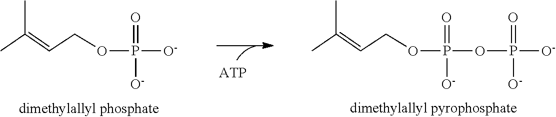 | 2.7.4- | Phos- phate Kinase/ Phos- photrans- ferase | Methanothermo- bacter thermautotro- phicus ipk Thermoplasma acidophilum ipk Enterococcus | AAB84554.1 WP_010900530.1 Q9F067 |
| | | | | faecalis mvaK2 | |
| | | | | Streptococcus | A0A0I7UH23 |
| | | | | pneumoniae | |
| | | | | mvaK2 | |
| | | | | Staphylococcus | A0A0E8GDF5 |
| | | | | aureus mvaK2 | |
| | | | | Methanocaldo- | 3K4Y_A |
| | | | | coccus | |
| | | | | jannaschii ipk | |
| 3-methyl-2- butenol → dimethylallyl pyrophos- phate | 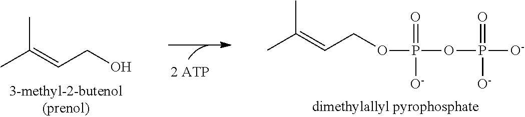 | 2.7.6- | Alcohol diphos- phokinase | Escherichia coli Prs Mycoplasma pneumoniae M129 PrsA Arabidopsis thaliana col | NP_415725.1 NP_109761.1 BAH19964.1 |
| | | | | TPK1 | |
| | | | | Arabidopsis | BAH57065.1 |
| | | | | thaliana col | |
| | | | | TPK2 | |
| DMAPP ↔ IPP | 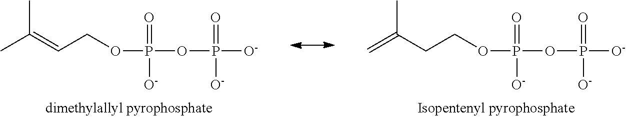 | 5.3.3.2 | iso- pentenyl diphos- phate isomerase | E. coli idi | Q46822 |
| DMAPP + IPP → GPP | 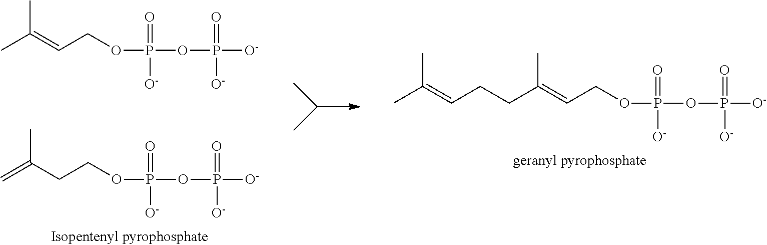 | 2.5.1- | Geranyl pyrophos- phate synthase | E. coli ispA Abies grandis GPPS2 | P22939 Q8LKJ2 |

TABLE D
Example reactions and enzymes of the novel isoprenoid precursor synthesis pathway via 3-hydroxy-3-
methylbutyryl-CoA and prenol based on Claisen condensation reactions

| | | | | Source organism | Protein |
|---|---|---|---|---|---|
| | | EC | Enzyme | and gene/enzyme | Accession |
| Reaction | Illustration | Numbers | names | name | Numbers |
| 2 acetyl- CoA → acetoacetyl- CoA | 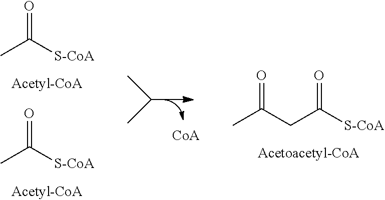 | 2.3.1.- [symbol] | Thiolase | E. coli atoB E. coli yqeF E. coli fadA E. coli fadI Ralstonia eutropha bktB Pseudomonas sp. Strain B13 catF E coli paaJ Pseudomonas putida pcaF Rhodococcus opacus pcaF | NP_416728.1 NP_417321.2 YP_026272.1 NP_416844.1 AAC38322.1 AAL02407.1 NP_415915.1 AAA85138.1 YP_002778248.1 |
| | | | | Streptomyces sp. | AAD22035.1 |
| | | | | pcaF | |
| | | | | Ralstonia | AEI80291.1 |
| | | | | eutropha | |
| | | | | phaA | |
| | | | | Clostridium | AAC26023.1 |
| | | | | acetobutylicum | |
| | | | | thIA | |
| | | | | Clostridium | AAC26026.1 |
| | | | | acetobutylicum | |
| | | | | thIB | |
| | | | | Pseudomonas | AAK18168.1 |
| | | | | putida fadA | |
| | | | | P. putida fadAx | AAK18171.1 |
| | | | | Acinetobacter sp. | CAG68532.1 |
| | | | | ADP1 dcaF | |
| | | | | E. coli paaJ | NP_415915.1 |
| Acetyl-CoA + malonyl- CoA → acetoacetyl- CoA + CO₂ | 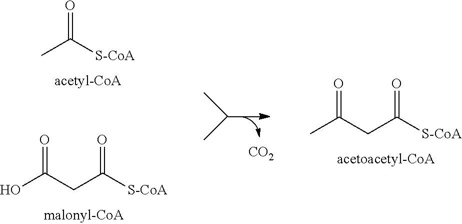 | 2.3.1.- [symbol] | Ketoacyl- CoA synthase | Gluconobacter oxydans GOX0115 Pseudomonas aeruginosa FabH2 Streptomyces sp. MMG1121 PRK09352 Streptomyces tendae Acs2 | AAW59909.1 NP_252023.1 WP_053666104.1 AFS18568.1 |
| | | | | Streptomyces sp. | BAJ10048.1 |
| | | | | strain CL190 | |
| | | | | NphT7 | |
| | | | | Physaria fendleri | AAK62348.1 |
| | | | | KCS3 | |
| | | | | Saccharomyces | NP_009963.1 |
| | | | | cerevisiae | |
| | | | | ELO2 | |
| | | | | Arabidopsis | NP_171620.2 |
| | | | | thaliana col | |
| | | | | KCS1 | |
| | | | | Arabidopsis | NP_195178.1 |
| | | | | thaliana col | |
| | | | | FAE1 | |
| | | | | Arabidopsis | NP_177020.1 |
| | | | | thaliana col | |
| | | | | CER6 | |
| Acetoacetyl- CoA → Acetoacetic acid + CoA 3-methyl-2- butenoul- CoA → 3- methyl-2- butenoate | 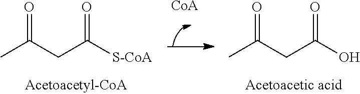 | 3.1.2.- [symbol] | Thioes- terase | E. coli tesA E. coli tesB E. coli yciA E. coli fadM E. coli ydiI E. coli ybgC E. coli paaI Mus musculus acot8 Lycopersicon hirsutum f glabratum mk52 Alcanivorax borkumensis tesB2 | NP_415027.1 NP_414986.1 NP_415769.1 NP_414977.1 NP_416201.1 NP_415264.1 NP_415914.1 P58137.1 ADK38536.1 YP_692749.1 |
| | | | | Fibrobacter | YP_005822012.1 |
| | | | | succinogenes | |
| | | | | Fs2108 | |
| | | | | Prevotella | YP_003574018.1 |
| | | | | ruminicola Pr655 | |
| | | | | Prevotella | YP_003574982.1 |
| | | | | ruminicola Pr1687 | |
| | | 2.8.3- [symbol] | CoA | E. coli atoD | NP_416725.1 |
| | | | trans- | E. coli atoA | NP_416726.1 |
| | | | ferase | E. coli scpC | NP_417395.1 |
| | | | | Clostridium kluyveri | AAA92346.1 |
| | | | | cat1 | |
| | | | | Clostridium kluyveri | AAA92344.1 |
| | | | | cat2 | |
| | | | | Clostridium | NP_149326.1, |
| | | | | acetobutylicum | NP_149327.1 |
| | | | | ctfAB | |
| | | | | Pseudomonas | NP_746081.1 |
| | | | | putida pcalJ | NP_746082.1 |
| | | | | Megasphaera | WP_014015705.1 |
| | | | | elsdenii pct | |
| | | | | Acidaminococcus | CAA57199.1 |
| | | | | fermentans gctAB | CAA57200.1 |
| | | | | Acetobacter aceti | AGG68319.1 |
| | | | | aarC | |
| | | | | E. coli ydiF | NP_416209.1 |
| | | 2.3.1.-; | Phos- | Clostridium | NP_349676.1 |
| | | 2.7.2.1; | photrans- | acetobutylicum ptb | |
| | | 2.7.2.15 | acylase + | Enterococcus | AAD55374.1 |
| | | | Carbox- | faecalis ptb | |
| | | | ylate | Salmonella enterica | AAD39011.1 |
| | | | kinase | pduL | |
| | | | | Clostridium | AAK81015.1 |
| | | | | acetobutylicum buk | |
| | | | | Enterococcus | AAD55375.1 |
| | | | | faecalis buk | |
| | | | | Salmonella enterica | AAD39021.1 |
| | | | | pduW | |
| Acetoacetic acid → acetone + CO₂ | 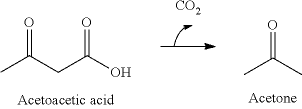 | 4.1.1.56; | Decar- boxylase | Lycopersicon hirsutum f glabratum mks1 Clostridium acetobutylicum adc | ADK38535.1 AAA63761.1 |
| Acetone + acetyl- CoA → 3- hydroxy-3- methylbutyryl- CoA | 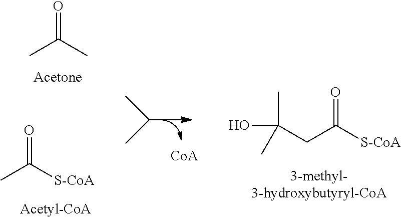 | 2.3.3.- [symbol] | Hydrox- ymethyl butyryl- CoA synthase | Staphylococcus aureus HMGS Saccharomyces cerevisiae HMGS Enterococcus aecalis mvaS Ustilago maydis hcs1 Arabidopsis thaliana BAP1 | BAU36102.1 NP_013580.1 AAG02438.1 KIS66367.1 AAD00297.1 |
| | | | | Homo sapiens | NP_001317592.1 |
| | | | | HMGCS1 | |
| | | | | Homo sapiens | NP_001159579 |
| | | | | HMGCS2 | |
| 3-hydroxy-3- methylbutyryl- CoA → 3- methyl-2- butenoyl- CoA | 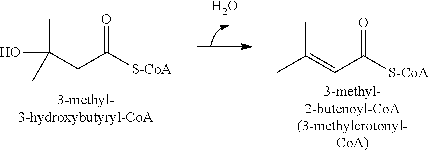 | 4.2.1.17; 4.2.1.119 | Enoyl- CoA hydratase | Myxococcus xanthus liuC E. coli fadB Aeromonas punctata phaJ E. coli fadJ E. coli paaF P. putida fadB P. putida fadB1x | WP_011553770.1 NP_418288.1 O32472.1 NP_416843.1 NP_415911.1 AAK18167.2 AAK18173.1 |
| | | | | Acinetobacter sp. | CAG68535.1 |
| | | | | ADP1 dcaE | |
| | | | | Clostridium | AAA95967.1 |
| | | | | acetobutylicum crt | |
| | | | | Aeromonas caviae | O32472.1 |
| | | | | phaJ | |
| | | | | E. coli fabA | NP_415474.1 |
| | | | | E. coli fabZ | NP_414722.1 |
| 3-methyl-2- butenoate → 3-methyl-2- butenal | 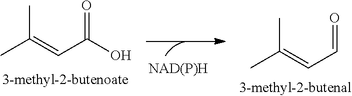 | 1.2.1.- [symbol] | Carbox- ylic Acid (Carbox- ylate) reductase | E. coli PaoABC Mycobacterium marinum Car Nocardia iowensis | NP_414820.1, NP_414819.1, NP_414818.1 WP_012393886.1 AAR91681.1 |
| | | | | Car | |
| | | | | Segniliparus | WP_013138593.1 |
| | | | | rotundus Car | |
| 3-methyl-2- butenoyl- CoA → 3-methyl- 2-butenol | 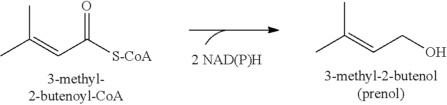 | 1.2.1.- [symbol] | Alcohol- forming Acyl- CoA reductase | Clostridium acetobutylicum adhE2 Arabidopsis thaliana At3g11980 Arabidopsis | YP_009076789.1 AEE75132.1 AEE77915.1 |
| | | | | thaliana At3g44560 | |
| | | | | Arabidopsis | AEE79553.1 |
| | | | | thaliana At3g56700 | |
| | | | | Arabidopsis | AED93034.1 |
| | | | | thaliana At5g22500 | |
| | | | | Arabidopsis | AEE86278.1 |
| | | | | thaliana CER4 | |
| | | | | Marinobacter | YP_959486.1 |
| | | | | aquaeolei VT8 | |
| | | | | maqu_2220 | |
| | | | | Marinobacter | YP_959769.1 |
| | | | | aquaeolei VT8 | |
| | | | | maqu_2507 | |
| 3-methyl-2- butenoyl- CoA → 3-methyl- 2-butenal | 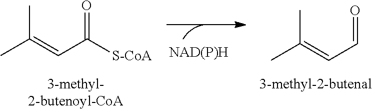 | 1.2.1.- [symbol] | Aldehyde forming CoA reductase | Acinetobacter calcoaceticus acr1 Acinetobacter sp Strain M-1 acrM Clostridium beijerinckii ald E. coli eutE | AAC45217.1 BAB85476.1 AAT66436.1 NP_416950.1 |
| | | | | Salmonella enterica | AAA80209.1 |
| | | | | eutE | |
| | | | | E. coli mhpF | NP_414885.1 |
| 3-methyl-2- butenal → 3-methyl-2- butenol | 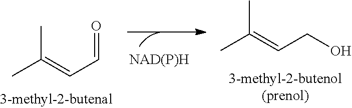 | 1.1.1.- [symbol] | Alcohol dehydro- genase | E. coli betA E. coli dkgA E. coli eutG E. coli fucO E. coli ucpA E. coli yahK E. coli ybbO | NP_414845.1 NP_417485.4 NP_416948.4 NP_417279.2 NP_416921.4 NP_414859.1 NP_415026.1 |
| | | | | E. coli ybdH | NP_415132.1 |
| | | | | E. coli yiaY | YP_026233.1 |
| | | | | E. coli yjgB | NP_418690.4 |
| | | | | Acinetobacter sp. | AAG10028.1 |
| | | | | SE19 ChnD | |
| | | | | Marinobacter | YP_959769.1 |
| | | | | aquaeolei VT8 | |
| | | | | maqu_2507 | |
| | | | | Saccharomyces | Q04894.1 |
| | | | | cerevisiae ADH6 | |
| | | | | Clostridium kluyveri | EDK35022.1 |
| | | | | 4hbD | |
| 3-methyl-2- butenol → dimethylallyl phosphate | 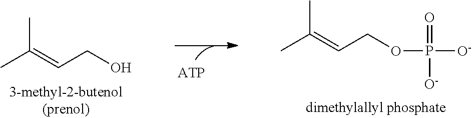 | 2.7.1.- [symbol] | Alcohol Kinase/ Phos- photrans- ferase | Saccharomyces cerevisiae ERG12 Saccharomyces cerevisiae ERG8 Arabidopsis thaliana At5g58560 Mentha x piperita | P07277 P24521 Q67ZM7 P56848 |
| | | | | ipk | |
| | | | | Methanocaldo- | Q58487 |
| | | | | coccus | |
| | | | | jannaschii mvk | |
| | | | | Arabidopsis | AT5G27450.1 |
| | | | | thaliana mvk | |
| | | | | E. coli ychB | NP_415726.1 |
| | | | | E. coli glpK | P0A6F3 |
| | | | | Methanothermo- | AAB84554.1 |
| | | | | bacter | |
| | | | | thermautotrophicus | |
| | | | | ipk | |
| | | | | Thermoplasma | WP_010900530.1 |
| | | | | acidophilum ipk | |
| | | | | Methanocaldo- | 3K4Y_A |
| | | | | coccus | |
| | | | | jannaschii ipk | |
| dimethylallyl phosphate → dimethylallyl pyrophos- phate | 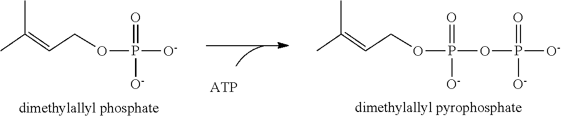 | 2.7.4- [symbol] | Phos- phate Kinase/ Phos- photrans- ferase | Methanothermo- bacter thermautotrophicus ipk Thermoplasma acidophilum ipk Enterococcus | AAB84554.1 WP_010900530.1 Q9F067 |
| | | | | faecalis mvaK2 | |
| | | | | Streptococcus | A0A0I7UH23 |
| | | | | pneumoniae | |
| | | | | mvaK2 | |
| | | | | Staphylococcus | A0A0E8GDF5 |
| | | | | aureus mvaK2 | |
| | | | | Methanocaldo- | 3K4Y_A |
| | | | | coccus | |
| | | | | jannaschii ipk | |
| 3-methyl-2- butenol → dimethylallyl pyrophos- phate | 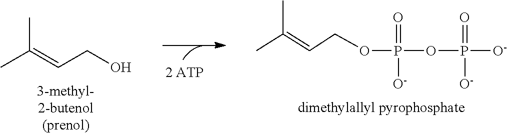 | 2.7.6- [symbol] | Alcohol diphos- phokin- ase | Escherichia coli Prs Mycoplasma pneumoniae M129 PrsA Arabidopsis thaliana col TPK1 Arabidopsis | NP_415725.1 NP_109761.1 BAH19964.1 BAH57065.1 |
| | | | | thaliana col TPK2 | |
| DMAPP ↔ IPP | 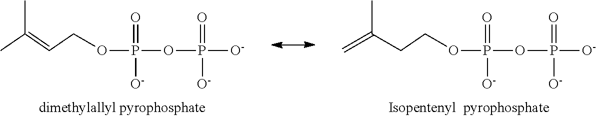 | 5.3.3.2 | isopen- tenyl diphos- phate isomerase | E. coli idi | Q46822 |
| DMAPP + IPP → GPP | 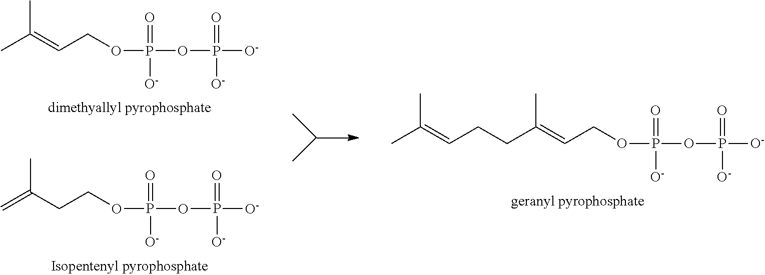 | 2.5.1- [symbol] | Geranyl pyrophos- phate synthase | E. coli ispA Abies grandis GPPS2 | P22939 Q8LKJ2 |

[symbol]

indicates data missing or illegible when filed

TABLE E
Example reactions and enzymes of the pathway for isoprenoid precursor synthesis based on acyloin
condensation via 2-hydroxyisovaleric acid and prenol through utilization of 2-hydroxyacid/2-hydroxyacyl-CoA
dehydratase

| | | | | Source organism | Protein |
|---|---|---|---|---|---|
| | | EC | Enzyme | and gene/enzyme | Accession |
| Reaction | Illustration | Numbers | names | name | Numbers |
| 2 pyruvate → acetolactate | 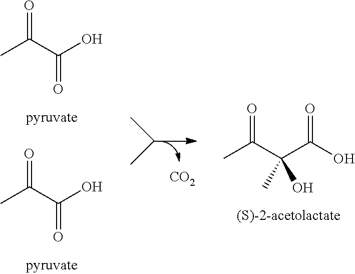 | 2.2.1.6 | Aceto- lactate synthase | E. coli ilvBN E. coli ilvH B. subtilis alsS | P08142, P0ADF8 P00893, P00894 Q04789 |
| aceto- lactate → 2,3- dihydroxy- 3-methyl- butanoate | 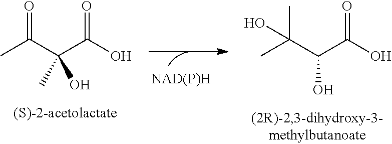 | 1.1.1.86 | Acetohy- droxy acid isomerore- ductase | E. coli ilvC | P05793 |
| 2,3- dihydroxy- 3- methyl- butanoate → 3-methyl-2- oxo- butanoate | 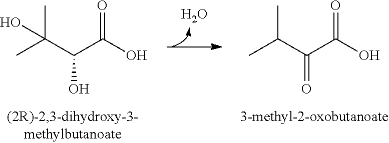 | 4.2.1.9 | Dihydroxy- acid dehy- dratase | E. coli ilvD | P05791 |
| 3-methyl-2- oxo butanoate → 3-methyl- 2-hydroxy- butanoate | 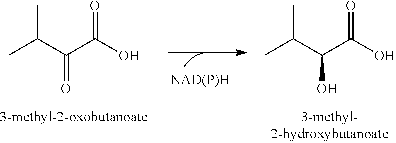 | 1.1.1.- | 2-hydroxy- acid dehydro- genase | Acidaminococcus fermentans hgdH Methanocaldo- coccus jannaschii mdh M. jannaschii comC E. coli mdh E. coli serA E. coli idhA Haloferax | D2RMS7 Q60176 Q58820 P61889 P0A9T0 P52643 Q2VEQ7 |
| | | | | mediterranei ddh | |
| | | | | Lactococcus lactis | A0A089XK51 |
| | | | | panE | |
| 3-methyl-2- hydroxy- butanoate → 3-methyl-2- butenoate | 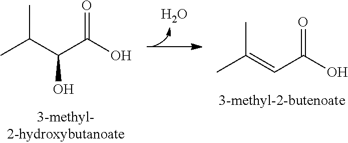 | 4.2.1.- 4.2.1- | 2-hydroxy- acid dehy- dratase 2-hydroxy- acyl- CoA dehydratase | E. coli fumA E. coli fumB E. coli fumC Acidaminococcus fermentans hgdCAB Clostridium symbiosum hgdCAB | P0AC33 P14407 P05042 P11568, P11569, P11570, Q9X5B6, Q9X5B7, Q9X5B8, |
| | | | | Fusobacterium | A5TT16, |
| | | | | nucleatum | A5TT17, |
| | | | | hgdCAB | A5TT18, |
| | | | | Peptoclostridium | AAV40818.1, |
| | | | | difficile hadIBC | AAV40819.1, |
| | | | | | AAV40820.1 |
| | | | | Clostridium | G3KIM3, |
| | | | | propionicum | G3KIM4, |
| | | | | IcdCAB | G3KIM5 |
| 3-methyl-2- hydroxybu- tanoate → 3-methyl-2- hydrox- ybutanoyl- CoA 3-methyl-2- butenoate → 3-methyl-2- butenoyl- CoA | 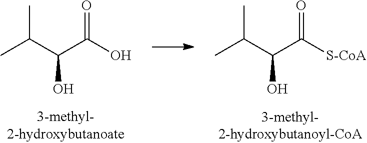 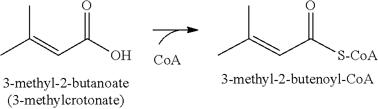 | 6.2.1.- | Acyl-CoA synthetase | E. coli paaK E. coli sucCD E. coli fadK E. coli fadD E. coli prpE E. coli menE Penicillium chrysogenum phl Salmonella typhimurium LT2 prpE Bacillus subtilis bioW Cupriavidus basilensis hmfD Rhodopseudo- monas | NP_415916.1 NP_415256.1 NP_415257.1 NP_416216.4 NP_416319.1 NP_414869.1 NP_416763.1 CAJ15517.1 AAL19325.1 AAC00261.1 ADE20402.1 CAJ18317.1 |
| | | | | palustris badA | |
| | | | | R. palustris hbaA | CAE26113.1 |
| | | | | Pseudomonas | NP_249687.1 |
| | | | | aeruginosa PAO1 | |
| | | | | pqsA | |
| | | | | Arabidopsis | Q42524.1 |
| | | | | thaliana 4cl | |
| | | 2.8.3- | CoA | E. coli atoD | NP_416725.1 |
| | | | transferase | E. coli atoA | NP_416726.1 |
| | | | | E. coli scpC | NP_417395.1 |
| | | | | Clostridium | AAA92346.1 |
| | | | | kluyveri cat1 | |
| | | | | Clostridium | AAA92344.1 |
| | | | | kluyveri cat2 | |
| | | | | Clostridium | NP_149326.1, |
| | | | | acetobutylicum | NP_149327.1 |
| | | | | ctfAB | |
| | | | | Pseudomonas | NP_746081.1 |
| | | | | putida pcalJ | NP_746082.1 |
| | | | | Megasphaera | WP_014015705.1 |
| | | | | elsdenii pct | |
| | | | | Acidaminococcus | CAA57199.1 |
| | | | | fermentans | CAA57200.1 |
| | | | | gctAB | |
| | | | | Acetobacter aceti | AGG68319.1 |
| | | | | aarC | |
| | | | | E. coli ydiF | NP_416209.1 |
| | | | | Clostridium | CA377207.1 |
| | | | | propionicum | |
| | | | | Pct540 | |
| | | 2.3.1.-; | Carboxy- | Clostridium | NP_349676.1 |
| | | 2.7.2.- | late | acetobutylicum | |
| | | | kinase + | ptb | |
| | | | Phos- | Enterococcus | AAD55374.1 |
| | | | photrans- | faecalis ptb | |
| | | | acylase | Salmonella | AAD39011.1 |
| | | | | enterica pduL | |
| | | | | Clostridium | AAK81015.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica pduW | |
| 3-methyl-2- hydrox- ybutanoyl- CoA → 3-methyl-2- butenoyl- CoA | 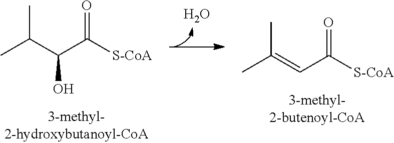 | 4.2.1- | 2-hydroxy- acyl- CoA dehy- dratase | Acidaminococcus fermentans hgdCAB Clostridium symbiosum hgdCAB Fusobacterium nucleatum hgdCAB | P11568, P11569, P11570, Q9X5B6, Q9X5B7, Q9X5B8, A5TT16, A5TT17, A5TT18, |
| | | | | Peptoclostridium | AAV40818.1, |
| | | | | difficile hadIBC | AAV40819.1, |
| | | | | | AAV40820.1 |
| | | | | Clostridium | G3KIM3, |
| | | | | propionicum | G3KIM4, |
| | | | | IcdCAB | G3KIM5 |
| 3-methyl-2- butenoyl- CoA → 3- methyl-2- butenoate 3-methyl-2- hydroxy- butanoyl- CoA → 3-methyl-2- hydroxy- butanoate | 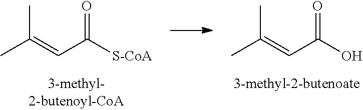 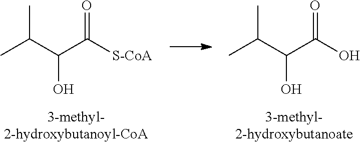 | 3.1.2.- | Thioes- terase | E. coli tesA E. coli tesB E. coli yciA E. coli fadM E. coli ydiI E. coli ybgC E. coli paaI Mus musculus acot8 Lycopersicon hirsutum f glabratum mks2 Alcanivorax borkumensis tesB2 Fibrobacter succinogenes | NP_415027.1 NP_414986.1 NP_415769.1 NP_414977.1 NP_416201.1 NP_415264.1 NP_415914.1 P58137.1 ADK38536.1 YP_692749.1 YP_005822012.1 |
| | | | | Fs2108 | |
| | | | | Prevotella | YP_003574018.1 |
| | | | | ruminicola | |
| | | | | Pr655 | |
| | | | | Prevotella | YP_003574982.1 |
| | | | | ruminicola | |
| | | | | Pr1687 | |
| | | 2.8.3.8 | Acyl- | E. coli atoD | NP_416725.1 |
| | | | CoA:acyl- | Clostridium | AAA92344.1 |
| | | | CoA | kluyveri cat2 | |
| | | | transferase | Clostridium | NP_149326.1, |
| | | | | acetobutylicum | NP_149327.1 |
| | | | | ctfAB | |
| | | | | E. coli ydiF | NP_416209.1 |
| | | 2.3.1.-; | Phos- | Clostridium | NP_349676.1 |
| | | 2.7.2.1; | photrans- | acetobutylicum | |
| | | 2.7.2.15 | acylase + | ptb | |
| | | | Carbox- | Enterococcus | AAD55374.1 |
| | | | ylate | faecalis ptb | |
| | | | kinase | Salmonella | AAD39011.1 |
| | | | | enterica pduL | |
| | | | | Clostridium | AAK81015.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica | |
| | | | | pduW | |
| 3-methyl-2- butenoate → 3-methyl-2- butenal 3-methyl-2- hydroxy- butanoate → 3-methyl-2- hydroxy- butanal | 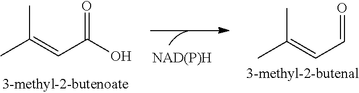 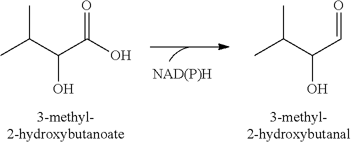 | 1.2.1.- | Carboxy- lic Acid (Carboxyl- ate) reductase | E. coli PaoABC Mycobacterium marinum Car Nocardia iowensis Car Segniliparus rotundus Car | NP_414820.1, NP_414819.1, NP_414818.1 WP_012393886.1 AAR91681.1 WP_013138593.1 |
| 3-methyl-2- butenoyl- CoA → 3- methyl-2- butenol 3-methyl-2- hydrox- ybutanoyl- CoA → 3- methyl-1,2- butandiol | 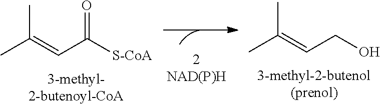 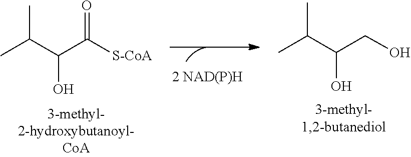 | 1.2.1.- | Alcohol- forming Acyl-CoA reductase | Clostridium acetobutylicum adhE2 Arabidopsis thaliana At3g11980 Arabidopsis thaliana At3g44560 Arabidopsis thaliana At3g56700 Arabidopsis thaliana At5g22500 Arabidopsis thaliana CER4 Marinobacter aquaeolei VT8 maqu_2220 Marinobacter | YP_009076789.1 AEE75132.1 AEE77915.1 AEE79553.1 AED93034.1 AEE86278.1 YP_959486.1 YP_959769.1 |
| | | | | aquaeolei VT8 | |
| | | | | maqu_2507 | |
| 3-methyl-2- butenoyl- CoA → 3- methyl-2- butenal | 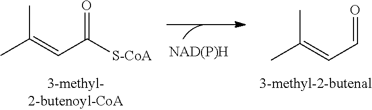 | 1.2.1.- | Aldehyde forming CoA reductase | Acinetobacter calcoaceticus acr1 Acinetobacter sp Strain M-1 acrM Clostridium beijerinckii ald | AAC45217.1 BAB85476.1 AAT66436.1 |
| 3-methyl-2- hydroxy- butanoyl- CoA → 3- methyl-2- hydrox- ybutanal 3-methyl- butanal → isovaleryl- CoA | 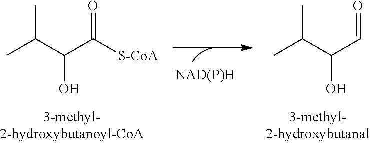 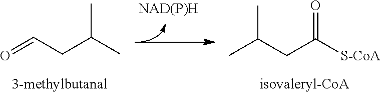 | | | E. coli eutE Salmonella enterica eutE E. coli mhpF | NP_416950.1 AAA80209.1 NP_414885.1 |
| 3-methyl-2- butenal → 3- methyl-2- butenol 3-methyl-2- hydrox- ybutanal → 3-methyl- 1,2- butandiol | 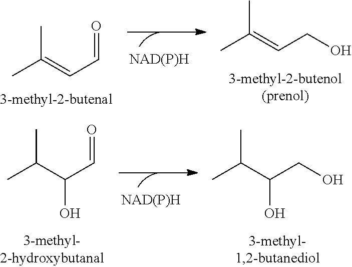 | 1.1.1.- | Alcohol dehy- drogenase | E. coli betA E. coli dkgA E. coli eutG E. coli fucO E. coli ucpA E. coli yahK E. coli ybbO E. coli ybdH E. coli yiaY E. coli yjgB Acinetobacter sp. SE19 ChnD Marinobacter aquaeolei VT8 maqu_2507 Saccharomyces | NP_414845.1 NP_417485.4 NP_416948.4 NP_417279.2 NP_416921.4 NP_414859.1 NP_415026.1 NP_415132.1 YP_026233.1 NP_418690.4 AAG10028.1 YP_959769.1 Q04894.1 |
| | | | | cerevisiae ADH6 | |
| | | | | Clostridium | EDK35022.1 |
| | | | | kluyveri 4hbD | |
| 3-methyl- 1,2- butandiol → 3-meth- ylbutanal | 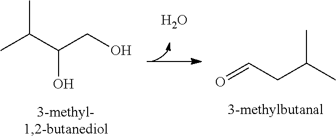 | 4.2.1.- | Diol dehy- dratase | Klebsiella ocytoca pddABC E. coli pduCDE S. enterica pduCDE | Q59470, Q59471, Q59472 CAS09680, CAS09681, CAS09682 NP_456590, NP_456591, NP_456592 |
| isovaleryl- CoA → 3- methyl-2- butenoyl- CoA | 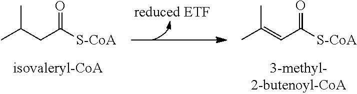 | 1.3.8.4 | Acyl-CoA dehy- drogenase | Pseudomonas aeruginosa liuA E. coli aidB E. coli fadE Streptomyces avermitilis acdH | APJ52511.1 NP_418608.6 NP_414756.2 AAD44196.1 |
| 3-methyl-2- butenol → dimethyl- allyl phosphate | 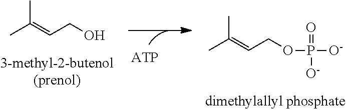 | 2.7.1.- | Alcohol Kinase/ Phospho- transferase | Saccharomyces cerevisiae ERG12 Saccharomyces cerevisiae ERG8 Arabidopsis thaliana At5g58560 Mentha x piperita ipk | P07277 P24521 Q67ZM7 P56848 |
| | | | | Methanocaldo- | Q58487 |
| | | | | coccus | |
| | | | | jannaschii mvk | |
| | | | | Arabidopsis | AT5G27450.1 |
| | | | | thaliana mvk | |
| | | | | E. coli ychB | NP_415726.1 |
| | | | | E. coli glpK | P0A6F3 |
| | | | | Methanothermo- | AAB84554.1 |
| | | | | bacter | |
| | | | | thermautotrophicus | |
| | | | | ipk | |
| | | | | Thermoplasma | WP_010900530.1 |
| | | | | acidophilum ipk | |
| | | | | Methanocaldo- | 3K4Y_A |
| | | | | coccus | |
| | | | | jannaschii ipk | |
| dimeth- ylallyl phosphate → dimeth- ylallyl pyrophos- phate | 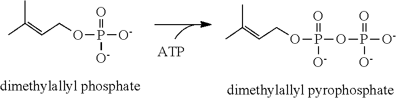 | 2.7.4- | Phosphate Kinase/ Phos- photrans- ferase | Methanothermo- bacter thermautotrophicus ipk Thermoplasma acidophilum ipk Enterococcus | AAB84554.1 WP_010900530.1 Q9F067 |
| | | | | faecalis mvaK2 | |
| | | | | Streptococcus | A0A0I7UH23 |
| | | | | pneumoniae | |
| | | | | mvaK2 | |
| | | | | Staphylococcus | A0A0E8GDF5 |
| | | | | aureus mvaK2 | |
| | | | | Methanocaldo- | 3K4Y_A |
| | | | | coccus | |
| | | | | jannaschii ipk | |
| 3-methyl-2- butenol → dimethyl- allyl pyrophos- phate | 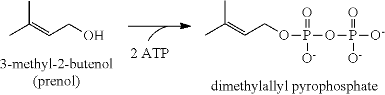 | 2.7.6- | Alcohol diphos- phokinase | Escherichia coli Prs Mycoplasma pneumoniae M129 PrsA Arabidopsis thaliana col TPK1 Arabidopsis thaliana col TPK2 | NP_415725.1 NP_109761.1 BAH19964.1 BAH57065.1 |
| DMAPP ↔ IPP | 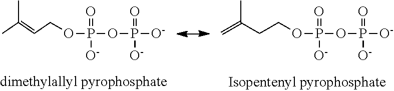 | 5.3.3.2 | iso- pentenyl diphos- phate isomerase | E. coli idi | Q46822 |
| DMAPP + IPP → GPP | 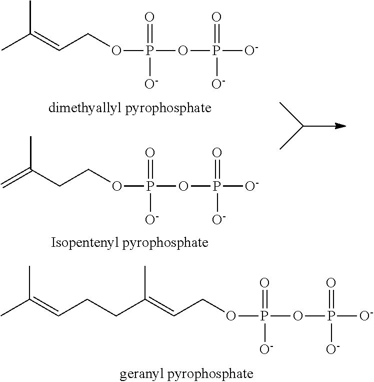 | 2.5.1- | Geranyl pyrophos- phate synthase | E. coli ispA Abies grandis GPPS2 | P22939 Q8LKJ2 |

TABLE F
Example reactions and enzymes of the pathway for isoprenoid precursor synthesis based on acyloin condensation via 2-oxoisovaleric acid, 2-oxoisocaproic acid and
prenol through alpha-keto acid pathway and branched alpha-keto acid dehydrogenase

| | | | | Source organism | |
|---|---|---|---|---|---|
| | | | | and | Protein |
| | | EC | Enzyme | gene/enzyme | Accession |
| Reaction | Illustration | Numbers | names | name | Numbers |
| 2 pyruvate → acetolactate | 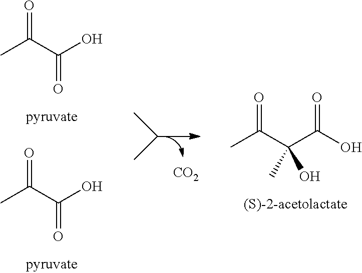 | 2.2.1.6 | Acetolactate synthase | E. coli ilvBN E. coli ilvIH B. subtilis alsS | P08142, P0ADF8 P00893, P00894 Q04789 |
| acetolactate → 2,3- dihydroxy-3- methylbutanoate | 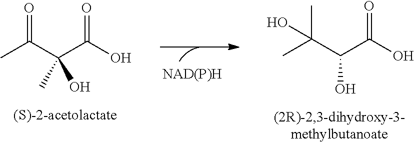 | 1.1.1.86 | Acetohydroxyacid isomeroreductase | E. coli ilvC | P05793 |
| 2,3-dihydroxy-3- methyl- butanoate → 3-methyl-2- oxobutanoate | 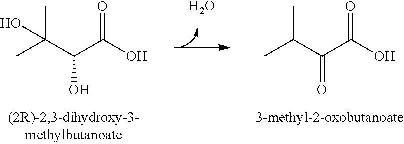 | 4.2.1.9 | Dihydroxyacid dehydratase | E. coli ilvD | P05791 |
| 3-methyl-2- oxobutanoate + acetyl-CoA → 2-isopropyl- malate + CoA | 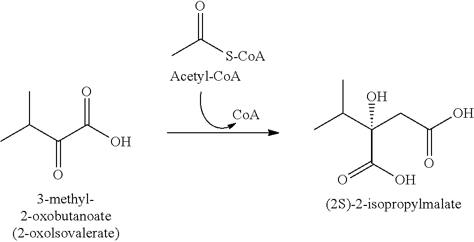 | 2.3.3.13 | Isopropylmalate synthase | E. coli leuA | NP_414616.1 |
| 2-isopropyl- malate → 3-isopropylmalate | 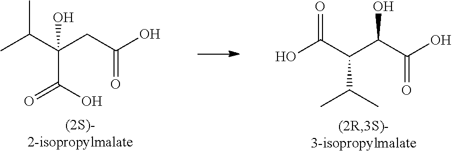 | 4.2.1.33 | Isopropylmalate isomerase | E. coli leuCD | NP_414614.1 NP_414613.1 |
| 3-isopropyl- malate → 4-methyl-2- oxopentanoate + CO₂ | 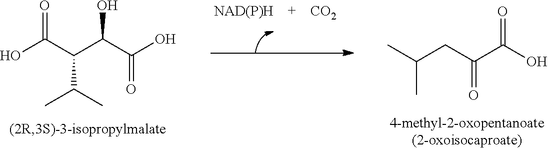 | 1.1.1.85 | Isopropylmalate dehydrogenase | E. coli leuB | NP_414615.4 |
| 4-methyl-2- oxopentanoate + CoA → isovaleryl-CoA + CO₂ | 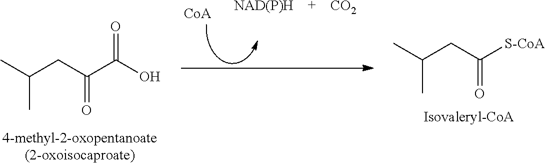 | 1.2.1.- | alpha-keto acid dehydrogenase complex | Streptomyces avermitilis bkdFGH- IpdA1 Homo sapiens BKDHAB- DBT-DLD | BAC72088.1 BAC72089.1 BAC72090.1 KUN54417.1 NP_000700.1 NP_000047.1 NP_001909.3 NP_000099.2 |
| | | | | Bacillus | WP_004398565.1 |
| | | | | subtilis | WP_004398638.1 |
| | | | | bfmBAA- | WP_003230323.1 |
| | | | | bfmBAB- | WP_003232309.1 |
| | | | | bfmBB-pdhD | |
| | | | | E. coli IpdA- | NP_414657.1 |
| | | | | aceEF | NP_414658.1 |
| | | | | | NP_414656.1 |
| Isovaleryl- COA → 3-methyl-2- butenoyl-CoA | 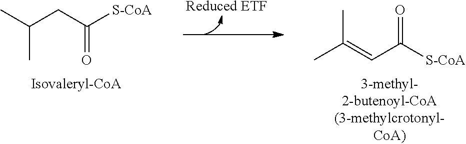 | 1.3.8.4 | Acyl-CoA dehydrogenase | Pseudomonas aeruginosa liuA E. coli aidB E. coli fadE Streptomyces avermitilis acdH | APJ525111 NP_418608.6 NP_414756.2 AAD44196.1 |
| 3-methyl-2- butenoyl- CoA → 3-methyl-2- butenoate | 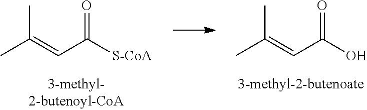 | 3.1.2.- | Thioesterase | E. coli tesA E. coli tesB E. coli yciA E. coli fadM E. coli ydil E. coli ybgC E. coli paal | NP_415027.1 NP_414986.1 NP_415769.1 NP_414977.1 NP_416201.1 NP_415264.1 NP_415914.1 |
| | | | | Mus | P58137.1 |
| | | | | musculus | |
| | | | | acot8 | |
| | | | | Lycopersicon | ADK38536.1 |
| | | | | hirsutum f | |
| | | | | glabratum | |
| | | | | mks2 | |
| | | | | Alcanivorax | YP_692749.1 |
| | | | | borkumensis | |
| | | | | tesB2 | |
| | | | | Fibrobacter | YP_005822012.1 |
| | | | | succinogenes | |
| | | | | Fs2108 | |
| | | | | Prevotella | YP_003574018.1 |
| | | | | ruminicola | |
| | | | | Pr655 | |
| | | | | Prevotella | YP_003574982.1 |
| | | | | ruminicola | |
| | | | | Pr1687 | |
| | | 2.8.3.8 | Acyl-CoA: | E. coli atoD | NP_416725.1 |
| | | | acetyl-CoA | Clostridium | AAA92344.1 |
| | | | transferase | kluyveri | |
| | | | | cat2 | |
| | | | | Clostridium | NP_149326.1, |
| | | | | acetobutylicum | NP_149327.1 |
| | | | | ctfAB | |
| | | | | E. coli ydiF | NP_416209.1 |
| | | 2.3.1.-; | Phosphotransacylase + | Clostridium | NP_349676.1 |
| | | 2.7.2.1; | Carboxylate kinase | acetobutylicum | |
| | | 2.7.2.15 | | ptb | |
| | | | | Enterococcus | AAD55374.1 |
| | | | | faecalis ptb | |
| | | | | Salmonella | AAD39011.1 |
| | | | | enterica pduL | |
| | | | | Clostridium | AAK81015.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica pduW | |
| | | | | E. coli | NP_414820.1, |
| | | | | PaoABC | |
| 3-methyl-2- butenoate → 3-methyl-2- butenal | 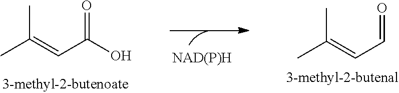 | 1.2.1.- | Carboxylic Acid (Carboxylate) reductase | Mycobacterium marinum Car Nocardia iowensis Car Segniliparus rotundus Car | NP_414819.1, NP_414818.1 WP_012393886.1 AAR91681.1 WP_013138593.1 |
| 3-methyl-2- butenoyl-CoA → 3-methyl-2- butenol | 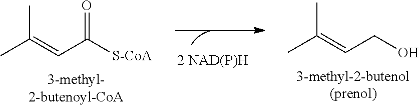 | 1.2.1.- | Alcohol-forming Acyl-CoA reductase | Clostridium acetobutylicum adhE2 Arabidopsis thaliana At3g11980 Arabidopsis thaliana At3g44560 | YP_009076789.1 AEE75132.1 AEE77915.1 |
| | | | | Arabidopsis | AEE79553.1 |
| | | | | thaliana | |
| | | | | At3g56700 | |
| | | | | Arabidopsis | AED93034.1 |
| | | | | thaliana | |
| | | | | At5g22500 | |
| | | | | Arabidopsis | AEE86278.1 |
| | | | | thaliana | |
| | | | | CER4 | |
| | | | | Marinobacter | YP_959486.1 |
| | | | | aquaeolei | |
| | | | | VT8 | |
| | | | | maqu_2220 | |
| | | | | Marinobacter | YP_959769.1 |
| | | | | aquaeolei | |
| | | | | VT8 | |
| | | | | maqu_2507 | |
| 3-methyl-2- butenoyl- CoA → 3- methyl-2- butenal | 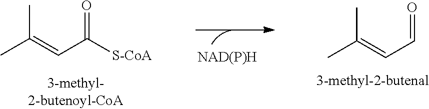 | 1.2.1.- | Aldehyde forming CoA reductase | Acinetobacter calcoaceticus acr1 Acinetobacter sp Strain M-1 acrM Clostridium | AAC45217.1 BAB85476.1 AAT66436.1 |
| | | | | beijerinckii | |
| | | | | ald | |
| | | | | E. coli eut E | NP_416950.1 |
| | | | | Salmonella | AAA80209.1 |
| | | | | enterica eutE | |
| | | | | E. coli mhpF | NP_414885.1 |
| 3-methyl-2- butenal → 3- methyl-2-butenol | 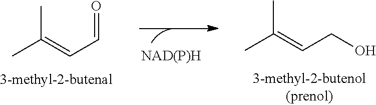 | 1.1.1.- | Alcohol dehydrogenase | E. coli betA E. coli dkgA E. coli eutG E. coli fucO E. coli ucpA E. coli yahK E. coli ybbO | NP_414845.1 NP_417485.4 NP_416948.4 NP_417279.2 NP_416921.4 NP_414859.1 NP_415026.1 |
| | | | | E. coli ybdH | NP_415132.1 |
| | | | | E. coli yiaY | YP_026233.1 |
| | | | | E. coli yjgB | NP_418690.4 |
| | | | | Acinetobacter | AAG10028.1 |
| | | | | sp. SE19 | |
| | | | | ChnD | |
| | | | | Marinobacter | YP_959769.1 |
| | | | | aquaeolei | |
| | | | | VT8 | |
| | | | | maqu_2507 | |
| | | | | Saccharomyces | Q04894.1 |
| | | | | cerevisiae | |
| | | | | ADH6 | |
| | | | | Clostridium | EDK35022.1 |
| | | | | kluyveri | |
| | | | | 4hbD | |
| 3-methyl-2- butenol → dimethylallyl phosphate | 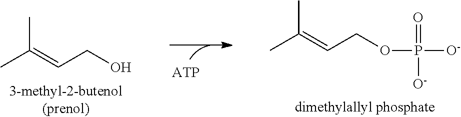 | 2.7.1.- | Alcohol Kinase/ Phosphotransferase | Saccharomyces cerevisiae ERG12 Saccharomyces cerevisiae ERG8 Arabidopsis thaliana At5g58560 | P07277 P24521 Q67ZM7 |
| | | | | Mentha x | P56848 |
| | | | | piperita ipk | |
| | | | | Methanocal | Q58487 |
| | | | | dococcus | |
| | | | | jannaschii | |
| | | | | mvk | |
| | | | | Arabidopsis | AT5G27450.1 |
| | | | | thaliana | |
| | | | | mvk | |
| | | | | E. coli ispE | P62615 |
| | | | | E. coli glpK | P0A6F3 |
| | | | | E. coli ychB | NP_415726.1 |
| | | | | Thermoplasma | WP_010900530.1 |
| | | | | acidophilum | |
| | | | | IPK | |
| dimethylallyl phosphate → dimethylallyl pyrophosphate | 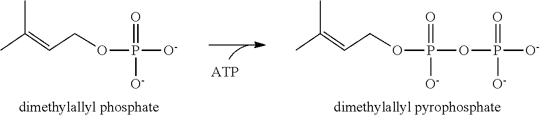 | 2.7.4- | Phosphate Kinase/ Phosphotransferase | Methanothermobacter thermautotrophicus ipk Thermoplasma acidophilum ipk Enterococcus faecalis mvaK2 | AAB84554.1 WP_010900530.1 Q9FD67 |
| | | | | Streptococcus | A0A017UH23 |
| | | | | pneumoniae | |
| | | | | mvaK2 | |
| | | | | Staphylococcus | A0A0E8GDF5 |
| | | | | aureus mvaK2 | |
| | | | | Methanocal | 3K4Y_A |
| | | | | dococcus jannaschii | |
| | | | | ipk | |
| | | | | Escherichia | NP_415725.1 |
| | | | | coli Prs | |
| 3-methyl-2- butenol → dimethylallyl pyrophosphate | 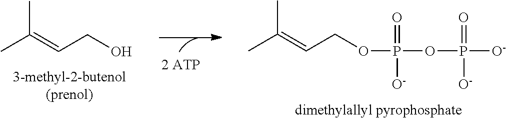 | 2.7.6- | Alcohol diphosphokinase | Mycoplasma pneumoniae M129 PrsA Arabidopsis thaliana col TPK1 Arabidopsis thaliana col TPK2 | NP_109761.1 BAH19964.1 BAH57065.1 |
| DMAPP ↔ IPP | 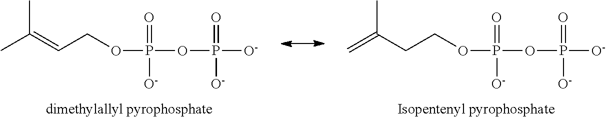 | 5.3.3.2 | isopentenyl diphosphate isomerase | E. coli idi | Q46822 |
| DMAPP + IPP → GPP | 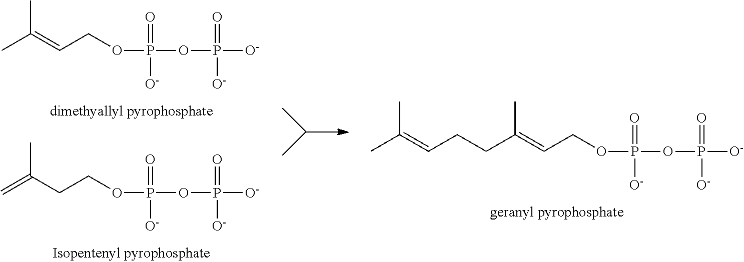 | 2.5.1- | Geranyl pyrophosphate synthase | E. coli ispA Abies grandis GPPS2 | P22939 Q8LKJ2 |

TABLE G
Example reactions and enzymes of the novel isoprenoid precursor synthesis pathway via 2-
hydroxyisovaleric acid and prenol starting from non-decarboxylative acyloin condensation between isobutanal
and formyl-CoA

| | | | | Source | |
|---|---|---|---|---|---|
| | | | | organism and | Protein |
| | | EC | Enzyme | gene/enzyme | Accession |
| Reaction | Illustration | Numbers | names | name | Numbers |
| pyruvate → formate + acetyl- CoA | 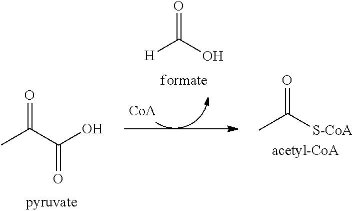 | 2.3.1.54 | Pruvate formate lyase | E. coli pflB E. coli tdcE Chlamydomonas reinhardtii pfl1 Streptococcus mutans pfl | NP_415423.1 YP_026205.1 CAF04129.1 WP_002262619.1 |
| formate + CoA → formyl- CoA | 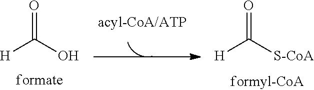 | 6.2.1.- | Acyl- CoA synthetase | E. coli paaK E. coli sucCD E. coli fadK E. coli fadD E. coli prpE E. coli menE | NP_415916.1 NP_415256.1 NP_415257.1 NP_416216.4 NP_414869.1 NP_416763.1 |
| | | | | Penicillium | CAJ15517.1 |
| | | | | chrysogenum | |
| | | | | phl | |
| | | | | E. coli acs | NP_418493.1 |
| | | | | Salmonella | AAL19325.1 |
| | | | | typhimurium | |
| | | | | LT2 prpE | |
| | | | | Bacillus | AAC00261.1 |
| | | | | subtilis bioW | |
| | | | | Cupriavidus | ADE20402.1 |
| | | | | basilensis | |
| | | | | hmfD | |
| | | | | Rhodopseudomonas | CAJ18317.1 |
| | | | | palustris | |
| | | | | badA | |
| | | | | R. palustris | CAE26113.1 |
| | | | | hbaA | |
| | | | | Pseudomonas | NP_249687.1 |
| | | | | aeruginosa | |
| | | | | PAO1 pqsA | |
| | | | | Arabidopsis | Q42524.1 |
| | | | | thaliana 4cl | |
| | | 2.8.3- | CoA | E. coli atoD | NP_416725.1 |
| | | | transferase | E. coli atoA | NP_416726.1 |
| | | | | E. coli scpC | NP_417395.1 |
| | | | | Clostridium | AAA923461.1 |
| | | | | kluyveri cat1 | |
| | | | | Clostridium | AAA92344.1 |
| | | | | kluyveri cat2 | |
| | | | | Clostridium | NP_149326.1, |
| | | | | acetobutylicum | NP_149327.1 |
| | | | | ctfAB | |
| | | | | Pseudomonas | NP_746081.1 |
| | | | | putida pcalJ | NP_746082.1 |
| | | | | Megasphaera | WP_014015705.1 |
| | | | | elsdenii pct | |
| | | | | Acidaminococcus | CAA57199.1 |
| | | | | fermentans | CAA57200.1 |
| | | | | gctAB | |
| | | | | Acetobacter | AGG68319.1 |
| | | | | aceti aarC | |
| | | | | E. coli ydiF | NP_416209.1 |
| | | | | Clostridium | CAB77207.1 |
| | | | | propionicum | |
| | | | | Pct540 | |
| | | 2.3.1.-; | Carboxylate | Clostridium | NP_349676.1 |
| | | 2.7.2.- | kinase + | acetobutylicum ptb | |
| | | | Phospho- | Enterococcus | AAD55374.1 |
| | | | transacylase | faecalis ptb | |
| | | | | Salmonella | AAD39011.1 |
| | | | | enterica pduL | |
| | | | | Clostridium | AAK810151.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica | |
| | | | | pduW | |
| acetyl- CoA + CO₂ → malonyl- CoA | 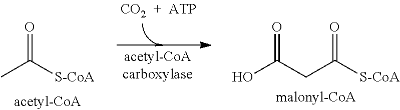 | 6.4.1.2 | Acetyl- CoA carboxylase | E. coli accABCD Saccharomyces cerevisiae HFA1 Homo sapiens | NP_414727.1 NP_417721.1 NP_417722.1 NP_416819.1 P32874.2 NP_001084.3 |
| | | | | ACC2 | |
| | | | | Acidianus | BAC55868.1 |
| | | | | brierleyi | BAC55867.1 |
| | | | | accBC + pccB | BAC55869.1 |
| | | | | Mycobacterium | WP_003900487.1 |
| | | | | tuberculosis | |
| | | | | H37Rv accD6 | |
| | | | | Streptomyces | AAD37851.1 |
| | | | | venezuelae | |
| | | | | ATCC 10712 | |
| | | | | jadJ | |
| 2 acetyl- CoA → acetoacetyl- CoA | 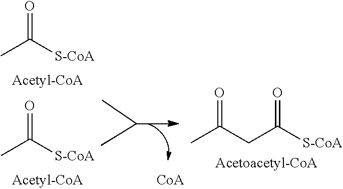 | 2.3.1.- | Thiolase | E. coli atoB E. coli yqeF E. coli fadA E. coli fadl Ralstonia eutropha bktB Pseudomonas sp. Strain B13 catF E coli paaJ Pseudomonas putida pcaF | NP_416728.1 NP_417321.2 YP_026272.1 NP_416844.1 AAC38322.1 AAL02407.1 NP_415915.1 AAA85138.1 |
| | | | | Rhodococcus | YP_002778248.1 |
| | | | | opacus pcaF | |
| | | | | Streptomyces | AAD22035.1 |
| | | | | sp. pcaF | |
| | | | | Ralstonia | AEI80291.1 |
| | | | | eutropha | |
| | | | | phaA | |
| | | | | Clostridium | AAC26023.1 |
| | | | | acetobutylicum | |
| | | | | thlA | |
| | | | | Clostridium | AAC26026.1 |
| | | | | acetobutylicum | |
| | | | | thlB | |
| | | | | Pseudomonas | AAK18168.1 |
| | | | | putida fadA | |
| | | | | P. putida | AAK18171.1 |
| | | | | fadAx | |
| | | | | Acinetobacter | CAG68532.1 |
| | | | | sp. ADP1 | |
| | | | | dcaF | |
| | | | | E. coli paaJ | NP_415915.1 |
| acetyl- CoA + malonyl- COA → acetoacetyl- CoA + CO₂ | 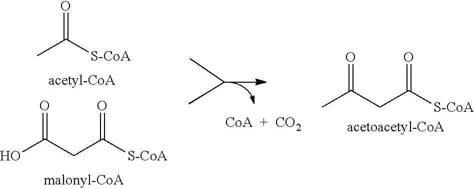 | 2.3.1.- | ketoacyl- CoA synthase | Gluconobacter oxydans GOX0115 Pseudomonas aeruginosa FabH2 Streptomyces sp. MMG1121 PRK09352 Streptomyces tendae Acs2 | AAW59909.1 NP_252023.1 WP_053666104.1 AFS185681 |
| | | | | Streptomyces | BAJ100481 |
| | | | | sp. CL190 | |
| | | | | nph T7 | |
| | | | | Physaria | AAK623481 |
| | | | | fendleri KCS3 | |
| | | | | Saccharomyces | NP_009963.1 |
| | | | | cerevisiae | |
| | | | | ELO2 | |
| | | | | Arabidopsis | NP_171620.2 |
| | | | | thaliana col | |
| | | | | KCS1 | |
| | | | | Arabidopsis | NP_195178.1 |
| | | | | thaliana col | |
| | | | | FAE1 | |
| | | | | Arabidopsis | NP_177020.1 |
| | | | | thaliana col | |
| | | | | CER6 | |
| Acetoacetyl- CoA → 3- hydroxybutyryl- CoA | 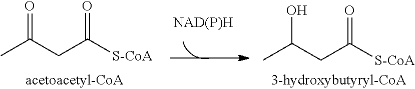 | 1.1.1.35; 1.1.1.36 | Hydroxy acyl- CoA dehydro genase | E. coli fadB E. coli fadJ E. coli paaH P. putida fadB P. putida fadB2x Acinetobacter | NP_418288.1 NP_416843.1 NP_415913.1 AAK18167.2 AAK18170.1 CAG68533.1 |
| | | | | sp. ADP1 | |
| | | | | dcaH | |
| | | | | Ralstonia | P14697.1 |
| | | | | eutrophus | |
| | | | | phaB | |
| | | | | Clostridium | AAA95971.1 |
| | | | | acetobutylicum | |
| | | | | hbd | |
| | | | 3-oxoacyl- | E. coli fabG | NP_415611.1 |
| | | | [acyl- | | |
| | | | carrier- | | |
| | | | protein] | | |
| | | | reductase | | |
| 3- hydroxybutyryl- CoA → crotonyl- CoA | 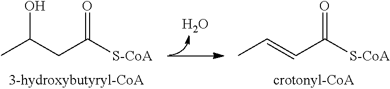 | 4.2.1.17; 4.2.1.119 | enoyl-CoA hydratase | E. coli fadB E. coli fadJ E. coli paaF P. putida fadB P. putida fadB1x | NP_418288.1 NP_416843.1 NP_415911.1 AAK18167.2 AAK18173.1 |
| | | | | Acinetobacter | CAG68535.1 |
| | | | | sp. ADP1 | |
| | | | | dcaE | |
| | | | | Clostridium | AAA95967.1 |
| | | | | acetobutylicum | |
| | | | | crt | |
| | | | | Aeromonas | O32472.1 |
| | | | | caviae phaJ | |
| | | | 3- | E. coli fabA | NP_415474.1 |
| | | | hydroxy | E. coli fabZ | NP_414722.1 |
| | | | acyl- | | |
| | | | [acyl- | | |
| | | | carrier- | | |
| | | | protein] | | |
| | | | dehydratase | | |
| Crotonyl- COA → butyryl- CoA | 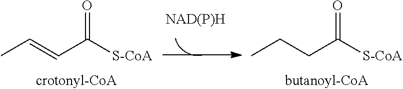 | 1.3.1.44 | enoyl-CoA reductase | Euglena gracilis TER Treponema denticola TER Clostridium | Q5EU90.1 4GGO_A 4EUH_A |
| | | | | acetobutylicum | |
| | | | | TER | |
| | | | enoyl- | E. coli fabl | NP_415804.1 |
| | | | [acyl- | Enterococcus | NP_816503.1 |
| | | | carrier-protein] | faecalis fabK | |
| | | | reductase | Bacillus | KFK80655.1 |
| | | | | subtilis fabL | |
| | | | | Vibrio | ABX38717.1 |
| | | | | cholerae fabV | |
| | | | acyl- | E. coli fadE | NP_414756.2 |
| | | | CoA | E. coli ydiO | NP_416210.4 |
| | | | dehydro | | |
| | | | genase | | |
| Butyryl- COA → isobutyryl- CoA | 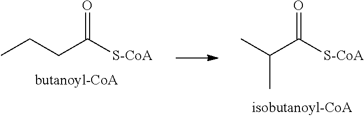 | 5.4.99.- | mutase | Aquincola tertiaricarbonis ibmF | 4R3U_A |
| Isobutanal + formyl- CoA → 3- methyl-2- hydroxybutanoyl- CoA | 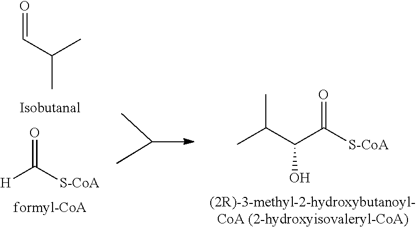 | 4.1.-.- | 2- hydroxy acyl- CoA lyase | Homo sapiens hacl1 Rattus norvegicus hacl1 Dictyostelium discoideum hacl1 Mus musculus hacl1 | Q9UJ83 Q8CHM7 Q54DA9 Q9QXE0 |
| 3-methyl- 2- hydroxy- butanoyl- CoA→3- methyl-2- butenoyl- CoA | 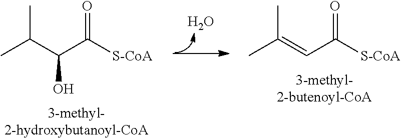 | 4.2.1- | 2- hydroxy acyl- CoA dehydratase | Acidaminococcus fermentans hgdCAB Clostridium symbiosum hgdCAB Fusobacterium nucleatum hgdCAB | P11568, P11569, P11570, Q9X5B6, Q9X5B7, Q9X5B8, A5TT16, A5TT17, A5TT18, |
| | | | | Peptoclostridium | AAV40818.1, |
| | | | | difficile | AAV40819.1, |
| | | | | hadlBC | AAV40820.1 |
| | | | | Clostridium | G3KIM3, |
| | | | | propionicum | G3KIM4, |
| | | | | lcdCAB | G3KIM5 |
| 3-methyl- 2- butenoyl- CoA→3- methyl-2- butenoate | 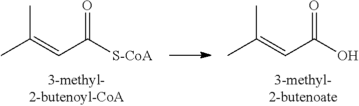 | 3.1.2.- | Thioesterase | E. coli tesA E. coli tesB E. coli yciA E. coli fadM E. coli ydiI E. coli ybgC | NP_415027.1 NP_414986.1 NP_415769.1 NP_414977.1 NP_416201.1 NP_415264.1 |
| 3-methyl- 2- hydroxybutanoyl- CoA→3- methyl-2- hydroxy- butanoate | 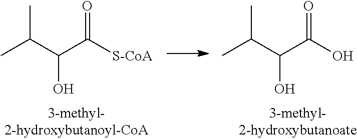 | | | E. coli paal Mus musculus acot8 Lycopersicon hirsutum f glabratum mks2 Alcanivorax borkumensis | NP_415914.1 P58137.1 ADK38536.1 YP_692749.1 |
| | | | | tesB2 | |
| | | | | Fibrobacter | YP_005822012.1 |
| | | | | succinogenes | |
| | | | | Fs2108 | |
| | | | | Prevotella | YP_003574018.1 |
| | | | | ruminicola | |
| | | | | Pr655 | |
| | | | | Prevotella | YP_003574982.1 |
| | | | | ruminicola | |
| | | | | Pr1687 | |
| | | 2.8.3- | CoA | E. coli atoD | NP_416725.1 |
| | | | transferase | E. coli atoA | NP_416726.1 |
| | | | | E. coli scpC | NP_417395.1 |
| | | | | Clostridium | AAA92346.1 |
| | | | | kluyveri cat1 | |
| | | | | Clostridium | AAA92344.1 |
| | | | | kluyveri cat2 | |
| | | | | Clostridium | NP_149326.1, |
| | | | | acetobutylicum | NP_149327.1 |
| | | | | ctfAB | |
| | | | | Pseudomonas | NP_746081.1 |
| | | | | putida pcalJ | NP_746082.1 |
| | | | | Megasphaera | WP_014015705.1 |
| | | | | elsdenii pct | |
| | | | | Acidaminococcus | CAA57199.1 |
| | | | | fermentans | CAA57200.1 |
| | | | | gctAB | |
| | | | | Acetobacter | AGG68319.1 |
| | | | | aceti aarC | |
| | | | | E. coli ydiF | NP_416209.1 |
| | | 2.3.1.-; | Phosphotrans- | Clostridium | NP_349676.1 |
| | | 2.7.2.1; | acylase + | acetobutylicum | |
| | | 2.7.2.15 | Carboxylate | ptb | |
| | | | kinase | Enterococcus | AAD55374.1 |
| | | | | faecalis ptb | |
| | | | | Salmonella | AAD39011.1 |
| | | | | enterica pduL | |
| | | | | Clostridium | AAK81015.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica | |
| | | | | pduW | |
| 3-methyl- 2- butenoate →3- methyl-2- butenal | 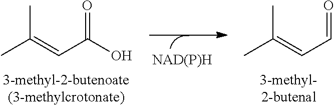 | 1.2.1.- | Carboxylic Acid (Carboxylate) reductase | E. coli PaoABC Mycobacterium marinum Car Nocardia iowensis Car | NP_414820.1, NP_414819.1, NP_414818.1 WP_012393886.1 AAR91681.1 |
| | | | | Segniliparus | WP_013138593.1 |
| | | | | rotundus Car | |
| 3-methyl- 2- butenoyl- COA → 3- methyl-2- butenal | 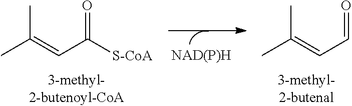 | 1.2.1.- | Aldehyde forming CoA reductase | Acinetobacter calcoaceticus acr1 Acinetobacter sp Strain M-1 acrM Clostridium | AAC45217.1 BAB85476.1 AAT66436.1 |
| Isobutyryl- COA → isobutanal | 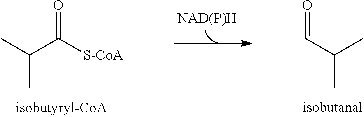 | | | beijerinckii ald E. coli eut E Salmonella enterica eutE E. coli mhpF | NP_416950.1 AAA80209.1 NP_414885.1 |
| 3- methyl- butanal → isovaleryl- CoA | 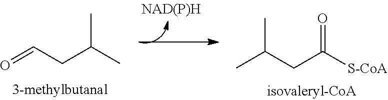 | | | | |
| 3-methyl- 2-butenal → 3- methyl-2- butenol | 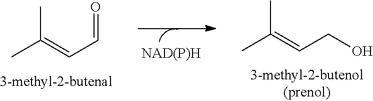 | 1.1.1.- | Alcohol dehydrogenase | E. coli betA E. coli dkgA E. coli eutG E. coli fucO E. coli ucpA E. coli yahK | NP_414845.1 NP_417485.4 NP_416948.4 NP_417279.2 NP_416921.4 NP_414859.1 |
| 3-methyl- 2- hydroxy- butanal → 3- methyl- 1,2- butandiol | 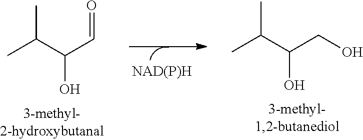 | | | E. coli ybbO E. coli ybdH E. coli yiaY E. coli yjgB Acinetobacter sp. SE19 ChnD Marinobacter aquaeolei | NP_415026.1 NP_415132.1 YP_026233.1 NP_418690.4 AAG10028.1 YP_959769.1 |
| | | | | VT8 | |
| | | | | maqu_2507 | |
| | | | | Saccharomyces | Q04894.1 |
| | | | | cerevisiae | |
| | | | | ADH6 | |
| | | | | Clostridium | EDK35022.1 |
| | | | | kluyveri 4hbD | |
| 3-methyl- 2- butenoyl- COA → 3- methyl-2- butenol | 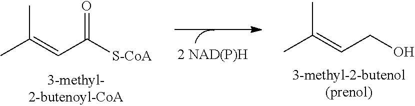 | 1.2.1.- | Alcohol- forming Acyl- CoA reductase | Salmonella enterica pduW Arabidopsis thaliana At3g11980 | AAD39021.1 AEE75132.1 |
| 3-methyl- 2- hydroxy- butanoyl- CoA → 3- methyl- 1,2- butandiol | 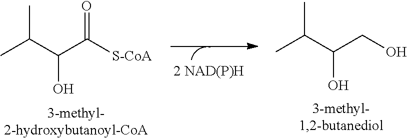 | | | Arabidopsis thaliana At3g44560 Arabidopsis thaliana At3g56700 Arabidopsis thaliana At5g22500 | AEE77915.1 AEE79553.1 AED93034.1 |
| | | | | Arabidopsis | AEE86278.1 |
| | | | | thaliana | |
| | | | | CER4 | |
| | | | | Marinobacter | YP_959486.1 |
| | | | | aquaeolei | |
| | | | | VT8 | |
| | | | | maqu_2220 | |
| | | | | Marinobacter | YP_959769.1 |
| | | | | aquaeolei | |
| | | | | VT8 | |
| | | | | maqu_2507 | |
| 3-methyl- 1,2- butandiol → 3- methylbutanal | 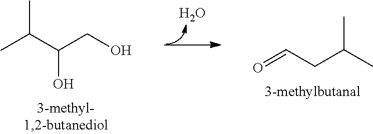 | 4.2.1.- | Diol dehydratase | Klebsiella ocytoca pddABC E. coli pduCDE S. enterica pduCDE | Q59470, Q59471, Q59472 CAS09680, CAS09681, CAS09682 NP_456590, NP_456591, |
| | | | | | NP_456592 |
| isovaleryl- COA→3- methyl-2- butenoyl- CoA | 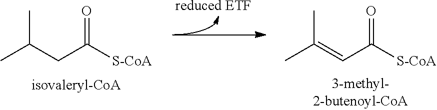 | 1.3.8.4 | Acyl- CoA dehydro genase | Pseudomonas aeruginosa liuA E. coli aidB E. coli fadE Streptomyces avermitilis acdH | APJ52511.1 NP_418608.6 NP_414756.2 AAD44196.1 |
| 3-methyl- 2-butenol → dimethylalyl phosphate | 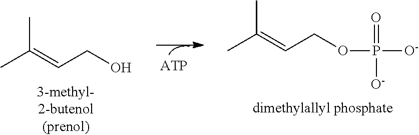 | 2.7.1.- | Alcohol Kinase/ Phospho- transferase | Saccharomyces cerevisiae ERG12 Saccharomyces cerevisiae ERG8 Arabidopsis thaliana At5g58560 | P07277 P24521 Q67ZM7 |
| | | | | Mentha × | P56848 |
| | | | | piperita ipk | |
| | | | | Arabidopsis | AT5G27450.1 |
| | | | | thaliana mvk | |
| | | | | E. coli ychB | NP_415726.1 |
| | | | | E. coli glpK | POA6F3 |
| | | | | Methanother | AAB84554.1 |
| | | | | mobacter | |
| | | | | therma- | |
| | | | | utotrophicus ipk | |
| | | | | Thermoplasma | WP_010900530.1 |
| | | | | acidophilum | |
| | | | | ipk | |
| | | | | Methanocal- | 3K4Y_A |
| | | | | dococcus | |
| | | | | jannaschii ipk | |
| dimethylallyl phosphate → dimethylallyl pyrophosphate | 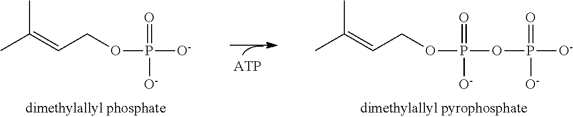 | 2.7.4- | Phosphate Kinase/ Phosph0- transferase | Methanothermo- bacter thermautotrophicus ipk Thermoplasma acidophilum ipk Enterococcus | AAB84554.1 WP_010900530.1 Q9FD67 |
| | | | | faecalis | |
| | | | | mvaK2 | |
| | | | | Streptococcus | A0A017UH23 |
| | | | | pneumoniae | |
| | | | | mvaK2 | |
| | | | | Staphylococcus | A0A0E8GDF5 |
| | | | | aureus | |
| | | | | mvaK2 | |
| | | | | Methano- | 3K4Y_A |
| | | | | caldococcus | |
| | | | | jannaschii ipk | |
| 3-methyl- 2-butenol → dimethylallyl pyrophosphate | 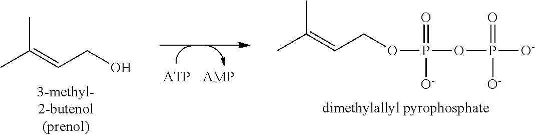 | 2.7.6.- | Alcohol diphospho- kinase | E. coli prs Mycoplasma pneumoniae M129 prsA Arabidopsis thaliana col TPK1 Arabidopsis thaliana col | NP_415725.1 NP_109761.1 BAH19964.1 BAH57065.1 |
| | | | | TPK2 | |
| DMAPP ↔ IPP | 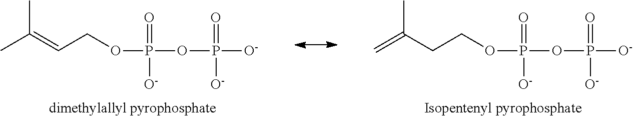 | 5.3.3.2 | isopentenyl diphosphate isomerase | E. coli idi | NP_417365.1 |
| DMAPP + IPP→GPP | 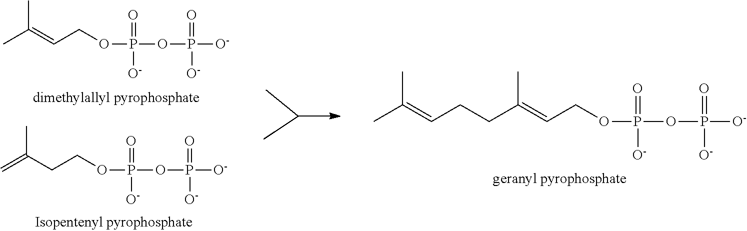 [SMILES: C=C(C)CCOP(=O)([O-])OP(=O)([O-])[O-] ; CC(C)=CCOP(=O)([O-])OP(=O)([O-])[O-] ; CC(C)=CCC/C(C)=C/COP(=O)([O-])OP(=O)([O-])[O-]] | 2.5.1- | Geranyl pyrophosphate synthase | E. coli ispA Abies grandis GPPS2 | NP_414955.1 AAN01134.1 |

TABLE H
Example reactions and enzymes of the pathway for isoprenoid precursor synthesis via 2-hydroxyisovaleric acid and prenol starting from aldol condensation between acetaldehyde and pyruvate

| | | | | Source | |
|---|---|---|---|---|---|
| | | | | organism | |
| | | | | and | Protein |
| | | EC | Enzyme | gene/enzyme | Accession |
| Reaction | Illustration | Number | names | name | Numbers |
| pyruvate → acetaldehyde | 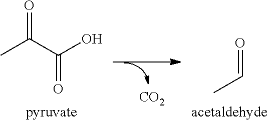 | 4.1.1.1 | alpha- ketoacid decarboxylase | Saccharomyces cerevisiae PDC1 Saccharomyces cerevisiae PDC5 Saccharomyces cerevisiae PDC6 | NP_013145.1 NP_013235.1 NP_011601.3 |
| | | | | Pisum | CAA91444.1 |
| | | | | sativum | |
| | | | | PDC1 | |
| | | | | Saccharomyces | NP_010668.3 |
| | | | | cerevisiae | |
| | | | | ARO10 | |
| | | | | Saccharomyces | |
| | | | | cerevisiae | NP_010203.1 |
| | | | | THI3 | |
| | | | | Lactococcuss | CAG34226.1 |
| | | | | lactis kivd | |
| | | | | Pantoea | WP_003848906.1 |
| | | | | agglomerans | |
| | | | | idpC | |
| | | | | Enterobacter | WP_073396207.1 |
| | | | | cloacae idpC | |
| pyruvate + acetaldehyde → 4- hydroxy-2- oxopentanoate | 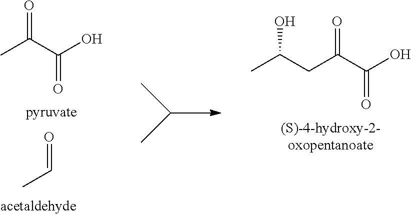 | 4.1.3.39 | aldolase | E. coli mhpE Pseudomonas putida xylK Pseudomonas sp. CF600 dmpG | NP_414886.1 WP_011005904.1 WP_017849278.1 |
| 4-hydroxy-2- oxopentanoate → 3-hydroxy-2- oxo-3- methylbutanate | 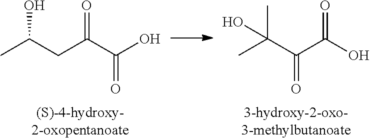 | 5.4.99.- | mutase | Streptomyces cinnamonensis icmAB Metallosphaera sedula Msed_0638, Msed_2055 Cupriavidus metallidurans icmF | AAC08713.1. CAB59633.1 A4YEG1, A4YIE3 Q1LRY0 |
| | | | | Kyrpidia tusciae | D5WTR7, |
| | | | | rcmAB | D5WTR8 |
| | | | | Rhodobacter | ABA80144.1 |
| | | | | sphaeroides | |
| | | | | meaA | |
| 2,3-dihydroxy-3- methylbutanoate → 3-methyl-2- oxobutanoate | 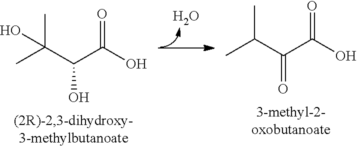 | 4.2.1.9 | Dihydroxyacid dehydratase | E. coli ilvD | P05791 |
| 3-hydroxy-2-oxo-3- methylbutanoate → 2,3-dihydroxy- 3-methylbutanoate | 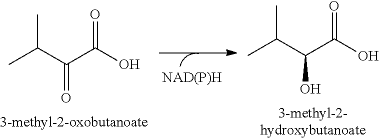 | 1.1.1.- | 2-hydroxyacid dehydrogenase | Acidaminococcus fermentans hgdH Methanocal dococcus jannaschii mdh | D2RMS7 Q60176 |
| 3-methyl-2- oxobutanoate → 3-methyl-2- hydroxybutanoate | 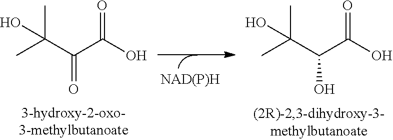 | | | M. jannaschii comC E. coli mdh E. coli serA E. coli IdhA Haloferax mediterranei ddh Lactococcus lactis panE | Q58820 P61889 P0A9T0 P52643 Q2VEQ7 A0A089XK51 |
| 3-methyl-2- hydroxybutanoate → 3-methyl-2- butenoate | 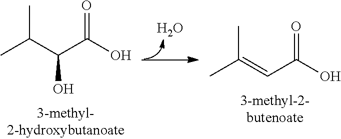 | 4.2.1.- 4.2.1- | 2- hydroxyacid dehydratase 2- hydroxyacyl- CoA dehydratase | E. coli fumA E. coli fumB E. coli fumC Acidaminococcus fermentans hgdCAB Clostridium symbiosum hgdCAB | P0AC33 P14407 P05042 P11568, P11569, P11570, Q9X5B6, Q9X5B7, Q9X5B8, |
| | | | | Fusobacterium | A5TT16, |
| | | | | nucleatum | A5TT17, |
| | | | | hgdCAB | A5TT18, |
| | | | | Peptoclostridium | AAV40818.1, |
| | | | | difficile hadIBC | AAV40819.1, |
| | | | | | AAV40820.1 |
| | | | | Clostridium | G3KIM3, |
| | | | | propionicum | G3KIM4, |
| | | | | IcdCAB | G3KIM5 |
| | | 6.2.1.- | Acyl- | E. coli paaK | NP_415916.1 |
| | | | CoA | E. coli | NP_415256.1 |
| | | | synthetase | sucCD | NP_415257.1 |
| | | | | E. coli fadK | NP_416216.4 |
| 3-methyl-2- hydroxybutanoate → 3-methyl-2- hydroxybutanoyl- CoA | 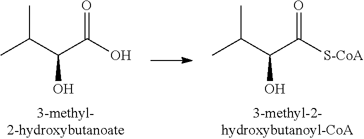 | | | E. coli fadD E. coli prpE E. coli menE Penicillium chrysogenum phl Salmonella typhimurium LT2 prpE | NP_416319.1 NP_414869.1 NP_416763.1 CAJ15517.1 AAL19325.1 |
| 3-methyl-2- butenoate → 3-methyl- 2-butenoyl- CoA | 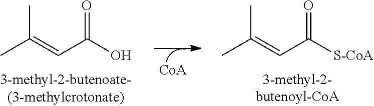 | | | Bacillus subtilis bioW Cupriavidus basilensis hmfD Rhodopseu domonas palustris badA R. palustris hbaA | AAC00261.1 ADE20402.1 CAJ18317.1 CAE26113.1 |
| | | | | Pseudomonas | NP_249687.1 |
| | | | | aeruginosa | |
| | | | | PAO1 pgsA | |
| | | | | Arabidopsis | Q42524.1 |
| | | | | thaliana 4cl | |
| | | 2.8.3- | CoA | E. coli atoD | NP_416725.1 |
| | | | transferase | E. coli atoA | NP_416726.1 |
| | | | | E. coli scpC | NP_417395.1 |
| | | | | Clostridium | AAA92346.1 |
| | | | | kluyveri | |
| | | | | cat1 | |
| | | | | Clostridium | AAA92344.1 |
| | | | | kluyveri | |
| | | | | cat2 | |
| | | | | Clostridium | NP_149326.1, |
| | | | | acetobutylicum | NP_149327.1 |
| | | | | ctfAB | |
| | | | | Pseudomonas | NP_746081.1 |
| | | | | putida | NP_746082.1 |
| | | | | pcalJ | |
| | | | | Megasphaera | WP_014015705.1 |
| | | | | elsdenii pct | |
| | | | | Acidaminococcus | CAA57199.1 |
| | | | | fermentans | CAA57200.1 |
| | | | | gctAB | |
| | | | | Acetobacter | AGG68319.1 |
| | | | | aceti aarC | |
| | | | | E. coli ydiF | NP_416209.1 |
| | | | | Clostridium | CAB77207.1 |
| | | | | propionicum | |
| | | | | Pct540 | |
| | | 2.3.1.-; | Carboxylate | Clostridium | NP_349676.1 |
| | | 2.7.2.- | kinase + | acetobutylicum | |
| | | | Phosphotransacylase | ptb | |
| | | | | Enterococcus | AAD55374.1 |
| | | | | faecalis ptb | |
| | | | | Salmonella | AAD39011.1 |
| | | | | enterica | |
| | | | | pduL | |
| | | | | Clostridium | AAK81015.1 |
| | | | | acetobutylicum | |
| | | | | buk | |
| | | | | Enterococcus | AAD55375.1 |
| | | | | faecalis | |
| | | | | buk | |
| | | | | Salmonella | AAD39021.1 |
| | | | | enterica | |
| | | | | pduW | |
| 3-methyl-2- hydroxybutanoyl- CoA → 3-methyl-2- butenoyl-CoA | 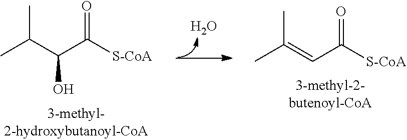 | 4.2.1- | 2-hydroxy acyl-CoA dehydratase | Acidaminococcus fermentans hgdCAB Clostridium symbiosum hgdCAB Fusobacterium nucleatum hgdCAB | P11568, P11569, P11570, Q9X5B6, Q9X5B7, Q9X5B8, A5TT16, A5TT17, A5TT18, |
| | | | | Peptoclostri | AAV40818.1, |
| | | | | dium difficile | AAV40819.1, |
| | | | | hadIBC | AAV40820.1 |
| | | | | Clostridium | G3KIM3, |
| | | | | propionicum | G3KIM4, |
| | | | | IcdCAB | G3KIM5 |
| 3-methyl-2- butenoyl- CoA → 3- methyl-2- butenoate | 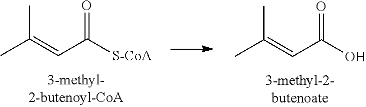 | 3.1.2.- | Thioesterase | E. coli tesA E. coli tesB E. coli yciA E. coli fadM E. coli ydil E. coli ybgC E. coli paal | NP_415027.1 NP_414986.1 NP_415769.1 NP_414977.1 NP_416201.1 NP_415264.1 NP_415914.1 |
| 3-methyl-2- hydroxybutanoyl- COA → 3- methyl-2- hydroxybuta noate | 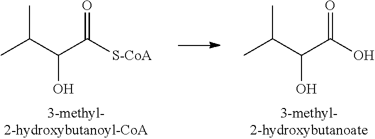 | | | Mus musculus acot8 Lycopersicon hirsutum f glabratum mks2 Alcanivorax borkumensis tesB2 Fibrobacter succinogenes Fs2108 Prevotella ruminicola Pr655 Prevotella ruminicola Pr1687 | P58137.1 ADK38536.1 YP_692749.1 YP_005822012.1 YP_003574018.1 YP_003574982.1 |
| | | 2.8.3.8 | Acyl-CoA: | E. coli atoD | NP_416725.1 |
| | | | acetyl-CoA | Clostridium | AAA92344.1 |
| | | | transferase | kluyveri cat2 | |
| | | | | Clostridium | NP_149326.1, |
| | | | | acetobutylicum | NP_149327.1 |
| | | | | ctfAB | |
| | | | | E. coli ydiF | NP_416209.1 |
| | | 2.3.1.-; | Phosphotransacylase + | Clostridium | NP_349676.1 |
| | | 2.7.2.1; | Carboxylate | acetobutylicum | |
| | | 2.7.2.15 | kinase | ptb | |
| | | | | Enterococcus | AAD55374.1 |
| | | | | faecalis ptb | |
| | | | | Salmonella | AAD39011.1 |
| | | | | enterica pduL | |
| | | | | Clostridium | AAK81015.1 |
| | | | | acetobutylicum | |
| | | | | buk | |
| | | | | Enterococcus | AAD55375.1 |
| | | | | faecalis buk | |
| | | | | Salmonella | AAD39021.1 |
| | | | | enterica pduW | |
| 3-methyl-2- butenoate → 3-methyl- 2-butenal | 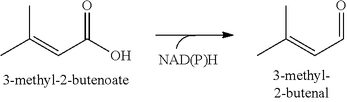 | 1.2.1.- | Carboxylic Acid (Carboxylate) reductase | E. coli PaoABC Mycobacterium marinum Car Nocardia iowensis Car | NP_414820.1, NP_414819.1, NP_414818.1 WP_012393886.1 AAR91681.1 |
| 3-methyl-2- hydroxybutanoate → 3-methyl-2- hydroxybutanal | 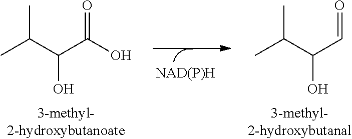 | | | Segniliparus rotundus Car | WP_013138593.1 |
| 3-methyl-2- butenoyl- COA → 3-methyl-2- butenol | 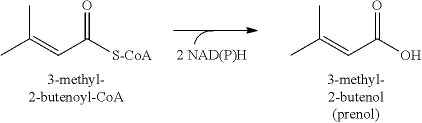 | 1.2.1.- | Alcohol-forming Acyl-CoA reductase | Clostridium acetobutylicum adhE2 Arabidopsis thaliana At3g11980 Arabidopsis thaliana At3g44560 Arabidopsis thaliana At3g56700 | YP_009076789.1 AEE75132.1 AEE77915.1 AEE79553.1 |
| 3-methyl-2- hydroxybutanoyl- CoA → 3-methyl- 1,2-butandiol | 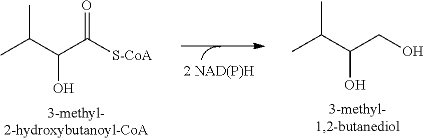 | | | Arabidopsis thaliana At5g22500 Arabidopsis thaliana CER4 Marinobacter aquaeolei VT8 maqu_2220 Marinobacter aquaeolei VT8 maqu_2507 | AED93034.1 AEE86278.1 YP_959486.1 YP_959769.1 |
| acetyl-CoA → acetaldehyde | 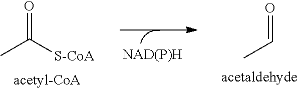 | 1.2.1.- | Aldehyde forming CoA reductase | Acinetobacter calcoaceticus acr1 Acinetobacter sp Strain M-1 acrM Clostridium beijerinckii ald | AAC45217.1 BAB85476.1 AAT66436.1 |
| 3-methyl-2- butenoyl- COA → 3- methyl-2- butenal | 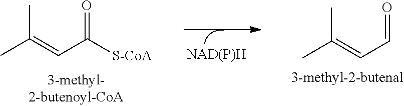 | | | E. coli eutE Salmonella enterica eutE E. coli mhpF | NP_416950.1 AAA80209.1 NP_414885.1 |
| 3-methyl-2- hydroxybutanoyl- CoA → 3-methyl-2- hydroxybutanal | 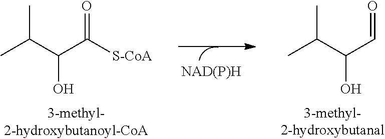 | | | | |
| 3- methylbutanal → isovaleryl- CoA | 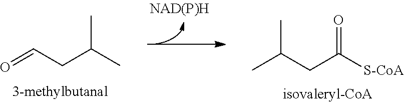 | | | | |
| 3-methyl-2- butenal → 3-methyl-2- butenol | 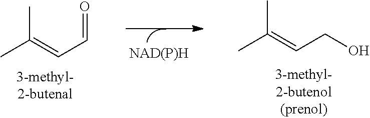 | 1.1.1.- | Alcohol dehydrogenase | E. coli betA E. coli dkgA E. coli eutG E. coli fucO E. coli ucpA E. coli yahK E. coli ybbO | NP_414845.1 NP_417485.4 NP_416948.4 NP_417279.2 NP_416921.4 NP_414859.1 NP_415026.1 |
| 3-methyl-2- hydroxybutanal → 3-methyl-1,2- butandiol | 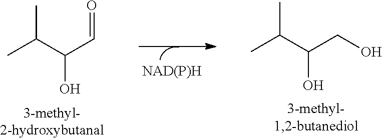 | | | E. coli ybdH E. coli yiaY E. coli yjgB Acinetobacter sp. SE19 ChnD Marinobacter aquaeolei VT8 maqu 2507 | NP_415132.1 YP_026233.1 NP_418690.4 AAG10028.1 YP_959769.1 |
| | | | | Saccharomyces | Q04894.1 |
| | | | | cerevisiae | |
| | | | | ADH6 | |
| | | | | Clostridium | EDK35022.1 |
| | | | | kluyveri | |
| | | | | 4hbD | |
| | | | | Klebsiella | Q59470, |
| | | | | ocytoca | Q59471, |
| | | | | pddABC | Q59472 |
| | | | | E. coli | CAS09680, |
| | | | | pduCDE | |
| 3-methyl-1,2- butandiol → 3- methylbutanal | 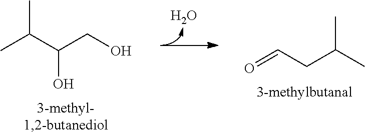 | 4.2.1.- | Diol dehydratase | S. enterica pduCDE | CAS09681, CAS09682 NP_456590, NP_456591, NP_456592 |
| isovaleryl- COA → 3-methyl- 2-butenoyl-CoA | 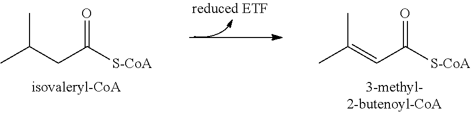 | 1.3.8.4 | Acyl-CoA dehydrogenase | Pseudomonas aeruginosa liuA E. coli aidB E. coli fadE Streptomyces avermitilis acdH Saccharomyces | APJ52511.1 NP_418608.6 NP_414756.2 AAD44196.1 P07277 |
| 3-methyl-2- butenol → dimethylallyl phosphate | 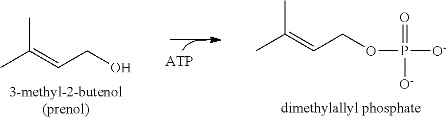 | 2.7.1.- | AlcoholKinase/ Phosphotransferase | cerevisiae ERG12 Saccharomyces cerevisiae ERG8 Arabidopsis thaliana At5g58560 Mentha x piperita ipk Methanocal dococcus jannaschii mvk | P24521 Q67ZM7 P56848 Q58487 |
| | | | | Arabidopsis | AT5G27450.1 |
| | | | | thaliana mvk | |
| | | | | E. coli ychB | NP_415726.1 |
| | | | | E. coli glpK | P0A6F3 |
| | | | | Methanothermobacter | AAB84554.1 |
| | | | | thermautotrophicus | |
| | | | | ipk | |
| | | | | Thermoplasma | WP_010900530.1 |
| | | | | acidophilum | |
| | | | | ipk | |
| | | | | Methanocal | 3K4Y_A |
| | | | | dococcus | |
| | | | | jannaschii ipk | |
| dimethylallyl phosphate → dimethylallyl pyrophosphate | 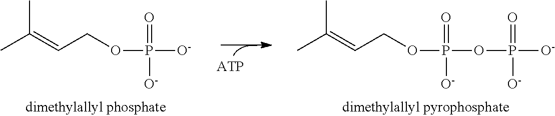 | 2.7.4- | Phosphate Kinase/ Phosphotransferase | Methanothermobacter thermautotrophicus ipk Thermoplasma acidophilum ipk Enterococcus faecalis mvaK2 Streptococcus | AAB84554.1 WP_010900530.1 Q9FD67 A0A0I7UH23 |
| | | | | pneumoniae | |
| | | | | mvaK2 | |
| | | | | Staphylococcus | A0A0E8GDF5 |
| | | | | aureus mvaK2 | |
| | | | | Methanocal | 3K4Y_A |
| | | | | dococcus jannaschil | |
| | | | | ipk | |
| 3-methyl-2- butenol → dimethylallyl pyrophosphate | 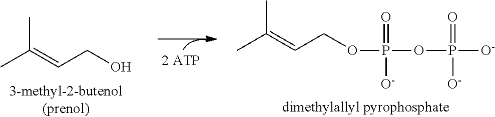 | 2.7.6- | Alcohol diphosphokinase | Escherichia coli Prs Mycoplasma pneumoniae M129 PrsA Arabidopsis thaliana col TPK1 Arabidopsis thaliana col TPK2 | NP_415725.1 NP_109761.1 BAH19964.1 BAH57065.1 |
| DMAPP ↔ IPP | 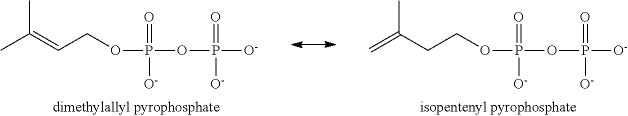 | 5.3.3.2 | isopentenyl diphosphate isomerase | E. coli idi | Q46822 |
| DMAPP + IPP → GPP | 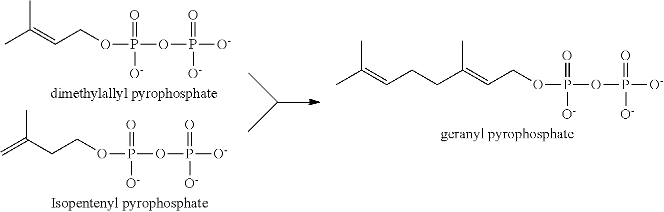 [SMILES: C=C(C)CCOP(=O)([O-])OP(=O)([O-])[O-] ; CC(C)=CCOP(=O)([O-])OP(=O)([O-])[O-] ; CC(C)=CCC/C(C)=C/COP(=O)([O-])OP(=O)([O-])[O-]] | 2.5.1- | Geranyl pyrophosphate synthase | E. coli ispA Abies grandis GPPS2 | P22939 Q8LKJ2 |

TABLE I
Example reactions and enzymes of the pathway for isoprenoid precursor synthesis via 2-oxoisovaleric acid, and 2-ocoisocaproic acid and prenol starting from aldol condensation between pyruvate and acetaldehyde

| | | | | Source | |
|---|---|---|---|---|---|
| | | | | organism | |
| | | | | and | Protein |
| | | EC | Enzyme | gene/enzyme | Accession |
| Reaction | Illustration | Numbers | names | name | Numbers |
| pyruvate → acetaldehyde | 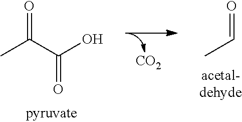 | 4.1.1.1 | alpha- ketoacidecarboxylase | Saccharomyces cerevisiae PDC1 Saccharomyces cerevisiae PDC5 Saccharomyces cerevisiae | NP_ 013145.1 NP_ 013235.1 NP_011601.3 |
| 4-methyl-2- oxopentano ate → 3- methylbutanal + CO₂ | 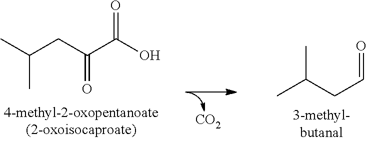 | | | PDC6 Pisum sativum PDC1 Saccharomyces cerevisiae ARO10 Saccharomyces cerevisiae THI3 | CAA91444.1 NP_010668.3 NP_010203.1 |
| | | | | Lactococcus | CAG34226.1 |
| | | | | lactis kivd | |
| | | | | Pantoea | WP_003848906.1 |
| | | | | agglomerans | |
| | | | | idpC | |
| | | | | Enterobacter | WP_073396207.1 |
| | | | | cloacae | |
| | | | | idpC | |
| pyruvate + acetaldehyde → 4-hydroxy-2- oxopentanoate | 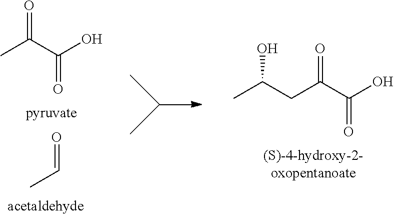 | 4.1.3.39 | aldolase | E. coli mhpE Pseudomonas putida xylK Pseudomonas sp. CF600 dmpG | NP_414886.1 WP_011005904.1 WP_017849278.1 |
| 4-hydroxy- 2- oxopentanoate → 3-hydroxy-2- oxo-3- methylbutanoate | 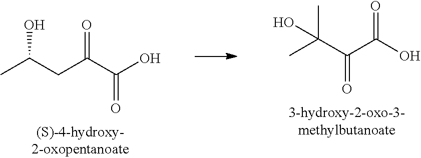 | 5.4.99.- | mutase | Streptomyces cinnamonensis icmAB Metallosphaera sedula Msed_0638, Msed_2055 Cupriavidus metallidurans icmF Kyrpidia tusciae rcmAB Rhodobacter sphaeroides meaA | AAC08713.1. CAB59633.1 A4YEG1, A4YIE3 Q1LRY0 D5WTR7, D5WTR8 ABA80144.1 |
| 3-hydroxy- 2-oxo-3- methylbutanoate → 2,3-dihydroxy- 3-methylbutanoate | 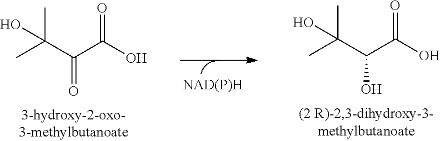 | 1.1.1.- | 2- hydroxyaciddehydrogenase | Acidaminococcus fermentans hgdH Methanocal dococcus jannaschii mdh M. jannaschii comC E. coli mdh E. coli serA E. coli IdhA Haloferax mediterrane i ddh Lactococcus lactis panE | D2RMS7 Q60176 Q58820 NP_417703.1 NP_417388.1 NP_415898.1 Q2VEQ7 A0A089XK51 |
| 2,3-dihydroxy-3- methylbutanoate→ 3-methyl-2- oxobutanoate | 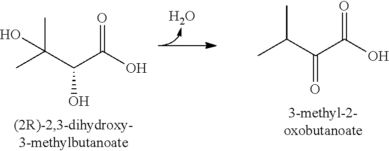 | 4.2.1.9 | Dihydroxyacid dehydratase | E. coli ilvD | P05791 |
| 3-methyl-2- oxobutanoate + acetyl-COA → 2-isopropylmalate + CoA | 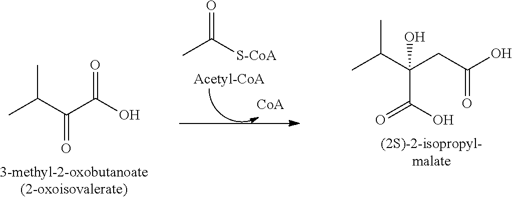 | 2.3.3.13 | Isopropylmalate synthase | E. coli leuA | NP_414616.1 |
| 2- isopropylmalate → 3-isopropylmalate | 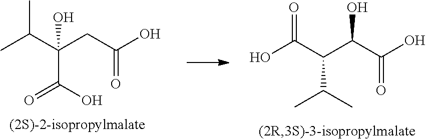 | 4.2.1.33 | Isopropylmalate isomerase | E. coli leuCD | NP_414614.1 NP_414613.1 |
| 3- isopropylmalate → 4-methyl-2- oxopentanoate + CO₂ | 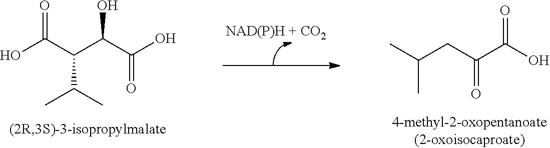 | 1.1.1.85 | Isopropylmalate dehydrogenase | E. coli leuB | NP_414615.4 |
| 4-methyl-2- oxopentanoate + CoA → isovaleryl- CoA + CO₂ | 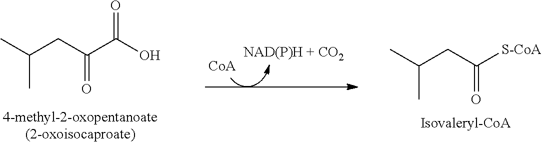 | 1.2.1.- | alpha- keto acid dehydrogenase complex | Streptomyces avermitilis bkdFGH- IpdA1 Homo sapiens BKDHAB- DBT-DLD Bacillus | BAC72088.1 BAC72089.1 BAC72090.1 KUN54417.1 NP_000700.1 NP_000047.1 NP_001909.3 NP_000099.2 WP_004398565.1 |
| | | | | subtilis | |
| | | | | bfmBAA- | |
| | | | | bfmBAB- | WP_004398638.1 |
| | | | | bfmBB- | |
| | | | | pdhD | WP_003230323.1 |
| | | | | | WP_003232309.1 |
| | | | | E. coli lpdA- | NP_414657.1 |
| | | | | aceEF | NP_414658.1 |
| | | | | | NP_414656.1 |
| Isovaleryl- CoA → 3- methyl-2- butenoyl-CoA | 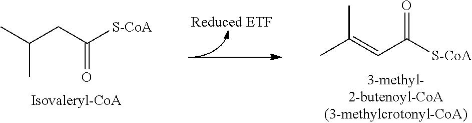 | 1.3.8.4 | Acyl-CoA dehydrogenase | Pseudomonas aeruginosa liuA E. coli aidB E. coli fadE Streptomyces avermitilis acdH | APJ525111 NP_418608.6 NP_414756.2 AAD44196.1 |
| 3-methyl-2- butenoyl- CoA → 3- methyl-2- butenoate | 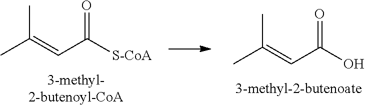 | 3.1.2.- | Thioesterase | E. coli tesA E. coli tesB E. coli yciA E. coli fadM E. coli ydil E. coli ybgC E. coli paal | NP_415027.1 NP_414986.1 NP_415769.1 NP_414977.1 NP_416201.1 NP_415264.1 NP_415914.1 |
| | | | | Mus | P58137.1 |
| | | | | musculus | |
| | | | | acot8 | |
| | | | | Lycopersicon | ADK38536.1 |
| | | | | hirsutum f | |
| | | | | glabratum | |
| | | | | mks2 | |
| | | | | Alcanivorax | YP_692749.1 |
| | | | | borkumensis | |
| | | | | tesB2 | |
| | | | | Fibrobacter | YP_005822012.1 |
| | | | | succinogenes | |
| | | | | Fs2108 | |
| | | | | Prevotella | YP_003574018.1 |
| | | | | ruminicola | |
| | | | | Pr655 | |
| | | | | Prevotella | YP_003574982.1 |
| | | | | ruminicola | |
| | | | | Pr1687 | |
| | | 2.8.3.8 | Acyl-CoA: | E. coli atoD | NP_416725.1 |
| | | | transferase | Clostridium | AAA92344.1 |
| | | | | kluyveri | |
| | | | | cat2 | |
| | | | | Clostridium | NP_149326.1, |
| | | | | acetobutylicum | NP_149327.1 |
| | | | | ctfAB | |
| | | | | E. coli ydiF | NP_416209.1 |
| | | 2.3.1.-; | Phosphotransacylase + | Clostridium | NP_349676.1 |
| | | 2.7.2.1; | Carboxylate kinase | acetobutylicum | |
| | | 2.7.2.15 | | ptb | |
| | | | | Enterococcus | AAD55374.1 |
| | | | | faecalis | |
| | | | | ptb | |
| | | | | Salmonella | AAD39011.1 |
| | | | | enterica | |
| | | | | pduL | |
| | | | | Clostridium | AAK81015.1 |
| | | | | acetobutylicum | |
| | | | | buk | |
| | | | | Enterococcus | AAD55375.1 |
| | | | | faecalis | |
| | | | | buk | |
| | | | | Salmonella | AAD39021.1 |
| | | | | enterica | |
| | | | | pduW | |
| 3-methyl-2- butenoate → 3-methyl- 2-butenal | 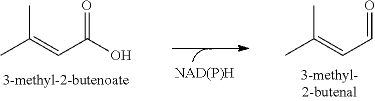 | 1.2.1.- | Carboxylic Acid (Carboxylate) reductase | E. coli PaoABC Mycobacterium marinum Car Nocardia iowensis Car Segniliparus rotundus Car | NP_414820.1, NP_414819.1, NP_414818.1 WP_012393886.1 AAR91681.1 WP_013138593.1 |
| 3-methyl-2- butenoyl- COA → 3- methyl-2- butenol | | 1.2.1.- | Alcohol-forming Acyl-CoA reductase | Clostridium acetobutylicum adhE2 Arabidopsis thaliana At3g11980 Arabidopsis thaliana At3g44560 Arabidopsis thaliana At3g56700 Arabidopsis thaliana | YP_009076789.1 AEE75132.1 AEE77915.1 AEE79553.1 AED93034.1 |
| | | | | At5g22500 | |
| | | | | Arabidopsis | AEE86278.1 |
| | | | | thaliana | |
| | | | | CER4 | |
| | | | | Marinobacter | YP_959486.1 |
| | | | | aquaeolei | |
| | | | | VT8 | |
| | | | | maqu_2220 | |
| | | | | Marinobacter | YP_959769.1 |
| | | | | aquaeolei | |
| | | | | VT8 | |
| | | | | maqu_2507 | |
| 3-methyl-2- butenoyl- COA → 3- methyl-2- butenal | 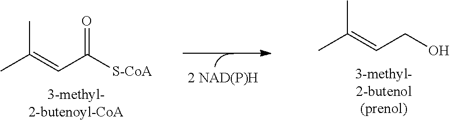 | 1.2.1.- | Aldehyde forming CoA reductase | Acinetobacter calcoaceticus acr1 Acinetobacter sp Strain M-1 acrM Clostridium beijerinckii ald | AAC45217.1 BAB85476.1 AAT66436.1 |
| acetyl-CoA → acetaldehyde | 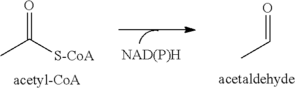 | | | E. coli eut E Salmonella enterica eutE E. coli mhpF | NP_416950.1 AAA80209.1 NP_414885.1 |
| 3-methyl-2- butenal → 3-methyl-2- butenol | 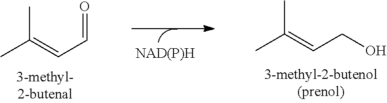 | 1.1.1.- | Alcohol dehydrogenase | E. coli betA E. coli dkgA E. coli eutG E. coli fucO₂ E. coli ucpA E. coli yahK E. coli ybbO₂ E. coli ybdH | NP_414845.1 NP_417485.4 NP_416948.4 NP_417279.2 NP_416921.4 NP_414859.1 NP_415026.1 NP_415132.1 |
| | | | | E. coli yiaY | YP_026233.1 |
| | | | | E. coli yjgB | NP_418690.4 |
| | | | | Acinetobacter | AAG10028.1 |
| | | | | sp. SE19 | |
| | | | | ChnD | |
| | | | | Marinobacter | YP_959769.1 |
| | | | | aquaeolei | |
| | | | | VT8 | |
| | | | | maqu_2507 | |
| | | | | Saccharomyces | Q04894.1 |
| | | | | cerevisiae | |
| | | | | ADH6 | |
| | | | | Clostridium | EDK35022.1 |
| | | | | kluyveri | |
| | | | | 4hbD | |
| 3-methyl-2- butenol → dimethylallyl phosphate | 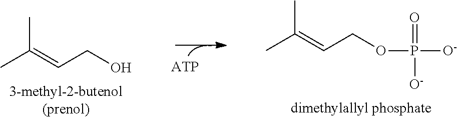 | 2.7.1.- | Alcohol Kinase/ Phosphotransferase | Saccharomyces cerevisiae ERG12 Saccharomyces cerevisiae ERG8 Arabidopsis thaliana At5g58560 Mentha x piperita ipk | P07277 P24521 Q67ZM7 P56848 |
| | | | | Methanocal | Q58487 |
| | | | | dococcus | |
| | | | | jannaschil | |
| | | | | mvk | |
| | | | | Arabidopsis | AT5G27450.1 |
| | | | | thaliana | |
| | | | | mvk | |
| | | | | E. coli ispE | P62615 |
| | | | | E. coli glpK | P0A6F3 |
| | | | | E. coli ychB | NP_415726.1 |
| | | | | Thermoplasma | WP_010900530.1 |
| | | | | acidophilum | |
| | | | | IPK | |
| dimethylallyl phosphate → dimethylallyl pyrophosphate | 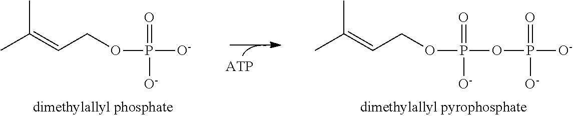 | 2.7.4- | Phosphate Kinase/ Phosphotransferase | Methanothermobacter thermautotrophicus ipk Thermoplasma acidophilum ipk Enterococcus faecalis mvaK2 | AAB84554.1 WP_010900530.1 Q9FD67 |
| | | | | Streptococcus | A0A017UH23 |
| | | | | pneumoniae | |
| | | | | mvaK2 | |
| | | | | Staphylococcus | A0A0E8GDF5 |
| | | | | aureus | |
| | | | | mvaK2 | |
| | | | | Methanocal | 3K4Y_A |
| | | | | dococcus | |
| | | | | jannaschii | |
| | | | | ipk | |
| 3-methyl-2- butenol → dimethylallyl pyrophosphate | 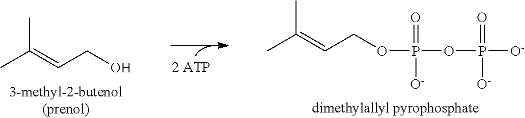 | 2.7.6- | Alcohol diphospho kinase | Escherichia coli Prs Mycoplasma pneumoniae M129 PrsA Arabidopsis thaliana col TPK1 | NP_415725.1 NP_109761.1 BAH19964.1 |
| | | | | Arabidopsis | BAH57065.1 |
| | | | | thaliana col | |
| | | | | TPK2 | |
| DMAPP ↔ IPP | 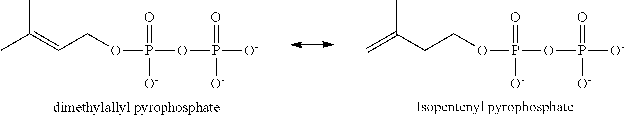 | 5.3.3.2 | isopentenyl diphosphate isomerase | E. coli idi | Q46822 |
| DMAPP + IPP → GPP | 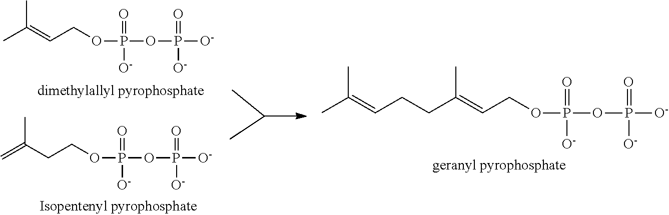 [SMILES: C=C(C)CCOP(=O)([O-])OP(=O)([O-])[O-] ; CC(C)=CCOP(=O)([O-])OP(=O)([O-])[O-] ; CC(C)=CCC/C(C)=C/COP(=O)([O-])OP(=O)([O-])[O-]] | 2.5.1- | Geranyl pyrophosphate synthase | E. coli ispA Abies grandis GPPS2 | P22939 Q8LKJ2 |

TABLE J
Reactions of the isoprenoid precursor synthesis pathway via 4-methyl-2-oxopent-4-enoic acid and
isoprenol starting from aldol condensation between acetaldehyde and 2-oxobutyric acid

| | | | | Source | |
|---|---|---|---|---|---|
| | | | | organism | |
| | | | | and | Protein |
| | | EC | Enzyme | gene/enzyme | Accession |
| Reaction | Illustration | Numbers | names | name | Numbers |
| acetyl-CoA → acetal- dehyde | 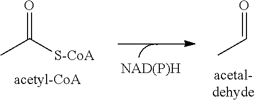 | 1.2.1 .- | Aldehyde forming CoA reductase | Acinetobacter calcoaceticus acr1 | AAC45217.1 |
| 3-methyl-2- butenoyl- CoA -> 3- methyl-2- butenal | 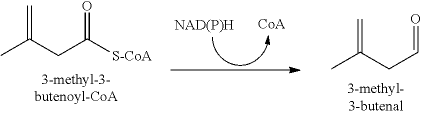 | | | Acinetobacter sp Strain M-1 acrM Clostridium beijerinckiiald E. coli eut E | BAB85476.1 AAT66436.1 NP_416950.1 |
| | | | | Salmonella | AAA80209.1 |
| | | | | enterica | |
| | | | | eutE | |
| | | | | E. coli | NP_414885.1 |
| | | | | mhpF | |
| pyruvate → acetal- dehyde | 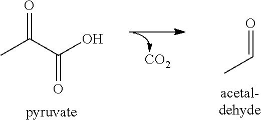 | 4.1.1.1 | alpha- ketoacid decarbo- xylase | Saccharomyces cerevisiae PDC1 Saccharomyces cerevisiae PDC5 | NP_013145.1 NP_013235.1 |
| 4-methyl-2- oxopent-4- enoate →3- methyl-3- butenal | 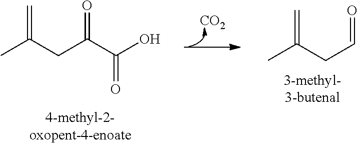 | | | Saccharomyces cerevisiae PDC6 Pisum sativum PDC1 | NP_011601.3 CAA91444.1 |
| | | | | Saccharomyces | NP_010668.3 |
| | | | | cerevisiae | |
| | | | | ARO10 | |
| | | | | Saccharomyces | NP_010203.1 |
| | | | | cerevisiae | |
| | | | | THI3 | |
| | | | | Lactococcus | CAG34226.1 |
| | | | | lactis kivd | |
| | | | | Pantoea | WP_003848906.1 |
| | | | | agglomerans | |
| | | | | idpC | |
| | | | | Enterobacter | WP_073396207.1 |
| | | | | cloacae | |
| | | | | idpC | |
| Pyruvate + acetyl-CoA → citramalate | 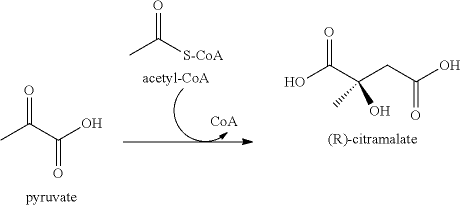 | 2.3.1.182 | Citramalate synthase | Methanocaldococcus jannaschil cimA Leptospira interrogans serovar Lai str. 56601 cimA | WP_010870909.1 NP_712531.1 |
| Citramalate → 3- methyl- malate | 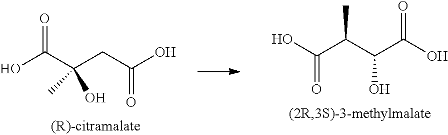 | 4.2.1.35 | Citramalate isomerase | E. coli leuCD Methanocaldococcus jannaschii leuCD | NP_414614.1 NP_414613.1 AAB98487.1 AAB99283.1 |
| | | | | Leptospira | NP_712276.1 |
| | | | | interrogans | NP_712277.1 |
| | | | | serovar Lai | |
| | | | | str. 56601 | |
| | | | | leuCD | |
| 3- methyl- malate → 2- oxobutyric acid + CO₂ | 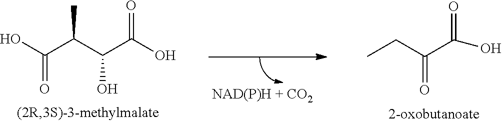 | 1.2.1 .- | 3- methylmalate dehydro genase | Methanocal dococcus jannaschii MJ0270 | WP_010870225.1 |
| | | | | Leptospira | NP_712333.1 |
| | | | | interrogans | |
| | | | | serovar Lai | |
| | | | | str. 56601 | |
| | | | | leuB | |
| 2- oxobutyric acid + acetal- dehyde → 4- hydroxy-2- oxo-3- methyl- pentanoate | 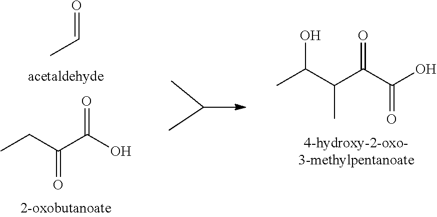 | 4.1.3 .- | aldolase | E. coli mhpE Pseudomonas putida xylK Pseudomonas sp. CF600 dmpG | NP_414886.1 WP_011005904.1 WP_017849278.1 |
| 4-hydroxy- 2-oxo-3- methyl- pentanoate →4- hydroxy-2- oxo-4- methyl- pentanoate | 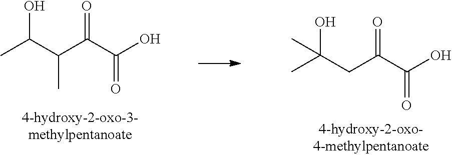 | 5.4.99 .- | mutase | Streptomyces cinnamonensis icmAB Metallosphaera sedula Msed_0638, Msed_2055 | AAC08713.1. CAB59633.1 A4YEG1, A4YIE3 |
| | | | | Cupriavidus | Q1LRY0 |
| | | | | metallidurans | |
| | | | | icmF | |
| | | | | Kyrpidia | D5WTR7, |
| | | | | tusciae | D5WTR8 |
| | | | | rcmAB | |
| | | | | Rhodobacter | ABA8014 |
| | | | | sphaeroides | 4.1 |
| | | | | meaA | |
| 4-hydroxy- 2-oxo-4- methyl- pentanoate →4- methyl-2- oxopent-4- enoate | 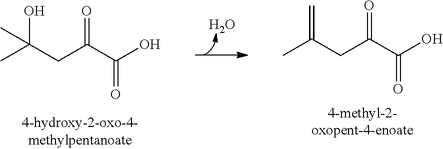 | 4.2.1 .- | 2- oxopent- 4-enoate hydratase | E. coli mhpD Comamonas testosteroni CNB-1 cnbE Pseudomonas putida xylJ | NP 414884.2 WP_012478201.1 WP_011005906.1 |
| 4-methyl-2- oxopent-4- enoate →3- methyl-3- butenoyl- CoA | 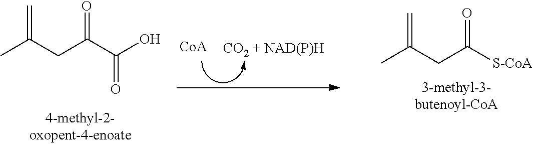 | 1.2.1.,- | alpha- keto acid dehydro genase | Streptomyces avermitilis bkdFGH- lpdA1 | BAC72088.1 BAC72089.1 BAC72090.1 KUN54417.1 |
| | | | | Homo | NP_000700.1 |
| | | | | sapiens | NP_000047.1 |
| | | | | BKDHAB- | NP_001909.3 |
| | | | | DBT-DLD | NP_000099.2 |
| | | | | Bacillus | WP_004398565.1 |
| | | | | subtilis | WP_004398638.1 |
| | | | | bfmBAA- | WP_003230323.1 |
| | | | | bfmBAB- | WP_003232309.1 |
| | | | | bfmBB- | |
| | | | | pdhD | |
| | | | | E. coli lpdA- | NP_414657.1 |
| | | | | aceEF | NP_414658.1 |
| | | | | | NP_414656.1 |
| 3-methyl-3- butenoyl- CoA→3- methyl-3- butenoate | 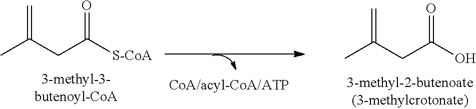 | 3.1.2 .- | Thioesterase | E. coli tesA E. coli tesB E. coli yciA E. coli fadM E. coli ydil E. coli ybgC | NP_415027.1 NP_414986.1 NP_415769.1 NP_414977.1 NP_416201.1 NP_415264.1 |
| | | | | E. coli paal | NP_415914.1 |
| | | | | Mus | P58137.1 |
| | | | | musculus | |
| | | | | acot8 | |
| | | | | Lycopersicon | ADK38536.1 |
| | | | | hirsutum f | |
| | | | | glabratum | |
| | | | | mks2 | |
| | | | | Alcanivorax | YP_692749.1 |
| | | | | borkumensis | |
| | | | | tesB2 | |
| | | | | Fibrobacter | YP_005822012.1 |
| | | | | succinogen | |
| | | | | es Fs2108 | |
| | | | | Prevotella | YP_003574018.1 |
| | | | | ruminicola | |
| | | | | Pr655 | |
| | | | | Prevotella | YP_003574982.1 |
| | | | | ruminicola | |
| | | | | Pr1687 | |
| | | 2.8.3~ | CoA | E. coli atoD | NP_416725.1 |
| | | | transferase | E. coli atoA | NP_416726.1 |
| | | | | E. coli scpC | NP_417395.1 |
| | | | | Clostridium | AAA92346.1 |
| | | | | kluyveri | |
| | | | | cat1 | |
| | | | | Clostridium | AAA92344.1 |
| | | | | kluyveri | |
| | | | | cat2 | |
| | | | | Clostridium | NP_149326.1, |
| | | | | acetobutylicum | |
| | | | | ctfAB | NP_149327.1 |
| | | | | Pseudomonas | NP_746081.1 |
| | | | | putida | |
| | | | | pcalJ | NP_746082.1 |
| | | | | Megasphaera | WP_014015705.1 |
| | | | | elsdenii | |
| | | | | pct | |
| | | | | Acidaminococcus | CAA57199.1 |
| | | | | fermentans | CAA57200.1 |
| | | | | gctAB | |
| | | | | Acetobacter | AGG68319.1 |
| | | | | aceti aarC | |
| | | | | E. coli ydiF | NP_416209.1 |
| | | 2.3.1 .-; | Phospho | Clostridium | NP_349676.1 |
| | | 2.7.2.1; | transacylase + | acetobutylicum | |
| | | 2.7.2.15 | Carboxylate | ptb | |
| | | | kinase | Enterococcus | AAD55374.1 |
| | | | | faecalis | |
| | | | | ptb | |
| | | | | Salmonella | AAD39011.1 |
| | | | | enterica | |
| | | | | pduL | |
| | | | | Clostridium | AAK81015.1 |
| | | | | acetobutylicum | |
| | | | | buk | |
| | | | | Enterococcus | AAD55375.1 |
| | | | | faecalis | |
| | | | | buk | |
| | | | | Salmonella | AAD39021.1 |
| | | | | enterica | |
| | | | | pduW | |
| 3-methyl-3- butenoate →3~ methyl-3- butenal | 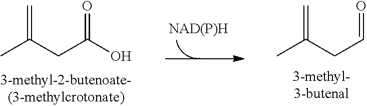 | 1.2.99.6 | Carboxylic Acid (Carboxylate) reductase | E. coli PaoABC | NP_414820.1, NP_414819.1, NP_414818.1 |
| | | | | Mycobacterium | WP_012393886.1 |
| | | | | marinum | |
| | | | | Car | |
| | | | | Nocardia | AAR91681.1 |
| | | | | iowensis | |
| | | | | Car | |
| | | | | Segniliparus | WP_013138593.1 |
| | | | | rotundus | |
| | | | | Car | |
| 3-methyl-3- butenal → 3-methyl-3- butenol | 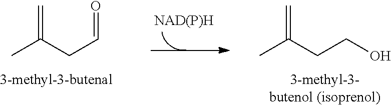 | 1.1.1 .- | Alcohol dehydro genase | E. coli betA E. coli dkgA E. coli eutG E. coli fucO E. coli ucpA E. coli yahK | NP_414845.1 NP_417485.4 NP_416948.4 NP_417279.2 NP_416921.4 NP_414859.1 |
| | | | | E. coli ybbO | NP_415026.1 |
| | | | | E. coli ybdH | NP_415132.1 |
| | | | | E. coli yiaY | YP_026233.1 |
| | | | | E. coli yjgB | NP_418690.4 |
| | | | | Acinetobacter | AAG10028.1 |
| | | | | sp. SE19 | |
| | | | | ChnD | |
| | | | | Marinobacter | YP_959769.1 |
| | | | | aquaeolei | |
| | | | | VT8 | |
| | | | | maqu_2507 | |
| | | | | Saccharomyces | Q04894.1 |
| | | | | cerevisiae | |
| | | | | ADH6 | |
| | | | | Clostridium | EDK35022.1 |
| | | | | kluyveri | |
| | | | | 4hbD | |
| 3-methyl-3- butenoyl- CoA → 3- methyl-3- butenol | 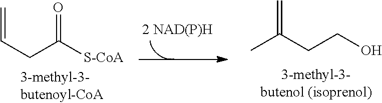 | 1.2.1 .- | Alcohol- forming Acyl- CoA reductase | Salmonella enterica pduW Arabidopsis thaliana At3g11980 | AAD39021.1 AEE75132.1 |
| | | | | Arabidopsis | AEE77915.1 |
| | | | | thaliana | |
| | | | | At3g44560 | |
| | | | | Arabidopsis | AEE79553.1 |
| | | | | thaliana | |
| | | | | At3g56700 | |
| | | | | Arabidopsis | AED93034.1 |
| | | | | thaliana | |
| | | | | At5g22500 | |
| | | | | Arabidopsis | AEE86278.1 |
| | | | | thaliana | |
| | | | | CER4 | |
| | | | | Marinobacter | YP_959486.1 |
| | | | | aquaeolei | |
| | | | | VT8 | |
| | | | | maqu 2220 | |
| | | | | Marinobacter | YP_959769.1 |
| | | | | aquaeolei | |
| | | | | VT8 | |
| | | | | maqu_2507 | |
| 3-methyl-3- butenol → Isopentenyl phosphate | 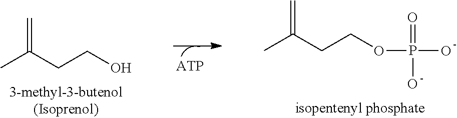 | 2.7.1 .- | Alcohol Kinase/ Phospho- transferase | Saccharomyces cerevisiae ERG12 Saccharomyces cerevisiae ERG8 | P07277 P24521 |
| | | | | Arabidopsis | Q67ZM7 |
| | | | | thaliana | |
| | | | | At5g58560 | |
| | | | | Mentha x | P56848 |
| | | | | piperita ipk | |
| | | | | Arabidopsis | AT5G27450.1 |
| | | | | thaliana | |
| | | | | mvk | |
| | | | | E. coli ychB | N_415726.1 |
| | | | | E. coli glpK | P0A6F3 |
| | | | | Methanothermobacter | AAB84554.1 |
| | | | | thermautotrophicus | |
| | | | | ipk | |
| | | | | Thermoplasma | WP_010900530.1 |
| | | | | acidophilum | |
| | | | | ipk | |
| | | | | Methanocaldococcus | 3K4Y_A |
| | | | | jannaschii | |
| | | | | ipk | |
| isopentenyl phosphate isopentenyl pyro- phosphate | 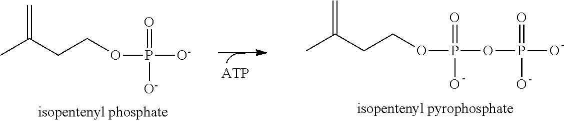 | 2.7.4- | Phosphate Kinase/ Phospho- transferase | Methanothermobacter thermautotrophicus ipk Thermoplasma acidophilum ipk | AAB84554.1 WP_010900530.1 |
| | | | | Enterococcus | Q9FD67 |
| | | | | faecalis | |
| | | | | mvaK2 | |
| | | | | Streptococcus | A0A017UH23 |
| | | | | pneumoniae | |
| | | | | mvaK2 | |
| | | | | Staphylococcus | A0A0E8GDF5 |
| | | | | aureus | |
| | | | | mvaK2 | |
| | | | | Methanocaldococcus | 3K4Y_A |
| | | | | jannaschii | |
| | | | | ipk | |
| 3-methyl-3- butenol → Isopentenyl pyro- phosphate | 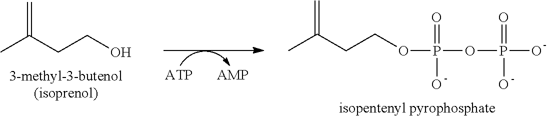 | 2.7.6 .- | Alcohol di- phos- phokinase | E. coli prs Mycoplasma pneumoniae M129 prsA Arabidopsis thaliana col TPK1 | NP_415725.1 NP_109761.1 BAH19964.1 |
| | | | | Arabidopsis | BAH57065.1 |
| | | | | thaliana col | |
| | | | | TPK2 | |
| IPP↔ DMAPP | 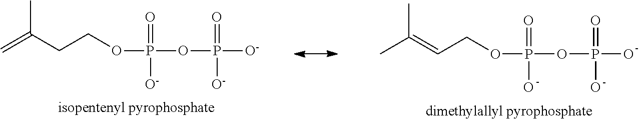 | 5.3.3.2 | isopentenyl diphosphate isomerase | E. coli idi | NP_417365.1 |
| | | | | E. coli ispA | NP_414955.1 |
| DMAPP + IPP→GPP | 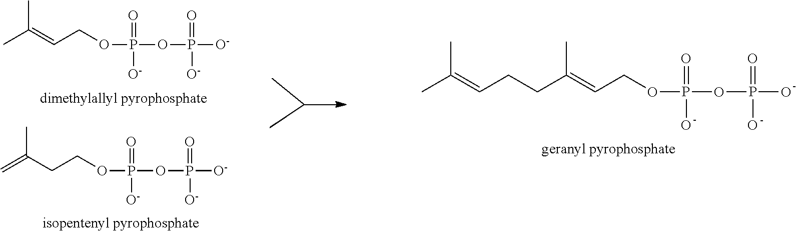 [SMILES: CC(C)=CCOP(=O)([O-])OP(=O)([O-])[O-] ; CC(C)=CCC/C(C)=C/COP(=O)(O)OP(=O)(O)O ; C=C(C)CCOP(=O)([O-])OP(=O)([O-])[O-]] | 2.5.1- | Geranyl pyro- phosphate synthase | Abiesgrandis GPPS2 | AAN01134.1 |

TABLE K
Example reactions and enzymes for the synthesis of isoprenoids

| | | | | Source | |
|---|---|---|---|---|---|
| | | | | organism | |
| | | | | and | Protein |
| | | EC | Enzyme | gene | Accession |
| Reaction | Illustration | Numbers | names | name | Numbers |
| GPP → limonene | 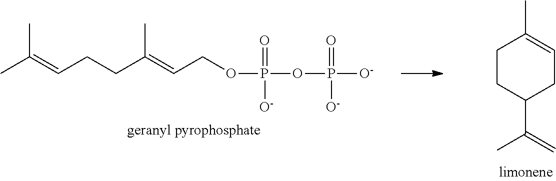 | 4.2.3.16 | limonene synthase | Mentha spitaca LS Ocimum basilicum FES Abies grandis g-lim1 Abies grandis g-pin2 | AGN90914.1 AAV63790.1 AAB70907.1 AAF61455.1 |
| | | | | Arabidopsis | NP_179998.1 |
| | | | | thaliana | |
| | | | | col | |
| | | | | TPS10 | |
| | | | | Picea | AAO73863.1 |
| | | | | abies | |
| | | | | TPS-car | |
| | | | | Picea | AAS47694.1 |
| | | | | abies | |
| | | | | TPS-lim | |
| GPP → pinene | 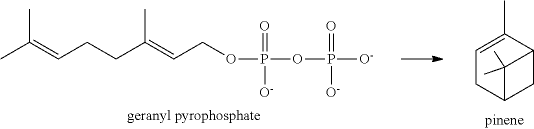 | 4.2.3.119 4.2.3.120 4.2.3.121 4.2.3.122 | pinene synthase | Pinus taeda pt30 Abies grandis g-pin1 Abies grandis g-lim1 | AAO61228.1 AAB71085.1 AAB70907.1 |
| | | | | Abies | AAF61455.1 |
| | | | | grandis | |
| | | | | g-pin2 | |
| | | | | Picea | AAO73863.1 |
| | | | | abies | |
| | | | | TPS-car | |
| | | | | Picea | AAS47694.1 |
| | | | | abies | |
| | | | | TPS-lim | |
| | | | | Ocimum | AAV63790.1 |
| | | | | basilicum | |
| | | | | FES | |
| GPP + IPP → FPP | 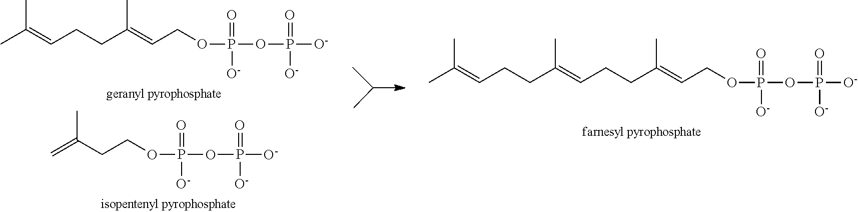 [SMILES: CC(C)=CCC/C(C)=C/COP(=O)([O-])OP(=O)([O-])[O-] ; C=C(C)CCOP(=O)([O-])OP(=O)([O-])[O-] ; CC(C)=CCC/C(C)=C/CC/C(C)=C/COP(=O)([O-])OP(=O)([O-])[O-]] | 2.5.1.10 | farnesyl di- phosphate synthase | E. coli ispA Sacc- haromyces cerevisiae FPP1 Solanum lycopersicum lefps1 Arabidopsis thaliana col FPS1 Capsicum annuum GGPPS | NP_414955.1 NP_012368.1 NP_001234068.1 NP_199588.1 XP_01656864.1 |
| | | | | Ginkgo | AAR27053.1 |
| | | | | biloba | |
| | | | | FPS | |
| | | | | Hevea | BAB60678.1 |
| | | | | brasiliensis | |
| | | | | hbgg | |
| | | | | Homo | NP_ |
| | | | | sapiens | 001129293.1 |
| | | | | FDPS | |
| | | | | Methano | WP_013295358.1 |
| | | | | thermobacter | |
| | | | | marburgensis | |
| | | | | Marburg | |
| | | | | idsA | |
| | | | | Myco- | WP_003417957.1 |
| | | | | bacterium | |
| | | | | tuberculosis | |
| | | | | H37Rv | |
| | | | | rv3398c | |
| FPP → beta- caryo- phyllene | 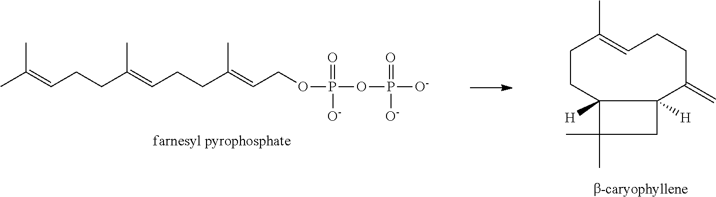 | 4.2.3.57 | beta- caryo- phyllene synthase | Artemisia annua QHS1 Arabidopsis thaliana col AT5G23 960 Cucumis sativus bcp Oryza sativa | AAL79181.1 NP_197784.2 NP_001292628.1 XP_015648213.1 |
| | | | | osTPS3 | |
| | | | | Zingiber | BAG12020.1 |
| | | | | zerumbet | |
| | | | | zss1 | |
| FPP → valencene | 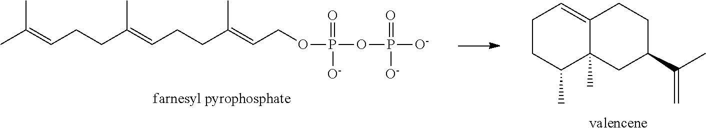 | 4.2.3.73 | valencene synthase | Callitropsis nootkatensis VALC Citrus sinensis tps1 Vitis vinifera valcs | AFN21429.1 AAQ04608.1 AAS66358.1 |
| FPP → veti- spiradiene | 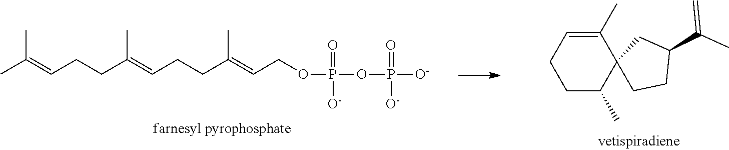 | 4.2.3.21 | veti- spiradiene synthase | Hyoscyamus muticus VS1 | Q39978.2 |
| FPP → amor- phadiene | 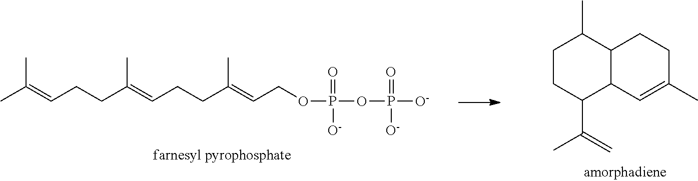 | 4.2.3.24 | amor- phadiene synthase | Artemisia annua ads | AAF61439.1 |
| FPP → far- nesene | 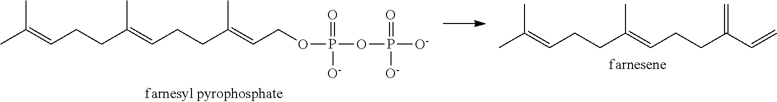 | 4.2.3.46 4.2.3.47 | farnesene synthase | Malus domestica FS Arabidopsis thaliana col TPS03 Cucumis | NP_001280822.1 NP_567511.3 NP_001267674.1 |
| | | | | sativus | |
| | | | | afs | |
| | | | | Picea | AAS47697.1 |
| | | | | abies | |
| | | | | TPS-far | |
| | | | | Arabidopsis | NP_199276.1 |
| | | | | thaliana | |
| | | | | col | |
| | | | | AT5G44 | |
| | | | | 630 | |
| | | | | Artemisia | CAC12731.1 |
| | | | | annua | |
| | | | | FS | |
| | | | | Citrus | AAK54279.1 |
| | | | | junos | |
| | | | | CJFS | |
| | | | | Mentha × | AAB95209.1 |
| | | | | piperita | |
| | | | | tspa11 | |
| | | | | Picea | AAS47695.1 |
| | | | | abies | |
| | | | | TPS-lon | |
| | | | | Zea | NP_001105850.2 |
| | | | | mays | |
| | | | | TPS10 | |
| | | | | Zea | NP_001105097.1 |
| | | | | mays | |
| | | | | TPS1 | |

TABLE L
Example reactions for the production of prenylated aromatic compounds and example enzymes

| | | | | Source | |
|---|---|---|---|---|---|
| | | | | organism | |
| | | | | and | Protein |
| | | EC | Enzyme | gene/enzyme | Accession |
| Reaction | Illustration | Numbers | names | name | Numbers |
| Olivetolic acid + GPP → cannabigerolic acid | 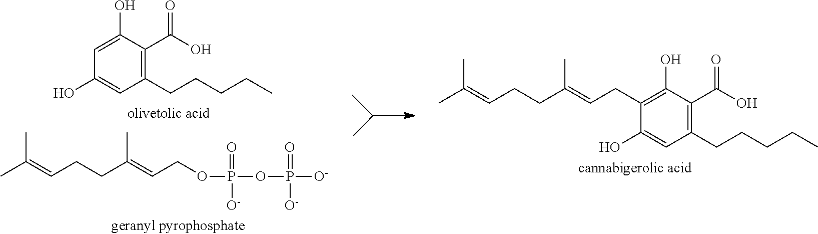 [SMILES: CC(C)=CCC/C(C)=C/COP(=O)([O-])OP(=O)([O-])[O-] ; CCCCCc1cc(O)cc(O)c1C(=O)O ; CCCCCc1cc(O)c(C/C=C(\C)CCC=C(C)C)c(O)c1C(=O)O] | 2.5.1.- | geranylpyro- phosphate:olivetolate geranyltransferase | Lithospermum erythrorhizon PGT-1 Lithospermum erythrorhizon PGT-2 E. coli ubiA Arabidopsis thaliana PPT1 Schizo- saccharomyces pombe coq2 Cannabis sativa | Q8W405 Q8W404 P0AGK1 Q93YP7 Q10252 |
| | | | | CsPT1 | |
| | | | | Streptomyces | BAE00106.1 |
| | | | | sp. strain | |
| | | | | CL190 nphB | |
| | | | | Streptomyces | BAE00107.1 |
| | | | | Coelicolor | |
| | | | | SCO7190 | |
| | | | | Streptomyces | AKH84817.1 |
| | | | | sp. CNQ- | |
| | | | | 509 cnqp3 | |
| | | | | Phleum | ABB78007.1 |
| | | | | pretense phl | |
| | | | | p4 | |

TABLE M
Plasmids and primers used for in vivo tiglic acid production

Plasmids
pCDF-P1-pct-fadAx
pET-P1-fadB2x-fadB1x
pET-P1-fadB2x-fadB1x-P2-ydil

Primers

| Name | SEQ ID NO: | Sequence |
|---|---|---|
| pct-f1 | 1 | 5′-AGGAGATATACCATGAGAAAAGTAGAAATCATTAC-3′ |
| pct-r1 | 2 | 5′-CGCCGAGCTCGAATTCTTATTTTTTCAGTCCCATGGGAC-3′ |
| fadAx-f1 | 3 | 5′-GAAAAAATAAGAATTTAAGGAGGAATAAACC ATGACCCTGGCAAATGATCC-3′ |
| fadAx-r1 | 4 | 5′-CGCCGAGCTCGAATTCTTAATACAGACATTCAACTGCC-3′ |
| fadB2x-f1 | 5 | 5′-AGGAGATATACCATGCATATCGCCAACAAACAC-3′ |
| fadB2x-r1 | 6 | 5′-CGCCGAGCTCGAATTCTTATTTTGCTGCCATGCGCAG-3′ |
| fadB1x-f1 | 7 | 5′-AGCAAAATAAGAATTTAAGGAGGAATAAACC ATGGCCTTTGAAACCATTCTG-3′ |
| fadB1x-r1 | 8 | 5′-CGCCGAGCTCGAATTCTTAGCGATCTTTAAACTGTGC-3′ |
| ydil-f1 | 9 | 5′-AAGGAGATATACATATGATATGGAAACGGAAAATCAC-3′ |
| ydil-r1 | 10 | 5′-TTGAGATCTGCCATATGTCACAAAATGGCGGTCGTC-3′ |

TABLE N
plasmids and primers used for conversion of prenol to GPP and geraniol

Plasmids
pET-P1-idi-trGPPS2-P2-ges-ychB-mtipk
pET-P1-idi-trGPPS2-P2-ges-thaipk-mtipk

Primers

| Name | SEQ ID NO: | Sequence |
|---|---|---|
| idi-f1 | 11 | AGGAGATATACCATGCAAACGGAACACGTCATTT |
| idi-r1 | 12 | TGCGCTATATGCCATGGTTTATTCCTCCTTAAATTATTTAAGCTGGGTAAATGCAGATA |
| trgpps2-f1 | 13 | ATGGCATATAGCGCAATGGC |
| trgpps2-r1 | 14 | GTGATGGCTGCTGCCTTAGTTCTGACGAAATGCAACAT |
| ges-f1 | 15 | AAGGAGATATACATAATGGAAGAAAGCAGCAGCAAA |
| ges-r1 | 16 | TTACCAGACTCGAGGTTACTGGGTAAAAAACAGGGC |
| ychB-f1 | 17 | ACCCAGTAACCTCGAAAGGAGGAATAAGGC ATGCGGACACAGTGGCCCT |
| ychB-r1 | 18 | TTTCAGGATGATCATTTGTTATTCCTCCTTAAGGTCTTAAAGCATGGCTCTGTGCAA |
| mtipk-f1 | 19 | ATGATCATCCTGAAACTGGGT |
| mtipk-r1 | 20 | CTTTACCAGACTCGAGTTAGTGTTTACCTGTAATACGTG |
| mtipk-f2 | 21 | GATCCGCTAACTCGATAAGGAGGAATAACAA ATGATCATCCTGAAACTGGGT |
| taipk-f1 | 22 | ACCCAGTAACCTCGAAAGGAGGAATAAGGCATGATGATCCTGAAAATTGGTG |
| taipk-r1 | 23 | CTTTACCAGACTCGAGTTAGCGGATCACGGTGCCA |

TABLE O
List of primers

| | SEQ | | |
|---|---|---|---|
| | ID | | |
| Name | NO: | Sequence | Description |
| maqu_2507- | 24 | GCCAGGATCCGAATTCGAA | maqu_2507 |
| f1 | | CTACTTTCTGACCGGTGG | forward |
| maqu_2507- | 25 | CGCCGAGCTCGAATTCTTA | maqu_2507 |
| r1 | | CCAGTAAATGCCACGCA | reverse |
| cbjALD-f1 | 26 | GCCAGGATCCGAATTCGAA | cbjALD |
| | | TAAAGACACACTAATACCT | forward |
| | | AC | |
| cbjALD-r1 | 27 | CGCCGAGCTCGAATTCTTA | cbjALD |
| | | GCCGGCAAGTACACATC | reverse |
| chnD-f1 | 28 | GCCAGGATCCGAATTCGCA | chnD |
| | | CTGCTATTGTGTTACCCAC | forward |
| chnD-r1 | 29 | CGCCGAGCTCGAATTCTCA | chnD |
| | | ATTTTCGTGCATCAGAAC | reverse |

TABLE P
Measured activities of different alcohol
dehydrogenases on oxidization of prenol.

| | Specific activity |
|---|---|
| Enzyme (cofactor) | (μmol/mg/min) |
| FucO (NAD⁺) | N.D. |
| YqhD (NADP⁺) | N.D. |
| YjgB (NADP⁺) | 0.30 ± 0.03 |
| YahK (NADP⁺) | 0.167 ± 0.005 |
| YiaY (NAD⁺) | N.D. |
| ChnD (NAD⁺) | 0.123 ± 0.007 |

TABLE Q
plasmids and primers used for in vivo 2,3-dihydroxybutyric acid production

Plasmids
pET-P1-bktB-phaB1-P2-phaJ
pCDF-P1-pct-P2-tdter

Primers

| | SEQ ID | |
|---|---|---|
| Name | NO: | Sequence |
| tdTer-f1 | 30 | 5′-AAGGAGATATACATATGATTGTTAAGCCGATGGTCC-3′ |
| tdTer-r1 | 31 | 5′-TTGAGATCTGCCATATGTTAGATGCGGTCAAAACGTTCA-3′ |
| pct-f1 | 32 | 5′-AGGAGATATACCATGAGAAAAGTAGAAATCATTAC-3′ |
| pct-r1 | 33 | 5′-CGCCGAGCTCGAATTCTTATTTTTTCAGTCCCATGGGAC-3′ |
| bktB-f1 | 34 | 5′-AGGAGATATACCATGATGACGCGTGAAGTGGTAGT-3′ |
| bktB-r1 | 35 | 5′-CGCCGAGCTCGAATTCTCAGATACGCTCGAAGATGG-3′ |
| phaB1-f1 | 36 | 5′-GCGTATCTGAGAATTAGGAGGCTCTCTATGACTCAGCGCATTGCGTA |
| phaB1-r1 | 37 | 5′-CGCCGAGCTCGAATTCTCAGCCCATGTGCAGGCC-3′ |
| phaJ-f1 | 38 | 5′-AAGGAGATATACATATGTCGGCACAAAGCCTG-3′ |
| phaJ-r1 | 39 | 5′-TTGAGATCTGCCATATGTTACGGCAGTTTCACCACC-3′ |

TABLE R
List of primers used in example of GPP BIOSYNTHESIS VIA
2-HYDROXYISOVALERIC ACID AND PRENOL STARTING FROM
DECARBOXYLATIVE ACYLOIN CONDENSATION BETWEEN TWO
PYRUVATES

| | SEQ | | |
|---|---|---|---|
| | ID | | |
| Name | NO: | Sequence | Description |
| pct540- | 40 | GAGGAATAAACCATGCGTAAAGTGCCGATTAT | pct540 |
| f1 | | TA | forward |
| pct540- | 41 | GATGATGATGGTCGACGCTTTTCATTTCTTTCA | pct540 |
| r1 | | GGCC | reverse |
| pct-f2 | 42 | TAGAAGGAGGAGATCTATGAGAAAAGTAGAAA | pct forward |
| | | TCATTACAG | |
| pct-r2 | 43 | GGGGGACCAGCTCGAGTTTTTTCAGTCCCAT | pct reverse |
| | | GGGACC | |
| alsS-f1 | 44 | AAGGAGATATACATATGACCAAAGCAACCAAA | alsS |
| | | GAA | forward |
| alsS-r1 | 45 | AATGGTAATACGCATGTTAATTTCCTCCTAGAA | alsS |
| | | TTACAGGGCTTTGGTTTTCAT | reverse |
| panE-f1 | 46 | ATGCGTATTACCATTGCCGG | panE |
| r1 | | | forward |
| panE- | 47 | TTGAGATCTGCCATATTATTTGGCTTTCAGCA | panE |
| | | GTTCTT | reverse |
| ilvC-f1 | 48 | AGGAGATATACCATGGCTAACTACTTCAATAC | ilvC |
| | | | forward |
| ilvC-r1 | 49 | ACGGTACTTAGGCATGGTTTATTCCTCCTTAA | ilvC |
| | | ACTCTTAACCCGCAACAGCAATAC | reverse |
| ilvD-f1 | 50 | ATGCCTAAGTACCGTTCCG | ilvD |
| | | | forward |
| ilvD-r1 | 51 | CGCCGAGCTCGAATTCTTAACCCCCCAGTTTC | ilvD |
| | | GATTT | reverse |

TABLE S
plasmids and primers used for in vivo butyric acid
production through beta-oxidation reversal

Plasmids
pTH-atoB-fadB-egter
pZS-fadM
pZS-tesA
pZS-tesB
pZS-yciA
pZS-ybgC
pZS-ydil
pCDF-P1-pct-P2-tdter

Primers

| | SEQ ID | |
|---|---|---|
| Name | NO: | Sequence |
| fadM-f1 | 129 | 5′-TTAAAGAGGAGAAAGGTACCATGCAAACACAAATCAAAGT-3′ |
| fadM-r1 | 52 | 5′-TGCCTCTAGCACGCGTCGTTTACTTAACCATCTGCTCCA-3′ |
| tesA-f1 | 53 | 5′-TTAAAGAGGAGAAAGGTACCATGATGAACTTCAACAATGTTTTC-3′ |
| tesA-r1 | 54 | 5′-TGCCTCTAGCACGCGTTCCGTTGCTTTATGAGTCATG-3 |
| tesB-f1 | 55 | 5′-TTAAAGAGGAGAAAGGTACCATGAGTCAGGCGCTAAAAAA-3′ |
| tesB-r1 | 56 | 5′-TGCCTCTAGCACGCGTAACAGCCGGACGGTTTTC-3 |
| ybgC-f1 | 57 | 5′-TTAAAGAGGAGAAAGGTACCGTGAATACAACGCTGTTTCGAT-3′ |
| ybgC-r1 | 58 | 5′-TGCCTCTAGCACGCGTTCACTGCTTAAACTCCGCGA-3′ |
| yciA-f1 | 59 | 5′-TTAAAGAGGAGAAAGGTACCATGTCTACAACACATAACGTCCC-3′ |
| yciA-r1 | 60 | 5′-TGCCTCTAGCACGCGTTTCAGTAAGCAGAAAGTCAAAAGC-3′ |
| ydil-f1 | 61 | 5′-TTAAAGAGGAGAAAGGTACCATGATATGGAAACGGAAAATCA-3′ |
| ydil-r1 | 62 | 5′-TGCCTCTAGCACGCGTGGTGACAACGTCACAAAATGG-3′ |
| atoB-f1 | 63 | 5′-GAGGAATAAACCATGAAAAATTGTGTCATCGTCA-3′ |
| atoB-r1 | 64 | 5′-CCCAAGCTTCGAATTCTTAATTCAACCGTTCAATCAC-3′ |
| fadB-f1 | 65 | 5′-TAAGAATTCGAAGCTGCGGATTCAGGAGACTGACA-3′ |
| fadB-r1 | 66 | 5′-GTTCGGGCCCAAGCTTTAAGCCGTTTTCAGGTCGC-3′ |
| egter-f1 | 67 | 5′-AAACGGCTTAAAGCTAATAAGGAGGAATAAACCATGGCAATGTTTACCACGAC-3′ |
| egter-r1 | 68 | 5′-GTTCGGGCCCAAGCTTGCGGCCGCTTATTGCTGTGCTGCGGAC-3′ |

TABLE T
plasmids and primers required for GPP biosynthesis via 2-oxoisovaleric acid,
2-oxoisocaproic acid and prenol starting from
decarboxylative acyloin condensation between two pyruvates

Plasmids
pET-P1-ilvC-ilvD-P2-alsS-liuA
pCDF-P1-bkdF-bkdG-bkdH-P2-lplA-lpdA1
pRSF-P1-leuA(G462D)-leuB-P2-leuC-leuD

Primers

| | SEQ ID | |
|---|---|---|
| Name | NO: | Sequence |
| alsS-f1 | 44 | AAGGAGATATACATATGACCAAAGCAACCAAAGAA |
| alsS-r2 | 69 | GCTCGGATAGGTCATGTGATATTCCTCCTAGCTATGTTACAGGGCTTTGGTTTT |
| | | CATC |
| ilvC-f1 | 48 | AGGAGATATACCATGGCTAACTACTTCAATAC |
| ilvC-r1 | 49 | ACGGTACTTAGGCATGGTTTATTCCTCCTTAAACTCTTAACCCGCAACAGCAAT |
| | | AC |
| ilvD-f1 | 50 | ATGCCTAAGTACCGTTCCG |
| ilvD-r1 | 51 | CGCCGAGCTCGAATTCTTAACCCCCCAGTTTCGATTT |
| liuA-f1 | 70 | ATGACCTATCCGAGCCTGAA |
| liuA-r1 | 71 | TTACCAGACTCGAGGGTACCTTAGCGGGTTTCATTAAACAGT |
| bkdF-f1 | 72 | AGGAGATATACCATGACCGTTGAAAGCACCGC |
| bkdF-r1 | 73 | CATTTTTTCTGCCATGAGTTATTCCTCCTACAACTCTTAATTACCACCTTGACCG |
| | | G |
| bkdG-f1 | 74 | ATGGCAGAAAAAATGGCAATCG |
| bkdG-r1 | 75 | GCTTGCTTCGGTCATGCTTTATTCCTCCTTTAATTGTTAATATGCCAGGCTACGA |
| | | TC |
| bkdH-f1 | 76 | ATGACCGAAGCAAGCGTTCG |
| bkdH-r1 | 77 | TTACCAGACTCGAGGGTACCTTAGCGGGTTTCATTAAACAGT |
| lplA-f1 | 78 | AAGGAGATATACATATGTCCACATTACGCCTGCT |
| lplA-r1 | 79 | TGCATCATTTGCCATCCATTATTCCTCCTTGGGTAACTACCTTACAGCCC |
| lpdA1-f1 | 80 | ATGGCAAATGATGCAAGCAC |
| lpdA1-r1 | 81 | TTACCAGACTCGAGGGTACCTTAATCATGGCTATGCAGCGG |
| leuA(G462 | 82 | AGGAGATATACCatgAGCCAGCAAGTCAT |
| D)B-f11 | | |
| leuA(G462 | 83 | CACCTGgtCaAGCGCATCTTTACCGTGGC |
| D)B-r11 | | |
| leuA(G462 | 84 | AAGATGCGCT tGacCAGGTG GATATCGTCGCTAA |
| D)B-f12 | | |
| leuA(G462 | 85 | CGCCGAGCTCGAATTCTTACACCCCTTCTGCTACATA |
| D)B-r12 | | |
| leuA(G462 | 86 | AGGAGATATACCatgAGCCAG |
| D)B-f2 | | |
| leuA(G462 | 87 | CGCCGAGCTCGAATTCTTAC |
| D)B-r2 | | |
| leuCD-f1 | 88 | CGATCGCTGACGTCGatgGCTAAGACGTTATACGAAAA |
| leuCD-r1 | 89 | TTACCAGACTCGAGGGTACCttaATTCATAAACGCAGGTTGTT |

TABLE U
List of primers used in the construction
of strain JST06(DE3) atoB^{CT5}

| | SEQ | |
|---|---|---|
| | ID | |
| Name | NO: | Sequence |
| kan-homatoE-L | 90 | TTGGTTTAACGCTGTTCTGACGG |
| | | CACCCCTACAAACAGAAGGAATA |
| | | TAAACATATGAATATCCTCCTTA |
| kan-ovcymatoB- | 91 | TCTGAAATTCTGCCTCGTGAGTG |
| R | | TAGGCTGGAGCTGCTTCG |
| cym-pCTC- | 92 | CGAAGCAGCTCCAGCCTACACTC |
| atoB-ovkan-L | | ACGAGGCAGAATTTCAGA |
| atoBintrecomb- | 93 | GCCAGCCCGCTTTTTAAC |
| R | | |

TABLE V
Strains and plasmids

| Host strain | Plasmid 1 | Plasmid 2 | Plasmid 3 | Product |
|---|---|---|---|---|
| JST06(DE3) | pCDF-P1- | pET-P2- | | Prenol |
| atoB^{CT5} ΔfadB | HMGS-aibAB- | maqu_2507 | | |
| | P2-liuC | | | |
| JST06(DE3) | pCDF-P1- | pET-P2- | | Prenol |
| atoB^{CT5} ΔfadB | HMGS-aibAB- | adhE2 | | |
| | P2-liuC | | | |
| JST06(DE3) | pCDF-P1- | pET-P2- | | Prenol |
| atoB^{CT5} ΔfadB | HMGS-aibAB- | cbjALD | | |
| | P2-liuC | | | |
| JST06(DE3) | pCDF-P1- | | | Prenol |
| atoB^{CT5} ΔfadB | HMGS-aibAB- | | | |
| | P2-cbjALD- | | | |
| | liuC | | | |
| JST06(DE3) | pCDF-P1- | | | Prenol |
| atoB^{CT5} ΔfadB | HMGS-aibAB- | | | |
| | P2- | | | |
| | maqu_2507- | | | |
| | liuC | | | |
| JST06(DE3) | pCDF-P1- | | | Prenol |
| atoB^{CT5} ΔfadB | HMGS-aibAB- | | | |
| | P2-cbjALD- | | | |
| | liuC-chnD | | | |
| JST06(DE3) | pCDF-P1- | | | Prenol |
| atoB^{CT5} ΔfadB | HMGS-aibAB- | | | |
| | P2-cbjALD- | | | |
| | liuC-yjgB | | | |
| JST06(DE3) | pCDF-P1- | | | Prenol |
| atoB^{CT5} ΔfadB | HMGS-aibAB- | | | |
| | P2-cbjALD- | | | |
| | liuC-yahK | | | |
| JST06(DE3) | pCDF-P1- | pRSF-P2- | pET-P1-idi- | Geraniol |
| atoB^{CT5} ΔfadB | HMGS-aibAB- | adhE2 | trGPPS2-P2- | |
| | P2-liuC | | ges-ychB- | |
| | | | mtipk | |
| JST06(DE3) | pCDF-P1- | pRSF-P2- | pET-P1-idi- | Geraniol |
| atoB^{CT5} ΔfadB | HMGS-aibAB- | cbjALD | trGPPS2-P2- | |
| | P2-liuC | | ges-ychB- | |
| | | | mtipk | |
| JST06(DE3) | pCDF-P1- | pET-P1-idi- | | Geraniol |
| atoB^{CT5} ΔfadB | HMGS-aibAB- | trGPPS2-P2- | | |
| | P2-cbjALD- | ges-ychB- | | |
| | liuC | mtipk | | |
| JST06(DE3) | pCDF-P1- | pET-P1-idi- | | Geraniol |
| atoB^{CT5} ΔfadB | HMGS-aibAB- | trGPPS2-P2- | | |
| | P2-cbjALD- | ges-ychB- | | |
| | liuC-yahK | mtipk | | |

TABLE W
Primers used

| | SEQ | | |
|---|---|---|---|
| | ID | | |
| Name | NO: | Sequence | Description |
| maqu_ | 94 | AAGGAGATATACATATGAACTACTTTCTGACC | maqu_2507 |
| 2507-f2 | | GGT | forward |
| maqu_ | 95 | TTGAGATCTGCCATATGTTACCAGTAAATGCC | maqu_2507 |
| 2507- | | ACGCAT | reverse |
| r2 | | | |
| maqu | 96 | TTAAATTCCGGCATAATTACTCCTTCACTGCCA | maqu_2507 |
| 2507- | | TATTACCAGTAAATGCCACGCAT | reverse |
| r3 | | | |
| cbjALD | 97 | AAGGAGATATACATATGAATAAAGACACACTA | cbjALD |
| -f2 | | ATACCTA | forward |
| cbjALD | 98 | TTGAGATCTGCCATATGTTAGCCGGCAAGTAC | cbjALD |
| -r2 | | ACATC | reverse |
| cbjALD | 99 | TTAAATTCCGGCATAATTACTCCTTCACTGCCA | cbjALD |
| -r3 | | TATTAGCCGGCAAGTACACATC | reverse |
| adhE2- | 100 | AAGGAGATATACATATGAAAGTTACAAATCAAA | adhE2 |
| f1 | | AAGAAC | forward |
| adhE2- | 101 | TTGAGATCTGCCATATGTTAAAATGATTTTATA | adhE2 |
| r1 | | TAGATATCCTTAAG | reverse |
| hmgs- | 102 | AGGAGATATACCATGACCATCGGCATCGATAA | hmgs |
| f1 | | G | forward |
| hmgs- | 103 | CGCCGAGCTCGAATTCTTATTCCGGACGATGA | hmgs |
| r1 | | TATTCG | reverse |
| aibA-f1 | 104 | TCCGGAATAAGAATTGTAGGAGGAATACTACA | aibA |
| | | TGAAAACCGCACGTTGGTG | forward |
| aibA-r1 | 105 | CAGGGTTGCGCTCATGGTTTATTCCTCCTTAA | aibA |
| | | AATCTTATGCTGCACGACGGGTCA | reverse |
| aibB-f1 | 106 | ATGAGCGCAACCCTGGATAT | aibB |
| | | | forward |
| aibB-r1 | 107 | CCTGCAGGCGCGCCGAGCTCTTATGCACCAA | aibB |
| | | CCAGTGCAT | reverse |
| liuC-f1 | 108 | AAGGAGATATACATATGCCGGAATTTAAAGTT | liuC forward |
| | | GATG | |
| liuC-r1 | 109 | TTGAGATCTGCCATATTAACGACCTTTATAAAC | liuC reverse |
| | | CGGT | |
| chnD- | 110 | TTAATATGGCAGATCAGGAGGAATAGCTGATG | chnD |
| f2 | | CACTGCTATTGTGTTACC | forward |
| chnD- | 111 | CTTTACCAGACTCGAGTCAATTTTCGTGCATC | chnD |
| r2 | | AGAAC | reverse |
| yjgB-f1 | 112 | TTAATATGGCAGATCAGGAGGAATAGCTGATG | yjgB |
| | | TCGATGATAAAAAGCTATG | forward |
| yjgB-r1 | 113 | CTTTACCAGACTCGAGTCAAAAATCGGCTTTC | yjgB |
| | | AACAC | reverse |
| yahK- | 114 | TTAATATGGCAGATCAGGAGGAATAGCTGATG | yahK |
| f1 | | AAGATCAAAGCTGTTGGTG | forward |
| yahK- | 115 | CTTTACCAGACTCGAGTCAGTCTGTTAGTGTG | yahK |
| r1 | | CGATT | reverse |

TABLE X
Plasmids for the synthesis of isoprenoids

Name
pACYC-P1-ls
pACYC-P1-pt30
pACYC-P1-qhs1-P2-ispA
pACYC-P1-valc-P2-ispA
pACYC-P1-vs1-P2-ispA
pACYC-P1-fs-P2-ispA

TABLE Y
List of primers used in the construction of strain JST06(DE3)
ΔfadE bktB^{CT5} ΔatoB fadB^{CT5} ΔfadA egter^{CT5}

| Construct/ | SEQ | |
|---|---|---|
| PCR | ID | |
| Product | NO: | Sequences |
| kan-cymR- | 130, | F- |
| p^{CT5}-atoB- | 116 | GATGTTCAAGAAAACACCCGATAACTTTCGCTATCGGGTGTTTTTATTGAATCA |
| (cat-sacB) | | AAGGGAAAACTGTCCATAT |
| cat-sacB | | R- |
| cassette | | GCATTGGCGGCGGTCAGGGAATTGCGATGGTGATTGAACGGTTGAATTAAAA |
| with atoB | | AATGAGACGTTGATCGGC |
| homology | | |
| kan-cymR- | 117, | F- |
| P^{CT5}-bktB | 118 | CAACAAACAGACAATCTGGTCTGTTTGTATTATGAACGAAGGAGAGATCTATG |
| ΔatoB | | ACGCGTGAAGTGGTAGT |
| bktB for | | R- |
| replacement | | GATGTTCAAGAAAACACCCGATAACTTTCGCTATCGGGTGTTTTTATTGATCAG |
| of atoB-cat- | | ATACGCTCGAAGATGG |
| sacB | | |
| cassette | | |
| cat-cymR- | 119, | F- |
| P^{CT5}-egTER | 120 | TTGACGGCGGTTTCAGCATTGCTGCAATGAACGAACTCGAACTGAAATAAGTG |
| @ fabl | | TAGGCTGGAGCTGCTTCG |
| cat-cymR- | | R- |
| P^{CT5}- | | AACAGAGATAACGGGCGGCAGAACGCCGCCCATCTTTACCAACAGAACGATT |
| egTER with | | ATTTCAGTTCGAGTTCGTTTTATTGCTGTGCTGCGGAC |
| fabl | | |
| homology | | |
| cat-cymR- | 121, | F- |
| P^{CT5}-egTER | 122 | CAACAAACAGACAATCTGGTCTGTTTGTATTATGAACGAAGGAGAGATCTATG |
| @ fadBA | | GCAATGTTTACCACGAC |
| ΔfadBA | | R- |
| egTER for | | TTAAACCCGCTCAAACACCGTCGCAATACCCTGACCCAGACCGATACACATTA |
| replacement | | TTGCTGTGCTGCGGAC |
| of fadB- | | |
| (kansacB)- | | |
| fadA | | |
| cassette | | |
| cat-cymR- | 123, | F1- |
| P^{CT5}--fadB- | 124, | ATCCTCCGGTTGAGCCAGCCCGTCCGGTTGGCGACCTGAAAACGGCTTAAAT |
| (kan-sacB)- | 125, | GATTGAACAAGATGGATTGC |
| fadA @ | 126 | R1- |
| fadBA | | TAAGGGGTGACGCCAAAGTATCAGAAGAACTCGTCAAGAAGG |
| Overlap | | F2- |
| extension of | | CCTTCTTGACGAGTTCTTCTGATACTTTGGCGTCACCCCTTA |
| kan and | | R2- |
| sacB | | |
| with fadBA | | ATCGGGGTGCGAATTGCATCGACAATGACAACCTGTTCCATTGTGACTCCATC |
| junction | | AAAGGGAAAACTGTCCATAT |
| homology | | |
| fadA | 127, | F- |
| deletion | 128 | TTGAGCCAGCCCGTCCGGTTGGCGACCTGAAAACGGCTTAAGGAGTCACAAT |
| | | GGCCAAGTTGACCAGTG |
| | | R- |
| | | TTAAACCCGCTCAAACACCGTCGCAATACCCTGACCCAGACCGATACACATCA |
| | | GTCCTGCTCCTCTGC |

TABLE Z
Plasmids for expression of olivetolic acid synthesis pathways

| Name | Synthesis pathway |
|---|---|
| pRSF-P1-OLS-P2-OAC | Olivetol synthase with olivetolic acid |
| | cyclase |
| pCDF-P1-OLS-P2-OAC | Olivetol synthase with olivetolic acid |
| | cyclase |
| pET-P1-OLS-P2-OAC | Olivetol synthase with olivetolic acid |
| | cyclase |
| pET-P1-OLS-P2-OAC(Y27F) | Olivetol synthase with olivetolic acid |
| | cyclase |
| pRSF-P1-STC | Polyketide synthase without cyclase |
| pET-P1-STCS | Polyketide synthase without cyclase |
| pET-P1-STCS (T135S) | Polyketide synthase without cyclase |
| pET-P1-STCS (T198M) | Polyketide synthase without cyclase |
| pET-P1-STCS (I200C) | Polyketide synthase without cyclase |
| pET-P1-STCS (T135S T198M) | Polyketide synthase without cyclase |
| pET-P1-STCS (T135S I200C) | Polyketide synthase without cyclase |
| pET-P1-STCS (T198M I200C) | Polyketide synthase without cyclase |
| pET-P1-STCS (T135S T198M | Polyketide synthase without cyclase |
| I200C) | |
| pET-P1-dcaF-P2-OAC | Polyketoacyl-CoA thiolase with |
| | olivetol cyclase |
| pET-P1-fadAx-P2-OAC | Polyketoacyl-CoA thiolase with |
| | olivetol cyclase |
| pET-P1-ScfadA-P2-OAC | Polyketoacyl-CoA thiolase with |
| | olivetol cyclase |
| pET-P1-dcaF-P2-OAC | Polyketoacyl-CoA thiolase with |
| | olivetol cyclase |
| pET-P1-bktB-P2-OAC | Polyketoacyl-CoA thiolase with |
| | olivetol cyclase |

TABLE AA
Primers for constructions of plasmids used for demonstration of
in vivo production of CBGA in E. coli.

| | SEQ | |
|---|---|---|
| | ID | |
| Name | NO: | Sequence |
| MK-IF- | 131 | AGGAGATATACCATGAGCCTGCCGTTTCTG |
| fwd | | |
| MK-IF- | 132 | CGCCGAGCTCGAATTCTTAGCTGGTCCACGGCAG |
| rev | | |
| PMK-IF- | 133 | GACCAGCTAAGAATTTAGGAGGAATAACTCATGAGCG |
| fwd | | |
| PMK-IF- | 134 | CGCCGAGCTCGAATTCATTCCTCCTTTAATTGTTATTTGTC |
| rev | | |
| PMD-IF- | 135 | AAGGAGGAATGAATTATGACCGTTTATACCGCAAG |
| fwd | | |
| PMD-IF- | 136 | CGCCGAGCTCGAATTCTTATTCTTTCGGCAGACC |
| rev | | |
| idi-GB- | 137 | GTTTAACTTTAAGAAGGAGATATACatgCAAACGGAACACGTC |
| fwd | | |
| idi-GB- | 138 | ATGGTTTATTCCTCCTTAAAttaTTTAAGCTGGGTAAATGCAG |
| rev | | |
| trGPPS | 139 | TTTAAGGAGGAATAAACCATGGTGGAATTTGACTTTAACAAATAT |
| 2-IF-fwd | | AT |
| GPPS2- | 140 | GTGATGGCTGCTGCCTTAGTTCTGACGAAATGCAAC |
| GB-rev | | |
| CymR- | 141 | CTGCAGGTCGACAAGCTTGCAGGCGTATCACGAGGCAG |
| GB-fwd | | |
| CymR- | 142 | CATCTGCTGCTTCGCTCATATGAGATCTCTCCTTCGTTCATAATA |
| GB-rev | | CAAAC |
| NphB- | 143 | TCTCATATGAGCGAAGCAGCAGATG |
| cumate- | | |
| GB-fwd | | |
| NphB- | 144 | AGCAGCGGTTTCTTTACCAGACTCGAGGTCAATCTTCCAGGCTA |
| cumate- | | TCAA |
| GB-rev | | |
| NphB- | 145 | AAGGAGATATACATAATGAGCGAAGCAGCAGAT |
| IF-fwd | | |
| NphB- | 146 | TTACCAGACTCGAGGTCAATCTTCCAGGCTATCAA |
| IF-rev | | |

TABLE AB
Plasmids for expression of prenyltransferase
and CBGA in vivo synthesis in E. coli

Name
pET-P1-idi-trGPPS2-P2-NphB
pET-P1-idi-trGPPS2-CT5-NphB
pET-P1-idi-trGPPS2-CT5-CnqP3
pET-P1-idi-trGPPS2-CT5-CphB
pET-P1-idi-trGPPS2-CT5-SCO7190
pET-P1-idi-trGPPS2-CT5-SCO7190(R65S)
pET-P1-idi-trGPPS2-CT5-SCO7190(E278G)

1.-63. (canceled)

64. A recombinant bacteria producing an isoprenoid precursor(s), or optionally an isoprenoid(s) or a derivative(s) thereof.

65. The recombinant bacteria of claim 64, said recombinant bacteria comprising:
a) one or more condensation product(s) selected from:
a. a beta-ketoacyl-CoA produced by an enzyme-catalyzed non-decarboxylative Claisen condensation of an acyl-CoA plus a second acyl-CoA;
b. a beta-ketoacyl-CoA produced by an enzyme-catalyzed decarboxylative Claisen condensation of an acyl-CoA plus a beta-carboxylic acyl-CoA;
c. an aldol produced by an enzyme-catalyzed aldol condensation of an aldehyde plus a ketone, or an aldehyde plus a second aldehyde, or an aldehyde plus a carboxylic acid;
d. an acyloin produced by an enzyme-catalyzed non-decarboxylative acyloin condensation of a ketone and an aldehyde, or an aldehyde and a second aldehyde; or
e. an acyloin produced by an enzyme-catalyzed decarboxylative acyloin condensation of a ketone and an alpha-keto acid, or an aldehyde and an alpha-keto acid, or an alpha-keto acid and a second alpha-keto acid;
b) three or more enzymes to convert said condensation product(s) to an isoprenoid alcohol(s), with at least three of said enzymes selected from an acetohydroxy acid isomeroreductase, an acetoacetate decarboxylate, an acyl-CoA dehydrogenase, an acyl-CoA reductase, an acyl-CoA synthase, an acyl-CoA transferase, an alcohol dehydratase, an alcohol dehydrogenase, an aldehyde decarboxylase, an alpha-keto acid decarboxylase, an alpha-keto acid dehydrogenase, a carboxylate kinase, a carboxylate reductase, a dehydratase, a dihydroxyacid dehydratase, a diol dehydratase, an enoate hydratase, an enoyl-CoA hydratase, an enoyl-CoA reductase, a glutaconyl-CoA decarboxylase, an hydroxyacid dehydratase, an hydroxyacid dehydrogenase, an hydroxyacyl-CoA dehydratase, an hydroxyacyl-CoA dehydrogenase, an hydroxymethylacyl-CoA synthase, an isomeroreductase, an isopropylmalate dehydrogenase, an isopropylmalate isomerase, an isopropylmalate synthase, a mutase, an omega-oxidation enzyme, a phosphotransacylase, a thioesterase, or a thiolase, where said conversion optionally proceeds through an isoprenoid acyl-CoA;
c) one or more phosphorylation enzyme(s) to convert said isoprenoid alcohol(s) to an isoprenoid precursor(s); and
d) optionally one or more enzyme(s) to convert said isoprenoid precursor(s) to another isoprenoid precursor(s) or an isoprenoid(s) or a derivative(s) thereof;
wherein one or more of said enzyme(s) is heterologous.

66. The recombinant bacteria of claim 64, said recombinant bacteria comprising:
a) one or more condensation product(s) selected from:
a. a beta-ketoacyl-CoA produced by an enzyme-catalyzed non-decarboxylative Claisen condensation of an acyl-CoA plus a second acyl-CoA;
b. a beta-ketoacyl-CoA produced by an enzyme-catalyzed decarboxylative Claisen condensation of an acyl-CoA plus a beta-carboxylic acyl-CoA;
c. an aldol produced by an enzyme-catalyzed aldol condensation of an aldehyde plus a ketone, or an aldehyde plus a second aldehyde, or an aldehyde plus a carboxylic acid;
d. an acyloin produced by an enzyme-catalyzed non-decarboxylative acyloin condensation of a ketone and an aldehyde, or an aldehyde and a second aldehyde; or
e. an acyloin produced by an enzyme-catalyzed decarboxylative acyloin condensation of a ketone and an alpha-keto acid, or an aldehyde and an alpha-keto acid, or an alpha-keto acid and a second alpha-keto acid;
b) one or more enzymes to convert said condensation product(s) to an intermediate product(s) selected from 2 hydroxy-isovalerate, 2-oxo-isocaproate, 3-methyl-3-hydroxybutyryl-CoA, 4-hydroxy-2-methylbutanoyl-CoA, 3-methylcrotonyl-CoA, 3-methyl-3-butenoyl-CoA, 3-methyl-2-buten-1-al, geranyl-CoA, 3-methyl-2-buten-1-ol, 3-methyl-3-buten-1-ol, 4-hydroxy-2-oxo-4-methylpentanoate, geraniol, or dimethylallyl phosphate;
c) one or more enzyme(s) to convert said intermediate product(s) to an isoprenoid alcohol(s), except in the case of intermediate products 3-methyl-2-buten-1-ol or 3-methyl-3-buten-1-ol which are isoprenoid alcohols and proceed directly to step d), or dimethylallyl phosphate which proceeds directly to step e);
d) one or more phosphorylation enzyme(s) to convert said isoprenoid alcohol(s) to an isoprenoid precursor(s); and
e) optionally one or more enzyme(s) to convert said isoprenoid precursor(s) to another isoprenoid precursor(s) or an isoprenoid(s) or a derivative(s) thereof;
wherein one or more of said enzyme(s) is heterologous.

67. The recombinant bacteria of claim 64, said recombinant bacteria comprising:
a. a thiolase or a ketoacetyl-CoA synthase enzyme catalyzing a condensation of an acyl-CoA plus a second acyl-CoA to form a beta-ketoacyl CoA, each said acyl-CoA selected from acetyl-CoA, glycolyl-CoA, propionyl-CoA, malonyl-CoA, an unsubstituted acyl-CoA, or a functionalized acyl-CoA;
b. optionally one or more iteration(s) wherein said beta-ketoacyl CoA is modified using one or more enzymes and then used as an acyl-CoA primer unit for a new condensation iteration of step a);
c. three or more enzyme(s) to convert said beta-ketoacyl CoA to an isoprenoid alcohol, said enzyme(s) comprising a beta-reduction enzyme(s), and an alcohol forming termination enzyme(s);
d. one or more phosphorylation enzyme(s) to convert said isoprenoid alcohol(s) to an isoprenoid precursor(s); and
e. optionally one or more enzyme(s) to convert said isoprenoid precursor(s) to another isoprenoid precursor(s) or an isoprenoid(s) or a derivative(s) thereof;
wherein one or more said enzyme(s) is heterologous.

68. The recombinant bacteria of claim 64, said recombinant bacteria comprising:
a. an acetolactate synthase enzyme catalyzing a decarboxylative condensation between two pyruvates to form 2-acetolactate;
b. an acetohydroxy acid isomeroreductase plus a dihydroxy acid dehydratase to convert said 2-acetolactate to 2-oxoisovalerate;
c. four or more enzymes to convert said 2-oxoisovalerate to an isoprenoid alcohol(s), with at least four of said enzymes selected from an acetohydroxy acid isomeroreductase, an acetoacetate decarboxylate, an acyl-CoA dehydrogenase, an acyl-CoA reductase, an acyl-CoA synthase, an acyl-CoA transferase, an alcohol dehydratase, an alcohol dehydrogenase, an aldehyde decarboxylase, an alpha-keto acid decarboxylase, an alpha-keto acid dehydrogenase, a carboxylate kinase, a carboxylate reductase, a dehydratase, a dihydroxyacid dehydratase, a diol dehydratase, an enoate hydratase, an enoyl-CoA hydratase, an enoyl-CoA reductase, a glutaconyl-CoA decarboxylase, an hydroxyacid dehydratase, an hydroxyacid dehydrogenase, an hydroxyacyl-CoA dehydratase, an hydroxyacyl-CoA dehydrogenase, an hydroxymethylacyl-CoA synthase, an isomeroreductase, an isopropylmalate dehydrogenase, an isopropylmalate isomerase, an isopropylmalate synthase, a mutase, an omega-oxidation enzyme, a phosphotransacylase, a thioesterase, or a thiolase, where said conversion optionally proceeds through an isoprenoid acyl-CoA;
d. one or more phosphorylation enzyme(s) to convert said isoprenoid alcohol to an isoprenoid precursor(s); and
e. optionally one or more enzyme(s) to convert said isoprenoid precursor(s) to another isoprenoid precursor(s) or an isoprenoid(s) or a derivative(s) thereof;
wherein one or more said enzyme(s) is heterologous.

69. The recombinant bacteria of claim 64, said recombinant bacteria comprising:
a. an aldolase catalyzing conversion of pyruvate and acetaldehyde to 4-hydroxy-2-oxopentanoate;
b. a mutase catalyzing conversion of said 4-hydroxy-2-oxopentanoate to 3-hydroxy-2-oxo-3-methylbutanoate;
c. a 2-hydroxyacid dehydrogenase catalyzing conversion of said 3-hydroxy-2-oxo-3-methylbutanoate to 2,3-dihydroxy-3-methylbutanoate;
d. a dihydroxyacid dehydratase catalyzing conversion of said 2,3-dihydroxy-3-methylbutanoate to 3-methyl-2-oxobutanoate;
e. a 2-hydroxyacid dehydrogenase catalyzing conversion of said 3-methyl-2-oxobutanoate to 3-methyl-2-hydroxybutanoate;
f. one or more enzyme(s) catalyzing conversion of said 3-methyl-2-hydroxybutanoate to 3-methyl-2-buten-1-ol, wherein said enzymes(s) is selected from:
a. a 2-hydroxyacid dehydratase plus a carboxylate reductase plus an alcohol dehydrogenase;
b. a 2-hydroxyacid dehydratase plus an alcohol forming acyl-CoA reductase plus one or more enzyme(s) selected from thioesterase, acyl-CoA synthase, acyl-CoA transferase, or a carboxylate kinase plus a phosphotransacylase;
c. a 2-hydroxyacid dehydratase plus an aldehyde forming acyl-CoA reductase plus an alcohol dehydrogenase plus one or more enzyme(s) selected from a thioesterase, an acyl-CoA synthase, an acyl-CoA transferase, or a carboxylate kinase plus a phosphotransacylase; or
d. a 2-hydroxyacyl-CoA dehydratase plus an alcohol forming acyl-CoA reductase plus a diol dehydratase plus an aldehyde forming acyl-CoA reductase plus an acyl-CoA dehydrogenase plus one or more additional enzyme(s) selected from:
a. an acyl-CoA synthase;
b. an acyl-CoA transferase; or
c. a carboxylate kinase plus a phosphotransacylase,
plus one or more additional enzyme(s) selected from:
a. an alcohol forming acyl-CoA reductase;
b. an aldehyde forming acyl-CoA reductase plus an alcohol dehydrogenase; or
c. a carboxylate reductase plus an alcohol dehydrogenase plus one or more enzyme(s) selected from a thioesterase, an acyl-CoA synthase, an acyl-CoA transferase, or a carboxylate kinase plus a phosphotransacylase;
g. one or more enzyme(s) catalyzing conversion of said 3-methyl-2-buten-1-ol to DMAP or DMAPP, wherein said enzyme(s) is selected from:
a. an alcohol kinase;
b. an alcohol kinase plus a phosphate kinase; or
c. an alcohol diphosphokinase;
h. optionally, an isopentenyl diphosphate isomerase catalyzing conversion of said DMAPP to IPP; and
i. optionally a geranyl pyrophosphate synthase catalyzing conversion of said DMAPP and IPP to GPP.

70. The recombinant bacteria of claim 64, said recombinant bacteria comprising:
a. an aldolase catalyzing conversion of pyruvate and acetaldehyde to 4-hydroxy-2-oxopentanoate;
b. a mutase catalyzing conversion of said 4-hydroxy-2-oxopentanoate to 3-hydroxy-2-oxo-3-methylbutanoate;
c. a 2-hydroxyacid dehydrogenase catalyzing conversion of said 3-hydroxy-2-oxo-3-methylbutanoate to 2,3-dihydroxy-3-methylbutanoate;
d. a dihydroxyacid dehydratase catalyzing conversion of said 2,3-dihydroxy-3-methylbutanoate to 3-methyl-2-oxobutanoate;
e. an isopropylmalate synthase catalyzing conversion of said 3-methyl-2-oxobutanoate to 2-isopropylmalate;
f. an isopropylmalate isomerase catalyzing conversion of said 2-isopropylmalate to 3-isopropylmalate;
g. an isopropylmalate dehydrogenase catalyzing conversion of said 3-isopropylmalate to 4-methyl-2-oxopentanoate;
h. one or more enzyme(s) catalyzing conversion of said 4-methyl-2-oxopentanoate to isovaleryl-CoA, wherein said enzyme(s) is selected from:
a. an alpha-keto acid dehydrogenase; or
b. an alpha-keto acid decarboxylase plus an aldehyde forming acyl-CoA reductase;
i. an acyl-CoA dehydrogenase catalyzing conversion of said isovaleryl-CoA to 3-methyl-2-butenoyl-CoA;
j. one or more enzyme(s) catalyzing conversion of said 3-methyl-2-butenoyl-CoA to 3-methyl-2-buten-1-ol, wherein said enzyme(s) is selected from:
a. an alcohol-forming acyl-CoA reductase;
b. an aldehyde forming acyl-CoA reductase plus an alcohol dehydrogenase; or
c. a carboxylate reductase plus an alcohol dehydrogenase plus an enzyme(s) selected from an acyl-CoA synthase, an acyl-CoA transferase, a thioesterase, or a carboxylate kinase plus a phosphotransacylase;
k. one or more enzyme(s) catalyzing conversion of said 3-methyl-2-buten-1-ol to DMAP or DMAPP, wherein said enzyme(s) is selected from:
a. an alcohol kinase;
b. an alcohol kinase plus a phosphate kinase; or
c. an alcohol diphosphokinase;
l. optionally, an isopentenyl diphosphate isomerase catalyzing conversion of said DMAPP to IPP; and
m. optionally a geranyl pyrophosphate synthase catalyzing conversion of said DMAPP and IPP to GPP.

71. The recombinant bacteria of claim 64, said recombinant bacteria comprising:
a. an aldolase catalyzing conversion of 2-oxobutanoate and acetaldehyde to 4-hydroxy-2-oxo-3-methylpentanoate;
b. a mutase catalyzing conversion of said 4-hydroxy-2-oxo-3-methylpentanoate to 4-hydroxy-2-oxo-4-methylpentanoate;
c. a 2-oxopent-4-enoate hydratase catalyzing conversion of said 4-hydroxy-2-oxo-4-methylpentanoate to 4-methyl-2-oxopent-4-enoate;
d. one or more enzyme(s) catalyzing conversion of said 4-methyl-2-oxopent-4-enoate to 3-methyl-3-buten-1-ol, wherein said enzymes(s) is selected from:
a. an alpha-ketoacid decarboxylase plus an alcohol dehydrogenase;
b. an alpha-ketoacid dehydrogenase plus an alcohol forming acyl-CoA reductase;
c. an alpha-ketoacid dehydrogenase plus an aldehyde forming acyl-CoA reductase plus an alcohol dehydrogenase; or
d. an alpha-ketoacid dehydrogenase plus a carboxylate reductase plus an alcohol dehydrogenase plus an enzyme(s) selected from a thioesterase, an acyl-CoA synthase, an acyl-CoA transferase, or a carboxylate kinase plus a phosphotransacylase;
e. one or more enzyme(s) catalyzing conversion of said 3-methyl-3-buten-1-ol to IP or IPP, wherein said enzyme(s) is selected from:
a. an alcohol kinase;
b. an alcohol kinase plus a phosphate kinase; or
c. an alcohol diphosphokinase;
f. optionally, an isopentenyl diphosphate isomerase catalyzing conversion of said IPP to DMAPP; and
g. optionally a geranyl pyrophosphate synthase catalyzing conversion of said DMAPP and IPP to GPP.

72. The recombinant bacteria of claim 64, wherein said isoprenoid precursor(s) is selected from dimethylallyl phosphate (DMAP), isopentenyl phosphate (IP), isopentenyl pyrophosphate (IPP), dimethylallyl pyrophosphate (DMAPP), geranyl pyrophosphate (GPP), farnesyl pyrophosphate (FPP), or geranylgeranyl diphosphate (GGPP).

73. The recombinant bacteria of claim 64, said recombinant bacteria comprising one or more enzyme(s) selected from, a terpene synthase, or a terpene cyclase catalyzing conversion of said isoprenoid precursor(s) to an isoprenoid(s) or a derivative(s) thereof.

74. The recombinant bacteria of claim 64, wherein said isoprenoid(s) is selected from hemiterpenoid(s), monoterpenoid(s), sesquiterpenoid(s), diterpenoid(s), sesterterpenoid(s), triterpenoid(s), tetraterpenoid(s), polyterpenoid(s), or a derivative(s) thereof.

75. The recombinant bacteria of claim 64, said recombinant bacteria comprising one or more enzymes selected from:
a. a thiolase encoded by a gene(s) selected from E. coli atoB, E. coli yqeF, E. coli fadA, E. coli fadI, Ralstonia eutropha bktB, Pseudomonas sp. B13 catF, E coli paaJ, Rhodococcus opacus pcaF, Pseudomonas putida pcaF, Streptomyces sp. pcaF, P. putida fadAx, P. putida fadA, Ralstonia eutropha phaA, Acinetobacter sp. ADP1 dcaF, Clostridium acetobutylicum th1A, or Clostridium acetobutylicum th1B;
b. an acetolactate synthase or an acyloin condensation enzyme encoded by a gene(s) selected from E. coli ilvBN, B. subtilis alsS, or E. coli ilvIH;
c. a 2-hydroxyacyl-CoA lyase encoded by a gene(s) selected from Homo sapiens hacl1, Rattus norvegicus hacl1, Dictyostelium discoideum hacl1, or Mus musculus hacl1;
d. an aldolase encoded by a gene(s) selected from E. coli mhpE, Pseudomonas putida xylK, or Pseudomonas sp. CF600 dmpG;
e. a ketoacyl-CoA synthase encoded by a gene(s) selected from Gluconobacter oxydans GOX0115, Pseudomonas aeruginosa fabH2, Streptomyces sp. MMG1121 PRK09352, Streptomyces tendae acs2, Streptomyces sp. strain CL190 nphT7, Physaria fendleri KCS3, Saccharomyces cerevisiae ELO2, Arabidopsis thaliana col KCS1, Arabidopsis thaliana col FAEJ, or Arabidopsis thaliana col CER6;
wherein said enzyme(s) or gene(s) includes its homolog(s).

76. The recombinant bacteria of claim 64, said recombinant bacteria that further comprises reduced expression of gene(s) encoding one or more fermentation enzymes leading to reduced production of one or more of lactate, acetate, ethanol or succinate.

77. A method of producing an isoprenoid precursor(s), or optionally an isoprenoid(s) or a derivative(s) thereof, said method comprising growing the recombinant bacteria of claim 64 in a culture medium for a time and under conditions to produce an isoprenoid(s), an isoprenoid precursor(s), or a derivative(s) thereof.

78. The method of claim 77, wherein said isoprenoid precursor(s), isoprenoid(s), or derivative(s) thereof is isolated from the culture medium or said recombinant bacteria or both.


SEQUENCE LISTING (146 sequences)

SEQ ID NO: 1 - DNA, 35 bp, synthetic construct
  misc_feature 1..35: note = Synthetic: pct-f1
  source 1..35: mol_type = other DNA
aggagatataccatgagaaaagtagaaatcattac

SEQ ID NO: 2 - DNA, 39 bp, synthetic construct
  misc_feature 1..39: note = Synthetic: pct-r1
  source 1..39: mol_type = other DNA
cgccgagctcgaattcttattttttcagtcccatgggac

SEQ ID NO: 3 - DNA, 51 bp, synthetic construct
  misc_feature 1..51: note = Synthetic: fadAx-f1
  source 1..51: mol_type = other DNA
gaaaaaataagaatttaaggaggaataaaccatgaccctggcaaatgatcc

SEQ ID NO: 4 - DNA, 38 bp, synthetic construct
  misc_feature 1..38: note = Synthetic: fadAx-r1
  source 1..38: mol_type = other DNA
cgccgagctcgaattcttaatacagacattcaactgcc

SEQ ID NO: 5 - DNA, 33 bp, synthetic construct
  misc_feature 1..33: note = Synthetic: fadB2x-f1
  source 1..33: mol_type = other DNA
aggagatataccatgcatatcgccaacaaacac

SEQ ID NO: 6 - DNA, 37 bp, synthetic construct
  misc_feature 1..37: note = Synthetic: fadB2x-r1
  source 1..37: mol_type = other DNA
cgccgagctcgaattcttattttgctgccatgcgcag

SEQ ID NO: 7 - DNA, 52 bp, synthetic construct
  misc_feature 1..52: note = Synthetic: fadB1x-f1
  source 1..52: mol_type = other DNA
agcaaaataagaatttaaggaggaataaaccatggcctttgaaaccattctg

SEQ ID NO: 8 - DNA, 37 bp, synthetic construct
  misc_feature 1..37: note = Synthetic: fadB1x-r1
  source 1..37: mol_type = other DNA
cgccgagctcgaattcttagcgatctttaaactgtgc

SEQ ID NO: 9 - DNA, 37 bp, synthetic construct
  misc_feature 1..37: note = Synthetic: ydiI-f1
  source 1..37: mol_type = other DNA
aaggagatatacatatgatatggaaacggaaaatcac

SEQ ID NO: 10 - DNA, 36 bp, synthetic construct
  misc_feature 1..36: note = Synthetic: ydiI-r1
  source 1..36: mol_type = other DNA
ttgagatctgccatatgtcacaaaatggcggtcgtc

SEQ ID NO: 11 - DNA, 34 bp, synthetic construct
  misc_feature 1..34: note = Synthetic: idi-f1
  source 1..34: mol_type = other DNA
aggagatataccatgcaaacggaacacgtcattt

SEQ ID NO: 12 - DNA, 59 bp, synthetic construct
  misc_feature 1..59: note = Synthetic: idi-r1
  source 1..59: mol_type = other DNA
tgcgctatatgccatggtttattcctccttaaattatttaagctgggtaaatgcagata

SEQ ID NO: 13 - DNA, 20 bp, synthetic construct
  misc_feature 1..20: note = Synthetic: trgpps2-f1
  source 1..20: mol_type = other DNA
atggcatatagcgcaatggc

SEQ ID NO: 14 - DNA, 38 bp, synthetic construct
  misc_feature 1..38: note = Synthetic: trgpps2-r1
  source 1..38: mol_type = other DNA
gtgatggctgctgccttagttctgacgaaatgcaacat

SEQ ID NO: 15 - DNA, 36 bp, synthetic construct
  misc_feature 1..36: note = Synthetic: ges-f1
  source 1..36: mol_type = other DNA
aaggagatatacataatggaagaaagcagcagcaaa

SEQ ID NO: 16 - DNA, 36 bp, synthetic construct
  misc_feature 1..36: note = Synthetic: ges-r1
  source 1..36: mol_type = other DNA
ttaccagactcgaggttactgggtaaaaaacagggc

SEQ ID NO: 17 - DNA, 49 bp, synthetic construct
  misc_feature 1..49: note = Synthetic: ychB-f1
  source 1..49: mol_type = other DNA
acccagtaacctcgaaaggaggaataaggcatgcggacacagtggccct

SEQ ID NO: 18 - DNA, 57 bp, synthetic construct
  misc_feature 1..57: note = Synthetic: ychB-r1
  source 1..57: mol_type = other DNA
tttcaggatgatcatttgttattcctccttaaggtcttaaagcatggctctgtgcaa

SEQ ID NO: 19 - DNA, 21 bp, synthetic construct
  misc_feature 1..21: note = Synthetic: mtipk-f1
  source 1..21: mol_type = other DNA
atgatcatcctgaaactgggt

SEQ ID NO: 20 - DNA, 39 bp, synthetic construct
  misc_feature 1..39: note = Synthetic: mtipk-r1
  source 1..39: mol_type = other DNA
ctttaccagactcgagttagtgtttacctgtaatacgtg

SEQ ID NO: 21 - DNA, 52 bp, synthetic construct
  misc_feature 1..52: note = Synthetic: mtipk-f2
  source 1..52: mol_type = other DNA
gatccgctaactcgataaggaggaataacaaatgatcatcctgaaactgggt

SEQ ID NO: 22 - DNA, 52 bp, synthetic construct
  misc_feature 1..52: note = Synthetic: taipk-f1
  source 1..52: mol_type = other DNA
acccagtaacctcgaaaggaggaataaggcatgatgatcctgaaaattggtg

SEQ ID NO: 23 - DNA, 35 bp, synthetic construct
  misc_feature 1..35: note = Synthetic: taipk-r1
  source 1..35: mol_type = other DNA
ctttaccagactcgagttagcggatcacggtgcca

SEQ ID NO: 24 - DNA, 37 bp, synthetic construct
  misc_feature 1..37: note = Synthetic: maqu_2507- f1
  source 1..37: mol_type = other DNA
gccaggatccgaattcgaactactttctgaccggtgg

SEQ ID NO: 25 - DNA, 36 bp, synthetic construct
  misc_feature 1..36: note = Synthetic: maqu_2507- r1
  source 1..36: mol_type = other DNA
cgccgagctcgaattcttaccagtaaatgccacgca

SEQ ID NO: 26 - DNA, 40 bp, synthetic construct
  misc_feature 1..40: note = Synthetic: cbjALD-f1
  source 1..40: mol_type = other DNA
gccaggatccgaattcgaataaagacacactaatacctac

SEQ ID NO: 27 - DNA, 36 bp, synthetic construct
  misc_feature 1..36: note = Synthetic: cbjALD-r1
  source 1..36: mol_type = other DNA
cgccgagctcgaattcttagccggcaagtacacatc

SEQ ID NO: 28 - DNA, 38 bp, synthetic construct
  misc_feature 1..38: note = Synthetic: chnD-f1
  source 1..38: mol_type = other DNA
gccaggatccgaattcgcactgctattgtgttacccac

SEQ ID NO: 29 - DNA, 37 bp, synthetic construct
  misc_feature 1..37: note = Synthetic: chnD-r1
  source 1..37: mol_type = other DNA
cgccgagctcgaattctcaattttcgtgcatcagaac

SEQ ID NO: 30 - DNA, 36 bp, synthetic construct
  misc_feature 1..36: note = Synthetic: tdTer-f1
  source 1..36: mol_type = other DNA
aaggagatatacatatgattgttaagccgatggtcc

SEQ ID NO: 31 - DNA, 39 bp, synthetic construct
  misc_feature 1..39: note = Synthetic: tdTer-r1
  source 1..39: mol_type = other DNA
ttgagatctgccatatgttagatgcggtcaaaacgttca

SEQ ID NO: 32 - DNA, 35 bp, synthetic construct; identical to SEQ ID NO: 1
  misc_feature 1..35: note = Synthetic: pct-f1
  source 1..35: mol_type = other DNA

SEQ ID NO: 33 - DNA, 39 bp, synthetic construct; identical to SEQ ID NO: 2
  misc_feature 1..39: note = Synthetic: pct-r1
  source 1..39: mol_type = other DNA

SEQ ID NO: 34 - DNA, 35 bp, synthetic construct
  misc_feature 1..35: note = Synthetic: bktB-f1
  source 1..35: mol_type = other DNA
aggagatataccatgatgacgcgtgaagtggtagt

SEQ ID NO: 35 - DNA, 36 bp, synthetic construct
  misc_feature 1..36: note = Synthetic: bktB-r1
  source 1..36: mol_type = other DNA
cgccgagctcgaattctcagatacgctcgaagatgg

SEQ ID NO: 36 - DNA, 47 bp, synthetic construct
  misc_feature 1..47: note = Synthetic: phaB1-f1
  source 1..47: mol_type = other DNA
gcgtatctgagaattaggaggctctctatgactcagcgcattgcgta

SEQ ID NO: 37 - DNA, 34 bp, synthetic construct
  misc_feature 1..34: note = Synthetic: phaB1-r1
  source 1..34: mol_type = other DNA
cgccgagctcgaattctcagcccatgtgcaggcc

SEQ ID NO: 38 - DNA, 32 bp, synthetic construct
  misc_feature 1..32: note = Synthetic: phaJ-f1
  source 1..32: mol_type = other DNA
aaggagatatacatatgtcggcacaaagcctg

SEQ ID NO: 39 - DNA, 36 bp, synthetic construct
  misc_feature 1..36: note = Synthetic: phaJ-r1
  source 1..36: mol_type = other DNA
ttgagatctgccatatgttacggcagtttcaccacc

SEQ ID NO: 40 - DNA, 34 bp, synthetic construct
  misc_feature 1..34: note = Synthetic: pct540- f1
  source 1..34: mol_type = other DNA
gaggaataaaccatgcgtaaagtgccgattatta

SEQ ID NO: 41 - DNA, 37 bp, synthetic construct
  misc_feature 1..37: note = Synthetic: pct540- r1
  source 1..37: mol_type = other DNA
gatgatgatggtcgacgcttttcatttctttcaggcc

SEQ ID NO: 42 - DNA, 41 bp, synthetic construct
  misc_feature 1..41: note = Synthetic: pct-f2
  source 1..41: mol_type = other DNA
tagaaggaggagatctatgagaaaagtagaaatcattacag

SEQ ID NO: 43 - DNA, 37 bp, synthetic construct
  misc_feature 1..37: note = Synthetic: pct-r2
  source 1..37: mol_type = other DNA
gggggaccagctcgagttttttcagtcccatgggacc

SEQ ID NO: 44 - DNA, 35 bp, synthetic construct
  misc_feature 1..35: note = Synthetic: alsS-f1
  source 1..35: mol_type = other DNA
aaggagatatacatatgaccaaagcaaccaaagaa

SEQ ID NO: 45 - DNA, 54 bp, synthetic construct
  misc_feature 1..54: note = Synthetic: alsS-r1
  source 1..54: mol_type = other DNA
aatggtaatacgcatgttaatttcctcctagaattacagggctttggttttcat

SEQ ID NO: 46 - DNA, 20 bp, synthetic construct
  misc_feature 1..20: note = Synthetic: panE-f1
  source 1..20: mol_type = other DNA
atgcgtattaccattgccgg

SEQ ID NO: 47 - DNA, 38 bp, synthetic construct
  misc_feature 1..38: note = Synthetic: panE-r1
  source 1..38: mol_type = other DNA
ttgagatctgccatattatttggctttcagcagttctt

SEQ ID NO: 48 - DNA, 32 bp, synthetic construct
  misc_feature 1..32: note = Synthetic: ilvC-f1
  source 1..32: mol_type = other DNA
aggagatataccatggctaactacttcaatac

SEQ ID NO: 49 - DNA, 56 bp, synthetic construct
  misc_feature 1..56: note = Synthetic: ilvC-r1
  source 1..56: mol_type = other DNA
acggtacttaggcatggtttattcctccttaaactcttaacccgcaacagcaatac

SEQ ID NO: 50 - DNA, 19 bp, synthetic construct
  misc_feature 1..19: note = Synthetic: ilvD-f1
  source 1..19: mol_type = other DNA
atgcctaagtaccgttccg

SEQ ID NO: 51 - DNA, 37 bp, synthetic construct
  misc_feature 1..37: note = Synthetic: ilvD-r1
  source 1..37: mol_type = other DNA
cgccgagctcgaattcttaaccccccagtttcgattt

SEQ ID NO: 52 - DNA, 39 bp, synthetic construct
  misc_feature 1..39: note = Synthetic: fadM-r1
  source 1..39: mol_type = other DNA
tgcctctagcacgcgtcgtttacttaaccatctgctcca

SEQ ID NO: 53 - DNA, 44 bp, synthetic construct
  misc_feature 1..44: note = Synthetic: tesA-f1
  source 1..44: mol_type = other DNA
ttaaagaggagaaaggtaccatgatgaacttcaacaatgttttc

SEQ ID NO: 54 - DNA, 37 bp, synthetic construct
  misc_feature 1..37: note = Synthetic: tesA-r1
  source 1..37: mol_type = other DNA
tgcctctagcacgcgttccgttgctttatgagtcatg

SEQ ID NO: 55 - DNA, 40 bp, synthetic construct
  misc_feature 1..40: note = Synthetic: tesB-f1
  source 1..40: mol_type = other DNA
ttaaagaggagaaaggtaccatgagtcaggcgctaaaaaa

SEQ ID NO: 56 - DNA, 34 bp, synthetic construct
  misc_feature 1..34: note = Synthetic: tesB-r1
  source 1..34: mol_type = other DNA
tgcctctagcacgcgtaacagccggacggttttc

SEQ ID NO: 57 - DNA, 42 bp, synthetic construct
  misc_feature 1..42: note = Synthetic: ybgC-f1
  source 1..42: mol_type = other DNA
ttaaagaggagaaaggtaccgtgaatacaacgctgtttcgat

SEQ ID NO: 58 - DNA, 36 bp, synthetic construct
  misc_feature 1..36: note = Synthetic: ybgC-r1
  source 1..36: mol_type = other DNA
tgcctctagcacgcgttcactgcttaaactccgcga

SEQ ID NO: 59 - DNA, 43 bp, synthetic construct
  misc_feature 1..43: note = Synthetic: yciA-f1
  source 1..43: mol_type = other DNA
ttaaagaggagaaaggtaccatgtctacaacacataacgtccc

SEQ ID NO: 60 - DNA, 40 bp, synthetic construct
  misc_feature 1..40: note = Synthetic: yciA-r1
  source 1..40: mol_type = other DNA
tgcctctagcacgcgtttcagtaagcagaaagtcaaaagc

SEQ ID NO: 61 - DNA, 42 bp, synthetic construct
  misc_feature 1..42: note = Synthetic: ydiI-f1
  source 1..42: mol_type = other DNA
ttaaagaggagaaaggtaccatgatatggaaacggaaaatca

SEQ ID NO: 62 - DNA, 37 bp, synthetic construct
  misc_feature 1..37: note = Synthetic: ydiI-r1
  source 1..37: mol_type = other DNA
tgcctctagcacgcgtggtgacaacgtcacaaaatgg

SEQ ID NO: 63 - DNA, 34 bp, synthetic construct
  misc_feature 1..34: note = Synthetic: atoB-f1
  source 1..34: mol_type = other DNA
gaggaataaaccatgaaaaattgtgtcatcgtca

SEQ ID NO: 64 - DNA, 37 bp, synthetic construct
  misc_feature 1..37: note = Synthetic: atoB-r1
  source 1..37: mol_type = other DNA
cccaagcttcgaattcttaattcaaccgttcaatcac

SEQ ID NO: 65 - DNA, 35 bp, synthetic construct
  misc_feature 1..35: note = Synthetic: fadB-f1
  source 1..35: mol_type = other DNA
taagaattcgaagctgcggattcaggagactgaca

SEQ ID NO: 66 - DNA, 35 bp, synthetic construct
  misc_feature 1..35: note = Synthetic: fadB-r1
  source 1..35: mol_type = other DNA
gttcgggcccaagctttaagccgttttcaggtcgc

SEQ ID NO: 67 - DNA, 53 bp, synthetic construct
  misc_feature 1..53: note = Synthetic: egter-f1
  source 1..53: mol_type = other DNA
aaacggcttaaagctaataaggaggaataaaccatggcaatgtttaccacgac

SEQ ID NO: 68 - DNA, 43 bp, synthetic construct
  misc_feature 1..43: note = Synthetic: egter-r1
  source 1..43: mol_type = other DNA
gttcgggcccaagcttgcggccgcttattgctgtgctgcggac

SEQ ID NO: 69 - DNA, 58 bp, synthetic construct
  misc_feature 1..58: note = Synthetic: alsS-r2
  source 1..58: mol_type = other DNA
gctcggataggtcatgtgatattcctcctagctatgttacagggctttggttttcatc

SEQ ID NO: 70 - DNA, 20 bp, synthetic construct
  misc_feature 1..20: note = Synthetic: liuA-f1
  source 1..20: mol_type = other DNA
atgacctatccgagcctgaa

SEQ ID NO: 71 - DNA, 42 bp, synthetic construct
  misc_feature 1..42: note = Synthetic: liuA-r1
  source 1..42: mol_type = other DNA
ttaccagactcgagggtaccttagcgggtttcattaaacagt

SEQ ID NO: 72 - DNA, 32 bp, synthetic construct
  misc_feature 1..32: note = Synthetic: bkdF-f1
  source 1..32: mol_type = other DNA
aggagatataccatgaccgttgaaagcaccgc

SEQ ID NO: 73 - DNA, 56 bp, synthetic construct
  misc_feature 1..56: note = Synthetic: bkdF-r1
  source 1..56: mol_type = other DNA
cattttttctgccatgagttattcctcctacaactcttaattaccaccttgaccgg

SEQ ID NO: 74 - DNA, 22 bp, synthetic construct
  misc_feature 1..22: note = Synthetic: bkdG-f1
  source 1..22: mol_type = other DNA
atggcagaaaaaatggcaatcg

SEQ ID NO: 75 - DNA, 57 bp, synthetic construct
  misc_feature 1..57: note = Synthetic: bkdG-r1
  source 1..57: mol_type = other DNA
gcttgcttcggtcatgctttattcctcctttaattgttaatatgccaggctacgatc

SEQ ID NO: 76 - DNA, 20 bp, synthetic construct
  misc_feature 1..20: note = Synthetic: bkdH-f1
  source 1..20: mol_type = other DNA
atgaccgaagcaagcgttcg

SEQ ID NO: 77 - DNA, 42 bp, synthetic construct; identical to SEQ ID NO: 71
  misc_feature 1..42: note = Synthetic: bkdH-r1
  source 1..42: mol_type = other DNA

SEQ ID NO: 78 - DNA, 34 bp, synthetic construct
  misc_feature 1..34: note = Synthetic: lplA-f1
  source 1..34: mol_type = other DNA
aaggagatatacatatgtccacattacgcctgct

SEQ ID NO: 79 - DNA, 50 bp, synthetic construct
  misc_feature 1..50: note = Synthetic: lplA-r1
  source 1..50: mol_type = other DNA
tgcatcatttgccatccattattcctccttgggtaactaccttacagccc

SEQ ID NO: 80 - DNA, 20 bp, synthetic construct
  misc_feature 1..20: note = Synthetic: lpdA1-f1
  source 1..20: mol_type = other DNA
atggcaaatgatgcaagcac

SEQ ID NO: 81 - DNA, 41 bp, synthetic construct
  misc_feature 1..41: note = Synthetic: lpdA1-r1
  source 1..41: mol_type = other DNA
ttaccagactcgagggtaccttaatcatggctatgcagcgg

SEQ ID NO: 82 - DNA, 29 bp, synthetic construct
  misc_feature 1..29: note = Synthetic: leuA(G462 D)B-f11
  source 1..29: mol_type = other DNA
aggagatataccatgagccagcaagtcat

SEQ ID NO: 83 - DNA, 29 bp, synthetic construct
  misc_feature 1..29: note = Synthetic: leuA(G462 D)B-r11
  source 1..29: mol_type = other DNA
cacctggtcaagcgcatctttaccgtggc

SEQ ID NO: 84 - DNA, 34 bp, synthetic construct
  misc_feature 1..34: note = Synthetic: leuA(G462 D)B-f12
  source 1..34: mol_type = other DNA
aagatgcgcttgaccaggtggatatcgtcgctaa

SEQ ID NO: 85 - DNA, 37 bp, synthetic construct
  misc_feature 1..37: note = Synthetic: leuA(G462 D)B-r12
  source 1..37: mol_type = other DNA
cgccgagctcgaattcttacaccccttctgctacata

SEQ ID NO: 86 - DNA, 21 bp, synthetic construct
  misc_feature 1..21: note = Synthetic: leuA(G462 D)B-f2
  source 1..21: mol_type = other DNA
aggagatataccatgagccag

SEQ ID NO: 87 - DNA, 20 bp, synthetic construct
  misc_feature 1..20: note = Synthetic: leuA(G462 D)B-r2
  source 1..20: mol_type = other DNA
cgccgagctcgaattcttac

SEQ ID NO: 88 - DNA, 38 bp, synthetic construct
  misc_feature 1..38: note = Synthetic: leuCD-f1
  source 1..38: mol_type = other DNA
cgatcgctgacgtcgatggctaagacgttatacgaaaa

SEQ ID NO: 89 - DNA, 43 bp, synthetic construct
  misc_feature 1..43: note = Synthetic: leuCD-r1
  source 1..43: mol_type = other DNA
ttaccagactcgagggtaccttaattcataaacgcaggttgtt

SEQ ID NO: 90 - DNA, 69 bp, synthetic construct
  misc_feature 1..69: note = Synthetic: kan-homatoE-L
  source 1..69: mol_type = other DNA
ttggtttaacgctgttctgacggcacccctacaaacagaaggaatataaacatatgaatatcctcctta

SEQ ID NO: 91 - DNA, 41 bp, synthetic construct
  misc_feature 1..41: note = Synthetic: kan-ovcymatoB- R
  source 1..41: mol_type = other DNA
tctgaaattctgcctcgtgagtgtaggctggagctgcttcg

SEQ ID NO: 92 - DNA, 41 bp, synthetic construct
  misc_feature 1..41: note = Synthetic: cym-pCTC- atoB-ovkan-L
  source 1..41: mol_type = other DNA
cgaagcagctccagcctacactcacgaggcagaatttcaga

SEQ ID NO: 93 - DNA, 18 bp, synthetic construct
  misc_feature 1..18: note = Synthetic: atoBintrecomb- R
  source 1..18: mol_type = other DNA
gccagcccgctttttaac

SEQ ID NO: 94 - DNA, 35 bp, synthetic construct
  misc_feature 1..35: note = Synthetic: maqu_250 7-f2
  source 1..35: mol_type = other DNA
aaggagatatacatatgaactactttctgaccggt

SEQ ID NO: 95 - DNA, 38 bp, synthetic construct
  misc_feature 1..38: note = Synthetic: maqu_250 7-r2
  source 1..38: mol_type = other DNA
ttgagatctgccatatgttaccagtaaatgccacgcat

SEQ ID NO: 96 - DNA, 56 bp, synthetic construct
  misc_feature 1..56: note = Synthetic: maqu_250 7-r3
  source 1..56: mol_type = other DNA
ttaaattccggcataattactccttcactgccatattaccagtaaatgccacgcat

SEQ ID NO: 97 - DNA, 39 bp, synthetic construct
  misc_feature 1..39: note = Synthetic: cbjALD-f2
  source 1..39: mol_type = other DNA
aaggagatatacatatgaataaagacacactaataccta

SEQ ID NO: 98 - DNA, 37 bp, synthetic construct
  misc_feature 1..37: note = Synthetic: cbjALD-r2
  source 1..37: mol_type = other DNA
ttgagatctgccatatgttagccggcaagtacacatc

SEQ ID NO: 99 - DNA, 55 bp, synthetic construct
  misc_feature 1..55: note = Synthetic: cbjALD-r3
  source 1..55: mol_type = other DNA
ttaaattccggcataattactccttcactgccatattagccggcaagtacacatc

SEQ ID NO: 100 - DNA, 39 bp, synthetic construct
  misc_feature 1..39: note = Synthetic: adhE2-f1
  source 1..39: mol_type = other DNA
aaggagatatacatatgaaagttacaaatcaaaaagaac

SEQ ID NO: 101 - DNA, 47 bp, synthetic construct
  misc_feature 1..47: note = Synthetic: adhE2-r1
  source 1..47: mol_type = other DNA
ttgagatctgccatatgttaaaatgattttatatagatatccttaag

SEQ ID NO: 102 - DNA, 33 bp, synthetic construct
  misc_feature 1..33: note = Synthetic: hmgs-f1
  source 1..33: mol_type = other DNA
aggagatataccatgaccatcggcatcgataag

SEQ ID NO: 103 - DNA, 38 bp, synthetic construct
  misc_feature 1..38: note = Synthetic: hmgs-r1
  source 1..38: mol_type = other DNA
cgccgagctcgaattcttattccggacgatgatattcg

SEQ ID NO: 104 - DNA, 51 bp, synthetic construct
  misc_feature 1..51: note = Synthetic: aibA-f1
  source 1..51: mol_type = other DNA
tccggaataagaattgtaggaggaatactacatgaaaaccgcacgttggtg

SEQ ID NO: 105 - DNA, 56 bp, synthetic construct
  misc_feature 1..56: note = Synthetic: aibA-r1
  source 1..56: mol_type = other DNA
cagggttgcgctcatggtttattcctccttaaaatcttatgctgcacgacgggtca

SEQ ID NO: 106 - DNA, 20 bp, synthetic construct
  misc_feature 1..20: note = Synthetic: aibB-f1
  source 1..20: mol_type = other DNA
atgagcgcaaccctggatat

SEQ ID NO: 107 - DNA, 40 bp, synthetic construct
  misc_feature 1..40: note = Synthetic: aibB-r1
  source 1..40: mol_type = other DNA
cctgcaggcgcgccgagctcttatgcaccaaccagtgcat

SEQ ID NO: 108 - DNA, 36 bp, synthetic construct
  misc_feature 1..36: note = Synthetic: liuC-f1
  source 1..36: mol_type = other DNA
aaggagatatacatatgccggaatttaaagttgatg

SEQ ID NO: 109 - DNA, 37 bp, synthetic construct
  misc_feature 1..37: note = Synthetic: liuC-r1
  source 1..37: mol_type = other DNA
ttgagatctgccatattaacgacctttataaaccggt

SEQ ID NO: 110 - DNA, 50 bp, synthetic construct
  misc_feature 1..50: note = Synthetic: chnD-f2
  source 1..50: mol_type = other DNA
ttaatatggcagatcaggaggaatagctgatgcactgctattgtgttacc

SEQ ID NO: 111 - DNA, 37 bp, synthetic construct
  misc_feature 1..37: note = Synthetic: chnD-r2
  source 1..37: mol_type = other DNA
ctttaccagactcgagtcaattttcgtgcatcagaac

SEQ ID NO: 112 - DNA, 51 bp, synthetic construct
  misc_feature 1..51: note = Synthetic: yjgB-f1
  source 1..51: mol_type = other DNA
ttaatatggcagatcaggaggaatagctgatgtcgatgataaaaagctatg

SEQ ID NO: 113 - DNA, 37 bp, synthetic construct
  misc_feature 1..37: note = Synthetic: yjgB-r1
  source 1..37: mol_type = other DNA
ctttaccagactcgagtcaaaaatcggctttcaacac

SEQ ID NO: 114 - DNA, 51 bp, synthetic construct
  misc_feature 1..51: note = Synthetic: yahK-f1
  source 1..51: mol_type = other DNA
ttaatatggcagatcaggaggaatagctgatgaagatcaaagctgttggtg

SEQ ID NO: 115 - DNA, 37 bp, synthetic construct
  misc_feature 1..37: note = Synthetic: yahK-r1
  source 1..37: mol_type = other DNA
ctttaccagactcgagtcagtctgttagtgtgcgatt

SEQ ID NO: 116 - DNA, 70 bp, synthetic construct
  misc_feature 1..70: note = Synthetic: Reverse primer
  source 1..70: mol_type = other DNA
gcattggcggcggtcagggaattgcgatggtgattgaacggttgaattaaaaaatgagacgttgatcggc

SEQ ID NO: 117 - DNA, 70 bp, synthetic construct
  misc_feature 1..70: note = Synthetic: Forward primer
  source 1..70: mol_type = other DNA
caacaaacagacaatctggtctgtttgtattatgaacgaaggagagatctatgacgcgtgaagtggtagt

SEQ ID NO: 118 - DNA, 70 bp, synthetic construct
  misc_feature 1..70: note = Synthetic: Reverse primer
  source 1..70: mol_type = other DNA
gatgttcaagaaaacacccgataactttcgctatcgggtgtttttattgatcagatacgctcgaagatgg

SEQ ID NO: 119 - DNA, 71 bp, synthetic construct
  misc_feature 1..71: note = Synthetic: Forward primer
  source 1..71: mol_type = other DNA
ttgacggcggtttcagcattgctgcaatgaacgaactcgaactgaaataagtgtaggctggagctgcttcg

SEQ ID NO: 120 - DNA, 90 bp, synthetic construct
  misc_feature 1..90: note = Synthetic: Reverse primer
  source 1..90: mol_type = other DNA
aacagagataacgggcggcagaacgccgcccatctttaccaacagaacgattatttcagttcgagttcgttttattgctgtgctgcggac

SEQ ID NO: 121 - DNA, 70 bp, synthetic construct
  misc_feature 1..70: note = Synthetic: Forward primer
  source 1..70: mol_type = other DNA
caacaaacagacaatctggtctgtttgtattatgaacgaaggagagatctatggcaatgtttaccacgac

SEQ ID NO: 122 - DNA, 69 bp, synthetic construct
  misc_feature 1..69: note = Synthetic: Reverse primer
  source 1..69: mol_type = other DNA
ttaaacccgctcaaacaccgtcgcaataccctgacccagaccgatacacattattgctgtgctgcggac

SEQ ID NO: 123 - DNA, 72 bp, synthetic construct
  misc_feature 1..72: note = Synthetic: F1 primer
  source 1..72: mol_type = other DNA
atcctccggttgagccagcccgtccggttggcgacctgaaaacggcttaaatgattgaacaagatggattgc

SEQ ID NO: 124 - DNA, 42 bp, synthetic construct
  misc_feature 1..42: note = Synthetic: R1 primer
  source 1..42: mol_type = other DNA
taaggggtgacgccaaagtatcagaagaactcgtcaagaagg

SEQ ID NO: 125 - DNA, 42 bp, synthetic construct
  misc_feature 1..42: note = Synthetic: F2 primer
  source 1..42: mol_type = other DNA
ccttcttgacgagttcttctgatactttggcgtcacccctta

SEQ ID NO: 126 - DNA, 73 bp, synthetic construct
  misc_feature 1..73: note = Synthetic: R2 primer
  source 1..73: mol_type = other DNA
atcggggtgcgaattgcatcgacaatgacaacctgttccattgtgactccatcaaagggaaaactgtccatat

SEQ ID NO: 127 - DNA, 69 bp, synthetic construct
  misc_feature 1..69: note = Synthetic: Forward primer
  source 1..69: mol_type = other DNA
ttgagccagcccgtccggttggcgacctgaaaacggcttaaggagtcacaatggccaagttgaccagtg

SEQ ID NO: 128 - DNA, 68 bp, synthetic construct
  misc_feature 1..68: note = Synthetic: Reverse primer
  source 1..68: mol_type = other DNA
ttaaacccgctcaaacaccgtcgcaataccctgacccagaccgatacacatcagtcctgctcctctgc

SEQ ID NO: 129 - DNA, 40 bp, synthetic construct
  misc_feature 1..40: note = Synthetic: fadM-f1
  source 1..40: mol_type = other DNA
ttaaagaggagaaaggtaccatgcaaacacaaatcaaagt

SEQ ID NO: 130 - DNA, 73 bp, synthetic construct
  misc_feature 1..73: note = Synthetic: Forward primer
  source 1..73: mol_type = other DNA
gatgttcaagaaaacacccgataactttcgctatcgggtgtttttattgaatcaaagggaaaactgtccatat

SEQ ID NO: 131 - DNA, 30 bp, synthetic construct
  misc_feature 1..30: note = Synthetic: MK-IF-fwd
  source 1..30: mol_type = other DNA
aggagatataccatgagcctgccgtttctg

SEQ ID NO: 132 - DNA, 34 bp, synthetic construct
  misc_feature 1..34: note = Synthetic: MK-IF-rev
  source 1..34: mol_type = other DNA
cgccgagctcgaattcttagctggtccacggcag

SEQ ID NO: 133 - DNA, 37 bp, synthetic construct
  misc_feature 1..37: note = Synthetic: PMK-IF-fwd
  source 1..37: mol_type = other DNA
gaccagctaagaatttaggaggaataactcatgagcg

SEQ ID NO: 134 - DNA, 41 bp, synthetic construct
  misc_feature 1..41: note = Synthetic: PMK-IF-rev
  source 1..41: mol_type = other DNA
cgccgagctcgaattcattcctcctttaattgttatttgtc

SEQ ID NO: 135 - DNA, 35 bp, synthetic construct
  misc_feature 1..35: note = Synthetic: PMD-IF-fwd
  source 1..35: mol_type = other DNA
aaggaggaatgaattatgaccgtttataccgcaag

SEQ ID NO: 136 - DNA, 34 bp, synthetic construct
  misc_feature 1..34: note = Synthetic: PMD-IF-rev
  source 1..34: mol_type = other DNA
cgccgagctcgaattcttattctttcggcagacc

SEQ ID NO: 137 - DNA, 43 bp, synthetic construct
  misc_feature 1..43: note = Synthetic: idi-GB-fwd
  source 1..43: mol_type = other DNA
gtttaactttaagaaggagatatacatgcaaacggaacacgtc

SEQ ID NO: 138 - DNA, 43 bp, synthetic construct
  misc_feature 1..43: note = Synthetic: idi-GB-rev
  source 1..43: mol_type = other DNA
atggtttattcctccttaaattatttaagctgggtaaatgcag

SEQ ID NO: 139 - DNA, 47 bp, synthetic construct
  misc_feature 1..47: note = Synthetic: trGPPS2-IF-fwd
  source 1..47: mol_type = other DNA
tttaaggaggaataaaccatggtggaatttgactttaacaaatatat

SEQ ID NO: 140 - DNA, 36 bp, synthetic construct
  misc_feature 1..36: note = Synthetic: GPPS2-GB-rev
  source 1..36: mol_type = other DNA
gtgatggctgctgccttagttctgacgaaatgcaac

SEQ ID NO: 141 - DNA, 38 bp, synthetic construct
  misc_feature 1..38: note = Synthetic: CymR-GB-fwd
  source 1..38: mol_type = other DNA
ctgcaggtcgacaagcttgcaggcgtatcacgaggcag

SEQ ID NO: 142 - DNA, 50 bp, synthetic construct
  misc_feature 1..50: note = Synthetic: CymR-GB-rev
  source 1..50: mol_type = other DNA
catctgctgcttcgctcatatgagatctctccttcgttcataatacaaac

SEQ ID NO: 143 - DNA, 25 bp, synthetic construct
  misc_feature 1..25: note = Synthetic: NphB- cumate-GB- fwd
  source 1..25: mol_type = other DNA
tctcatatgagcgaagcagcagatg

SEQ ID NO: 144 - DNA, 48 bp, synthetic construct
  misc_feature 1..48: note = Synthetic: NphB- cumate-GB- rev
  source 1..48: mol_type = other DNA
agcagcggtttctttaccagactcgaggtcaatcttccaggctatcaa

SEQ ID NO: 145 - DNA, 33 bp, synthetic construct
  misc_feature 1..33: note = Synthetic: NphB-IF-fwd
  source 1..33: mol_type = other DNA
aaggagatatacataatgagcgaagcagcagat

SEQ ID NO: 146 - DNA, 35 bp, synthetic construct
  misc_feature 1..35: note = Synthetic: NphB-IF-rev
  source 1..35: mol_type = other DNA
ttaccagactcgaggtcaatcttccaggctatcaa